(19) United States
(12) Patent Application Publication
(10) Pub. No.: US 2023/0203469 A1
(43) Pub. Date: Jun. 29, 2023

(54) ADAMTS13 VARIANT, COMPOSITIONS, AND USES THEREOF

(71) Applicant: TAKEDA PHARMACEUTICAL COMPANY LIMITED, Osaka (JP)

(72) Inventors: Friedmund BACHMANN, Gross-Enzersdorf (AT); Birgit SEYFRIED, Vienna (AT); Michael GRANINGER, Vienna (AT); Bjorn MELLGARD, Cambridge, MA (US)

(73) Assignee: TAKEDA PHARMACEUTICAL COMPANY LIMITED, Osaka (JP)

(21) Appl. No.: 17/916,441

(22) PCT Filed: Apr. 2, 2021
(86) PCT No.: PCT/IB2021/000210

§ 371 Date: Sep. 30, 2022

Related U.S. Application Data
(60) Provisional application No. 63/004,389, filed on Apr. 2, 2020.

(51) Int. Cl. C12N 9/64; A61K 38/48; A61P 7/04; C12N 15/85

(52) U.S. Cl. CPC C12N 9/6489; C12Y 304/24087; A61K 38/4886; A61P 7/04; C12N 15/85

(57) ABSTRACT

This invention relates to ADAMTS13 variants and methods of administering ADAMTS13 variants to a treat a disease or condition associated with ADAMTS13 and VWF dysfunction.

### CROSS-REFERENCE TO RELATED APPLICATIONS

This application is a 371 National Stage Application of the International Application No. PCT/IB2021/000210, filed Apr. 2, 2021, which claims priority to U.S. Provisional Application No. 63/004,389, filed Apr. 2, 2020, the disclosure of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Jun. 23, 2021, is named 250478_002078_SL.txt and is 64,951 bytes in size.

### BACKGROUND OF THE INVENTION

The ADAMTS (A Disintegrin-like And Metalloprotease with Thrombospondin type I motifs) proteins are a family of metalloproteinases containing a number of conserved domains, including a zinc-dependent catalytic domain, a cysteine-rich domain, a disintegrin-like domain, and at least one, and in most cases multiple, thrombospondin type I repeats (for review, see Nicholson et al., BMC Evol Biol. 2005 Feb. 4; 5(1):11, which is incorporated herein by reference in its entirety for all purposes). These proteins, which are evolutionarily related to the ADAM and MMP families of metalloproteinases (Jones G C, Curr Pharm Biotechnol. 2006 February; 7(1):25-31, which is incorporated herein by reference in its entirety for all purposes), are secreted enzymes that have been linked to a number of diseases and conditions including thrombotic thrombocytopenic purpura (TTP) (Moake J L, Semin Hematol. 2004 January; 41(1):4-14, which is incorporated herein by reference in its entirety for all purposes), connective tissue disorders, cancers, inflammation (Nicholson et al.), and severe Plasmodium falciparum malaria (Larkin et al., PLoS Pathog. 2009 March; 5(3):e1000349, which is incorporated herein by reference in its entirety for all purposes). Because of these associations, the ADAMTS enzymes have been recognized as potential therapeutic targets for a number of pathologies (Jones G C, Curr Pharm Biotechnol. 2006 February; 7(1):25-31, which is incorporated herein by reference in its entirety for all purposes).

One ADAMTS family member, ADAMTS13, cleaves von Willebrand factor (vWF) at the Tyr⁸⁴²-Met⁸⁴³ bond in the central A2 domain of the mature vWF subunit (i.e., Tyr¹⁶⁰⁵-Met¹⁶⁰⁶ in vWF UniProt Id P04275, which is incorporated herein by reference in its entirety for all purposes) and requires zinc or calcium for activity (Dent J A, et al., Proc Natl Acad Sci USA. 1990; 87:6306-6310, which is incorporated herein by reference in its entirety for all purposes). vWF synthesized in megakaryocytes and endothelial cells is stored in platelet-granules and Weibel-Palade bodies, respectively, as ultra large vWF (UL-vWF) (Moake J L, et al., N Engl J Med. 1982; 307:1432-1435; Wagner D D, et al., J Cell Biol. 1982; 95:355-360; Wagner D D, et al., Mayo Clin Proc. 1991; 66:621-627; Sporn L A, et al., Blood. 1987; 69:1531-1534; Tsai H M, et al., Biochem Biophys Res Commun. 1989; 158:980-985; Tsai H M, et al., Blood. 1989; 73:2074-2076, each of which is incorporated herein by reference in their entirety for all purposes). Once secreted from endothelial cells, these UL-vWF multimers are cleaved by ADAMTS13 in circulation into a series of smaller multimers at specific cleavage sites within the vWF molecule (Tsai H M, et al., Biochem Biophys Res Commun. 1989; 158:980-985; Dent J A, et al., J Clin Invest. 1991; 88:774-782; Furlan M, et al., Proc Natl Acad Sci USA. 1993; 90:7503-7507, each of which is incorporated herein by reference in their entirety for all purposes).

Loss of ADAMTS13 activity or increased levels of vWF have been linked to a number of conditions, such as TTP (Moake J L, Semin Hematol. 2004 January; 41(1):4-14), acute and chronic inflammation (Chauhan et al., J Exp Med. 2008 Sep. 1; 205(9):2065-74), severe Plasmodium falciparum malaria (Larkin et al., PLoS Pathog. 2009 March; 5(3):e1000349), sickle cell disease (SCD) associated acute vaso-occlusive events, acute lung injury, cardiovascular disease (Sonneveld et al., Arterioscler Thromb Vasc Biol 2016 DOI:10.1161/ATVBAHA.116.308225), and ischemic stroke (Sonneveld et al. 2016, each publication in this paragraph is incorporated herein by reference in their entirety for all purposes).

The ADAMTS13 protease is a 190 kDa glycosylated protein produced predominantly by the liver (Levy G G, et al., Nature. 2001; 413:488-494; Fujikawa K, et al., Blood. 2001; 98:1662-1666; Zheng X, et al., J Biol Chem. 2001; 276:41059-41063; Soejima K, et al., J Biochem (Tokyo). 2001; 130:475-480; Gerritsen H E et al., Blood. 2001; 98:1654-1661, each of which is incorporated herein by reference in their entirety for all purposes). ADAMTS13 is expressed as a precursor with an N-terminal propeptide. The mature ADAMTS13 comprises a metalloprotease (M) domain, a disintegrin-like (D) domain, a thrombospondin type1 (T) repeat, a cysteine-rich (C) domain, and a spacer (S) domain, followed by seven consecutive TSP1 repeats (T2-T8) and two CUB domains as illustrated in FIG. 4. Structural information of different domains has been reported on ADAMTS family proteins, including a structure of human ADAMTS13 DTCS (residues 287-685) (Akiyama M., Takeda S., Kokame K., Takagi J., Miyata T. 2009 Crystal structures of the noncatalytic domains of ADAMTS13 reveal multiple discontinuous exosites for von Willebrand factor, Proceedings of the National Academy of Sciences 106: 19274-19279, which is incorporated herein by reference in its entirety for all purposes). Structural analyses indicate that ADAMTS family members share sequence conservation and structure similarity of MDTCS domains (Akiyama et al., 2009 supra; Mosyak L., Georgiadis K., Shane T., Svenson K. et al. 2008 Crystal structures of the two major aggrecan degrading enzymes, ADAMTS4 and ADAMTS5, Protein Science 17: 16-21, each of which is incorporated herein by reference in their entirety for all purposes). The VWF-proteolytic activity of ADAMTS13 is highly dependent on divalent cations, which has also been observed in other the metalloprotease domains in this ADAMTS family (Zheng et al., 2001 supra; Gardner M. D., Chion C. K., de Groot R., Shah A., Crawley J. T. et al. 2009 A functional calcium-binding site in the metalloprotease domain of ADAMTS13, Blood 113: 1149-1157, which is incorporated herein by reference in its entirety for all purposes). Moreover, ADAMTS13 activity undergoes allosteric regulation by binding to VWF and interactions between N-terminal MDTCS and C-terminal CUB domains (Muia J., Zhu J., Gupta G., Haberichter S. L., Friedman K. D. et al. 2014 Allosteric activation of ADAMTS13 by von Willebrand factor, Proceedings of the National Academy of Sciences 111: 18584-18589; South K., Luken B. M., Crawley J. T. B., Phillips R., Thomas M., et al. 2014 Conformational activation of ADAMTS13, Proceedings of the National Academy of Sciences 111: 18578-18583, each of which is incorporated herein by reference in their entirety for all purposes).

Described herein are variants of ADAMTS13, including the use of variants of ADAMTS13 in compositions and methods of treating a disease or condition associated with ADAMTS13 and VWF dysfunction.

### BRIEF SUMMARY OF THE INVENTION

The disclosure includes variants of ADAMTS13. The disclosure also includes pharmaceutical compositions and/or uses of ADAMTS13 variants for the preparation of medicaments. The disclosure also includes a method for treating, ameliorating, and/or preventing a disease or disorder associated with an ADAMTS13 deficiency, high vWF levels, and/or high vWF antigen levels with ADAMTS13 variants. Other related aspects are also provided in the disclosure.

In certain embodiments, the present disclosure provides variants of ADAMTS13. In certain embodiments, the ADAMTS13 variant comprises at least one amino acid substitution as compared to (i.e., relative to) an ADAMTS13 protein amino acid (e.g., SEQ ID NO: 1). In certain embodiments, the single amino acid substitution is within the catalytic domain of ADAMTS13 (e.g., amino acids 80 to 286 of SEQ ID NO: 1). In certain embodiments, the single amino acid substitution is at least one of I⁷⁹M, V⁸⁸M, H⁹⁶D, Q⁹⁷R, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13. In certain embodiments, the single amino acid substitution is not I⁷⁹M, V⁸⁸M, H⁹⁶D, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13. In certain embodiments, the ADAMTS13 variant comprises a single amino acid substitution at Q⁹⁷ as denoted in SEQ ID NO: 1, or the equivalent amino acid in an ADAMTS13. In certain embodiments, the amino acid change is from a Q to a D, E, K, H, L, N, P, or R. In certain embodiments, the amino acid change is from a Q to an R. In certain embodiments, the ADAMTS13 variant is ADAMTS13 Q⁹⁷R (SEQ ID NO: 2), or an amino acid sequence having at least 80% sequence identity thereof.

In certain embodiments, the present disclosure provides pharmaceutical compositions comprising at least one ADAMTS13 variant. In certain embodiments, the present disclosure provides pharmaceutical compositions comprising at least one ADAMTS13 variant and a pharmaceutically acceptable carrier or excipient. In certain embodiments, the pharmaceutical composition comprises a combination of at least one ADAMTS13 variant and at least one ADAMTS13 protein (e.g., wildtype). In certain embodiments, the pharmaceutical composition comprises a combination of at least one ADAMTS13 variant and at least one ADAMTS13 protein (e.g., wildtype) and a pharmaceutically acceptable carrier or excipient. In certain embodiments, the ratio of ADAMTS13 variant to wildtype ADAMTS13 (e.g., SEQ ID NO: 1) is about 4:1 to about 1:4. In certain embodiments, the ratio of ADAMTS13 variant to ADAMTS13 wildtype is about 3:1. In certain embodiments, the ratio of ADAMTS13 variant to ADAMTS13 wildtype is about 2:1. In certain embodiments, the ratio of ADAMTS13 variant to ADAMTS13 wildtype is about 1:1. In certain embodiments, the ratio of ADAMTS13 variant to ADAMTS13 wildtype is about 1:2. In certain embodiments, the ratio of ADAMTS13 variant to ADAMTS13 wildtype is about 2:3. In certain embodiments, the ratio of ADAMTS13 variant to ADAMTS13 wildtype is about 3:2. In certain embodiments, the ADAMTS13 variant comprises a single amino acid substitution at Q⁹⁷ as denoted in SEQ ID NO: 1, or the equivalent amino acid position in an ADAMTS13. In certain embodiments, the ADAMTS13 variant is ADAMTS13 Q⁹⁷R (SEQ ID NO: 2), or an amino acid sequence having at least 80% sequence identity thereof. In certain embodiments, the wildtype ADAMTS13 is human ADAMTS13 or a biologically active derivative or fragment thereof as described in U.S. Patent Application Publication No. 2011/0229455, which is incorporated herein by reference for all purposes. In one embodiment, the amino acid sequence of hADAMTS13 is that of GenBank accession number NP_620594. In certain embodiments, the hADAMTS13 is SEQ ID NO: 1.

In certain embodiments, the pharmaceutical composition comprises a combination of at least one ADAMTS13 variant and at least one ADAMTS13 protein (e.g., wildtype). In certain embodiments, the pharmaceutical composition comprises a combination of at least one ADAMTS13 variant and at least one ADAMTS13 protein (e.g., wildtype) and a pharmaceutically acceptable carrier or excipient. In certain embodiments, the ADAMTS13 variant constitutes between about 52% to about 72% or between about 47% to about 84% of total amount of all ADAMTS13 proteins and variants in the composition. In certain embodiments, the ADAMTS13 variant comprises a single amino acid substitution at Q⁹⁷ as denoted in SEQ ID NO: 1, or the equivalent amino acid position in an ADAMTS13. In certain embodiments, the ADAMTS13 variant is ADAMTS13 Q⁹⁷R (SEQ ID NO: 2), or an amino acid sequence having at least 80% sequence identity thereof. In certain embodiments, the wildtype ADAMTS13 is human ADAMTS13 or a biologically active derivative or fragment thereof as described in U.S. Patent Application Publication No. 2011/0229455, which is incorporated herein by reference for all purposes. In one embodiment, the amino acid sequence of hADAMTS13 is that of GenBank accession number NP_620594. In certain embodiments, the hADAMTS13 is SEQ ID NO: 1.

In certain embodiments, the relative abundance, percentage, and/or ratio is determined by a peptide mapping method. In certain embodiments, the relative abundance, percentage, and/or ratio is determined by a peptide mapping method as described in Example 3. In certain embodiments, the relative abundance, percentage, and/or ratio is determined by HPLC analysis of tryptic peptides separated by liquid chromatography followed by mass spectrometry analysis. In certain embodiments, the relative abundance, percentage, and/or ratio is based on intensities in extracted ion chromatograms. In certain embodiments, the relative abundance, percentage, and/or ratio is determined based on the peak area of tryptic peptides of the ADAMTS13 variant (e.g., Q⁹⁷R ADAMTS13 variant) in relation to the sum of the peak areas of all ADAMTS13 proteins and variants in the composition (e.g., sum total of Q⁹⁷R ADAMTS13 variant and Q⁹⁷ ADAMTS13 protein). In certain embodiments, the tryptic peptides of all ADAMTS13 proteins and variants in the composition being measured are specific to the at least one amino acid difference between the ADAMTS13 variant as compared to all other ADAMTS13 proteins and variants in the composition. For example, the tryptic peptide(s) that can be measured for the Q⁹⁷R ADAMTS13 variant can be AAGGILHLELLVAVGPDVFQAHR (SEQ ID NO: 115) or a combination of AAGGILHLELLVAVGPDVFQAHR (SEQ ID NO: 115) and EDTER (SEQ ID NO: 5) and the tryptic peptide measured for the Q⁹⁷ ADAMTS13 protein can be AAGGILHLELLVAVGPDVFQAHQEDTER (SEQ ID NO: 6).

In certain embodiments, the relative abundance, percentage, and/or ratio is determined based on total weight of ADAMTS13 variant in relation to the sum total weight of all ADAMTS13 proteins and variants in the composition.

In certain embodiments, the ADAMTS13 variant and/or wildtype is recombinant. In certain embodiments, the wildtype ADAMTS13 variant and/or wildtype is plasma derived.

In one aspect, the present disclosure provides a method for treating or preventing a blood clotting disorder in a subject, the method comprising administering to the subject in need thereof a therapeutically effective amount of an ADAMTS13 variant or composition thereof, including one comprising an ADAMTS13 protein.

In certain embodiments of the methods provided herein, the clotting disorder is selected from the group consisting of inherited TTP (also referred to as congenital TTP, hereditary TTP, familial TTP and Upshaw-Schulman syndrome), acquired TTP (also referred to as immune-mediated TTP), infarction, cerebral infarction, myocardial infarction, ischemic/reperfusion injury, deep vein thrombosis, and sepsis-related disseminated intravascular coagulation. In one embodiment of the methods provided herein, the clotting disorder is inherited TTP. In one embodiment of the methods provided herein, the clotting disorder is acquired TTP.

In one aspect, the present disclosure provides a method for treating a bleeding episode in a subject, the method comprising administering to the subject in need thereof a therapeutically effective amount of an ADAMTS13 variant or composition thereof, including one comprising an ADAMTS13 protein.

In certain embodiments, the bleeding episode is associated with inherited TTP, acquired TTP, infarction, myocardial infarction, cerebral infarction, and/or ischemia reperfusion injury.

In one aspect, the present disclosure provides a method for treating or preventing a vaso-occlusive crisis in a subject suffering from sickle cell disease, the method comprising administering to the subject in need thereof a therapeutically effective amount of an ADAMTS13 variant or composition thereof, including a composition comprising an ADAMTS13 protein.

In one aspect, the present disclosure provides a method for treating or preventing a vaso-occlusive crisis in a subject suffering from acute lung injury (ALI) and/or acute respiratory distress syndrome (ARDS), the method comprising administering to the subject in need thereof a therapeutically effective amount of an ADAMTS13 variant or composition thereof, including a composition comprising an ADAMTS13 protein.

In one aspect, the present disclosure provides a method for recanalization of an occluded blood vessel in a subject having a cerebral infarction, the method comprising administering to the subject in need thereof a therapeutically effective amount of an ADAMTS13 variant or composition thereof, including a composition comprising an ADAMTS13 protein.

In one aspect, the present disclosure provides a method of improving the recovery of sensorimotor function in a subject that has experienced a cerebral infarction, the method comprising administering to the subject in need thereof a therapeutically effective amount of an ADAMTS13 variant or composition thereof, including a composition comprising an ADAMTS13 protein.

In one aspect, the present disclosure provides a method for treating or preventing a blood clotting disorder associated with cardiovascular disease in a subject, the method comprising administering to the subject in need thereof a therapeutically effective amount of an ADAMTS13 variant or composition thereof, including a composition comprising an ADAMTS13 protein.

In one aspect, the present disclosure provides a method for treating or preventing hematologic disease in a subject, the method comprising administering to the subject in need thereof a therapeutically effective amount of an ADAMTS13 variant or composition thereof, including a composition comprising an ADAMTS13 protein.

In certain embodiments of the methods provided herein, the ADAMTS13 variant or composition, including a composition comprising ADAMTS13, is administered in a single bolus injection, monthly, every two weeks, weekly, twice a week, daily, every 12 hours, every 8 hours, every six hours, every four hours, every two hours, or every hour.

In certain embodiments, the ADAMTS13 variant or composition, including a composition comprising ADAMTS13, is administered intravenously or subcutaneously.

In certain embodiments, the ADAMTS13 variant(s) and/or ADAMTS13 protein (e.g., wildtype) is recombinant. In certain embodiments, the ADAMTS13 variant(s) and/or ADAMTS13 protein is recombinantly produced by HEK293 cells. In certain embodiments, the ADAMTS13 variant(s) and/or ADAMTS13 protein is recombinantly produced by CHO cells. In certain embodiments, the ADAMTS13 variant(s) and/or ADAMTS13 protein is plasma derived.

In certain embodiments, the subject is a mammal. In certain embodiments, the subject is a human.

In certain embodiments, the composition is a stable aqueous solution ready for administration. In certain embodiments, the composition is lyophilized. In certain embodiments, the composition is reconstituted with a pharmaceutically acceptable vehicle suitable for injection prior to administration.

In one aspect, the present disclosure provides a nucleic acid molecule encoding an ADAMTS13 variant as disclosed herein (e.g., SEQ ID NO: 4).

In one aspect, the present disclosure provides a vector comprising a nucleic acid molecule encoding an ADAMTS13 variant as disclosed herein (e.g., SEQ ID NO: 4). In certain embodiments, the vector is an expression vector wherein the polynucleotide sequence encoding the ADAMTS13 variant is operably linked to a promoter that is capable of mediating expression of the ADAMTS13 variant in a host cell.

In one aspect, the present disclosure provides a host cell comprising a nucleic acid molecule encoding an ADAMTS13 variant as disclosed herein (e.g., SEQ ID NO: 4) or a vector as disclosed herein.

In one aspect, the present disclosure provides a host cell line comprising cells modified to express the ADAMTS13 variant as disclosed herein (e.g., SEQ ID NO: 2) and at least one ADAMTS13 protein (e.g., SEQ ID NO: 1). In certain embodiments, the ADAMTS13 variant and the ADAMTS13 protein are expressed in different cells in the host cell line. the ADAMTS13 variant and the ADAMTS13 protein are expressed in the same cell in the host cell line.

In certain embodiments, the host cell or host cell line is a CHO, COS, HEK 293, BHK, SK-Hep, or HepG2 cell or cell line. In certain embodiments, the CHO cell is a CHO DBX-11 or CHOZN cell line. In certain embodiments, the CHOZN cell is a CHO DBX-11 cell line. In certain embodiments, the CHOZN cell is a CHOZN glutamine synthetase (GS) cell line.

### DESCRIPTION OF THE DRAWINGS

The priority application file contains at least one drawing executed in color, which is accessible via United States Patent and Trademark Office's Patent Examination Research Dataset (PAIR).

FIG. 1A-1C shows the alignment between wildtype ADAMTS13 (SEQ ID NO: 1) and ADAMTS13 Q⁹⁷R variant (SEQ ID NO: 2).

FIG. 2A-2C shows the alignment between wildtype ADAMTS13 (SEQ ID NO: 1) and wildtype gorilla ADAMTS13 (SEQ ID NO: 3).

FIG. 3 is a boxplot of the specific activity (U/mg Ag) of the plasma derived ADAMTS13 (pdADAMTS13) and a combination of wildtype and Q⁹⁷R variant recombinant ADAMTS13 (rADAMTS13). The pdADAMTS data examines at least 80 samples from different donors and the rADAMTS13 combination examines 35 different batches of rADAMTS13, wherein the abundance of variant is 52 to 72%.

FIG. 4 shows a three-dimensional model of ADAMTS13, including the location of Q⁹⁷, which is exposed on the M proteas domain, and located in a short two residue turn in a stable helix-turn-helix structure.

FIG. 5 is a UV chromatogram of a composition comprising a combination of wildtype ADAMTS13 and the Q⁹⁷R variant. The insert shows that the tryptic peptides representing the two variants are well separated.

FIG. 6 is a total ion chromatogram and extracted ion chromatograms of tryptic peptides from native variant and Q⁹⁷R variant in a composition. The tryptic peptide of the native variant eluted before the correctly cleaved tryptic peptide of the Q⁹⁷R variant. The miss-cleaved peptide of the Q⁹⁷R variant eluted before the two other peptides. The area of the peaks represents the relative abundance of the peptides, and the variants, respectively. FIG. 6 discloses SEQ ID NOS 115, 6, and 7, respectively, in order of appearance.

FIG. 7 is a scatter plot of the specific activity (U/mg Ag) of rADAMTS13 compositions comprising different ratios of Q⁹⁷R rADAMTS13 variant to wildtype rADAMTS13. A total of 35 different rADAMTS13 compositions were tested.

FIG. 8 is a scatter plot of the VWF cleavage of rADAMTS13 compositions comprising different ratios of Q⁹⁷R rADAMTS13 variant to wildtype rADAMTS13. A total of 35 different rADAMTS13 compositions were tested.

FIG. 9 is a flamingo fluorescent stained CDC gel of Q⁹⁷R rADAMTS13 and the Q⁹⁷R rADAMTS13 variant. The Flamingo fluorescence staining represents the total protein amount. The gel image shows the following band pattern for both ADAMTS13 samples: one major band at approximately 190 kDa, which represents a full-length molecule; a weaker band at approximately 150 kDa, which indicates a truncated form; and two even weaker bands between 150 and 75 kDa, which indicates that the proteins was partially degraded. The band pattern, however, was similar in presence and intensity for both samples indicating comparable total protein composition. 1: control sample; 2: Q⁹⁷ rADAMTS13; and 3: Q⁹⁷R rADAMTS13 variant.

FIG. 10 is a western blot analysis of Q⁹⁷R rADAMTS13 and the Q⁹⁷R rADAMTS13 variant. For the Western blot analysis, an anti-ADAMTS13 antibody was used to visualize the ADAMTS13 protein forms. The image of the membrane shows a comparable band pattern for both samples: full-length protein was present as main signal at approximately 190 kDa; the truncated form was present at approximately 150 kDa as weaker band, and a very weak band was visible at approximately 125 kDa. 1: control sample; 2: Q⁹⁷ rADAMTS13; and 3: Q⁹⁷R rADAMTS13 variant.

FIG. 11 is an overlay of chromatograms of wildtype ADAMTS13 and Q⁹⁷R rADAMTS13 variant produced in CHOZN GS^{-/-} cell line.

FIG. 12 is an enlarged version of FIG. 11 presenting an overlay of chromatograms of wildtype ADAMTS13 and Q⁹⁷R rADAMTS13 variant produced in CHOZN GS^{-/-} cell line.

FIG. 13 is an overlay of chromatograms of wildtype ADAMTS13 and Q⁹⁷R rADAMTS13 variant produced in CHO DBX-11 cell line.

FIG. 14A-14B shows the amino acid sequence for ADAMTS13 (SEQ ID NO: 4).

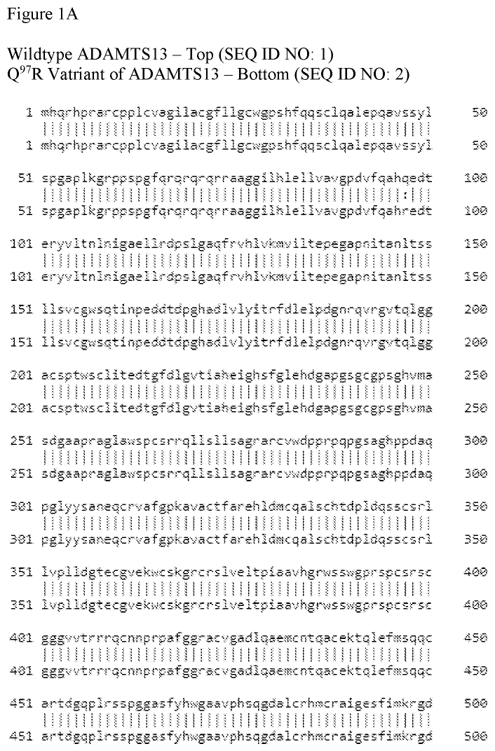

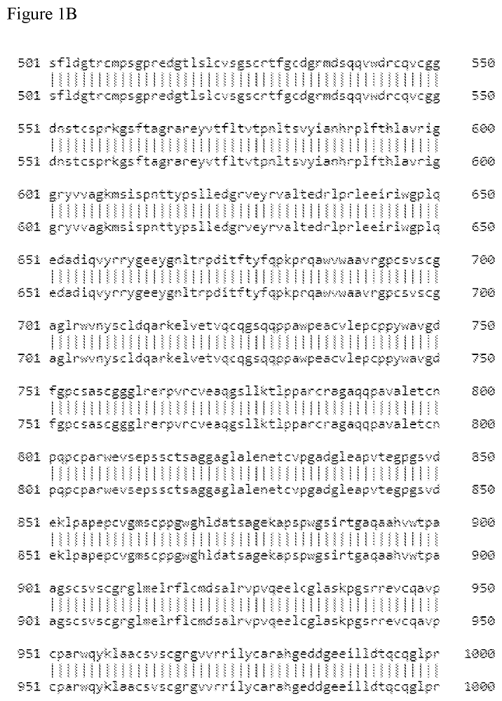

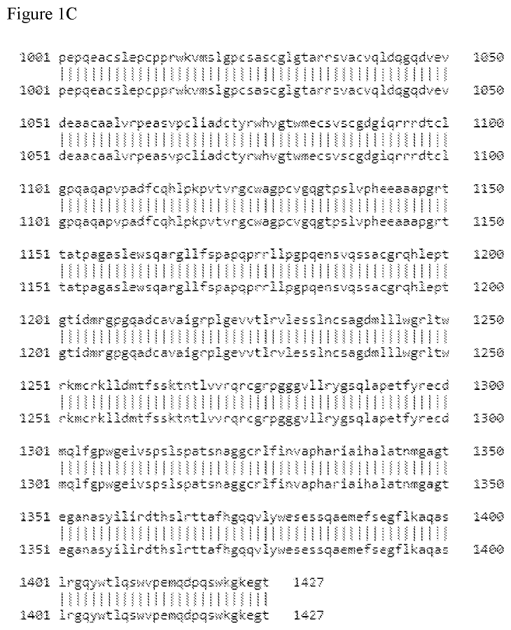

### DETAILED DESCRIPTION OF THE INVENTION

### I. Introduction

Ischemic events, such as heart attack and stroke, are a leading cause of death and disability around the world. Thrombolytic therapy with tissue plasminogen activator (tPA), which leads to fibrin degradation and promotes clot lysis, can be used to treat ischemia, but tPA use is restricted to the first few hours after the ischemic event. In addition, tPA can increase incidence and severity of hemorrhage and edema formation. Thus, there remains a clear need to identify new therapeutic agents for minimizing the effects of ischemia. In addition to its effect on coagulation, such agents can also target platelet adhesion and the inflammatory process that follows ischemic events.

von Willebrand Factor (VWF) is a large multimeric glycoprotein that is present in blood plasma and plays a major role in blood coagulation. VWF is stored in an ultra large form (UL-VWF, >20 million Da) in platelet α-granules and Weibel-Palade bodies of endothelial cells from which it is released during injury or inflammation. If not immediately consumed for platelet adhesion, the UL-VWF is cleaved by ADAMTS 13 to smaller less adhesive multimers that circulate in plasma. Ischemia, such as occurs after thrombolysis, is a potent inducer of Weibel-Palade body secretion, thus making the infarct area highly thrombogenic.

The basic VWF monomer is a 2050-amino acid protein that includes a number of specific domains with a specific function: (1) the D7D3 domain, which binds to Factor VIII; (2) the A1 domain, which binds to platelet GPlb-receptor, heparin, and possibly collagen; (3) the A3 domain, which binds to collagen; (4) the C1 domain, in which the R-G-D motif binds to platelet integrin αIIbβ3 when this is activated; and (5) the “cysteine knot” domain located at the C-terminus, which VWF shares with platelet-derived growth factor (PDGF), transforming growth factor-β (TGFβ), and β-human chorionic gonadotropin (βHCG). Multimers of VWF can be extremely large, consisting of over 80 monomers with molecular weight exceeding 20,000 kDa. These large VWF multimers are most biologically functional, capable of mediating the adhesion of platelets to sites of vascular injury, as well as binding and stabilizing the procoagulant protein Factor VIII. Deficiency in VWF or altered VWF is known to cause various bleeding disorders.

The biological breakdown of VWF is largely mediated by a protein termed ADAMTS13 (A Disintegrin-like And Metalloprotease with Thrombospondin type I motif No. 13), a 190 kDa glycosylated protein produced predominantly by the liver. ADAMTS 13 is a plasma metalloprotease that cleaves VWF between tyrosine at position 1605 and methionine at position 1606, breaking down the VWF multimers into smaller units, which are further degraded by other peptidases. VWF is also shown to play a role in infarction, a process in which tissue undergoes necrosis due to insufficient blood supply. For example, when VWF level is suppressed, infarct volume is reduced; whereas increased level of VWF leads to larger infarct volume.

Low levels of ADAMTS13 is associated with clotting disorders such as inherited thrombotic thrombocytopenic purpura (TTP) (also referred to as congenital TTP, hereditary TTP, familial TTP and Upshaw-Schulman syndrome), acquired TTP (also referred to as immune-mediated TTP), cerebral infarction, myocardial infarction, ischemic/reperfusion injury, deep vein thrombosis, and disseminated intravascular coagulation (DIC) such as sepsis-related DIC. ADAMTS13 deficiency is also associated with bleeding episodes, such as those associated with inherited TTP, acquired TTP, cerebral infarction, myocardial infarction, ischemic/reperfusion injury, deep vein thrombosis, and/or sepsis-related disseminated intravascular coagulation, as well as vaso-occlusive crises in a subject suffering from sickle cell disease and lung injury in a subject suffering from or at risk of suffering from acute lung injury (ALI) and/or acute respiratory distress syndrome (ARDS). ADAMTS13 supplementation also has the potential to improve the recovery after coronary artery occlusion (myocardial infarction) and of sensorimotor function in a subject that has experienced a cerebral infarction through recanalization of blood vessels.

The present invention is based in part on the discovery of that ADAMTS13 variants, such as ADAMTS13 Q⁹⁷R (SEQ ID NO: 2) are useful for the treatment of the same disease and disorders as ADAMTS13 (e.g., SEQ ID NO: 1). In certain embodiments, the ADAMTS13 variant(s) can be used in combination with other ADAMTS13 proteins (e.g., wildtype) in the methods described herein.

SEQ ID NO: 1:
MHQRHPRARCPPLCVAGILACGFLLGCWGPSHFQQSCLQALEPQAVSSY
LSPGAPLKGRPPSPGFQRQRQRQRRAAGGILHLELLVAVGPDVFQAHQE
DTERYVLTNLNIGAELLRDPSLGAQFRVHLVKMVILTEPEGAPNITANL
TSSLLSVCGWSQTINPEDDTDPGHADLVLYITRFDLELPDGNRQVRGVT
QLGGACSPTWSCLITEDTGFDLGVTIAHEIGHSFGLEHDGAPGSGCGPS
GHVMASDGAAPRAGLAWSPCSRRQLLSLLSAGRARCVWDPPRPQPGSAG
HPPDAQPGLYYSANEQCRVAFGPKAVACTFAREHLDMCQALSCHTDPLD
QSSCSRLLVPLLDGTECGVEKWCSKGRCRSLVELTPIAAVHGRWSSWGP
RSPCSRSCGGGVVTRRRQCNNPRPAFGGRACVGADLQAEMCNTQACEKT
QLEFMSQQCARTDGQPLRSSPGGASFYHWGAAVPHSQGDALCRHMCRAI
GESFIMKRGDSFLDGTRCMPSGPREDGTLSLCVSGSCRTFGCDGRMDSQ
QVWDRCQVCGGDNSTCSPRKGSFTAGRAREYVTFLTVTPNLTSVYIANH
RPLFTHLAVRIGGRYVVAGKMSISPNTTYPSLLEDGRVEYRVALTEDRL
PRLEEIRIWGPLQEDADIQVYRRYGEEYGNLTRPDITFTYFQPKPRQAW
VWAAVRGPCSVSCGAGLRWVNYSCLDQARKELVETVQCQGSQQPPAWPE
ACVLEPCPPYWAVGDFGPCSASCGGGLRERPVRCVEAQGSLLKTLPPAR
CRAGAQQPAVALETCNPQPCPARWEVSEPSSCTSAGGAGLALENETCVP
GADGLEAPVTEGPGSVDEKLPAPEPCVGMSCPPGWGHLDATSAGEKAPS
PWGSIRTGAQAAHVWTPAAGSCSVSCGRGLMELRFLCMDSALRVPVQEE
LCGLASKPGSRREVCQAVPCPARWQYKLAACSVSCGRGVVRRILYCARA
HGEDDGEEILLDTQCQGLPRPEPQEACSLEPCPPRWKVMSLGPCSASCG
LGTARRSVACVQLDQGQDVEVDEAACAALVRPEASVPCLIADCTYRWHV
GTWMECSVSCGDGIQRRRDTCLGPQAQAPVPADFCQHLPKPVTVRGCWA
GPCVGQGTPSLVPHEEAAAPGRTTATPAGASLEWSQARGLLFSPAPQPR
RLLPGPQENSVQSSACGRQHLEPTGTIDMRGPGQADCAVAIGRPLGEVV
TLRVLESSLNCSAGDMLLLWGRLTWRKMCRKLLDMTFSSKTNTLVVRQR
CGRPGGGVLLRYGSQLAPETFYRECDMQLFGPWGEIVSPSLSPATSNAG
GCRLFINVAPHARIAIHALATNMGAGTEGANASYILIRDTHSLRTTAFH
GQQVLYWESESSQAEMEFSEGFLKAQASLRGQYWTLQSWVPEMQDPQSW
KGKEGT
SEQ ID NO: 1 includes the signal peptide
(bolded and underlined) and the propeptide
(bolded). The R97 protein variant carries an
amino acid exchange from “Q” to “R” at position
97 (bolded and italicized).

### II. Definitions

As used herein, “ADAMTS13” or “A13” refer to a metalloprotease of the ADAMTS (a disintegrin and metalloproteinase with thrombospondin type I motifs) family that cleaves von Willebrand factor (vWF) between residues Tyr 1605 and Met 1606. In the context of the present invention, an “ADAMTS13” embraces any ADAMTS13 protein, for example, ADAMTS13 from a mammal such as a primate, human (NP620594), monkey, rabbit, pig, bovine (XP610784), rodent, mouse (NP001001322), rat (XP342396), hamster, gerbil, canine, feline, frog (NP001083331), chicken (XP415435), and fragments thereof. As used herein, “ADAMTS13 proteins” refer to recombinantly produced and plasma derived ADAMTS13 proteins. In certain embodiments, the ADAMTS13 protein is wildtype human ADAMTS13 (hADAMTS13) or fragment thereof as described in U.S. Patent Application Publication No. 2012/0229455, which is incorporated herein by reference for all purposes. In certain embodiments, the amino acid sequence of hADAMTS13 is that of GenBank accession number NP_620594. In certain embodiments, the amino acid sequence of hADAMTS13 is SEQ ID NO: 1.

The term “ADAMTS13 variant” refers to a polypeptide substantially similar in structure and having the same biological activity, albeit in certain instances to a differing degree, to a wildtype molecule (e.g., SEQ ID NO: 1). Variants differ in the composition of their amino acid sequences compared to the wildtype polypeptide from which the variant is derived, based on one or more mutations involving (i) deletion of one or more amino acid residues at one or more termini of the polypeptide (including fragments as described above) and/or one or more internal regions of the wildtype polypeptide sequence, (ii) insertion or addition of one or more amino acids at one or more termini (typically an “addition” variant) of the polypeptide and/or one or more internal regions (typically an “insertion” variant) of the wildtype polypeptide sequence or (iii) substitution of one or more amino acids for other amino acids in the wildtype polypeptide sequence. Substitutions are conservative or non-conservative based on the physico-chemical or functional relatedness of the amino acid that is being replaced and the amino acid replacing it. A variant includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987, incorporated herein by reference for all purposes).

Human ADAMTS13 proteins include, without limitation, polypeptides comprising the amino acid sequence of GenBank accession number NP_620594 or a processed polypeptide thereof, for example a polypeptide in which the signal peptide (amino acids 1 to 29) and/or propeptide (amino acids 30-74) have been removed. Many natural variants of human ADAMTS13 are known in the art, and are embraced by the compositions of the present invention, some of which include mutations selected from R⁷W, V⁸⁸M, H⁹⁶D, R¹⁰²C, R¹⁹³w, T¹⁹⁶I, H²³⁴Q, A²⁵⁰V, R²⁶⁸P, W³⁹⁰C, R³⁹⁸H, Q⁴⁴⁸E, Q⁴⁵⁶H, P⁴⁵⁷L, P⁴⁷⁵S, C⁵⁰⁸Y, R⁵²⁸G, P⁶¹⁸A, R⁶²⁵H, I⁶⁷³F, R⁶⁹²C, A⁷³²V, E⁷⁴⁰K, A⁹⁰⁰V, S⁹⁰³L, C⁹⁰⁸Y, C⁹⁵¹G, G⁹⁸²R, C¹⁰²⁴G, A¹⁰³³T, R¹⁰⁹⁵W, R¹⁰⁹⁵W, R¹¹²³C, C¹²¹³Y, T¹²²⁶1, G¹²³⁹V, and R¹³³⁶W. Preferably, amino acids essential to the enzymatic activity of ADAMTS13 will not be mutated. These include, for example, residues known or presumed to be essential for metal binding such as residues 83, 173, 224, 228, 234, 281, and 284, and residues found in the active site of the enzyme, e.g., residue 225. Similarly, in the context of the present invention, ADAMTS13 proteins include alternate isoforms, for example, isoforms lacking amino acids 275 to 305 and/or 1135 to 1190 of the full-length human protein.

“Conservatively modified variants” applies to both amino acid and nucleic acid sequences. With respect to particular nucleic acid sequences, conservatively modified nucleic acids refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variants. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

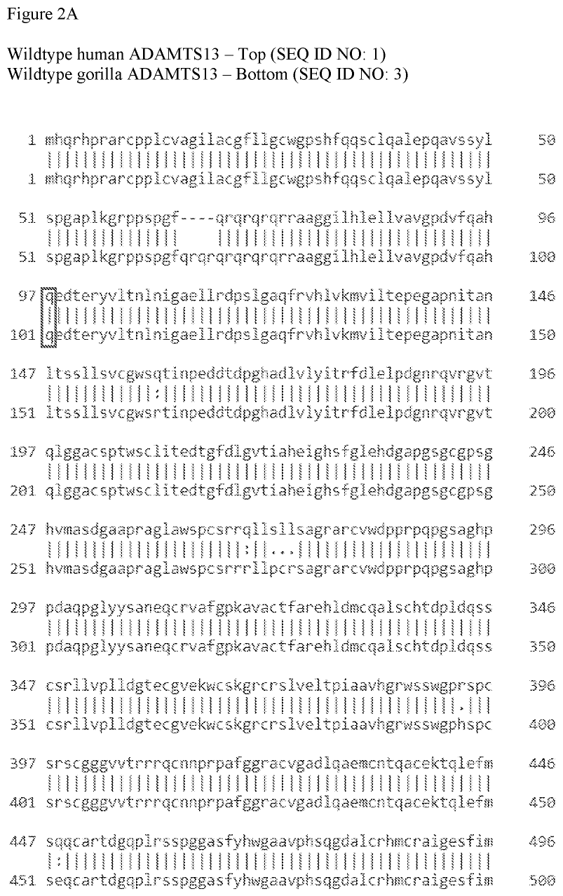

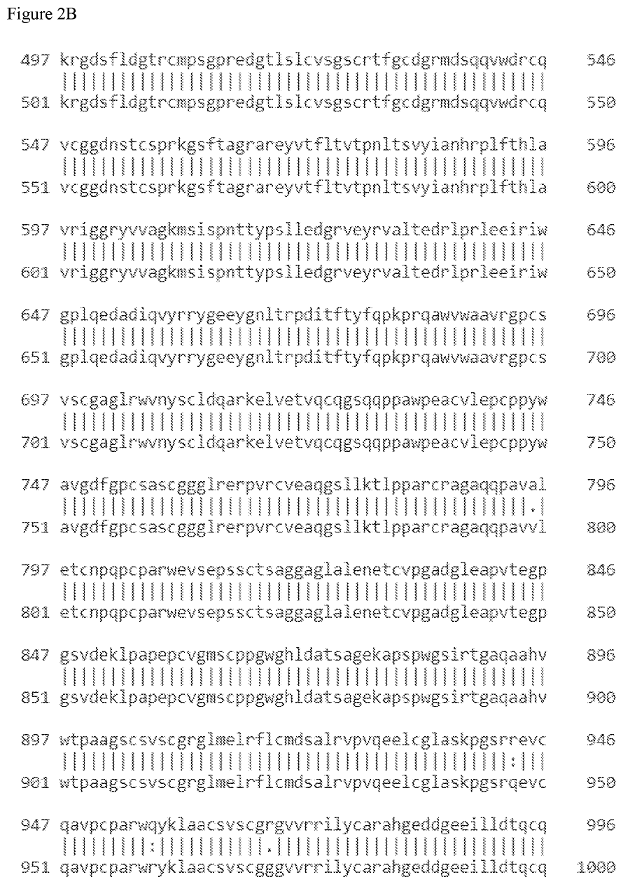

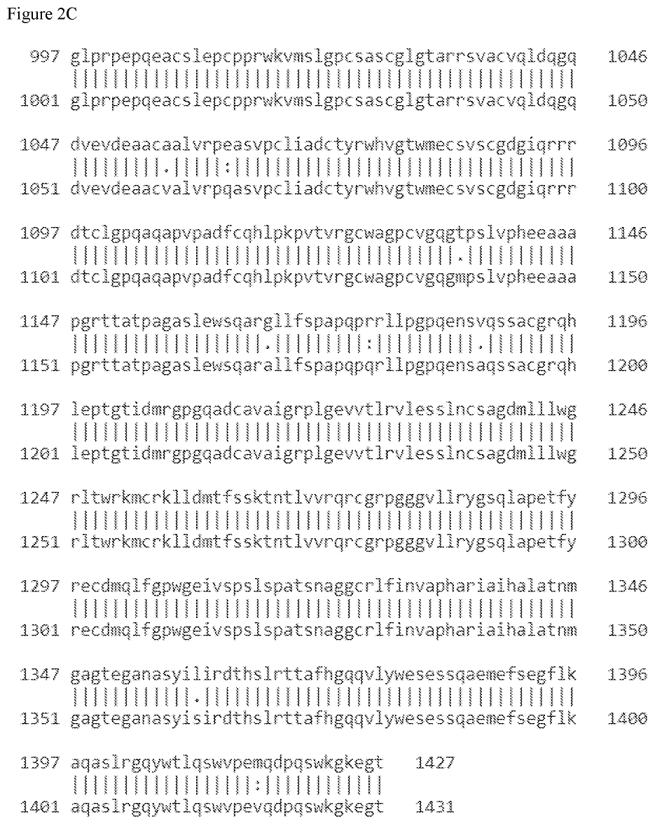

As used herein, an “equivalent position” (for example, an “equivalent amino acid position” or “equivalent residue position”) is defined herein as a position (such as, an amino acid position or a residue position) of an amino acid sequence which aligns with a corresponding position of a reference amino acid sequence (e.g., SEQ ID NO: 1), using an alignment algorithm (e.g., Clustal Needleman-Wunsch algorithm, Vector NTI). The equivalent amino acid position of the amino acid sequence need not have the same numerical position number as the corresponding position of the reference amino acid sequence. As an example, FIG. 2 shows the sequence of a human wildtype ADAMTS13 (SEQ ID NO: 1) aligned with a gorilla wildtype ADAMTS13 (SEQ ID NO: 3). In this example, amino acid position number 97 of SEQ ID NO:1 is considered to be an equivalent amino acid position to (i.e. is “equivalent to”) that of amino acid position number 101 of SEQ ID NO: 3, as amino acid number 97 of SEQ ID NO: 1 aligns with amino acid number 101 of SEQ ID NO: 3. In other words, amino acid position 97 of SEQ ID NO: 1 corresponds to amino acid position 101 of SEQ ID NO: 3.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example at least 80% identity, preferably 85%, 90%, 91%, 92%, 93, 94%, 95%, 96%, 97%, 98%, 99%, or 100% identity, to a reference sequence, e.g., SEQ ID NO:1), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

As used herein, phrases “total amount of ADAMTS13” or “total ADAMTS13” in a composition includes the sum total of all ADAMTS13 variant(s) and/or ADAMTS13 proteins (e.g., wildtype SEQ ID NO: 1) in the composition. For example, if a composition comprises Q⁹⁷ ADAMTS13 and Q⁹⁷R ADAMTS13, the “total amount of ADAMTS13” or “total ADAMTS13” would be the sum total of Q⁹⁷ ADAMTS13 and Q⁹⁷R ADAMTS13 in the composition. Likewise, if a composition comprises only Q⁹⁷R ADAMTS13, the total amount of ADAMTS13 or total ADAMTS13 would be the sum total of Q⁹⁷R ADAMTS13 in the composition.

As to amino acid sequences, one of skill will recognize that individual substitutions, insertions, deletions, additions, or truncations to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

As used herein, a “fragment” of a polypeptide refers to any portion of the polypeptide smaller than the full-length polypeptide or protein expression product. Fragments are typically deletion analogs of the full-length polypeptide, wherein one or more amino acid residues have been removed from the amino terminus and/or the carboxy terminus of the full-length polypeptide. Accordingly, “fragments” are a subset of deletion analogs described below.

The term “recombinant” or “recombinant expression system” when used with reference, e.g., to a cell, indicates that the cell has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

ADAMTS13 proteins and variants may be further modified, for example, by post-translational modifications (e.g., glycosylation at one or more amino acids selected from human residues 142, 146, 552, 579, 614, 667, 707, 828, 1235, 1354, or any other natural or engineered modification site) or by ex vivo chemical or enzymatic modification, including without limitation, glycosylation, modification by water soluble polymer (e.g., PEGylation, sialylation, HESylation, etc.), tagging, and the like. For example, the ADAMTS13 protein or variant may comprise tags that facilitate purification, detection, or both. The ADAMTS13 proteins described herein may further be modified with a therapeutic moiety or a moiety suitable imaging in vitro or in vivo.

As used herein, the term “glycosylated” or “glycosylated forms of ADAMTS13” refers to ADAMTS13 proteins that are post-translationally modified by the addition of carbohydrate or glycan residues. An ADAMTS13 protein having more than one glycosylation site can have the same glycan residue attached to each glycosylation site, or can have different glycan residues attached to different glycosylation sites. In this manner, different patterns of glycan attachment can yield different glycoforms of an ADAMTS13 protein. The predominant sugars found on a glycosylated ADAMTS13 are glucose (Glc), galactose (Gal), mannose (Man), fucose (Fuc), N-acetylgalactosamine (GalNAc), N-acetylglucosamine (GlcNAc), and sialic acid (e.g., N-acetyl-neuraminic acid (NeuAc or NANA)). Hexose (Hex) and HexNAc are generic terms that represent classes of monosaccharides such as Man, Glc, and Gal residues, and GlcNAc and GalNAc residues, respectively.

The term “glycosylation” includes the formation of ADAMTS13 glycoproteins where glycan residues are attached either to the side chain of an asparagine (Asn) residue (i.e., N-linked), or a serine (Ser) or threonine (Thr) residue (i.e., O-linked), or a tryptophan (Trp) residue (i.e., C-linked and/or C-mannosylation) of a protein.

The term “N-glycosylation site” refers to any amino acid sequence that includes an amino acid residue having a nitrogen atom, e.g., the amide nitrogen of an asparagine residue. The N-glycans attached to glycoproteins differ with respect to the number of branches (antennae) comprising peripheral sugars (e.g., GlcNAc, Gal, Fuc, and NeuAc) that are added to a common core pentasaccharide: Man₃GlcNAc₂, that contains a “trimannose” (Mani) component and “chitobiose” (GlcNAc₂) component. N-glycans are commonly classified according to their branched constituents (e.g., high mannose, hybrid or complex). A “high-mannose” type N-glycan contains unsubstituted terminal mannose sugars. These glycans typically contain between five and nine mannose residues attached to the chitobiose core. “Hybrid” type N-glycans can contain both unsubstituted terminal mannose residues and substituted mannose residues with a GlcNAc linkage. A$“complex” type N-glycan typically has at least one GlcNAc attached to an α1,3 mannose arm and at least one GlcNAc attached to an α1,6 mannose arm of the trimannose core. Complex N-glycans may also have Gal or GalNAc sugar residues that are optionally modified with NeuAc residues. Complex N-glycans may also have intrachain substitutions comprising “bisecting” GlcNAc and core Fuc residues. Complex N-glycans may also have multiple antennae on the trimannose core, often referred to as “multiple antennary glycans.”

“O-linked glycosylation” refers to a form of glycosylation where a carbohydrate residue (e.g., GalNAc, Gal) is added to a hydroxyl amino acid, e.g., serine or threonine. O-linked glycans commonly comprise an O-fucosylation bearing the disaccharide Fuc-Glc or mucin-type structures containing HexNAc-Hex-NeuAc₀₋₂. The term “O-glycosylation site” refers to any amino acid sequence that includes an amino acid residue having a hydroxyl group (e.g., serine, threonine or tyrosine side chains).

“C-linked glycosylation” refers to a form of glycosylation where a carbohydrate residue (e.g., Man) is added to a carbon on a tryptophan side chain. The term “C-glycosylation site” or “C-mannosylation site” refers to any amino acid sequence that includes an amino acid residue having a carbon atom, e.g., a carbon atom on a tryptophan side chain.

As used herein, the term “glycosimilarity index” or “glycan index” or “N-glycan index” refers to the conformity degree of a reference glycosylation profile compared to a given target profile.

As used herein, “blood clotting disorder” is defined as a disorder that includes dysfunctional platelet recruitment as well as dysfunctional neutrophil recruitment. Non-limiting examples of “blood clotting disorders” include inherited thrombotic thrombocytopenic purpura (TTP), acquired TTP, cerebral infarction, myocardial infarction, ischemic/reperfusion injury, deep vein thrombosis, and disseminated intravascular coagulation (DIC) such as sepsis-related DIC.

As used herein, “ADAMTS13 activity” includes the cleavage of full-length VWF, VWF fragments, or a VWF substrate (e.g., FRETS-VWF73 substrate (Kokame et al., Br J Haematol. 2005 April; 129(1):93-100)). “ADAMTS13 activity” may refer to the activity of the ADAMTS13 variant, ADAMTS13 protein (e.g., wildtype), or combinations thereof. In certain embodiments, when the composition is a mixture of ADAMTS13 variant(s) and/or a ADAMTS13 (e.g., wildtype), “ADAMTS13 activity” refers to the activity of total ADAMTS13 in the composition.

As used herein, “one unit of ADAMTS13 activity” is defined as the amount of activity in 1 ml of pooled normal human plasma, regardless of the assay being used. For example, one unit of ADAMTS13 FRETS-VWF73 activity is the amount of activity needed to cleave the same amount of FRETS-VWF73 substrate (Kokame et al., Br J Haematol. 2005 April; 129(1):93-100, incorporated herein by reference in its entirety for all purposes) as is cleaved by one ml of pooled normal human plasma. Additional activity assays can also be used to determine the activity of one unit of ADAMTS13. For example, direct ADAMTS13 activity assays can be performed to detect the cleavage of either full-length VWF molecules or VWF fragments using SDS agarose gel electrophoresis and indirect detection of ADAMTS13 activity can be detected with collagen binding assays. The term “one unit of ADAMTS13 activity” can be used interchangeably with “Activity unit”, “U”, “international unit”, “IU”, or “UFV73”. In certain embodiments, the international unit is based on the use of a WHO standard that was calibrated against plasma using the VWF FRETS assay (i.e., “UFV73” or “IU”).

As used herein, the term “thrombus” refers to a blood clot, especially a platelet-comprising blood clot, a microthrombus, and/or an embolus. Said thrombus may be attached to an arterial or venous blood vessel or not, and may partially or completely occlude or block the blood flow in an arterial or venous blood vessel.

The term “sickle cell disease (SCD),” as used herein, describes a group of inherited red blood cell disorders that exists in multiple forms. Some forms of SCD are Hemoglobin SS, Hemoglobin SC, Hemoglobin Sβ⁰ thalassemia, Hemoglobin Sβ⁺ thalassemia, Hemoglobin SD, and Hemoglobin SE. Although Hemoglobin SC disease and hemoglobin Sβ thalassemia are two common forms of SCD, the disclosure relates to and includes all forms of SCD.

The term “vaso-occlusive crisis (VOC),” as used herein, is an attack of sudden severe pain, which can occur without warning. VOC, also known as pain crisis or sickle cell crisis, is a common painful complication of SCD in adolescents and adults. VOC is initiated and sustained by interactions among sickle cells, endothelial cells and plasma constituents. Vaso-occlusion is responsible for a wide variety of clinical complications of SCD, including pain syndromes, stroke, leg ulcers, spontaneous abortion, and/or renal insufficiency.

The terms “acute lung injury” (ALI) and “acute respiratory distress syndrome” (ARDS) describe clinical syndromes of acute respiratory failure with substantial morbidity and mortality (Johnson et al., J. Aerosol Med. Pulmon. Drug Deliv. 23:243-52, 2010). Both ALI and the more severe ARDS represent a spectrum of lung disease characterized by the sudden onset of inflammatory pulmonary edema secondary to myriad local or systemic insults, including bilateral, inflammatory pulmonary infiltrates and impaired oxygenation or hypoxemia (Walkey et al., Clinical Epidemiology 4:159-69, 2012). Although ALI and ARDS are two clinical syndromes of lung injury or disease, the disclosure relates to and includes the use of ADAMTS13 variant(s) and/or ADAMTS13 protein in treating, preventing, or ameliorating, not only ALI and ARDS, but all forms of lung injury and lung disease, especially lung disease associated with impaired oxygenation.

The term “recanalization” refers to the restoration of the lumen of a blood vessel following an occlusion by restoration of lumen or by the formation of one or more new channels. The term “recanalizing” means restoring of the lumen of a blood vessel following an occlusion by restoration of lumen or by the formation of one or more new channels. In certain embodiments described herein, recanalization is related to an occluded blood vessel associated with an infarction (e.g., a cerebral infarction). Recanalization can be determined using any suitable method known in the art. In some embodiments where the recanalization is of an occluded cerebral blood vessel, recanalization is determined by the restoration of regional cerebral blood flow (rCBF).

“Regional cerebral blood flow” and “rCBF” refer to the amount of blood flow to a specific region of the brain in a given time. Regional cerebral blood flow can be measure using any suitable technique known in the art including, for example, using laser Doppler flow monitoring techniques described herein.

The term “bleeding episodes” refers to internal bleeding in the associated with an ADAMTS13 deficiency. Increased clotting activity, such as those occurring in small vessels and other locations, over consumes available platelets and clotting factors, thereby increasing the chance of serious internal and external bleeding by depleting the available source of platelets and clotting factors. This bleeding can be seen in capillaries and other microvasculature, and can lead to organ damage and/or ischemia.

The term “reduces the severity,” when referring to a symptom means that the symptom has delayed onset, reduced severity, reduced frequency, or causes less damage to the subject. Generally, severity of a symptom is compared to a control, e.g., a subject that does not receive an active prophylactic or therapeutic composition, or as compared to the severity of the symptom prior to administration of the therapeutic. For example, a composition can be said to reduce the severity of a symptom of any of the indications listed herein, if the symptom is reduced by about 10%, about 15%, about 20%, about 25%, about 30%, about 35%, about 40%, about 50%, about 60%, about 70%, about 80%, about 90%, or about 100% (i.e., essentially eliminated), as compared to the control level of the symptom. In certain aspects, a composition can be said to reduce the severity of a symptom if the symptom is reduced between about 10% to about 100%, about 20% to about 90%, about 30% to about 80%, about 40% to about 70% or about 50% to about 60%, as compared to the control level of the symptom. In certain aspects, a composition can be said to reduce the severity of a symptom of if the symptom is reduced between about 10% to about 30%, about 20% to about 40%, about 30% to about 50%, about 40% to about 60%, about 50% to about 70%, about 60% to about 80%, about 70% to about 90% or about 80% to about 100%, as compared to the control level of the symptom.

A “patient” or “subject” for the purposes of the present invention includes both humans and other animals, particularly mammals. Thus, the compositions/formulations and methods are applicable to both human therapy and veterinary applications. In certain embodiment, the patient is a mammal, and in one embodiment, is a human. Other known treatments and therapies for conditions associated with ADAMTS13 or VWF dysfunction can be used in combination with the compositions and methods provided by the invention.

As used herein, the terms “vitamin B3”, “nicotinamide”, “niacinamide”, “niacin”, and “nicotinic acid” may be used interchangeably to refer to any member of the B3 family of vitamins.

As used herein, a “therapeutically effective amount or dose” or “sufficient amount or dose” refers to a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, a “physiological concentration” of salt refers to a salt concentration of between about 100 mM and about 200 mM of a pharmaceutically acceptable salt. Non-limiting examples of pharmaceutically acceptable salts include, without limitation, sodium and potassium chloride, sodium and potassium acetate, sodium and potassium citrate, sodium and potassium phosphate.

As used herein, a “sub-physiological concentration” of salt refers to a salt concentration of less than about 100 mM of a pharmaceutically acceptable salt. In certain embodiments, a sub-physiological concentration of salt is less than about 80 mM of a pharmaceutical salt. In certain embodiments, a sub-physiological concentration of salt is less than about 60 mM of a pharmaceutical salt.

As used herein, the term “chemically defined medium” refers to a synthetic growth medium in which the identity and concentration of all the components are known. Chemically defined mediums do not contain bacterial, yeast, animal, or plant extracts, although they may or may not include individual plant or animal-derived components (e.g., proteins, polypeptides, etc.). Non-limiting examples of commercially available chemically defined mediums include, various EX-CELL® mediums (SAFC Biosciences, Inc), various Dulbecco's Modified Eagle's (DME) mediums (Sigma-Aldrich Co; SAFC Biosciences, Inc), Ham's Nutrient Mixture (Sigma-Aldrich Co; SAFC Biosciences, Inc), and the like. Methods of preparing chemically defined culture mediums are known in the art, for example in U.S. Pat. Nos. 6,171,825 and 6,936,441, WO 2007/077217, and U.S. Patent Application Publication Numbers 2008/0009040 and 2007/0212770, the disclosures of which are incorporated herein by reference in their entireties for all purposes.

As used herein, the term “oligopeptide-free culture medium” refers to a protein-free medium that does not comprise oligopeptides, such as, e.g., oligopeptides derived from a protein hydrolysate. In one embodiment, the medium does not comprise oligopeptides having twenty or more amino acids. In one embodiment of the present invention, the medium does not comprise oligopeptides having fifteen or more amino acids. In another embodiment of the invention, the medium does not comprise oligopeptides having ten or more amino acids. In one embodiment, the medium does not comprise oligopeptides having seven or more amino acids. In another embodiment, the medium does not comprise oligopeptides having five or more amino acids. In still another embodiment the medium does not comprise oligopeptides having three or more amino acids. According to a further embodiment of the present invention, the medium does not comprise oligopeptides having two or more amino acids. Methods of preparing oligopeptide-free culture medium are known in the art, for example in U.S. Pat. Nos. 6,171,825 and 6,936,441, WO 2007/077217, and U.S. Patent Application Publication Numbers 2008/0009040 and 2007/0212770, the disclosures of which are incorporated herein by reference in their entireties for all purposes.

As used herein, the term “serum-free culture medium” refers to a culture medium that is not supplemented with an animal serum. Although oftentimes serum-free mediums are chemically defined mediums, serum-free mediums may be supplemented with discrete animal or plant proteins or protein fractions. Methods of preparing serum-free culture medium are known in the art, for example in U.S. Pat. Nos. 6,171,825 and 6,936,441, WO 2007/077217, and U.S. Patent Application Publication Numbers 2008/0009040 and 2007/0212770, the disclosures of which are incorporated herein by reference in their entireties for all purposes. As used herein, the term “animal protein-free culture medium” refers to a culture medium that is not supplemented with an animal serum, protein, or protein fraction. Although oftentimes animal protein-free culture mediums are chemically defined mediums, animal protein-free culture mediums may contain plant or yeast hydrolysates. Methods of preparing animal protein-free culture medium are known in the art, for example in U.S. Pat. Nos. 6,171,825 and 6,936,441, WO 2007/077217, and U.S. Patent Application Publication Numbers 2008/0009040 and 2007/0212770, the disclosures of which are incorporated herein by reference in their entireties for all purposes. In certain embodiments, a chemically defined medium is used without any animal or plant derived protein or protein fractions.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, the term “about” denotes an approximate range of plus or minus 10% from a specified value. For instance, the language “about 20%” encompasses a range of 18-22%. As used herein, about also includes the exact amount. Hence “about 20%” means “about 20%” and also “20%.”

If aspects of the disclosure are described as “comprising”, or versions thereof (e.g., comprises), a feature, embodiments also are contemplated “consisting of” or “consisting essentially of” the feature.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as about 1% to 50%, it is intended that values such as 2% to 40%, 10% to 30%, or 1% to 3%, etc., are expressly enumerated in this specification. The values listed above are only examples of what is specifically intended.

### III. ADAMTS13 Variant Compositions

In one aspect, the present invention provides ADAMTS13 variants. An ADAMTS13 variant can include one or more amino acid substitutions, deletions, insertions and/or frame shifts as compared to the amino acid sequence of a natural/wildtype ADAMTS13 (e.g., SEQ ID NO: 1). For example, the ADAMTS13 variant can include at least one single amino acid substitution as compared to (i.e., relative to) a wildtype ADAMTS13. The amino acid substitution(s) can be within the catalytic domain, the disintegrin domain, and/or the first thrombospondin type 1 domains (C1 and C2).

For example, the amino acid substitution can be at least one of I⁷⁹M, V⁸⁸M, H⁹⁶D, Q⁹⁷R, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13 (in other words, SEQ ID NO: 1 is used as a reference sequence to provide context as to the location in which the identified amino acid residue to be altered (e.g., M⁷⁹, M⁸⁸, D⁹⁶, R⁹⁷, C¹⁰², F¹¹⁹, T¹⁷⁸, W¹⁹³, I¹⁹⁶, P²⁰³, Q²³², Q²³⁴, H²³⁵, V²⁵⁰, C²⁶³, and/or P²⁶⁸) is located in an ADAMTS13 protein). In certain embodiments, the single amino acid substitution is not I⁷⁹M, V⁸⁸M, H⁹⁶D, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13.

In certain embodiments, the ADAMTS13 variant comprises a single amino acid substitution at Q⁹⁷ as denoted in SEQ ID NO: 1, or the equivalent amino acid position in an ADAMTS13. In certain embodiments, the amino acid change is from a Q to a D, E, K, H, L, N, P, or R. In certain embodiments, the amino acid change is from a Q to an R. In certain embodiments, the ADAMTS13 variant is ADAMTS13 Q⁹⁷R (SEQ ID NO: 2, or an amino acid sequence having at least 80% sequence identity thereof while still maintaining R⁹⁷). In some embodiments, the ADAMTS13 variant comprises the amino acid sequence set forth in SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the nucleotide sequence that encodes the ADAMTS13 variant comprises the nucleotide sequence that encodes the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the ADAMTS13 variant comprises the amino acid sequence set forth in SEQ ID NO: 2. In certain embodiments, the ADAMTS13 variant consists of the amino acid sequence set forth in SEQ ID NO: 2. In certain embodiments, the ADAMTS13 variant consists essentially of the amino acid sequence set forth in SEQ ID NO: 2.

In certain embodiments, the ADAMTS13 variant is R⁷W, Q⁴⁴X, T¹⁶⁷M, Y³⁰⁴C, C³¹¹Y, T³³⁹R, P³⁴¹L, C³⁴⁷S, R³⁴⁹C, P³⁵³L, W³⁹⁰X, W³⁹⁰C, R³⁹⁸H, Q⁴⁴⁸E, Q⁴⁴⁹X, Q⁴⁵⁶H, P⁴⁵⁷L, P⁴⁷⁵S, R⁵⁰⁷Q, C⁵⁰⁸Y, G⁵²⁵D, R⁵²⁸G, A⁵⁹⁶V, A⁶⁰⁶P, P⁶¹⁸A, R⁶²⁵H, P⁶⁷¹L, I⁶⁷³F, R⁶⁹²C, Q⁷²³K, A⁷³²V, E⁷⁴⁰K, C⁷⁵⁸R, V⁸³²M, A⁹⁰⁰V, S⁹⁰³L, C⁹⁰⁸S, C⁹⁰⁸Y, R⁹¹⁰X, Q⁹²⁹X, C⁹⁵¹G, G⁹⁸²R, A¹⁰³³T, W¹⁰¹⁶X, C¹⁰²⁴G, A¹⁰³³T, R¹⁰³⁴X, S¹⁰³⁶X, R¹⁰⁶⁰W, R¹¹²³C, R¹¹⁴⁹W, R¹²⁰⁶X, C¹²¹³Y, I¹²¹⁷T, R¹²¹⁹W, T¹²²⁶I, G¹²³⁹V, W¹²⁴⁵X, Q¹³⁰²X, S¹³¹⁴L, and/or R¹³³⁶W. In certain embodiments, the ADAMTS13 variant is not R⁷W, Q⁴⁴X, T¹⁶⁷M, Y³⁰⁴C, C³¹¹Y, T³³⁹R, P³⁴¹L, C³⁴⁷S, R³⁴⁹C, P³⁵³L, W³⁹⁰X, W³⁹⁰C, R³⁹⁸H, Q⁴⁴⁸E, Q⁴⁴⁹X, Q⁴⁵⁶H, P⁴⁵⁷L, F⁴⁷⁵S, R⁵⁰⁷Q, C⁵⁰⁸Y, G⁵²⁵D, R⁵²⁸G, A⁵⁹⁶V, A⁶⁰⁶P, P⁶¹⁸A, R⁶²⁵H, P⁶⁷¹L, I⁶⁷³F, R⁶⁹²C, Q⁷²³K, A⁷³²V, E⁷⁴⁰K, C⁷⁵⁸R, V⁸³²M, A⁹⁰⁰V, S⁹⁰³L, C⁹⁰⁸S, C⁹⁰⁸Y, R⁹¹⁰X, Q⁹²⁹X, C⁹⁵¹G, G⁹⁸²R, A¹⁰³³T, W¹⁰¹⁶X, C¹⁰²⁴G, A¹⁰³³T, R¹⁰³⁴X, S¹⁰³⁶X, R¹⁰⁶⁰W, R¹¹²³C, R¹¹⁴⁹W, R¹²⁰⁶X, C¹²¹³Y, I¹²¹⁷T, R¹²¹⁹W, T¹²²⁶I, G¹²³⁹V, W¹²⁴⁵X, Q¹³⁰²X, S¹³¹⁴L, and/or R¹³³⁶W as denoted in SEQ ID NO: 1, or the equivalent amino acid position in an ADAMTS13.

In certain embodiments, the ADAMTS13 variants provided herein retain significant ADAMTS13 activity. In certain embodiments, the ADAMTS13 variants provided equal ADAMTS13 activity as a wildtype ADAMTS13. In certain embodiments, the ADAMTS13 variants provided greater ADAMTS13 activity than a wildtype ADAMTS13 on its own.

In certain embodiments, the present invention provides compositions of ADAMTS13 variant(s), such as compositions with the constituents described in U.S. Patent Application Publication No. 2011/0229455 and/or in U.S. Patent Application Publication No. 2014/0271611, each of which are incorporated herein by reference in their entirety and for all purposes. In other aspects, the present invention provides compositions of ADAMTS13 variant(s) in combination with plasma derived ADAMTS13 and/or wildtype recombinant ADAMTS13 (rADAMTS13) proteins. In one embodiment, the amino acid sequence of hADAMTS13 is that of GenBank accession number NP_620594. In another embodiment, the amino acid sequence of hADAMTS13 comprises amino acids 75 to 1427 of NP_620594, a natural or conservative variant thereof, or a biologically active fragment thereof. In certain embodiments, the ADAMTS13 variant is ADAMTS13 Q⁹⁷R (SEQ ID NO: 2), or an amino acid sequence having at least 80% sequence identity thereof while still maintaining R⁹⁷.

In certain embodiments, the compositions are liquid or lyophilized compositions. In other embodiments, a lyophilized composition is lyophilized from a liquid composition as described in U.S. Patent Application Publication No. 2011/0229455 and/or in U.S. Patent Application Publication No. 2014/0271611, each of which are incorporated herein by reference in their entirety and for all purposes.

In certain embodiments, the pharmaceutical composition comprises a combination of at least one ADAMTS13 variant and ADAMTS13 protein (e.g., wildtype). In certain embodiments, the relative abundance (e.g., percentage) of ADAMTS13 variant present in the total amount of ADAMTS13 in the composition (i.e., including all ADAMTS13 variant(s) and wildtype) is between about 5% to about 95%, about 10% to about 90%, about 15% to about 85%, about 20% to about 80%, about 25% to about 75%, about 30% to about 70%, about 35% to about 65%, about 40% to about 60%, to about 45% to about 55%. In certain embodiments, the percentage of ADAMTS13 variant present in the total amount of ADAMTS13 in the composition is between about 40% to about 90%, about 40% to about 80%, about 45% to about 75%, about 50% to about 80%, about 50% to about 70%, or about 55% to about 65%. In certain embodiments, the percentage of ADAMTS13 variant present in the total amount of ADAMTS13 in the composition is between about 50% to about 75%, about 52% to about 72%, about 55% to about 70%, about 59% to about 72%. In certain embodiments, the percentage of ADAMTS13 variant present in the total amount of ADAMTS13 in the composition is between about 45% to about 85% or about 47% to about 84%. In certain embodiments, the percentage of ADAMTS13 variant present in the total amount of ADAMTS13 in the composition is between about 47% to about 84%. In certain embodiments, the percentage of ADAMTS13 variant present in the total amount of ADAMTS13 in the composition is about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In certain embodiments, the percentage of ADAMTS13 variant present in the total amount of ADAMTS13 in the composition is about 52%, about 60%, about 61%, about 62%, about 63%, about 64%, about 65%, about 66%, or about 72%.

In certain embodiments, the pharmaceutical composition comprises a combination of at least one ADAMTS13 variant and ADAMTS13 protein. In certain embodiments, the ratio of ADAMTS13 variant to ADAMTS13 protein is about 4:1 to about 1:4, about 3:1 to about 1:3, about 2:1 to about 1:2. In certain embodiments, the ratio of ADAMTS13 variant to ADAMTS13 protein is about 3:1 to about 1:3, about 2:1 to about 1:2, or about 2:1 to about 1:3, or about 1:1 to about 1:3, or about 1:1.1 to about 1:2.9, or about 1:1.2 to about 1:2.8, or about 1:1.3 to about 1:2.7, or about 1:1.4 to about 1:2.6, or about 1:1.5 to about 1:2.5, or about 1:1.6 to about 1:2.4, or about 1:1.7 to about 1:2.3, or about 1:1.8 to about 1:2.2, or about 1:1.9 to about 1:2.1. In certain embodiments, the ratio of ADAMTS13 variant to ADAMTS13 protein is about 1.1:1 to about 2.9:1, or about 1.2:1 to about 2.8:1, or about 1.3:1 to about 2.7:1, or about 1.4:1 to about 2.6:1, or about 1.5:1 to about 2.5:1, or about 1.6:1 to about 2.4:1, or about 1.7:1 to about 2.3:1, or about 1.8:1 to about 2.2:1, or about 1.9:1 to about 2.1:1. In certain embodiments, the ratio of ADAMTS13 variant to ADAMTS13 wildtype is about 1:1 to about 1:3. In certain embodiments, the ratio of ADAMTS13 variant to ADAMTS13 wildtype is about 3:1 to about 1:1. In certain embodiments, the ratio of ADAMTS13 variant to ADAMTS13 wildtype is about 1:1.1 to about 1:2.5. In certain embodiments, the ratio of ADAMTS13 variant to ADAMTS13 wildtype is about 4:1, about 4:1.5, about 4:2, about 4:2.5, about 4:3, about 4:3.5, about 3:1, about 3:1.5, about 3:2, about 3:2.5, about 2:1, or about 2:1.5. In certain embodiments, the ratio of ADAMTS13 variant to ADAMTS13 wildtype is about 1:1.5, about 1:2, about 1:2.5, about 1:3, about 1:3.5, about 1:4, about 2:2.5, about 2:3, about 2:3.5, about 2:4, about 3:3.5, or about 3:4. In certain embodiments, the ratio of ADAMTS13 variant to ADAMTS13 wildtype is about 1:3. In certain embodiments, the ratio of ADAMTS13 variant to ADAMTS13 wildtype is about 3:1. In certain embodiments, the ratio of ADAMTS13 variant to ADAMTS13 wildtype is about 2:1. In certain embodiments, the ratio of ADAMTS13 variant to ADAMTS13 wildtype is about 1:2. In certain embodiments, the ratio of ADAMTS13 variant to ADAMTS13 wildtype is about 1:1. In certain embodiments, the ratio of ADAMTS13 variant to ADAMTS13 wildtype is about 3:2.

In certain embodiments, the relative abundance, percentage, and/or ratio is determined by a peptide mapping method. In certain embodiments, the relative abundance, percentage, and/or ratio is determined by a peptide mapping method as described in Example 3. In certain embodiments, the relative abundance, percentage, and/or ratio is determined by HPLC analysis of tryptic peptides separated by liquid chromatography followed by mass spectrometry analysis. In certain embodiments, the relative abundance, percentage, and/or ratio is based on intensities in extracted ion chromatograms. In certain embodiments, the relative abundance, percentage, and/or ratio is determined based on the peak area of tryptic peptides of the ADAMTS13 variant (e.g., Q⁹⁷R ADAMTS13 variant) in relation to the sum of the peak areas of all ADAMTS13 proteins and variants in the composition (e.g., sum total of Q⁹⁷R ADAMTS13 variant and Q⁹⁷ ADAMTS13 protein). In certain embodiments, the tryptic peptides of all ADAMTS13 proteins and variants in the composition being measured are specific to the at least one amino acid difference between the ADAMTS13 variant as compared to all other ADAMTS13 proteins and variants in the composition. For example, the tryptic peptide(s) that can be measured for the Q⁹⁷R ADAMTS13 variant can be AAGGILHLELLVAVGPDVFQAHR (SEQ ID NO: 115) or a combination of AAGGILHLELLVAVGPDVFQAHR (SEQ ID NO: 115) and EDTER (SEQ ID NO: 5) and the tryptic peptide measured for the Q⁹⁷ ADAMTS13 protein can be AAGGILHLELLVAVGPDVFQAHQEDTER (SEQ ID NO: 6).

In certain embodiments, the relative abundance, percentage, and/or ratio is determined based on total weight of ADAMTS13 variant in relation to the sum total weight of all ADAMTS13 proteins and variants in the composition.

The composition of the disclosure is, in various aspects, administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. In some embodiments, administration is subcutaneous. Administration by intravenous, intradermal, intramuscular, intramammary, intraperitoneal, intrathecal, retrobulbar, intrapulmonary injection and or surgical implantation at a particular site is contemplated as well. In some embodiments, administration is intravenous. Generally, compositions are essentially free of pyrogens, as well as other impurities that could be harmful to the recipient.

In certain embodiments, the subcutaneous composition is administered by subcutaneous injection. In specific embodiments, the subcutaneous composition is subcutaneously injected into the same site of a patient (e.g., administered to the upper arm, anterior surface of the thigh, lower portion of the abdomen, or upper back) for repeat or continuous injections. In other embodiments, the subcutaneous composition is subcutaneously injected into the different or rotating sites of a patient.

In certain embodiments, the subcutaneous composition is administered by subcutaneously implanted device. In certain embodiments, the implanted device provides a timed release of a composition. In certain embodiments, the implanted device provides a continuous release of a composition.

Formulation of the composition or pharmaceutical composition will vary according to the route of administration selected (e.g., solution or emulsion). An appropriate composition comprising the composition to be administered is prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles, in certain embodiments, include sodium chloride solution, Ringer's dextrose, dextrose and sodium chloride, lactated Ringer's or fixed oils. Intravenous vehicles, in certain aspects, include various additives, preservatives, or fluid, nutrient or electrolyte replenishers.

Compositions or pharmaceutical compositions useful in the compounds and methods of the disclosure containing at least one ADAMTS13 variant as an active ingredient contain, in various aspects, pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form.

A variety of aqueous carriers, e.g., water, buffered water, 0.4% saline, 0.3% glycine, or aqueous suspensions contain, in various aspects, the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents, in some instances, are a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions, in certain embodiments, contain one or more preservatives, for example ethyl, or n-propyl, p-hydroxybenzoate.

In some certain embodiments, ADAMTS13 variant(s) or ADAMTS13 variant compositions, including compositions with other ADAMTS13 proteins, are lyophilized for storage, and reconstituted in a suitable carrier prior to use. Any suitable lyophilization and reconstitution techniques known in the art are employed. It is appreciated by those skilled in the art that lyophilization and reconstitution leads to varying degrees of protein activity loss and that use levels are often adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

In certain embodiments, the ADAMTS13 variant compositions, including compositions with ADAMTS13, provided herein may further comprise one or more pharmaceutically acceptable excipients, carriers, and/or diluents as described in U.S. Patent Application No. 20110229455 and/or in U.S. Patent Application Publication No. 2014/0271611, each of which are incorporated herein by reference in their entirety for all purposes.

In certain embodiments, the ADAMTS13 variant compositions, including compositions with other ADAMTS13 proteins, provided herein will have a tonicity in a range as described in U.S. Patent Application Publication No. 2011/0229455 and/or in U.S. Patent Application Publication No. 2014/0271611, each of which are incorporated herein by reference in their entirety for all purposes.

In one embodiment, the present invention provides compositions of ADAMTS13 variant(s), including compositions with other ADAMTS13 proteins, comprising a therapeutically effective amount or dose of at least one ADAMTS13 variant or therapeutically effective amount of total ADAMTS13, a sub-physiological to physiological concentration of a pharmaceutically acceptable salt, a stabilizing concentration of one or more sugars and/or sugar alcohols, a non-ionic surfactant, a buffering agent providing a neutral pH to the compositions and optionally a calcium and/or zinc salt. Generally, the stabilized compositions provided herein are suitable for pharmaceutical administration.

In certain embodiments, the compositions are liquid compositions. In other embodiments, the compositions are lyophilized compositions that are lyophilized from a liquid composition as described in U.S. Patent Application Publication No. 2011/0229455.

In certain embodiments, the ADAMTS13 variant is provided in a therapeutically effective dose between about 0.01 mg/mL and about 10 mg/mL. In certain embodiments, the ADAMTS13 variant is provided in a therapeutically effective dose between about 0.05 mg/mL and about 10 mg/mL. In other embodiments, the ADAMTS13 variant is present at a concentration of between about 0.1 mg/mL and about 10 mg/mL. In yet other embodiments, the ADAMTS13 variant is present at a concentration of between about 0.1 mg/mL and about 5 mg/mL. In yet other embodiments, the ADAMTS13 variant is present at a concentration of between about 0.03 mg/mL and about 0.4 mg/mL. In another embodiment, the ADAMTS13 variant is present at a concentration of between about 0.1 mg/mL and about 2 mg/mL. In yet other embodiments, the ADAMTS13 variant may be present at about 0.01 mg/mL, or at about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, or a higher concentration. In one embodiment, the concentration of a relatively pure ADAMTS13 variant may be determined by spectroscopy (i.e., total protein measured at A280) or other bulk determination (e.g., Bradford assay, SDS-PAGE in combination with various staining methods like Coomassie staining or silver stain, weight of a lyophilized powder, etc.). In other embodiments, the concentration of ADAMTS13 variant may be determined by an ADAMTS13 ELISA assay (e.g., mg/mL antigen).

In certain embodiments, the ADAMTS13 variant(s) together with ADAMTS13 protein (e.g., wildtype ADAMTS13) is provided in a therapeutically effective dose between about 0.01 mg/mL and about 10 mg/mL total ADAMTS13 (i.e., total amount of both together). In certain embodiments, the ADAMTS13 variant(s) together with ADAMTS13 protein is provided in a therapeutically effective dose between about 0.05 mg/mL and about 10 mg/mL total ADAMTS13 (i.e., total amount of both together). In other embodiments, the ADAMTS13 variant together with ADAMTS13 protein is present at a concentration of between about 0.1 mg/mL and about 10 mg/mL. In yet other embodiments, the ADAMTS13 variant together with ADAMTS13 protein is present at a concentration of between about 0.1 mg/mL and about 5 mg/mL. In other embodiments, the ADAMTS13 variant together with ADAMTS13 protein is present at a concentration of between about 0.03 mg/ml to about 0.4 mg/ml. In another embodiment, the ADAMTS13 variant together with ADAMTS13 protein is present at a concentration of between about 0.1 mg/mL and about 2 mg/mL. In yet other embodiments, the ADAMTS13 variant together with ADAMTS13 protein may be present at about 0.01 mg/mL, or at about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, or a higher concentration. In one embodiment, the concentration of a relatively pure ADAMTS13 variant and ADAMTS13 protein may be determined by spectroscopy (i.e., total protein measured at A280) or other bulk determination (e.g., Bradford assay, silver stain, weight of a lyophilized powder, etc.). In other embodiments, the concentration of ADAMTS13 variant and ADAMTS13 protein may be determined by an ADAMTS13 ELISA assay (e.g., mg/mL antigen). In certain embodiments, the ADAMTS13 variant and ADAMTS13 protein is detected separately (i.e., distinguishable from each other). In certain embodiments, the ADAMTS13 variant and ADAMTS13 protein is detected together (i.e., indistinguishable from each other).

In yet another embodiment, the concentration of ADAMTS13 variant(s) and/or ADAMTS13 protein in a composition provided by the present invention may be expressed as a level of enzymatic activity. For example, in one embodiment a formulation may contain between about 0.01 units of FRETS-VWF73 activity and about 10,000 units of FRETS-VWF73 activity or other suitable ADAMTS13 enzymatic unit (IU). In another embodiment a formulation may contain between about 0.1 units of FRETS-VWF73 activity and about 10,000 units of FRETS-VWF73 activity or other suitable ADAMTS13 enzymatic unit (IU). In another embodiment a formulation may contain between about 1 unit of FRETS-VWF73 activity and about 10,000 units of FRETS-VWF73 activity or other suitable ADAMTS13 enzymatic unit (IU). In another embodiment a formulation may contain between about 10 units of FRETS-VWF73 activity and about 10,000 units of FRETS-VWF73 activity or other suitable ADAMTS13 enzymatic unit (IU). In other embodiments, the formulation may contain between about 1 unit of FRETS-VWF73 (U_{FV73}) activity and about 8,000 units of FRETS-VWF73 activity, or between about 30 U_{FV73} and about 6,000 U_{FV73}, or between about 40 U_{FV73} and about 4,000 U_{FV73}, or between about 50 U_{FV73} and about 3,000 U_{FV73}, or between about 75 U_{FV73} and about 2,500 U_{FV73}, or between about 100 U_{FV73} and about 2,000 U_{FV73}, or between about 200 U_{FV73} and about 1,500 U_{FV73}, or between about other ranges therein. In certain embodiments, a formulation provided herein contains between about 20 and about 10,000. U_{FV73}. In certain embodiments, a formulation contains about 0.01 units of FRETS-VWF73 activity, or about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more units of FRETS-VWF73 activity.

Similarly, in certain embodiments, the concentration of ADAMTS13 variant(s) and/or ADAMTS13 protein may be expressed as an enzymatic activity per unit volume, for example, ADAMTS13 enzymatic units per mL (IU/mL). For example, in one embodiment a formulation may contain between about 0.01 IU/mL and about 10,000 IU/mL. In another embodiment a formulation may contain between about 0.1 IU/mL and about 10,000 IU/mL. In another embodiment a formulation may contain between about 1 IU/mL and about 10,000 IU/mL. In another embodiment a formulation may contain between about 10 IU/mL and about 10,000 IU/mL. In other embodiments, the formulation may contain between about 1 IU/mL and about 10,000 IU/mL, or between about 20 IU/mL and about 8,000 IU/mL, or between about 30 IU/mL and about 6,000 IU/mL, or between about 40 IU/mL and about 4,000 IU/mL, or between about 50 IU/mL and about 3,000 IU/mL, or between about 75 IU/mL and about 2,500 IU/mL, or between about 100 IU/mL and about 2,000 IU/mL, or between about 200 IU/mL and about 1,500 IU/mL, or between about other ranges therein. In certain embodiments, a formulation provided herein contains between about 150 IU/mL and about 600 IU/mL. In certain embodiments, a formulation provided herein contains between about 100 IU/mL and about 1,000 IU/mL. In certain embodiments, a formulation contains about 0.01 IU/mL, or about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more IU/mL.

In certain embodiments, the disclosure provides compositions of ADAMTS13 variants, including compositions with ADAMTS13, comprising the exemplary compositions described in Section III (“ADAMTS13 Compositions and Formulations”) of U.S. Patent Application Publication No. 2011/0229455. The methods of ADAMTS13 production and compositions thereof as described in U.S. Patent Application Publication No. 2011/0229455 and/or in U.S. Patent Application Publication No. 2014/0271611, which are incorporated herein by reference in their entirety for all purposes. Additionally, actual methods for preparing parenterally administrable compositions and compositions are known or are apparent to those skilled in the art and are described in more detail in, for example, Remington's Pharmaceutical Science, 15th ed., Mack Publishing Company, Easton, Pa. (1980).

In certain embodiments, the compositions of ADAMTS13 variants, including compositions with ADAMTS13, are produced and comprise the additives. The methods of ADAMTS13 variant(s) and/or ADAMTS13 protein production and compositions thereof as described in U.S. Patent Application Publication No. 2011/0229455, Sections IV and V, which are incorporated herein by reference for all purposes.

In various aspects, the pharmaceutical compositions are in the form of a sterile injectable aqueous, oleaginous suspension, dispersions or sterile powders for the extemporaneous preparation of sterile injectable solutions or dispersions. The suspension, in certain embodiments, is formulated according to the known art using those suitable dispersing or wetting agents and suspending agents which have been mentioned above. The sterile injectable preparation, in certain aspects, is a sterile injectable solution or suspension in a non-toxic parenterally-acceptable diluent or solvent, for example as a solution in 1,3-butane diol. In some embodiments, the carrier is a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose, any bland fixed oil is employed, in various aspects, including synthetic mono- or diglycerides. In addition, fatty acids such as oleic acid find use in the preparation of injectables.

In all cases the form must be sterile and must be fluid to the extent that easy syringability exists. The proper fluidity is maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms is brought about by various antibacterial and antifungal agents, for example, parabens, chlorobutanol, phenol, sorbic acid, thimerosal, and the like. In many cases, it will be desirable to include isotonic agents, for example, sugars or sodium chloride. In certain aspects, prolonged absorption of the injectable compositions is brought about by the use in the compositions of agents delaying absorption, for example, aluminum monostearate and gelatin.

Compositions useful for administration, in certain aspects, are formulated with uptake or absorption enhancers to increase their efficacy. Such enhancers include, for example, salicylate, glycocholate/linoleate, glycolate, aprotinin, bacitracin, SDS, caprate and the like. See, e.g., Fix (J. Pharm. Sci., 85:1282-1285, 1996) and Oliyai et al. (Ann. Rev. Pharmacol. Toxicol., 32:521-544, 1993), each of which are incorporated herein by reference in their entirety and for all purposes.

In addition, the properties of hydrophilicity and hydrophobicity of the compositions used in the compositions and methods of the disclosure are well balanced, thereby enhancing their utility for both in vitro and especially in vivo uses, while other compositions lacking such balance are of substantially less utility. Specifically, compositions in the disclosure have an appropriate degree of solubility in aqueous media which permits absorption and bioavailability in the body, while also having a degree of solubility in lipids which permits the compounds to traverse the cell membrane to a putative site of action.

In certain embodiments, ADAMTS13 variant(s) and/or ADAMTS13 protein are provided in a pharmaceutically acceptable (i.e., sterile and non-toxic) liquid, semisolid, or solid diluent that serves as a pharmaceutical vehicle, excipient, or medium. Any diluent known in the art is used. Exemplary diluents include, but are not limited to, polyoxyethylene sorbitan monolaurate, magnesium stearate, methyl- and propylhydroxybenzoate, talc, alginates, starches, lactose, sucrose, dextrose, sorbitol, mannitol, gum acacia, calcium phosphate, mineral oil, cocoa butter, and oil of theobroma.

The composition is packaged in forms convenient for delivery. The composition is enclosed within a capsule, caplet, sachet, cachet, gelatin, paper, or other container. These delivery forms are preferred when compatible with delivery of the composition into the recipient organism and, particularly, when the composition is being delivered in unit dose form. The dosage units are packaged, e.g., in vials, tablets, capsules, suppositories, or cachets.

In certain embodiments, the ADAMTS13 variant(s) and/or ADAMTS13 protein formulations may contain a sub-physiological to physiological salt concentration, for example, between and 0 mM and about 200 mM of a pharmaceutically acceptable salt. In one embodiment, an ADAMTS13 variant(s) and/or ADAMTS13 protein formulation will contain a physiological concentration of salt, for example, between about 100 mM and about 200 mM of a pharmaceutically acceptable salt. In one embodiment, an ADAMTS13 variant(s) and/or ADAMTS13 protein formulation will contain a physiological concentration of salt, for example, between about 0 mM and about 60 mM of a pharmaceutically acceptable salt. In other embodiments, an ADAMTS13 variant(s) and/or ADAMTS13 protein formulation will contain about 0 mM, or about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 110 mM, 120 mM, 130 mM, 140 mM, 150 mM, 160 mM, 170 mM, 180 mM, 190 mM, 200 mM, or more of a pharmaceutically acceptable salt. In certain embodiments, the salt chloride is sodium or potassium chloride.

Advantageously, it has been found that ADAMTS13 variant(s) and/or ADAMTS13 protein formulations containing a sub-physiological concentration of a pharmaceutically acceptable salt form compact lyocakes with smooth surfaces. Furthermore, it has been found that low salt lyophilized formulations of ADAMTS13 variant(s) and/or ADAMTS13 protein proteins reduce protein aggregation as compared to formulations prepared with physiological concentrations of salt. Accordingly, in certain embodiments, the present invention provides low salt formulations of ADAMTS13 variant(s) and/or ADAMTS13 protein containing a sub-physiological concentration of a pharmaceutically acceptable salt, for example, less than about 100 mM of a pharmaceutically acceptable salt. In one embodiment, a low salt ADAMTS13 variant(s) and/or ADAMTS13 protein formulation provided herein contains less than about 100 mM of a pharmaceutical salt. In certain embodiments, a low salt ADAMTS13 variant(s) and/or ADAMTS13 protein formulation provided herein contains less than about 80 mM of a pharmaceutical salt. In certain embodiments, a low salt ADAMTS13 variant(s) and/or wildtype ADAMTS13 formulation provided herein contains less than about 60 mM of a pharmaceutical salt (i.e., between about 0 mM and about 60 mM salt). In certain embodiments, a low salt ADAMTS13 formulation will contain between about 30 mM and about 60 mM of a pharmaceutically acceptable salt. In yet other embodiments, a low salt ADAMTS13 variant(s) and/or wildtype ADAMTS13 formulation will contain about 0 mM, or about 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM of a pharmaceutically acceptable salt. In certain embodiments, a low salt ADAMTS13 variant(s) and/or ADAMTS13 protein formulation is a lyophilized formulation. In certain embodiments, the salt is sodium chloride or potassium chloride.

It has also been found that the inclusion of moderate levels (i.e., between about 2% and about 6%) of one or more sugars and/or sugar alcohols assists in the preparation of compact lyocakes with smooth surfaces and helps to stabilize ADAMTS13 variant(s) and/or wildtype ADAMTS13 upon lyophilization. Accordingly, in one embodiment, the present invention provides ADAMTS13 variant(s) and/or ADAMTS13 protein formulations containing between about 2% and about 6% of one or more sugars and/or sugar alcohols. Any sugar such as mono-, di-, or polysaccharides, or water-soluble glucans, including for example fructose, glucose, mannose, sorbose, xylose, maltose, lactose, sucrose, dextran, trehalose, pullulan, dextrin, cyclodextrin, soluble starch, hydroxyethyl starch, and carboxymethylcellulose may be used. In a particular embodiment, sucrose or trehalose is used as a sugar additive. Sugar alcohols are defined as a hydrocarbon having between about 4 and about 8 carbon atoms and a hydroxyl group. Non-limiting examples of sugar alcohols that may be used in the ADAMTS13 variant(s) and/or ADAMTS13 protein formulations provided herein include, mannitol, sorbitol, inositol, galactitol, dulcitol, xylitol, and arabitol. In one embodiment, mannitol is used as a sugar alcohol additive. In certain embodiments, an ADAMTS13 variant(s) and/or ADAMTS13 protein formulation contains both a sugar and a sugar alcohol additive.

The sugars and sugar alcohols may be used individually or in combination. In some embodiments, the sugar, sugar alcohol, or combination thereof will be present in the formulation at a concentration of between about 0.5% and about 7%. In one embodiment, the sugar and/or sugar alcohol content of the formulation will be between about 0.5% and about 5%. In certain embodiments, the sugar, sugar alcohol, or combination thereof will be present at a concentration of between about 1% and about 5%. In certain embodiments, the sugar, sugar alcohol, or combination thereof will be present at a concentration of between about 2% and about 6%. In certain embodiments, the sugar, sugar alcohol, or combination thereof will be present at a concentration of between about 3% and about 5%. In certain embodiments, the final concentration may be about 0.5%, 1%, 1.5%, 2%, 2.5%, 3%, 3.5%, 4%, 4.5%, 5%, 5.5%, 6.0%, 6.5%, or 7.0% sugar, sugar alcohol, or combination thereof. In certain embodiments, a formulation provided herein may comprise a sugar at a concentration from about 0.5% to about 5.0% and a sugar alcohol at a concentration from about 0.5% to about 5.0%. Any combination of sugar and sugar alcohol concentrations may be used, e.g. a sugar present at a concentration of about 0.5%, 1%, 1.5%, 2%, 2.5%, 3%, 3.5%, 4%, 4.5%, 5%, 5.5%, 6.0%, 6.5%, or 7.0% and a sugar alcohol present at a concentration of about 0.5%, 1%, 1.5%, 2%, 2.5%, 3%, 3.5%, 4%, 4.5%, 5%, 5.5%, 6.0%, 6.5%, or 7.0%.

Advantageously, it was also found that the inclusion of a non-ionic surfactant substantially reduces the aggregation of ADAMTS13 variant(s) and/or ADAMTS13 protein formulations. Accordingly, in one embodiment, ADAMTS13 variant(s) and/or ADAMTS13 protein formulations containing a stabilizing concentration of a non-ionic detergent are provided. Pharmaceutically acceptable nonionic surfactants that may be used in the formulations of the present invention are known in the art of pharmaceutical science, and include, without limitation, Polysorbate 80 (Tween 80), Polysorbate 20 (Tween 20), and various poloxamers or pluronics, including Pluronic F-68, and BRIJ 35, or mixtures thereof. In certain embodiments, the nonionic surfactant used in the present pharmaceutical formulations is Polysorbate 80. In certain embodiments, a surfactant may be used in a formulation provided herein at a concentration between about 0.001% and about 0.2%. In certain embodiments, the surfactant is used at a concentration of between about 0.01% and about 0.1%. In certain embodiments, the surfactant is used at a concentration of about 0.05%. For example, in certain embodiments, the formulation may include a nonionic surfactant at a concentration of about 0.001%, 0.005%, 0.01%, 0.02%, 0.03%, 0.04%, 0.05%, 0.06%, 0.07%, 0.08%, 0.09%, 0.1%, 0.125%, 0.15%, 0.175%, 0.2%, and the like.

Furthermore, it was found that ADAMTS13 variant(s) and/or ADAMTS13 protein formulations were stabilized when formulated at a neutral pH between about 6.5 and about 7.5. Accordingly, in certain embodiments, ADAMTS13 variant(s) and/or ADAMTS13 protein formulations are provided that contain a buffering agent suitable to maintain the formulation at a neutral pH. Pharmaceutically acceptable buffering agents are well known in the art, and include without limitation, phosphate buffers, histidine, sodium citrate, HEPES, Tris, Bicine, glycine, N-glycylglycine, sodium acetate, sodium carbonate, glycylglycine, lysine, arginine, sodium phosphate, and mixtures thereof. In certain embodiments, the buffer is selected from histidine, phosphate buffer, HEPES, and sodium citrate. In certain embodiments, the buffer is histidine or HEPES. In a specific embodiment, the buffer in histidine. In another specific embodiment, the buffer is HEPES. In one embodiment, the pH of the formulations provided herein is between about 6.5 and about 9.0. In certain embodiments, the pH of the formulation is about 6.5 or about 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In certain embodiments, the pH of the ADAMTS13 variant(s) and/or ADAMTS13 protein formulation is between about 6.0 and about 8.0. In certain embodiments, the pH of the ADAMTS13 variant(s) and/or ADAMTS13 protein formulation is between about 6.5 and about 7.5. In a particular embodiment, the pH of the ADAMTS13 variant(s) and/or ADAMTS13 protein formulation is about 7.0. In another particular embodiment, the pH of the ADAMTS13 variant(s) and/or ADAMTS13 protein formulation is 7.0±0.2.

It is also demonstrated herein that the inclusion of calcium further stabilizes formulations of ADAMTS13 variant(s) and/or ADAMTS13. Accordingly, in certain embodiments, stabilized ADAMTS13 variant(s) and/or ADAMTS13 protein formulations are provided which contain between about 0.5 mM and about 20 mM calcium (e.g., calcium chloride). Any pharmaceutically acceptable calcium salt may be used in the formulations provided herein. Non-limiting examples of calcium salt that may be used include, for example, CaCl₂), CaCO₃, Ca(C₆H₁₁O₇)₂, Ca₃(PO₄)₂, Ca(C₁₈H₃₅O₂)₂, and the like. In one embodiment, calcium is present in an ADAMTS13 variant(s) and/or ADAMTS13 protein formulation of the invention at a concentration from about 0.5 mM to about 10 mM. In another embodiment, calcium is present in an ADAMTS13 variant(s) and/or ADAMTS13 protein formulation at a concentration between about 2 mM and about 5 mM. In certain embodiments, calcium is present in an ADAMTS13 variant(s) and/or ADAMTS13 protein formulation at a concentration from about 2 mM to about 4 mM. In certain embodiments, the concentration of calcium is about 0.5 mM, or about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In a particular embodiment, the concentration of calcium is about 2 mM. In another embodiment, the concentration of calcium is about 3 mM. In yet another embodiment, the concentration of calcium is about 4 mM.

Similarly, it has been found that under certain conditions, the inclusion of zinc further stabilizes an ADAMTS13 variant(s) and/or ADAMTS13 protein formulation as provided herein. For example, FIG. 34 shows that inclusion of between about 2 μM and about 10 μM zinc further stabilized calcium containing ADAMTS13 variant(s) and/or ADAMTS13 protein formulations. Any pharmaceutically acceptable zinc salt may be used in the formulations provided herein. Non-limiting examples of zinc salt that may be used include, for example, ZnSO₄·7H₂O, ZnSO₃·2H₂O, Zn₃(PO₄)₂, and (C₆H₅O₇)₂Zn₃·2H₂O, and the like. In one embodiment, ZnSO₄ is used in the ADAMTS13 variant(s) and/or ADAMTS13 protein formulations provided herein. In some embodiments, zinc is present in an ADAMTS13 variant(s) and/or ADAMTS13 protein formulation of the invention at a concentration from about 0.5 μM to about 20.0 μM. In certain embodiments, zinc is included in an ADAMTS13 variant(s) and/or ADAMTS13 protein formulation at a concentration of between about 0.5 μM to about 10.0 μM. In certain embodiments, the concentration of zinc is about 0.5 μM, or about 1 μM, 2 μM, 3 μM, 4 μM, 5 μM, 6 μM, 7 μM, 8 μM, 9 μM, or 10 μM.

In one embodiment, the ADAMTS13 variant(s) and/or ADAMTS13 protein formulations provided herein will have a tonocity in a range between about 200 mOsmol/L and about 400 mOsmol/L, or in a range between about 250 and about 350 mOsmol/L. In certain embodiments, an ADAMTS13 variant(s) and/or ADAMTS13 protein formulation provided herein will have a tonocity, for example, of about 200 mOsmol/L, or of about 210 mOsmol/L, 220 mOsmol/L, 230 mOsmol/L, 240 mOsmol/L, 250 mOsmol/L, 260 mOsmol/L, 270 mOsmol/L, 280 mOsmol/L, 290 mOsmol/L, 300 mOsmol/L, 310 mOsmol/L, 320 mOsmol/L, 330 mOsmol/L, 340 mOsmol/L, 350 mOsmol/L, 360 mOsmol/L, 370 mOsmol/L, 380 mOsmol/L, 390 mOsmol/L, or 400 mOsmol/L.

Examples of tonocity agents that may be used in the formulations provided herein include, without limitation, sodium chloride, dextrose, sucrose, xylitol, fructose, glycerol, sorbitol, mannitol, trehalose, potassium chloride, mannose, calcium chloride, magnesium chloride, other inorganic salts, other sugars, other sugar alcohols, and combinations thereof. In certain embodiments, an ADAMTS13 variant(s) and/or ADAMTS13 protein formulation may comprise at least one tonocity agent, or at least two, three, four, five, or more tonocity agents.

The ADAMTS13 variant(s) and/or ADAMTS13 protein formulations provided herein may be formulated for administration via known methods, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. In certain embodiments, the ADAMTS13 variant(s) and/or ADAMTS13 protein formulations provided herein can be administered either systemically or locally. Systemic administration includes, without limitation: oral, subdermal, intraperitioneal, subcutaneous, transnasal, sublingual, or rectal routes of administration. Local administration includes, without limitation: topical, subcutaneous, intramuscular, and intraperitoneal routes of administration.

In one aspect of the invention, a composition of monomeric ADAMTS13 variant(s) and/or ADAMTS13 protein protein is provided. In certain embodiments, the composition of monomeric ADAMTS13 variant(s) and/or ADAMTS13 protein protein is substantially free of aggregated ADAMTS13 variant(s) and/or ADAMTS13, dimeric ADAMTS13 variant(s) and/or ADAMTS13, or both aggregated and dimeric ADAMTS13 variant(s) and/or ADAMTS13. In some embodiments, the monomeric composition has a higher specific activity than a similar composition containing aggregated and/or dimeric ADAMTS13 variant(s) and/or ADAMTS13 protein. In a particular embodiment, the monomeric ADAMTS13 variant(s) and/or ADAMTS13 protein composition is produced by a method comprising gel filtration or size exclusion chromatography. In one particular embodiment, the ADAMTS13 variant(s) and/or ADAMTS13 protein is a human ADAMTS13 variant(s) and/or ADAMTS13 protein or recombinant human ADAMTS13 variant(s) and/or ADAMTS13, or a biologically active derivative or fragment thereof.

In certain embodiments, the present invention provides formulations of ADAMTS13 variant(s) and/or ADAMTS13 protein comprising from about 0.01 mg/ml to about 10.0 mg/ml total ADAMTS13 protein, from about 0 mM to about 200 mM of a pharmaceutically acceptable salt, a sugar and/or sugar alcohol, a non-ionic surfactant, and a buffering agent. In certain embodiments, the formulations may further comprise calcium and/or zinc. In other embodiments, the formulation may be buffered at a pH of between about 6.5 and 9.0. In certain embodiments, the ADAMTS13 variant(s) and/or ADAMTS13 protein formulations are suitable for pharmaceutical administration.

In certain embodiments, the present invention provides a stabilized formulation of ADAMTS13 variant(s) and/or ADAMTS13, comprising: 0.01 mg/ml to 10.0 mg/ml total ADAMTS13; 0 mM to 200 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5. In certain embodiments, the pharmaceutically acceptable salt is sodium chloride or potassium chloride.

In one embodiment, an ADAMTS13 variant(s) and/or ADAMTS13 protein formulation is provided comprising 0.01 mg/ml to 10.0 mg/ml total ADAMTS13; 0 mM to 200 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5 contains between about 50 units per mL and about 1000 units per mL of ADAMTS13 activity. In certain embodiments, the pharmaceutically acceptable salt is sodium chloride or potassium chloride.

In another embodiment, the present invention provides stabilized ADAMTS13 variant(s) and/or ADAMTS13 protein formulations comprising 0.01 mg/ml to 10.0 mg/ml total ADAMTS13; 0 mM to 200 mM of a pharmaceutically acceptable salt; 1 mM to 10 mM calcium; a sugar and/or sugar alcohol; a nonionic surfactant; and a buffering agent for maintaining a pH of between about 6.5 and about 7.5. In certain embodiments, the formulation contains between about 2 mM and about 4 mM calcium. In certain embodiments, the pharmaceutically acceptable salt is sodium chloride or potassium chloride.

In another embodiment, the present invention provides stabilized ADAMTS13 variant(s) and/or ADAMTS13 protein formulations comprising 0.01 mg/ml to 10.0 mg/ml total ADAMTS13; 0 mM to 200 mM of a pharmaceutically acceptable salt; 0.5 mM to 20 mM calcium; between about 2% and about 6% of a sugar and/or sugar alcohol; a nonionic surfactant; and a buffering agent for maintaining a pH of between about 6.5 and about 7.5. In certain embodiments, the sugar and/or sugar alcohol is selected from the group consisting of sucrose, trehalose, mannitol, and a combination thereof. In certain embodiments, the sugar and/or sugar alcohol is a mixture of sucrose and mannitol. In a particular embodiment, the mixture of sucrose and mannitol consists of about 1% sucrose and about 3% mannitol. In certain embodiments, the formulation comprises between about 1 mM and about 10 mM calcium, or between about 2 mM and about 4 mM calcium. In certain embodiments, the pharmaceutically acceptable salt is sodium chloride or potassium chloride.

In another embodiment, the present invention provides stabilized ADAMTS13 variant(s) and/or ADAMTS13 protein formulations comprising 0.01 mg/ml to 10.0 mg/ml total ADAMTS13; 0 mM to 200 mM of a pharmaceutically acceptable salt; 0.5 mM to 20 mM calcium; a sugar and/or sugar alcohol; between about 0.01% and 0.1% of a nonionic surfactant; and a buffering agent for maintaining a pH of between about 6.5 and about 7.5. In certain embodiments, the surfactant is selected from the group consisting of Polysorbate 20, Polysorbate 80, Pluronic F-68, BRIJ 35, and a combination thereof. In certain embodiments, the sugar and/or sugar alcohol is a mixture of sucrose and mannitol. In a particular embodiment, the surfactant is Polysorbate 80. In certain embodiments, the formulation comprises between about 1 mM and about 10 mM calcium, or between about 2 mM and about 4 mM calcium. In certain embodiments, the pharmaceutically acceptable salt is sodium chloride or potassium chloride.

In another embodiment, the present invention provides stabilized ADAMTS13 variant(s) and/or ADAMTS13 protein formulations comprising 0.01 mg/ml to 10.0 mg/ml total ADAMTS13; 0 mM to 200 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5, wherein the buffering agent is histidine or HEPES. In certain embodiments, the buffering agent is present at a concentration between about 5 mM and about 100 mM, or between about 10 mM and about 50 mM. In another embodiment, the pH of the formulation is 7.0±0.2. In certain embodiments, the formulation comprises between about 1 mM and about 10 mM calcium, or between about 2 mM and about 4 mM calcium. In certain embodiments, the pharmaceutically acceptable salt is sodium chloride or potassium chloride.

In another embodiment, the present invention provides stabilized ADAMTS13 variant(s) and/or ADAMTS13 protein formulations comprising 0.01 mg/ml to 10.0 mg/ml total ADAMTS13; 0 mM to 200 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5. In certain embodiments, the formulation further comprises between about 0.5 μM and about 20 μM zinc. In certain embodiments, the formulation comprises between about 1 mM and about 10 mM calcium, or between about 2 mM and about 4 mM calcium. In certain embodiments, the pharmaceutically acceptable salt is sodium chloride or potassium chloride.

In certain embodiments, the present invention provides a stabilized ADAMTS13 variant(s) and/or ADAMTS13 protein formulation comprising 0.01 mg/ml to 10.0 mg/ml total ADAMTS13; 0 to 60 mM NaCl; 2 mM to 4 mM calcium; 2% to 4% mannitol; 0.5% to 2% sucrose; 0.025 to 0.1% Polysorbate 80; and 10 mM to 50 mM histidine (pH 7.0±0.2). In one embodiment, the formulation further comprises between about 0.5 μM and about 20 μM zinc.

In another embodiment, stabilized low salt ADAMTS13 variant(s) and/or ADAMTS13 protein formulations are provided comprising 0.01 mg/ml to 10.0 mg/ml total ADAMTS13; less than about 100 mM of a pharmaceutically acceptable salt; 0.5 mM to 20 mM calcium; a sugar and/or sugar alcohol; a nonionic surfactant; and a buffering agent for maintaining a pH of approximately between 6.5 and 7.5. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In one embodiment, a stabilized low salt ADAMTS13 variant(s) and/or ADAMTS13 protein formulation is provided comprising 0.01 mg/ml to 10.0 mg/ml total ADAMTS13; less than about 100 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5 contains between about 50 units per mL and about 1000 units per mL of ADAMTS13 activity. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In another embodiment, the present invention provides stabilized low salt ADAMTS13 variant(s) and/or ADAMTS13 protein formulations comprising 0.01 mg/ml to 10.0 mg/ml total ADAMTS13; less than about 100 mM of a pharmaceutically acceptable salt; 1 mM to 10 mM calcium; a sugar and/or sugar alcohol; a nonionic surfactant; and a buffering agent for maintaining a pH of between about 6.5 and about 7.5. In certain embodiments, the formulation contains between about 2 mM and about 4 mM calcium. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In another embodiment, the present invention provides stabilized low salt ADAMTS13 variant(s) and/or ADAMTS13 protein formulations comprising 0.01 mg/ml to 10.0 mg/ml total ADAMTS13; less than about 100 mM of a pharmaceutically acceptable salt; 0.5 mM to 20 mM calcium; between about 2% and about 6% of a sugar and/or sugar alcohol; a nonionic surfactant; and a buffering agent for maintaining a pH of between about 6.5 and about 7.5. In certain embodiments, the sugar and/or sugar alcohol is selected from the group consisting of sucrose, trehalose, mannitol, and a combination thereof. In certain embodiments, the sugar and/or sugar alcohol is a mixture of sucrose and mannitol. In a particular embodiment, the mixture of sucrose and mannitol consists of about 1% sucrose and about 3% mannitol. In certain embodiments, the formulation comprises between about 1 mM and about 10 mM calcium, or between about 2 mM and about 4 mM calcium. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In another embodiment, the present invention provides stabilized low salt ADAMTS13 variant(s) and/or ADAMTS13 protein formulations comprising 0.01 mg/ml to 10.0 mg/ml total ADAMTS13; less than about 100 mM of a pharmaceutically acceptable salt; 0.5 mM to 20 mM calcium; a sugar and/or sugar alcohol; between about 0.01% and 0.1% of a nonionic surfactant; and a buffering agent for maintaining a pH of between about 6.5 and about 7.5. In certain embodiments, the surfactant is selected from the group consisting of Polysorbate 20, Polysorbate 80, Pluronic F-68, BRIJ 35, and a combination thereof. In certain embodiments, the sugar and/or sugar alcohol is a mixture of sucrose and mannitol. In a particular embodiment, the surfactant is Polysorbate 80. In certain embodiments, the formulation comprises between about 1 mM and about 10 mM calcium, or between about 2 mM and about 4 mM calcium. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In another embodiment, the present invention provides stabilized low salt ADAMTS13 variant(s) and/or ADAMTS13 protein formulations comprising 0.01 mg/ml to 10.0 mg/ml total ADAMTS13; less than about 100 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5, wherein the buffering agent is histidine or HEPES. In certain embodiments, the buffering agent is present at a concentration between about 5 mM and about 100 mM, or between about 10 mM and about 50 mM. In another embodiment, the pH of the formulation is 7.0±0.2. In certain embodiments, the formulation comprises between about 1 mM and about 10 mM calcium, or between about 2 mM and about 4 mM calcium. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In another embodiment, the present invention provides stabilized low salt ADAMTS13 variant(s) and/or ADAMTS13 protein formulations comprising 0.01 mg/ml to 10.0 mg/ml total ADAMTS13; less than about 100 mM of a pharmaceutically acceptable salt; 0.5 mM to 20 mM calcium; a sugar and/or sugar alcohol; a nonionic surfactant; and a buffering agent for maintaining a pH of between about 6.5 and about 7.5. In certain embodiments, the formulation further comprises between about 0.5 μM and about 20 μM zinc. In certain embodiments, the formulation comprises between about 1 mM and about 10 mM calcium, or between about 2 mM and about 4 mM calcium. In certain embodiments, the pharmaceutically acceptable salt is sodium chloride or potassium chloride. In certain embodiments, the low salt ADAMTS13 variant(s) and/or ADAMTS13 protein formulation is a lyophilized formulation.

In certain embodiments, the present invention provides a stabilized low salt ADAMTS13 variant(s) and/or ADAMTS13 protein formulation comprising 0.01 mg/ml to 10.0 mg/ml total ADAMTS13; less than about 100 mM NaCl; 2 mM to 4 mM calcium; 2% to 4% mannitol; 0.5% to 2% sucrose; 0.025 to 0.1% Polysorbate 80; and 10 mM to 50 mM histidine (pH 7.0±0.2). In one embodiment, the formulation further comprises between about 0.5 μM and about 20 μM zinc. In certain embodiments, the low salt ADAMTS13 variant(s) and/or ADAMTS13 protein formulation is a lyophilized formulation.

In one embodiment, the present invention provides a formulation comprising (a) at least 0.01 units ADAMTS13 activity (i.e., FRETS-vWF73 activity) per mg ADAMTS13 variant or the combination of ADAMTS13 variant and ADAMTS13 (i.e., total ADAMTS13); (b) 0 mM to 200 mM of a pharmaceutically acceptable salt; (c) 0.5 mM to 20 mM calcium; (d) a sugar and/or sugar alcohol; (e) a nonionic surfactant; and (f) a buffering agent for maintaining a pH between 6.0 and 8.0. In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13. In one embodiment, the formulation comprises at least 200 units ADAMTS13 activity per mg ADAMTS13 variant or the combination of ADAMTS13 variant and ADAMTS13 (i.e., total ADAMTS13). In another embodiment, the stabilized formulation of ADAMTS13 variant(s) and/or ADAMTS13 protein comprises at least 400 units ADAMTS13 activity per mg total ADAMTS13. In certain embodiments, the stabilized formulation of ADAMTS13 variant(s) and/or ADAMTS13 protein comprises at least 600 units ADAMTS13 activity per mg total ADAMTS13. In a more embodiment, the stabilized formulation of ADAMTS13 variant(s) and/or ADAMTS13 protein comprises at least 800 units ADAMTS13 activity per mg total ADAMTS13. In yet another embodiment, the stabilized formulation of ADAMTS13 variant(s) and/or ADAMTS13 protein comprises at least 1000 units ADAMTS13 activity per mg total ADAMTS13. In one embodiment, the stabilized formulation of ADAMTS13 variant(s) and/or ADAMTS13 protein comprises between about 100 units and about 2000 units of ADAMTS13 activity per mg total ADAMTS13. In certain embodiments, the formulation is lyophilized, lyophilized from a liquid formulation described herein.

In one embodiment of the stabilized ADAMTS13 variant(s) and/or ADAMTS13 protein formulation, the formulation comprises between about 1.0 mM and about 10.0 mM calcium. In certain embodiments, the formulation contains between about 2.0 and about 4.0 mM calcium.

In another embodiment of the stabilized ADAMTS13 variant(s) and/or ADAMTS13 protein formulation, the formulation comprises between about 2% and about 6% of a sugar and/or sugar alcohol. In certain embodiments, the formulation comprises between about 3% and about 5% of a sugar and/or sugar alcohol. In a specific embodiment, the formulation comprises about 4% of a sugar and/or sugar alcohol. In one embodiment, the sugar and/or sugar alcohol is selected from the group consisting of sucrose, trehalose, mannitol, and a combination thereof. In certain embodiments, the sugar and/or sugar alcohol is a mixture of sucrose and mannitol.

In one embodiment of the stabilized ADAMTS13 variant(s) and/or ADAMTS13 protein formulation, the formulation comprises between about 0.01% and about 0.1% of a non-ionic surfactant. In certain embodiments, the formulation comprises about 0.05% of a non-ionic surfactant. In one embodiment, the surfactant is selected from the group consisting of Polysorbate 20, Polysorbate 80, Pluronic F-68, and BRIJ 35. In certain embodiments, the surfactant is Polysorbate 80.

In one embodiment of the stabilized ADAMTS13 variant(s) and/or ADAMTS13 protein formulation, the formulation comprises between about 5 mM and about 100 mM of a buffering agent. In certain embodiments, the formulation comprises between about 10 mM and about 50 mM of a buffering agent. In another embodiment, the buffering agent is histidine or HEPES. In certain embodiments, the buffering agent is histidine. In one embodiment, the pH of the formulation is between about 6.5 and 7.5. In certain embodiments, the pH of the formulation is 7.0±0.2.

In one embodiment of the stabilized ADAMTS13 variant(s) and/or ADAMTS13 protein formulation, the formulation further comprises between about 0.5 μM and 20 μM zinc.

In a specific embodiment, the present invention provides a stabilized formulation of ADAMTS13 variant(s) and/or ADAMTS13 protein comprising (a) at least 0.01 units ADAMTS13 activity per mg total ADAMTS13; (b) 0 to 200 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2).

In one embodiment, the present invention provides a formulation comprising (a) at least 0.01 units ADAMTS13 activity (i.e., FRETS-vWF73 activity) per mg ADAMTS13 variant or the combination of ADAMTS13 variant and ADAMTS13 (i.e., total ADAMTS13); (b) 0 mM to 100 mM of a pharmaceutically acceptable salt; (c) 0.5 mM to 20 mM calcium; (d) a sugar and/or sugar alcohol; (e) a nonionic surfactant; and (f) a buffering agent for maintaining a pH between 6.0 and 8.0. In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13. In one embodiment, the formulation comprises at least 200 units ADAMTS13 activity per mg total ADAMTS13. In another embodiment, the formulation comprises at least 400 units ADAMTS13 activity per mg total ADAMTS13. In another embodiment, the formulation comprises at least 600 units ADAMTS13 activity per mg total ADAMTS13. In another embodiment, the formulation comprises at least 800 units ADAMTS13 activity per mg total ADAMTS13. In yet another embodiment, the formulation comprises at least 1000 units ADAMTS13 activity per mg total ADAMTS13. In one embodiment, the formulation comprises between about 100 units and about 2000 units of ADAMTS13 activity per total ADAMTS13. In certain embodiments, the formulation is lyophilized, lyophilized from a liquid formulation described herein.

In one embodiment, the formulation comprises between about 1.0 mM and about 10.0 mM calcium. In another embodiment, the formulation contains between about 2.0 and about 4.0 mM calcium.

In another embodiment, the formulation comprises between about 2% and about 6% of a sugar and/or sugar alcohol. In another embodiment, the formulation comprises between about 3% and about 5% of a sugar and/or sugar alcohol. In certain embodiments, the formulation comprises about 4% of a sugar and/or sugar alcohol. In certain embodiments, the sugar and/or sugar alcohol is selected from the group consisting of sucrose, trehalose, mannitol, and a combination thereof. In another embodiment, the sugar and/or sugar alcohol is a mixture of sucrose and mannitol.

In another embodiment, the formulation comprises between about 0.01% and about 0.1% of a non-ionic surfactant. In another embodiment, the formulation comprises about 0.05% of a non-ionic surfactant. In certain embodiments, the surfactant is selected from the group consisting of Polysorbate 20, Polysorbate 80, Pluronic F-68, and BRIJ 35. In other embodiments, the surfactant is Polysorbate 80.

In another embodiment, the formulation comprises between about 5 mM and about 100 mM of a buffering agent. In another embodiment, the formulation comprises between about 10 mM and about 50 mM of a buffering agent. In certain embodiments, the buffering agent is histidine or HEPES. In another embodiment, the buffering agent is histidine. In yet another embodiment, the pH of the formulation is between about 6.5 and 7.5. In other embodiments, the pH of the formulation is 7.0±0.2.

In another embodiment, the formulation further comprises between about 0.5 μM and 20 μM zinc.

In certain embodiments, the present invention provides a formulation of ADAMTS13 variant(s) and/or wildtype ADAMTS13 comprising (a) at least 0.01 units ADAMTS13 activity per mg ADAMTS13 variant or the combination of ADAMTS13 variant and ADAMTS13 (i.e., total ADAMTS13); (b) 0 to 200 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13.

In certain embodiments, the present invention provides a formulation of ADAMTS13 variant(s) and/or ADAMTS13 protein comprising (a) at least 0.01 units ADAMTS13 activity per mg ADAMTS13 variant or total ADAMTS13; (b) 0 to 100 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13.

In another embodiment, the present invention provides a formulation of ADAMTS13 variant(s) and/or ADAMTS13 protein, comprising (a) at least 0.01 units ADAMTS13 activity per mg ADAMTS13 variant total ADAMTS13; (b) 0 to 60 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13.

In another embodiment, the present invention provides a lyophilized formulation of ADAMTS13 variant(s) and/or ADAMTS13 protein, wherein the formulation is lyophilized from a liquid formulation comprising (a) at least 0.01 units ADAMTS13 activity per mg ADAMTS13 variant or total ADAMTS13; (b) 0 to 200 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13.

In another embodiment, the present invention provides a lyophilized formulation of ADAMTS13 variant(s) and/or ADAMTS13 protein, wherein the formulation is lyophilized from a liquid formulation comprising (a) at least 0.01 units ADAMTS13 activity per mg ADAMTS13 variant or total ADAMTS13; (b) 0 to 100 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13.

In yet another embodiment, the present invention provides a low salt lyophilized formulation of ADAMTS13 variant(s) and/or ADAMTS13 protein, wherein the formulation is lyophilized from a liquid formulation comprising (a) at least 0.01 units ADAMTS13 activity per mg ADAMTS13 variant or total ADAMTS13; (b) 0 to 60 mM NaCl; (c) 2 mM to 4 mM calcium; (d) 2% to 4% mannitol; (e) 0.5% to 2% sucrose; (f) 0.025 to 0.1% Polysorbate 80; and (g) 10 mM to 50 mM histidine (pH 7.0±0.2). In one embodiment, the stabilized formulation of ADAMTS13 variant(s) and/or ADAMTS13 protein comprises at least 10 units ADAMTS13 activity per mg total ADAMTS13. In one embodiment, the stabilized formulation of ADAMTS13 variant(s) and/or ADAMTS13 protein comprises at least 100 units ADAMTS13 activity per mg total ADAMTS13.

### IV. Methods of Treatment

The compositions described herein can be administered for therapeutic or prophylactic treatments. Generally, for therapeutic applications, compositions are administered to a subject with a disease or condition associated with ADAMTS13 or VWF dysfunction or otherwise in need thereof, in a “therapeutically effective dose.” In certain embodiments, a composition described herein is used for the treatment and prophylaxis of thrombotic diseases and conditions. In certain embodiments, the composition described herein is used for the treatment and prophylaxis of an infarction. Compositions and amounts effective for these uses will depend upon the severity of the disease or condition and the general state of the patient's health. Single or multiple administrations of the compositions may be administered depending on the dosage and frequency as required and tolerated by the patient.

In one embodiment, the ADAMTS13 variant either alone or together with ADAMTS13 protein (i.e., total ADAMTS13) is administered at a dose of from 0.01 UFV73/kg body weight (i.e., IU/kg body weight) to 10000 UFV73/kg body weight (i.e., IU/kg body weight) of total ADAMTS13. In one embodiment, the ADAMTS13 variant either alone or together with ADAMTS13 protein is administered at a dose of from 0.1 UFV73/kg body weight to 10000 UFV73/kg body weight. In one embodiment, the ADAMTS13 variant either alone or together with ADAMTS13 protein is administered at a dose of from 0.1 UFV73/kg body weight to 6000 UFV73/kg body weight, from 0.1 UFV73/kg body weight to 5000 UFV73/kg body weight, from 0.1 UFV73/kg body weight to 4000 UFV73/kg body weight, from 0.1 UFV73/kg body weight to 3000 UFV73/kg body weight, from 0.1 UFV73/kg body weight to 2000 UFV73/kg body weight, from 0.1 UFV73/kg body weight to 1000 UFV73/kg body weight, from 0.1 UFV73/kg body weight to 500 UFV73/kg body weight, from 0.1 UFV73/kg body weight to 200 UFV73/kg body weight, from 0.1 UFV73/kg body weight to 160 UFV73/kg body weight, from 0.1 UFV73/kg body weight to 100 UFV73/kg body weight, from 0.1 UFV73/kg body weight to 80 UFV73/kg body weight, from 0.1 UFV73/kg body weight to 40 UFV73/kg body weight, from 0.1 UFV73/kg body weight to 20 UFV73/kg body weight, or from 0.1 UFV73/kg body weight to 10 UFV73/kg body weight. In one embodiment, the ADAMTS13 variant either alone or together with ADAMTS13 protein is administered at a dose of from 1 UFV73/kg body weight to 10000 UFV73/kg body weight, from 1 UFV73/kg body weight to 6000 UFV73/kg body weight, from 1 UFV73/kg body weight to 5000 UFV73/kg body weight, from 1 UFV73/kg body weight to 4000 UFV73/kg body weight, from 1 UFV73/kg body weight to 3000 UFV73/kg body weight, from 1 UFV73/kg body weight to 2000 UFV73/kg body weight, from 1 UFV73/kg body weight to 1000 UFV73/kg body weight, from 1 UFV73/kg body weight to 500 UFV73/kg body weight, from 1 UFV73/kg body weight to 200 UFV73/kg body weight, from 1 UFV73/kg body weight to 160 UFV73/kg body weight, from 1 UFV73/kg body weight to 100 UFV73/kg body weight, from 1 UFV73/kg body weight to 80 UFV73/kg body weight, from 1 UFV73/kg body weight to 40 UFV73/kg body weight, from 1 UFV73/kg body weight to 20 UFV73/kg body weight, or from 1 UFV73/kg body weight to 10 UFV73/kg body weight. In one embodiment, the ADAMTS13 variant either alone or together with ADAMTS13 protein is administered at a dose of from 5 UFV73/kg body weight to 10000 UFV73/kg body weight, from 5 UFV73/kg body weight to 6000 UFV73/kg body weight, from 5 UFV73/kg body weight to 5000 UFV73/kg body weight, from 5 UFV73/kg body weight to 4000 UFV73/kg body weight, from 5 UFV73/kg body weight to 3000 UFV73/kg body weight, from 5 UFV73/kg body weight to 2000 UFV73/kg body weight, from 5 UFV73/kg body weight to 1000 UFV73/kg body weight, from 5 UFV73/kg body weight to 500 UFV73/kg body weight, from 5 UFV73/kg body weight to 200 UFV73/kg body weight, from 5 UFV73/kg body weight to 160 UFV73/kg body weight, or from 5 UFV73/kg body weight to 100 UFV73/kg body weight, from 5 UFV73/kg body weight to 80 UFV73/kg body weight, from 5 UFV73/kg body weight to 40 UFV73/kg body weight, from 5 UFV73/kg body weight to 20 UFV73/kg body weight, or from 5 UFV73/kg body weight to 10 UFV73/kg body weight. In one embodiment, the ADAMTS13 variant either alone or together with ADAMTS13 protein is administered at a dose of from 10 UFV73/kg body weight to 10000 UFV73/kg body weight, from 10 UFV73/kg body weight to 6000 UFV73/kg body weight, from 10 UFV73/kg body weight to 5000 UFV73/kg body weight, from 10 UFV73/kg body weight to 4000 UFV73/kg body weight, from 10 UFV73/kg body weight to 3000 UFV73/kg body weight, from 10 UFV73/kg body weight to 2000 UFV73/kg body weight, from 10 UFV73/kg body weight to 1000 UFV73/kg body weight, from 10 UFV73/kg body weight to 500 UFV73/kg body weight, from 10 UFV73/kg body weight to 200 UFV73/kg body weight, from 10 UFV73/kg body weight to 160 UFV73/kg body weight, from 10 UFV73/kg body weight to 100 UFV73/kg body weight, from 10 UFV73/kg body weight to 80 UFV73/kg body weight, from 10 UFV73/kg body weight to 40 UFV73/kg body weight, or from 10 UFV73/kg body weight to 20 UFV73/kg body weight. In one embodiment, the ADAMTS13 variant either alone or together with ADAMTS13 protein is administered at a dose of from 20 UFV73/kg body weight to 10000 UFV73/kg body weight, from 20 UFV73/kg body weight to 6000 UFV73/kg body weight, from 20 UFV73/kg body weight to 5000 UFV73/kg body weight, from 20 UFV73/kg body weight to 4000 UFV73/kg body weight, from 20 UFV73/kg body weight to 3000 UFV73/kg body weight, from 20 UFV73/kg body weight to 2000 UFV73/kg body weight, from 20 UFV73/kg body weight to 1000 UFV73/kg body weight, from 20 UFV73/kg body weight to 500 UFV73/kg body weight, from 20 UFV73/kg body weight to 200 UFV73/kg body weight, from 20 UFV73/kg body weight to 160 UFV73/kg body weight, from 20 UFV73/kg body weight to 100 UFV73/kg body weight, from 20 UFV73/kg body weight to 80 UFV73/kg body weight, or from 20 UFV73/kg body weight to 40 UFV73/kg body weight. In one embodiment, the ADAMTS13 variant either alone or together with ADAMTS13 protein is administered at a dose of from 25 UFV73/kg body weight to 10000 UFV73/kg body weight, from 25 UFV73/kg body weight to 6000 UFV73/kg body weight, from 25 UFV73/kg body weight to 5000 UFV73/kg body weight, from 25 UFV73/kg body weight to 4000 UFV73/kg body weight, from 25 UFV73/kg body weight to 3000 UFV73/kg body weight, from 25 UFV73/kg body weight to 2000 UFV73/kg body weight, from 25 UFV73/kg body weight to 1000 UFV73/kg body weight, from 25 UFV73/kg body weight to 500 UFV73/kg body weight, from 25 UFV73/kg body weight to 400 UFV73/kg body weight, from 25 UFV73/kg body weight to 200 UFV73/kg body weight, from 25 UFV73/kg body weight to 160 UFV73/kg body weight, from 25 UFV73/kg body weight to 100 UFV73/kg body weight, from 25 UFV73/kg body weight to 80 UFV73/kg body weight, or from 25 UFV73/kg body weight to 40 UFV73/kg body weight. In one embodiment, the ADAMTS13 variant either alone or together with ADAMTS13 protein is administered at a dose of from 40 UFV73/kg body weight to 10000 UFV73/kg body weight, from 40 UFV73/kg body weight to 6000 UFV73/kg body weight, from 40 UFV73/kg body weight to 5000 UFV73/kg body weight, from 40 UFV73/kg body weight to 4000 UFV73/kg body weight, from 40 UFV73/kg body weight to 3000 UFV73/kg body weight, from 40 UFV73/kg body weight to 2000 UFV73/kg body weight, from 40 UFV73/kg body weight to 1000 UFV73/kg body weight, from 40 UFV73/kg body weight to 500 UFV73/kg body weight, from 40 UFV73/kg body weight to 200 UFV73/kg body weight, from 40 UFV73/kg body weight to 160 UFV73/kg body weight, from 40 UFV73/kg body weight to 100 UFV73/kg body weight, or from 40 UFV73/kg body weight to 80 UFV73/kg body weight. In one embodiment, the ADAMTS13 variant either alone or together with ADAMTS13 protein is administered at a dose of from 50 UFV73/kg body weight to 10000 UFV73/kg body weight, from 50 UFV73/kg body weight to 6000 UFV73/kg body weight, from 50 UFV73/kg body weight to 5000 UFV73/kg body weight, from 50 UFV73/kg body weight to 4000 UFV73/kg body weight, from 50 UFV73/kg body weight to 3000 UFV73/kg body weight, from 50 UFV73/kg body weight to 2000 UFV73/kg body weight, from 50 UFV73/kg body weight to 1000 UFV73/kg body weight, from 50 UFV73/kg body weight to 500 UFV73/kg body weight, from 50 UFV73/kg body weight to 200 UFV73/kg body weight, from 50 UFV73/kg body weight to 160 UFV73/kg body weight, or from 50 UFV73/kg body weight to 100 UFV73/kg body weight. In one embodiment, the ADAMTS13 variant either alone or together with ADAMTS13 protein is administered at a dose of from 100 UFV73/kg body weight to 10000 UFV73/kg body weight, from 100 UFV73/kg body weight to 6000 UFV73/kg body weight, from 100 UFV73/kg body weight to 5000 UFV73/kg body weight, from 100 UFV73/kg body weight to 4000 UFV73/kg body weight, from 100 UFV73/kg body weight to 3000 UFV73/kg body weight, from 100 UFV73/kg body weight to 2000 UFV73/kg body weight, from 100 UFV73/kg body weight to 1000 UFV73/kg body weight, from 100 UFV73/kg body weight to 500 UFV73/kg body weight, from 100 UFV73/kg body weight to 200 UFV73/kg body weight, or from 100 UFV73/kg body weight to 160 UFV73/kg body weight.

Similarly, in certain embodiments, the ADAMTS13 variant(s) and/or ADAMTS13 protein is administered at a dose between about 0.01 UFV73/kg body weight (i.e., IU/kg body weight) and about 10,000 UFV73/kg body weight of total ADAMTS13. In other embodiments, the dose may be between about 1 UFV73/kg body weight and about 10,000 UFV73/kg body weight, or between about 20 UFV73/kg body weight and about 8,000 UFV73/kg body weight, or between about 30 UFV73/kg body weight and about 6,000 UFV73/kg body weight, or between about 40 UFV73/kg body weight and about 4,000 UFV73/kg body weight, or between about 50 UFV73/kg body weight and about 3,000 UFV73/kg body weight, or between about 75 UFV73/kg body weight and about 2,500 UFV73/kg body weight, or between about 100 UFV73/kg body weight and about 2,000 UFV73/kg body weight, or between about 200 UFV73/kg body weight and about 1,500 UFV73/kg body weight, or between about other ranges therein of total ADAMTS13. In certain embodiments, the dose may be between about 150 UFV73/kg body weight and about 600 UFV73/kg body weight. In certain embodiments the dose may be between about 100 UFV73/kg body weight and about 1,000 UFV73/kg body weight of total ADAMTS13. In certain embodiments, the dose may be about 0.01 UFV73/kg body weight, or about 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 160, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more UFV73/kg body weight.

In other embodiments, the ADAMTS13 variant either alone or together with ADAMTS13 protein (i.e., total ADAMTS13) is administered at about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,100, 5,200, 5,300, 5,400, 5,500, 5,600, 5,700, 5,800, 5,900, or 6,000 UFV73/kg body weight, or at an intermediate concentration or concentration range thereof. In certain embodiments, the ADAMTS13 variant either alone or together with ADAMTS13 protein (i.e., total ADAMTS13) is administered at about 10, 20, 40, 80, or 160 UFV73/kg body weight.

In certain embodiments, the disclosure provides a method for treating or preventing a disease or condition, the method comprising administering to a subject in need thereof a composition according to any one of the compositions provided herein.

In some embodiments, the ADAMTS13 variant comprises the amino acid sequence set forth in SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the nucleotide sequence that encodes the ADAMTS13 variant comprises the nucleotide sequence that encodes the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the ADAMTS13 variant comprises the amino acid sequence set forth in SEQ ID NO: 2. In certain embodiments, the ADAMTS13 variant consists of the amino acid sequence set forth in SEQ ID NO: 2. In certain embodiments, the ADAMTS13 variant consists essentially of the amino acid sequence set forth in SEQ ID NO: 2.

In some embodiments, the ADAMTS13 protein comprises the amino acid sequence set forth in SEQ ID NO: 1, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In certain embodiments, the nucleotide sequence that encodes the ADAMTS13 protein comprises the nucleotide sequence that encodes the amino acid sequence of SEQ ID NO: 1, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In certain embodiments, the ADAMTS13 protein comprises the amino acid sequence set forth in SEQ ID NO: 1. In certain embodiments, the ADAMTS13 protein consists of the amino acid sequence set forth in SEQ ID NO: 1. In certain embodiments, the ADAMTS13 protein consists essentially of the amino acid sequence set forth in SEQ ID NO: 1.

In certain embodiments, the ADAMTS13 variant or composition thereof, including compositions with ADAMTS13, is administered to the subject to treat or prevent the disease or condition. For example, the ADAMTS13 variant, with or without ADAMTS13 protein, is administered in a singular bolus injection or in multiple doses to maintain a circulating level of total ADAMTS13 effective to treat or prevent the disease or condition. In such aspects, the composition comprising ADAMTS13 variant or composition thereof, including compositions with ADAMTS13, is administered monthly, every two weeks, weekly, twice a week, every other day, daily, every 12 hours, every 8 hours, every six hours, every four hours, every two hours, or every hour. In particular aspects, the injection is administered subcutaneously. In other aspects, the injection is administered intravenously.

In certain embodiments, an ADAMTS13 variant or composition thereof, including compositions with ADAMTS13, is administered immediately upon discovery of the disease or condition, e.g., within 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 110 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 or more hours, or any combination thereof.

In certain embodiments, the bioavailability of the ADAMTS13 variant(s) and/or ADAMTS13 protein after subcutaneous administration is at least about 40%, or at least about 45%, or at least about 50%, or at least about 51%, or at least about 52%, or at least about 53%, or at least about 54%, or at least about 55%, or at least about 56%, or at least about 57%, or at least about 58%, or at least about 59%, or at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85% as compared to intravenous administration normalized for the same dose.

In certain embodiments, the bioavailability of the ADAMTS13 variant(s) and/or ADAMTS13 protein after subcutaneous administration is between about 30% to about 90% or about 80% or about 50% as compared to intravenous administration normalized for the same dose. In certain embodiments, the bioavailability of the ADAMTS13 variant(s) and/or ADAMTS13 protein after subcutaneous administration is between about 60% and about 80% as compared to intravenous administration normalized for the same dose. In certain embodiments, the bioavailability of the ADAMTS13 variant(s) and/or ADAMTS13 protein after subcutaneous administration is between about 50% and about 70% as compared to intravenous administration normalized for the same dose. In certain embodiments, the bioavailability of the ADAMTS13 variant(s) and/or ADAMTS13 protein after subcutaneous administration is between about 55% and about 70% as compared to intravenous administration normalized for the same dose. In certain embodiments, the bioavailability of the ADAMTS13 variant(s) and/or ADAMTS13 protein after subcutaneous administration is between about 55% and about 65% as compared to intravenous administration normalized for the same dose. In certain embodiments, the bioavailability of the ADAMTS13 variant(s) and/or ADAMTS13 protein after subcutaneous administration is about 65% as compared to intravenous administration normalized for the same dose.

In certain embodiments, the bioavailability of the ADAMTS13 variant(s) and/or ADAMTS13 protein after subcutaneous administration is about 65% as compared to intravenous administration normalized for the same dose. Thus, in certain embodiments, if the therapeutically effective amount of total ADAMTS13 comprises at least 20-160 international units per kilogram (IU/kg) body weight via intravenous administration, and the bioavailability is 65%, if ±15% variation is applied, there would be 40-80% bioavailability resulting in a 25-400 international units range when administered subcutaneously.

In certain embodiments, the disclosure provides a method for treating or preventing a disease or condition with the formation and/or presence of a thrombus, the method comprising administering to a subject in need thereof a composition according to any one of the compositions provided herein.

In certain embodiments, the present disclosure provides a method for treating or preventing by way of example but not limitation, a blood clotting disorder (e.g., inherited TTP, acquired TTP, infarction, cerebral infarction, myocardial infarction, ischemic/reperfusion injury, deep vein thrombosis (DVT), sepsis-related disseminated intravascular coagulation (DIC)), a bleeding episode (e.g., associated with associated with inherited TTP, acquired TTP, infarction, myocardial infarction, cerebral infarction, ischemia reperfusion injury), myocardial infarction, cerebral infarction, deep vein thrombosis, ischemic/reperfusion injury, DIC, sickle cell disease, vaso-occlusive crisis, acute lung injury, acute respiratory distress syndrome, liver disease (e.g., liver failure, portal vein thrombosis, and Budd-Chiari syndrome), renal disease (e.g., hemolytic uremic syndrome, and renal vein thrombosis), organ transplant rejection in a subject, the method comprising administering to the subject in need thereof a therapeutically effective amount of an ADAMTS13 variant or composition thereof, including compositions with ADAMTS13.

In certain embodiments, the present disclosure provides a method for treating or preventing a blood clotting disorder in a subject, the method comprising administering to the subject in need thereof a therapeutically effective amount of an ADAMTS13 variant or composition thereof, including compositions with ADAMTS13. In certain embodiments of the methods provided herein, the blood clotting disorder includes, but is not limited to, inherited TTP, acquired TTP, infarction, cerebral infarction, myocardial infarction, ischemic/reperfusion injury, deep vein thrombosis (DVT), and sepsis-related disseminated intravascular coagulation (DIC). In one embodiment of the methods provided herein, the clotting disorder is inherited TTP. In one embodiment of the methods provided herein, the clotting disorder is acquired TTP.

In certain embodiments, the ADAMTS13 variant or composition thereof, including compositions with ADAMTS13, is administered to the subject to treat or prevent the blood clotting disorder. For example, the ADAMTS13 variant, with or without ADAMTS13 protein, is administered in a singular bolus injection or in multiple doses to maintain a circulating level of total ADAMTS13 effective to treat or prevent the blood clotting disorder. In such aspects, the composition comprising ADAMTS13 variant or composition thereof, including compositions with ADAMTS13, is administered monthly, every two weeks, weekly, twice a week, every other day, daily, every 12 hours, every 8 hours, every six hours, every four hours, every two hours, or every hour. In particular aspects, the injection is administered subcutaneously. In other aspects, the injection is administered intravenously.

In certain embodiments, an ADAMTS13 variant or composition thereof, including compositions with ADAMTS13, is administered immediately upon discovery of the blood clotting disorder, e.g., within 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 110 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 or more hours, or any combination thereof.

In one aspect, the present disclosure provides a method for treating a bleeding episode in a subject, the method comprising administering to the subject in need thereof a therapeutically effective amount of an ADAMTS13 variant or composition thereof, including compositions with ADAMTS13. In certain embodiments, the bleeding episode is associated with inherited TTP, acquired TTP, infarction, myocardial infarction, cerebral infarction, and/or ischemia reperfusion injury.

In certain embodiments, the ADAMTS13 variant or composition thereof, including compositions with ADAMTS13, is administered to the subject to treat or prevent the bleeding episode. For example, the ADAMTS13 variant, with or without ADAMTS13 protein, is administered in a singular bolus injection or in multiple doses to maintain a circulating level of total ADAMTS13 effective to treat or prevent the bleeding episode. In such aspects, the composition comprising ADAMTS13 variant or composition thereof, including compositions with ADAMTS13, is administered monthly, every two weeks, weekly, twice a week, every other day, daily, every 12 hours, every 8 hours, every six hours, every four hours, every two hours, or every hour. In particular aspects, the injection is administered subcutaneously. In other aspects, the injection is administered intravenously.

In certain embodiments, an ADAMTS13 variant or composition thereof, including compositions with ADAMTS13, is administered immediately upon discovery of the bleeding episode, e.g., within 5 minutes, 10 minutes, 15 minutes, 20 minutes, 25 minutes, 30 minutes, 35 minutes, 40 minutes, 45 minutes, 50 minutes, 55 minutes, 60 minutes, 90 minutes, 110 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 or more hours, or any combination thereof.

In certain embodiments, the disclosure provides a method for treating or preventing inherited TTP. Inherited TTP is due to genetic mutations of the ADAMTS13 gene. Inherited TTP can lead to neurologic manifestations (e.g., mental status, stroke, seizures, hemiplegia, paresthesias, visual disturbance, and aphasia), fatigue, and severe bleeding. If left untreated, acquired TTP can be fatal or can cause lasting physiological damage. Furthermore, because inherited TTP is due to a genetic mutation, life-long treatment is needed and patient compliance is required.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 1 to about 4000 units of FRETS-VWF73 activity per kilogram body weight of the mammal (IU/kg). In a specific embodiment, the mammal is a human. In one embodiment, the about 1 IU/kg to about 4000 IU/kg total ADAMTS13 is administered about once a month. In one embodiment, the about 1 IU/kg to about 4000 IU/kg total ADAMTS13 is administered about twice a month. In one embodiment, the about 1 IU/kg to about 4000 IU/kg total ADAMTS13 is administered about once a week. In one embodiment, the about 1 IU/kg to about 4000 IU/kg total ADAMTS13 is administered about twice a week. In one embodiment, the about 1 IU/kg to about 4000 IU/kg total ADAMTS13 is administered about three times a week. In one embodiment, the about 1 IU/kg to about 4000 IU/kg total ADAMTS13 is administered about once every 48 hours. In one embodiment, the about 1 IU/kg to about 4000 IU/kg total ADAMTS13 is administered about once every 24 hours. In one embodiment, the about 1 IU/kg to about 4000 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 5 to about 4000 units of FRETS-VWF73 activity per kilogram body weight of the mammal (IU/kg). In a specific embodiment, the mammal is a human. In one embodiment, the about 5 IU/kg to about 4000 IU/kg total ADAMTS13 is administered about once a month. In one embodiment, the about 5 IU/kg to about 4000 IU/kg total ADAMTS13 is administered about twice a month. In one embodiment, the about 5 IU/kg to about 4000 IU/kg total ADAMTS13 is administered about once a week. In one embodiment, the about 5 IU/kg to about 4000 IU/kg total ADAMTS13 is administered about twice a week. In one embodiment, the about 5 IU/kg to about 4000 IU/kg total ADAMTS13 is administered about three times a week. In one embodiment, the about 5 IU/kg to about 4000 IU/kg total ADAMTS13 is administered about once every 48 hours. In one embodiment, the about 5 IU/kg to about 4000 IU/kg total ADAMTS13 is administered about once every 24 hours. In one embodiment, the 5 IU/kg to about 4000 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 4000 units of FRETS-VWF73 activity per kilogram body weight of the mammal (IU/kg). In a specific embodiment, the mammal is a human. In one embodiment, the about 20 IU/kg to about 4000 IU/kg total ADAMTS13 is administered about once a month. In one embodiment, the about 20 IU/kg to about 4000 IU/kg total ADAMTS13 is administered about twice a month. In one embodiment, the about 20 IU/kg to about 4000 IU/kg total ADAMTS13 is administered about once a week. In one embodiment, the about 20 IU/kg to about 4000 IU/kg total ADAMTS13 is administered about twice a week. In one embodiment, the about 20 IU/kg to about 4000 IU/kg total ADAMTS13 is administered about three times a week. In one embodiment, the about 20 IU/kg to about 4000 IU/kg total ADAMTS13 is administered about once every 48 hours. In one embodiment, the about 20 IU/kg to about 4000 IU/kg total ADAMTS13 is administered about once every 24 hours. In one embodiment, the 20 IU/kg to about 4000 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 2000 units of FRETS-VWF73 activity per kilogram body weight of the mammal (IU/kg). In a specific embodiment, the mammal is a human. In one embodiment, the about 20 IU/kg to about 2000 IU/kg total ADAMTS13 is administered about once a month. In one embodiment, the about 20 IU/kg to about 2000 IU/kg total ADAMTS13 is administered about twice a month. In one embodiment, the about 20 IU/kg to about 2000 IU/kg total ADAMTS13 is administered about once a week. In one embodiment, the about 20 IU/kg to about 2000 IU/kg total ADAMTS13 is administered about twice a week. In one embodiment, the about 20 IU/kg to about 2000 IU/kg total ADAMTS13 is administered about three times a week. In one embodiment, the about 20 IU/kg to about 2000 IU/kg total ADAMTS13 is administered about once every 48 hours. In one embodiment, the about 20 IU/kg to about 2000 IU/kg total ADAMTS13 is administered about once every 24 hours. In one embodiment, the 20 IU/kg to about 2000 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 1000 units of FRETS-VWF73 activity per kilogram body weight of the mammal (IU/kg). In a specific embodiment, the mammal is a human. In one embodiment, the about 20 IU/kg to about 1000 IU/kg total ADAMTS13 is administered about once a month. In one embodiment, the about 20 IU/kg to about 1000 IU/kg total ADAMTS13 is administered about twice a month. In one embodiment, the about 20 IU/kg to about 1000 IU/kg total ADAMTS13 is administered about once a week. In one embodiment, the about 20 IU/kg to about 1000 IU/kg total ADAMTS13 is administered about twice a week. In one embodiment, the about 20 IU/kg to about 1000 IU/kg total ADAMTS13 is administered about three times a week. In one embodiment, the about 20 IU/kg to about 1000 IU/kg total ADAMTS13 is administered about once every 48 hours. In one embodiment, the about 20 IU/kg to about 1000 IU/kg total ADAMTS13 is administered about once every 24 hours. In one embodiment, the 20 IU/kg to about 1000 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 5 to about 500 units of FRETS-VWF73 activity per kilogram body weight of the mammal (IU/kg). In a specific embodiment, the mammal is a human. In one embodiment, the about 5 IU/kg to about 500 IU/kg total ADAMTS13 is administered about once a month. In one embodiment, the about 5 IU/kg to about 500 IU/kg total ADAMTS13 is administered about twice a month. In one embodiment, the about 5 IU/kg to about 500 IU/kg total ADAMTS13 is administered about once a week. In one embodiment, the about 5 IU/kg to about 500 IU/kg total ADAMTS13 is administered about twice a week. In one embodiment, the about 5 IU/kg to about 500 IU/kg total ADAMTS13 is administered about three times a week. In one embodiment, the about 5 IU/kg to about 500 IU/kg total ADAMTS13 is administered about once every 48 hours. In one embodiment, the about 5 IU/kg to about 500 IU/kg total ADAMTS13 is administered about once every 24 hours. In one embodiment, the 5 IU/kg to about 500 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 10 to about 1,500 units of FRETS-VWF73 activity per kilogram body weight of the mammal (IU/kg). In a specific embodiment, the mammal is a human. In one embodiment, the about 10 IU/kg to about 1,500 IU/kg total ADAMTS13 is administered about once a month. In one embodiment, the about 10 IU/kg to about 1,500 IU/kg total ADAMTS13 is administered about twice a month. In one embodiment, the about 10 IU/kg to about 1,500 IU/kg total ADAMTS13 is administered about once a week. In one embodiment, the about 10 IU/kg to about 1,500 IU/kg total ADAMTS13 is administered about twice a week. In one embodiment, the about 10 IU/kg to about 1,500 IU/kg total ADAMTS13 is administered about once every 48 hours. In one embodiment, the about 10 IU/kg to about 1,500 IU/kg total ADAMTS13 is administered about once every 24 hours. In one embodiment, the about 10 IU/kg to about 1,500 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 500 units of FRETS-VWF73 activity per kilogram body weight of the mammal (IU/kg). In a specific embodiment, the mammal is a human. In one embodiment, the about 20 IU/kg to about 500 IU/kg total ADAMTS13 is administered about once a month. In one embodiment, the about 5 IU/kg to about 500 IU/kg total ADAMTS13 is administered about twice a month. In one embodiment, the about 20 IU/kg to about 500 IU/kg total ADAMTS13 is administered about once a week. In one embodiment, the about 20 IU/kg to about 500 IU/kg total ADAMTS13 is administered about twice a week. In one embodiment, the about 20 IU/kg to about 500 IU/kg total ADAMTS13 is administered about three times a week. In one embodiment, the about 20 IU/kg to about 500 IU/kg total ADAMTS13 is administered about once every 48 hours. In one embodiment, the about 20 IU/kg to about 500 IU/kg total ADAMTS13 is administered about once every 24 hours. In one embodiment, the 20 IU/kg to about 500 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 200 units of FRETS-VWF73 activity per kilogram body weight of the mammal (IU/kg). In a specific embodiment, the mammal is a human. In one embodiment, the about 20 IU/kg to about 200 IU/kg total ADAMTS13 is administered about once a month. In one embodiment, the about 5 IU/kg to about 200 IU/kg total ADAMTS13 is administered about twice a month. In one embodiment, the about 20 IU/kg to about 200 IU/kg total ADAMTS13 is administered about once a week. In one embodiment, the about 20 IU/kg to about 200 IU/kg total ADAMTS13 is administered about twice a week. In one embodiment, the about 20 IU/kg to about 200 IU/kg total ADAMTS13 is administered about three times a week. In one embodiment, the about 20 IU/kg to about 200 IU/kg total ADAMTS13 is administered about once every 48 hours. In one embodiment, the about 20 IU/kg to about 200 IU/kg total ADAMTS13 is administered about once every 24 hours. In one embodiment, the 20 IU/kg to about 200 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 100 units of FRETS-VWF73 activity per kilogram body weight of the mammal (IU/kg). In a specific embodiment, the mammal is a human. In one embodiment, the about 20 IU/kg to about 100 IU/kg total ADAMTS13 is administered about once a month. In one embodiment, the about 5 IU/kg to about 100 IU/kg total ADAMTS13 is administered about twice a month. In one embodiment, the about 20 IU/kg to about 100 IU/kg total ADAMTS13 is administered about once a week. In one embodiment, the about 20 IU/kg to about 100 IU/kg total ADAMTS13 is administered about twice a week. In one embodiment, the about 20 IU/kg to about 100 IU/kg total ADAMTS13 is administered about three times a week. In one embodiment, the about 20 IU/kg to about 100 IU/kg total ADAMTS13 is administered about once every 48 hours. In one embodiment, the about 20 IU/kg to about 100 IU/kg total ADAMTS13 is administered about once every 24 hours. In one embodiment, the 20 IU/kg to about 100 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 40 to about 200 units of FRETS-VWF73 activity per kilogram body weight of the mammal (IU/kg). In a specific embodiment, the mammal is a human. In one embodiment, the about 40 IU/kg to about 200 IU/kg total ADAMTS13 is administered about once a month. In one embodiment, the about 5 IU/kg to about 200 IU/kg total ADAMTS13 is administered about twice a month. In one embodiment, the about 40 IU/kg to about 200 IU/kg total ADAMTS13 is administered about once a week. In one embodiment, the about 40 IU/kg to about 200 IU/kg total ADAMTS13 is administered about twice a week. In one embodiment, the about 40 IU/kg to about 200 IU/kg total ADAMTS13 is administered about three times a week. In one embodiment, the about 40 IU/kg to about 200 IU/kg total ADAMTS13 is administered about once every 48 hours. In one embodiment, the about 40 IU/kg to about 200 IU/kg total ADAMTS13 is administered about once every 24 hours. In one embodiment, the 40 IU/kg to about 200 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 10 to about 160 units of FRETS-VWF73 activity per kilogram body weight of the mammal (IU/kg). In a specific embodiment, the mammal is a human. In one embodiment, the about 10 IU/kg to about 160 IU/kg total ADAMTS13 is administered about once a month. In one embodiment, the about 10 IU/kg to about 160 IU/kg total ADAMTS13 is administered about twice a month. In one embodiment, the about 10 IU/kg to about 160 IU/kg total ADAMTS13 is administered about once a week. In one embodiment, the about 10 IU/kg to about 160 IU/kg total ADAMTS13 is administered about twice a week. In one embodiment, the about 10 IU/kg to about 160 IU/kg total ADAMTS13 is administered about once every 48 hours. In one embodiment, the about 10 IU/kg to about 160 IU/kg total ADAMTS13 is administered about once every 24 hours. In one embodiment, the about 10 IU/kg to about 160 IU/kg total ADAMTS13 is administered about once every 12 hours. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 160 units of FRETS-VWF73 activity per kilogram body weight of the mammal (IU/kg). In a specific embodiment, the mammal is a human. In one embodiment, the about 20 IU/kg to about 160 IU/kg total ADAMTS13 is administered about once a month. In one embodiment, the about 20 to about 160 IU/kg total ADAMTS13 is administered about twice a month. In one embodiment, the about 20 to about 160 IU/kg total ADAMTS13 is administered about once a week. In one embodiment, the about 20 to about 160 IU/kg total ADAMTS13 is administered about twice a week. In one embodiment, the about 20 to about 160 IU/kg total ADAMTS13 is administered about once every 48 hours. In one embodiment, the about 20 to about 160 IU/kg total ADAMTS13 is administered about once every 24 hours. In one embodiment, the about 20 to about 160 IU/kg total ADAMTS13 is administered about once every 12 hours. In certain embodiments, the total ADAMTS13 is about 20 IU/kg, about 40 IU/kg, about 50 IU/kg, about 60 IU/kg, about 70 IU/kg, about 75 IU/kg, about 80 IU/kg, about 90 IU/kg, about 100 IU/kg, about 100 IU/kg, about 120 IU/kg, about 125 IU/kg, about 130 IU/kg, about 140 IU/kg, about 150 IU/kg, about 160 IU/kg. In certain aspects, the injection is administered intravenously. In other aspects, the injection is administered subcutaneously.

In one embodiment, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 40 units of FRETS-VWF73 activity per kilogram body weight of the mammal (IU/kg). In a specific embodiment, the mammal is a human. In one embodiment, the about 20 IU/kg to about 40 IU/kg total ADAMTS13 is administered about once a month. In one embodiment, the about 20 to about 40 IU/kg total ADAMTS13 is administered about twice a month. In one embodiment, the about 20 to about 40 IU/kg total ADAMTS13 is administered about once a week. In one embodiment, the about 20 to about 40 IU/kg total ADAMTS13 is administered about twice a week. In one embodiment, the about 20 to about 40 IU/kg total ADAMTS13 is administered about once every 48 hours. In one embodiment, the about 20 to about 40 IU/kg total ADAMTS13 is administered about once every 24 hours. In one embodiment, the about 20 to about 40 IU/kg total ADAMTS13 is administered about once every 12 hours. In certain embodiments, the total ADAMTS13 is about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg. In certain embodiments, the total ADAMTS13 is about 20 IU/kg or about 40 IU/kg. In certain aspects, the injection is administered intravenously. In other aspects, the injection is administered subcutaneously.

In certain embodiments, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 40 units of FRETS-VWF73 activity per kilogram (IU/kg) body weight of the mammal. In certain embodiments, about 40 IU/kg total ADAMTS13 is administered about once a week. In certain embodiments, the treatment regimen is for therapeutic or prophylactic treatments of inherited TTP. In certain aspects, the injection is administered intravenously. In other aspects, the injection is administered subcutaneously.

In certain embodiments, the disclosure provides a method for treating inherited TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 40 units of FRETS-VWF73 activity per kilogram (IU/kg) body weight of the mammal. In certain embodiments, the treatment regimen is for treatment of acute inherited TTP. In certain embodiments, treatment begins with a loading dose of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg on day one. In certain embodiments, the loading dose is followed with a dose of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg daily until resolution of the event or 1 day or 2 days after resolution of the event. In certain embodiments, the loading dose is followed with about 20 IU/kg to about 40 IU/kg daily until resolution of the event or 1 day or 2 days after resolution of the event. In certain embodiments, the loading dose is about 20 IU/kg or about 40 IU/kg on day one and followed with a dose of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg daily until resolution of the event or 1 day or 2 days after resolution of the event. In certain embodiments, treatment for acute inherited TTP begins with a loading dose of about 40 IU/kg on day one followed by doses of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg daily until resolution of the event or 1 day or 2 days after resolution of the event. In certain embodiments, treatment is for acute inherited TTP begins with a loading dose of about 40 IU/kg on day one followed by doses of about 20 IU/kg to about 40 IU/kg daily until resolution of the event. In certain embodiments, treatment is for acute inherited TTP begins with a loading dose of about 40 IU/kg on day one followed by doses of about 20 IU/kg to about 40 IU/kg daily until 1 day after resolution of the event. In certain embodiments, treatment is for acute inherited TTP begins with a loading dose of about 40 IU/kg on day one followed by doses of about 20 IU/kg to about 40 IU/kg daily until 2 days after resolution of the event. In certain aspects, the injection is administered intravenously. In other aspects, the injection is administered subcutaneously. In certain embodiments, the disclosure provides a method for treating or preventing acquired TTP. In acquired TTP, patients have a low ADAMTS13 activity due to the development of autoimmune antibodies directed at ADAMTS13. Immune-complexed ADAMTS13 is inactivated, neutralized and/or cleared from the blood stream and patient plasma. Reduced ADAMTS13 activity leads to the accumulation of large uncleaved VWF multimers which can spontaneously adhere to platelets and leading to platelet-VWF-rich thrombi in the microcirculation. Like inherited TTP, acquired TTP can also lead to neurologic manifestations (e.g., mental status, stroke, seizures, hemiplegia, paresthesias, visual disturbance, and aphasia), fatigue, and severe bleeding. If left untreated, acquired TTP can be fatal or can cause lasting physiological damage.

In one embodiment, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 1 to about 4000 units of FRETS-VWF73 activity per kilogram body weight of the mammal (IU/kg). In a specific embodiment, the mammal is a human. In one embodiment, the about 1 IU/kg to about 4000 IU/kg total ADAMTS13 is administered about once a month. In one embodiment, the about 1 IU/kg to about 4000 IU/kg total ADAMTS13 is administered about twice a month. In one embodiment, the about 1 IU/kg to about 4000 IU/kg total ADAMTS13 is administered about once a week. In one embodiment, the about 1 IU/kg to about 4000 IU/kg total ADAMTS13 is administered about twice a week. In one embodiment, the about 1 IU/kg to about 4000 IU/kg total ADAMTS13 is administered about three times a week. In one embodiment, the about 1 IU/kg to about 4000 IU/kg total ADAMTS13 is administered about once every 48 hours. In one embodiment, the about 1 IU/kg to about 4000 IU/kg total ADAMTS13 is administered about once every 24 hours. In one embodiment, the about 1 IU/kg to about 4000 IU/kg total ADAMTS13 is administered about once every 12 hours. In certain aspects, the injection is administered intravenously. In other aspects, the injection is administered subcutaneously.

In one embodiment, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 5 to about 4000 units of FRETS-VWF73 activity per kilogram body weight of the mammal (IU/kg). In a specific embodiment, the mammal is a human. In one embodiment, the about 5 IU/kg to about 4000 IU/kg total ADAMTS13 is administered about once a month. In one embodiment, the about 5 IU/kg to about 4000 IU/kg total ADAMTS13 is administered about twice a month. In one embodiment, the about 5 IU/kg to about 4000 IU/kg total ADAMTS13 is administered about once a week. In one embodiment, the about 5 IU/kg to about 4000 IU/kg total ADAMTS13 is administered about twice a week. In one embodiment, the about 5 IU/kg to about 4000 IU/kg total ADAMTS13 is administered about three times a week. In one embodiment, the about 5 IU/kg to about 4000 IU/kg total ADAMTS13 is administered about once every 48 hours. In one embodiment, the about 5 IU/kg to about 4000 IU/kg total ADAMTS13 is administered about once every 24 hours. In one embodiment, the 5 IU/kg to about 4000 IU/kg total ADAMTS13 is administered about once every 12 hours. In certain aspects, the injection is administered intravenously. In other aspects, the injection is administered subcutaneously.

In one embodiment, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 10 to about 1,500 units of FRETS-VWF73 activity per kilogram body weight of the mammal (IU/kg). In a specific embodiment, the mammal is a human. In one embodiment, the about 10 IU/kg to about 1,500 IU/kg total ADAMTS13 is administered about once a month. In one embodiment, the about 10 IU/kg to about 1,500 IU/kg total ADAMTS13 is administered about twice a month. In one embodiment, the about 10 IU/kg to about 1,500 IU/kg total ADAMTS13 is administered about once a week. In one embodiment, the about 10 IU/kg to about 1,500 IU/kg total ADAMTS13 is administered about twice a week. In one embodiment, the about 10 IU/kg to about 1,500 IU/kg total ADAMTS13 is administered about once every 48 hours. In one embodiment, the about 10 IU/kg to about 1,500 IU/kg total ADAMTS13 is administered about once every 24 hours. In one embodiment, the about 10 IU/kg to about 1,500 IU/kg total ADAMTS13 is administered about once every 12 hours. In certain aspects, the injection is administered intravenously. In other aspects, the injection is administered subcutaneously.

In one embodiment, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 160 units of FRETS-VWF73 activity per kilogram body weight of the mammal (IU/kg). In a specific embodiment, the mammal is a human. In one embodiment, the about 20 IU/kg to about 160 IU/kg total ADAMTS13 is administered about once a month. In one embodiment, the about 20 to about 160 IU/kg total ADAMTS13 is administered about twice a month. In one embodiment, the about 20 to about 160 IU/kg total ADAMTS13 is administered about once a week. In one embodiment, the about 20 to about 160 IU/kg total ADAMTS13 is administered about twice a week. In one embodiment, the about 20 to about 160 IU/kg total ADAMTS13 is administered about once every 48 hours. In one embodiment, the about 20 to about 160 IU/kg total ADAMTS13 is administered about once every 24 hours. In one embodiment, the about 20 to about 160 IU/kg total ADAMTS13 is administered about once every 12 hours. In certain embodiments, the total ADAMTS13 is about 20 IU/kg, about 40 IU/kg, about 50 IU/kg, about 60 IU/kg, about 70 IU/kg, about 75 IU/kg, about 80 IU/kg, about 90 IU/kg, about 100 IU/kg, about 100 IU/kg, about 120 IU/kg, about 125 IU/kg, about 130 IU/kg, about 140 IU/kg, about 150 IU/kg, about 160 IU/kg. In certain aspects, the injection is administered intravenously. In other aspects, the injection is administered subcutaneously.

In one embodiment, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 80 units of FRETS-VWF73 activity per kilogram body weight of the mammal (IU/kg). In a specific embodiment, the mammal is a human. In one embodiment, the about 20 IU/kg to about 80 IU/kg total ADAMTS13 is administered about once a month. In one embodiment, the about 20 to about 80 IU/kg total ADAMTS13 is administered about twice a month. In one embodiment, the about 20 to about 80 IU/kg total ADAMTS13 is administered about once a week. In one embodiment, the about 20 to about 80 IU/kg total ADAMTS13 is administered about twice a week. In one embodiment, the about 20 to about 80 IU/kg total ADAMTS13 is administered about once every 48 hours. In one embodiment, the about 20 to about 80 IU/kg total ADAMTS13 is administered about once every 24 hours. In one embodiment, the about 20 to about 80 IU/kg total ADAMTS13 is administered about once every 12 hours. In certain embodiments, the total ADAMTS13 is about 20 IU/kg, about 40 IU/kg, about 50 IU/kg, about 60 IU/kg, about 70 IU/kg, about 75 IU/kg, or about 80 IU/kg. In certain embodiments, the dose is a divided dose administered twice in the same day. For example, if the dose is 80 IU/kg, it would be administered as 40 IU/kg twice in the same day. In certain aspects, the injection is administered intravenously. In other aspects, the injection is administered subcutaneously.

In one embodiment, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 40 units of FRETS-VWF73 activity per kilogram body weight of the mammal (IU/kg). In a specific embodiment, the mammal is a human. In one embodiment, the about 20 IU/kg to about 40 IU/kg total ADAMTS13 is administered about once a month. In one embodiment, the about 20 to about 40 IU/kg total ADAMTS13 is administered about twice a month. In one embodiment, the about 20 to about 40 IU/kg total ADAMTS13 is administered about once a week. In one embodiment, the about 20 to about 40 IU/kg total ADAMTS13 is administered about twice a week. In one embodiment, the about 20 to about 40 IU/kg total ADAMTS13 is administered about once every 48 hours. In one embodiment, the about 20 to about 40 IU/kg total ADAMTS13 is administered about once every 24 hours. In one embodiment, the about 20 to about 40 IU/kg total ADAMTS13 is administered about once every 12 hours. In certain embodiments, the total ADAMTS13 is about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg. In certain embodiments, the total ADAMTS13 is about 20 IU/kg or about 40 IU/kg. In certain embodiments, the total ADAMTS13 is about 20 IU/kg. In certain embodiments, the total ADAMTS13 is about 40 IU/kg. In certain aspects, the injection is administered intravenously. In other aspects, the injection is administered subcutaneously.

In certain embodiments, the disclosure provides a method for treating acquired TTP in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 40 units of FRETS-VWF73 activity per kilogram (IU/kg) body weight of the mammal. In certain embodiments, the treatment regimen is for treatment of acquired TTP. In certain embodiments, treatment begins with a loading dose of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg. In certain embodiments, the loading dose is followed with about 20 IU/kg to about 40 IU/kg daily or about 20 IU/kg to about 80 IU/kg daily or BID until resolution of the event or 1 day or 2 days after resolution of the event. In certain embodiments, the loading dose is about 20 IU/kg, about 40 IU/kg, or about 80 IU/kg on day one and followed with a dose of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg daily or BID until resolution of the event or 1 day or 2 days after resolution of the event. In certain embodiments, treatment is for acquired TTP begins with a loading dose of about 40 IU/kg on day one followed by doses of about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg daily or BID until resolution of the event or 1 day or 2 days after resolution of the event. In certain embodiments, treatment is for acquired TTP begins with a loading dose of about 40 IU/kg on day one followed by doses of about 20 IU/kg to about 40 IU/kg daily or BID until resolution of the event. In certain embodiments, treatment is for acquired TTP begins with a loading dose of about 40 IU/kg on day one followed by doses of about 20 IU/kg to about 40 IU/kg daily or BID until 1 day after resolution of the event. In certain embodiments, treatment is for acquired TTP begins with a loading dose of about 40 IU/kg on day one followed by doses of about 20 IU/kg to about 40 IU/kg daily or BID until 2 days after resolution of the event. In certain embodiments, treatment is for acquired TTP begins with a loading dose of about 40 IU/kg on day one followed by about 40 IU/kg daily or BID until resolution of the event. In certain embodiments, treatment is for acquired TTP begins with a loading dose of about 40 IU/kg on day one followed by about 40 IU/kg daily or BID until 1 day after resolution of the event. In certain embodiments, treatment is for acquired TTP begins with a loading dose of about 40 IU/kg on day one followed by about 40 IU/kg daily or BID until 2 days after resolution of the event. In certain aspects, the injection is administered intravenously. In other aspects, the injection is administered subcutaneously.

In one embodiment, the disclosure provides a method for treating acquired TTP in a mammal in need thereof, the method including administering a therapeutically effective amount of a composition comprising ADAMTS13 variant, with or without ADAMTS13 protein to the mammal once in remission, where the therapeutically effective amount of total ADAMTS13 is from about 20 to about 40 units of FRETS-VWF73 activity per kilogram body weight of the mammal (IU/kg). In a specific embodiment, the mammal is a human. In one embodiment, the about 20 IU/kg to about 40 IU/kg total ADAMTS13 is administered about once a month. In one embodiment, the about 20 to about 40 IU/kg total ADAMTS13 is administered about twice a month. In one embodiment, the about 20 to about 40 IU/kg total ADAMTS13 is administered about once a week. In one embodiment, the about 20 to about 40 IU/kg total ADAMTS13 is administered about twice a week. In one embodiment, the about 20 to about 40 IU/kg total ADAMTS13 is administered about three times a week. In one embodiment, the about 20 to about 40 IU/kg total ADAMTS13 is administered about once every 48 hours. In one embodiment, the about 20 to about 40 IU/kg total ADAMTS13 is administered about once every 24 hours. In one embodiment, the about 20 to about 40 IU/kg total ADAMTS13 is administered about once every 12 hours. In certain embodiments, the total ADAMTS13 is about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, or about 40 IU/kg. In certain embodiments, the total ADAMTS13 is about 20 IU/kg or about 40 IU/kg. In certain embodiments, the total ADAMTS13 is about 20 IU/kg. In certain embodiments, the total ADAMTS13 is about 40 IU/kg. In certain aspects, the injection is administered intravenously. In other aspects, the injection is administered subcutaneously.

In certain embodiments, the disclosure provides a method for treating or preventing myocardial infarction. In certain embodiments, an ADAMTS13 variant or composition thereof, including compositions with ADAMTS13, described herein is used for the treatment and prophylaxis of ischemic/reperfusion injury. Reperfusion is the restoration of blood supply to tissue that is ischemic, due to decrease in blood supply. Reperfusion is a procedure for treating infarction (e.g., myocardial infarction and cerebral infarction) or other ischemia, by enabling viable ischemic tissue to recover, thus limiting further necrosis. However, reperfusion can itself further damage the ischemic tissue, causing reperfusion injury. For example, acute myocardial infarction (AMI) is caused by thrombotic occlusion of a coronary artery. In addition to the immediate injury that occurs during deprivation of blood flow, ischemic/reperfusion injury involves tissue injury that occurs after blood flow is restored from the reperfusion.

Furthermore, it has been reported that ADAMTS13 has an anti-inflammatory effect that prevents or decreases secondary injury during ischemic reperfusion. De Meyer et al. (“Protective anti-inflammatory effect of ADAMTS13 on myocardial ischemia/reperfusion injury in mice,” Blood, 2012, 120(26):5217-5223, incorporated herein by reference in its entirety for all purposes). As described by De Meyer et al., VWF and ADAMTS13 are involved in platelet adhesion and thrombus formation because ADAMTS13 cleaves the most thrombogenic VWF multimers into smaller and less hemostatically active VWF fragments. De Meyer et al. also describe ADAMTS's role in down-regulating inflammatory responses. It has also been shown that ADAMTS13 can reduce thrombosis and inflammation (e.g., atherosclerosis). Chauhan et al. (“ADAMTS13: a new link between thrombosis and inflammation,” J Exp Med., 2008, 205:2065-2074); Chauhan et al. (“Systemic antithrombotic effects of ADAMTS13,” J Exp Med., 2006, 203:767-776; Gandhi et al. (“ADAMTS13 reduces vascular inflammation and the development of early atherosclerosis in mice,” Blood, 2012, 119(10):2385-2391), each of which are incorporated herein by reference in their entirety for all purposes.

De Meyer et al. suggest that ADAMTS13 prevents excessive VWF-mediated platelet and leukocyte recruitment in the ischemic myocardium by cleaving VWF. Based on this hypothesis, De Meyer et al. show that neutrophil infiltration in the myocardium of animals with induced myocardial infarction was nine times lower when the animals were treated ADAMTS13. Accordingly, De Meyer et al. show that ADAMTS13 reduces inflammatory responses in ischemic myocardium. This reduced inflammation also reduces reperfusion injury by preventing leukocyte infiltration and damage. Thus, the ADAMTS13 variants and compositions thereof, including compositions with ADAMTS13, disclosed herein can be used to reduce the inflammatory responses and to avoid inflammation that results in tissue damage during infarction (e.g., myocardial infarction and cerebral infarction) and reperfusion.

In certain embodiments, the disclosure provides a method for treating or preventing cerebral infarction. Cerebral infarction, commonly referred to as a stroke, occurs when blood flow to part of the brain is prevented. Cerebral infarctions can occur, for example, when a blood vessel that supplies blood to the brain is blocked by a blood clot. A cerebral infarction can also be the result of a blunt force trauma and mechanical injury. This can either be caused by a clot in an artery of the brain (thrombotic stroke) or by a clot from another part of the body that travels to the brain (embolic stroke). Accordingly, in some embodiments, the invention provides a method of improving the recovery of (or reducing the damage to) sensory and/or motor function in a patient after a cerebral infarction, comprising the step of administering to the individual a pharmaceutical composition comprising a therapeutically effective amount of an ADAMTS13 variant or composition thereof, including compositions with ADAMTS13, thereby improving the recovery of (or reducing the damage to) sensory and/or motor function in the individual post-cerebral infarction.

In certain embodiments, the disclosure provides a method for treating or preventing deep vein thrombosis (DVT). DVT is a blood clot that forms in a vein, deep in the body using an ADAMTS13 variant or composition thereof, including compositions with ADAMTS13. While most deep vein clots occur in the lower leg or thigh, they can occur throughout the body. DVT is a particularly dangerous disease because a blood clot can break off and travel through the bloodstream (an embolus) to the heart, lungs, or brain, for example. Such embolisms can cause damage to organs and may result in death. Accordingly, as described above, ADAMTS13 variants and compositions thereof, including compositions with ADAMTS13, can be used to treat DVT and resulting embolisms.

In certain embodiments, the disclosure provides a method for treating or preventing disseminated intravascular coagulation (DIC), specifically, sepsis-related DIC. DIC is a condition in which blood clots form throughout the body's small blood vessels. These blood clots can reduce or block blood flow throughout the body and can result in damage to tissues and organs. The blood clots in the small blood vessels results from an increase in clotting activity. This increase in activity over uses available platelets and clotting factors, thereby also increasing the chance of serious internal and external bleeding by depleting the available source of platelets and clotting factors. Accordingly, a patient with DIC will often suffer from blood clots and severe bleeding disorders.

Certain diseases such as sepsis, surgery/trauma, cancer, complications of childbirth/pregnancy, venomous snake bites (rattlesnakes and vipers), frostbite, and burns can cause clotting factors to become overactive and can lead to DIC. DIC can also be acute (developing quickly over hours or days) or chronic (developing over weeks or months). While both types of DIC require medical treatment, acute DIC must be treated immediately to prevent excessive blood clotting in the small blood vessels that quickly lead to severe bleeding.

In one embodiment, the disclosure provides a method for treating blood clotting disorders (such as, but not limited to, myocardial infarction, cerebral infarction, ischemic reperfusion injury, DVT, or DIC) and/or reduction of inflammatory events/responses in a mammal in need thereof, the method including administering a therapeutically effective amount of a composition comprising ADAMTS13 variant, with or without ADAMTS13 protein to the mammal, where the therapeutically effective amount of total ADAMTS13 is from about 1 to about 4000 units of FRETS-VWF73 activity per kilogram body weight of the mammal (IU/kg). In a specific embodiment, the mammal is a human. In one embodiment, the about 1 IU/kg to about 4000 IU/kg total ADAMTS13 is administered about once a month. In one embodiment, the about 1 IU/kg to about 4000 IU/kg total ADAMTS13 is administered about twice a month. In one embodiment, the about 1 IU/kg to about 4000 IU/kg total ADAMTS13 is administered about once a week. In one embodiment, the about 1 IU/kg to about 4000 IU/kg total ADAMTS13 is administered about twice a week. In one embodiment, the about 1 IU/kg to about 4000 IU/kg total ADAMTS13 is administered about three times a week. In one embodiment, the about 1 IU/kg to about 4000 IU/kg total ADAMTS13 is administered about once every 48 hours. In one embodiment, the about 1 IU/kg to about 4000 IU/kg total ADAMTS13 is administered about once every 24 hours. In one embodiment, the about 1 IU/kg to about 4000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating blood clotting disorders (such as, but not limited to, myocardial infarction, cerebral infarction, ischemic reperfusion injury, DVT, or DIC) and/or reduction of inflammatory events/responses in a mammal in need thereof, the method including administering a therapeutically effective amount of a composition comprising ADAMTS13 variant, with or without ADAMTS13 protein to the mammal, where the therapeutically effective amount of total ADAMTS13 is from about 5 to about 4000 units of FRETS-VWF73 activity per kilogram body weight of the mammal (IU/kg). In a specific embodiment, the mammal is a human. In one embodiment, the about 5 IU/kg to about 4000 IU/kg total ADAMTS13 is administered about once a month. In one embodiment, the about 5 IU/kg to about 4000 IU/kg total ADAMTS13 is administered about twice a month. In one embodiment, the about 5 IU/kg to about 4000 IU/kg total ADAMTS13 is administered about once a week. In one embodiment, the about 5 IU/kg to about 4000 IU/kg total ADAMTS13 is administered about twice a week. In one embodiment, the about 5 IU/kg to about 4000 IU/kg total ADAMTS13 is administered about three times a week. In one embodiment, the about 5 IU/kg to about 4000 IU/kg total ADAMTS13 is administered about once every 48 hours. In one embodiment, the about 5 IU/kg to about 4000 IU/kg total ADAMTS13 is administered about once every 24 hours. In one embodiment, the 5 IU/kg to about 4000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating blood clotting disorders (such as, but not limited to, myocardial infarction, cerebral infarction, ischemic reperfusion injury, DVT, or DIC) and/or reduction of inflammatory events/responses in a mammal in need thereof, the method including administering a therapeutically effective amount of a composition comprising ADAMTS13 variant, with or without ADAMTS13 protein to the mammal, where the therapeutically effective amount of total ADAMTS13 is from about 10 to about 2,000 units of FRETS-VWF73 activity per kilogram body weight of the mammal (IU/kg). In a specific embodiment, the mammal is a human. In one embodiment, the about 10 IU/kg to about 2,000 IU/kg total ADAMTS13 is administered about once a month. In one embodiment, the about 10 IU/kg to about 2,000 IU/kg total ADAMTS13 is administered about twice a month. In one embodiment, the about 10 IU/kg to about 2,000 IU/kg total ADAMTS13 is administered about once a week. In one embodiment, the about 10 IU/kg to about 2,000 IU/kg total ADAMTS13 is administered about twice a week. In one embodiment, the about 10 IU/kg to about 2,000 IU/kg total ADAMTS13 is administered about once every 48 hours. In one embodiment, the about 10 IU/kg to about 2,000 IU/kg total ADAMTS13 is administered about once every 24 hours. In one embodiment, the about 10 IU/kg to about 2,000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating blood clotting disorders (such as, but not limited to, myocardial infarction, cerebral infarction, ischemic reperfusion injury, DVT, or DIC) and/or reduction of inflammatory events/responses in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 1,500 units of FRETS-VWF73 activity per kilogram body weight of the mammal (IU/kg). In a specific embodiment, the mammal is a human. In one embodiment, the about 20 IU/kg to about 1,500 IU/kg total ADAMTS13 is administered about once a month. In one embodiment, the about 20 IU/kg to about 1,500 IU/kg total ADAMTS13 is administered about twice a month. In one embodiment, the about 20 IU/kg to about 1,500 IU/kg total ADAMTS13 is administered about once a week. In one embodiment, the about 20 IU/kg to about 1,500 IU/kg total ADAMTS13 is administered about twice a week. In one embodiment, the about 20 IU/kg to about 1,500 IU/kg total ADAMTS13 is administered about once every 48 hours. In one embodiment, the about 20 IU/kg to about 1,500 IU/kg total ADAMTS13 is administered about once every 24 hours. In one embodiment, the about 20 IU/kg to about 1,500 IU/kg total ADAMTS13 is administered about once every 12 hours. In certain embodiments, the total ADAMTS13 is about 20 IU/kg, about 21 IU/kg, about 22 IU/kg, about 23 IU/kg, about 24 IU/kg, about 25 IU/kg, about 26 IU/kg, about 27 IU/kg, about 28 IU/kg, about 29 IU/kg, about 30 IU/kg, about 31 IU/kg, about 32 IU/kg, about 33 IU/kg, about 34 IU/kg, about 35 IU/kg, about 36 IU/kg, about 37 IU/kg, about 38 IU/kg, about 39 IU/kg, about 40 IU/kg, about 45 IU/kg, about 50 IU/kg, about IU/kg, about 60 IU/kg, about 70 IU/kg, about 75 IU/kg, about 80 IU/kg, about 90 IU/kg, about 100 IU/kg, about 110 IU/kg, about 120 IU/kg, about 125 IU/kg, about 130 IU/kg, about 140 IU/kg, about 150 IU/kg, about 160 IU/kg, about 170 IU/kg, about 175 IU/kg, about 180 IU/kg, about 190 IU/kg, about 200 IU/kg, about 225 IU/kg, about 250 IU/kg, about 275 IU/kg, about 300 IU/kg, about 350 IU/kg, about 400 IU/kg, about 450 IU/kg, about 500 IU/kg, about 550 IU/kg, about 600 IU/kg, about 650 IU/kg, about 700 IU/kg, about 750 IU/kg, about 800 IU/kg, about 850 IU/kg, about 900 IU/kg, about 950 IU/kg, about 1000 IU/kg, about 1150 IU/kg, about 1200 IU/kg, about 1250 IU/kg, about 1300 IU/kg, about 1350 IU/kg, about 1400 IU/kg, about 1450 IU/kg, or about 1500 IU/kg. In certain embodiments, the dose is administered for treatment and/or prophylaxis. In certain aspects, the injection is administered intravenously. In other aspects, the injection is administered subcutaneously.

In certain embodiments, the disclosure provides a method for treating or preventing vaso-occlusive crisis (VOC) in sickle cell disease (SCD), as described in publication of International Application No. WO2018027169, which is incorporated herein in its entirety for all purposes. SCD is a worldwide hereditary red blood cell disorder caused by a point mutation in the β-globin gene resulting in the synthesis of pathological HbS, and abnormal HbS polymerization in hypoxic conditions. The two main clinical manifestations of SCD are chronic hemolytic anemia and acute VOC, which are the principal causes of hospitalization of SCD patients. Recent studies have underscored the central role of sickle vasculopathy in the generation of sickle cell-related acute events and chronic organ complications (Sparkenbaugh et al., Br. J. Haematol. 162:3-14, 2013; De Franceschi et al., Semin. Thromb. Hemost. 226-36, 2011; and Hebbel et al., Cardiovasc. Hematol. Disord. Drug Targets, 9:271-92, 2009, each of which are incorporated herein by reference for all purposes). The pathophysiology of these complications is based on intravascular sickling in capillaries and small vessels leading to VOC, impaired blood flow, vascular inflammation, and/or thrombosis with ischemic cell damage.

The most common clinical manifestation of SCD is VOC. A VOC occurs when the microcirculation is obstructed by sickled red blood cells, causing ischemic injury to the organ supplied and resultant pain. Pain crises constitute the most distinguishing clinical feature of SCD and are the leading cause of emergency department visits and/or hospitalizations for affected SCD subjects or patients.

Approximately half the SCD subjects or patients with homozygous HbS disease experience VOC. The frequency of crisis is extremely variable. Some SCD subjects or patients have as many as six or more episodes annually, whereas others may have episodes only at great intervals or none at all. Each subjects or patient typically has a consistent pattern for crisis frequency.

The disclosure includes methods for reducing at least one symptom of VOC including, but not limited to, ischemia and pain (e.g., dactylitis, priapism, abdominal, chest, and joint), jaundice, bone infarction, abnormal breathing (e.g., tachypnea and shortness of breath), hypoxia, acidosis, hypotension, and/or tachycardia associated with VOC. In certain aspects, VOC can be defined as a condition comprising one or more of these symptoms. Pain crises begin suddenly. The crisis may last several hours to several days and terminate as abruptly as it began. The pain can affect any body part and often involves the abdomen, appendages, chest, back, bones, joints, and soft tissue, and it may present as dactylitis (bilateral painful and swollen hands and/or feet in children), acute joint necrosis or avascular necrosis, or acute abdomen. With repeated episodes in the spleen, infarctions and autosplenectomy predisposing to life-threatening infection are usual. The liver also may infarct and progress to failure with time. Papillary necrosis is a common renal manifestation of VOC, leading to isosthenuria (i.e., inability to concentrate urine).

Severe deep pain is present in the extremities, involving long bones. Abdominal pain can be severe, resembling acute abdomen; it may result from referred pain from other sites or intra-abdominal solid organ or soft tissue infarction. Reactive ileus leads to intestinal distention and pain. The face also may be involved. Pain may be accompanied by fever, malaise, trouble breathing, painful erections, jaundice, and leukocytosis. Bone pain is often due to bone marrow infarction. Certain patterns are predictable, as pain tends to involve bones with the most bone marrow activity and because marrow activity changes with age. During the first 18 months of life, the metatarsals and metacarpals can be involved, presenting as dactylitis or hand-foot syndrome. Although the above patterns describe commonly encountered presentations, any area of the body of the subject with blood supply and sensory nerves can be affected in VOC.

Often, no precipitating cause can be identified for what causes a VOC. However, because deoxygenated HbS becomes semi-solid, the most likely physiologic trigger of VOC is hypoxemia. This may be due to acute chest syndrome or accompany respiratory complications. Dehydration also can precipitate pain, since acidosis results in a shift of the oxygen dissociation curve (Bohr effect), causing hemoglobin to desaturate more readily. Hemoconcentration also is a common mechanism. Another common trigger of VOC are changes in body temperature, whether an increase due to fever or a decrease due to environmental temperature change. Lowered body temperature likely leads to crises as the result of peripheral vasoconstriction.

In certain embodiments, VOC can be defined as having an increase in peripheral neutrophils as compared to a control. In certain embodiments, VOC can be defined as an increase in pulmonary vascular leakage (e.g., increased number of leukocytes in a bronchoalveolar lavage (BAL) and/or protein content (BAL protein (mg/mL)) as compared to a control.

In certain embodiments, increased levels of vascular activation (e.g., as measured by increased expression, levels, and/or activity of VCAM-1 and/or ICAM-1) in an organ, as compared to control, is a marker for VOC. In certain embodiments, increased levels of inflammatory vasculopathy in an organ, as compared to control, is a marker for VOC. In certain embodiments, increased levels of vascular activation and inflammatory vasculopathy in a tissue, as compared to control, is a marker for VOC. In certain embodiments, the organ is lung and/or kidney. In certain embodiments, the organ is kidney.

In certain embodiments, VOC can be defined as the increased expression, levels, and/or activation of at least one of NF-kB (wherein activation of NF-kB is measured by P-NF-kB or the ratio of P-NF-kB/NF-kB), VCAM-1 and ICAM-1 as compared to control. In certain embodiments, VOC can be defined as increased expression or level of at least one of endothelin-1 (ET-1), thromboxane synthase (TXAS), and heme-oxygenase-1 (HO-1) as compared to control. In certain embodiments, these increases are seen in lung tissue. In certain embodiments, these increases are seen in kidney tissue. In certain embodiments, increased expression and/or levels of TXAS, ET-1, and VCAM-1, and activation of NF-kB in the kidney tissue are markers for VOC.

In certain embodiments, VOC can be defined by hematology parameters. In certain embodiments, VOC can be defined as a decrease in the levels of at least one of Hct, Hb, MCV, and MCH as compared to control. In certain embodiments, VOC can be defined as a decrease in the levels of at least two of Hct, Hb, MCV, and MCH as compared to control. In certain embodiments, VOC can be defined as a decrease in the levels of at least three of Hct, Hb, MCV, and MCH as compared to control. In certain embodiments, VOC can be defined as an increase the levels of at least one of CHCM, HDW, neutrophil numbers, and LDH as compared to control. In certain embodiments, VOC can be defined as an increase the levels of at least two of CHCM, HDW, neutrophil numbers, and LDH as compared to control. In certain embodiments, VOC can be defined as an increase the levels of at least three of CHCM, HDW, neutrophil numbers, and LDH as compared to control. In certain embodiments, VOC can be defined as a decrease in Hct levels as compared to control. In certain embodiments, VOC can be defined as a decrease in Hb levels as compared to control. In certain embodiments, VOC can be defined as a decrease in MCV as compared to control. In certain embodiments, VOC can be defined as a decrease in MCH as compared to control. In certain embodiments, VOC can be defined as an increase in CHCM as compared to control. In certain embodiments, VOC can be defined as an increase in HDW as compared to control. In certain embodiments, VOC can be defined as an increase in neutrophil numbers as compared to control. In certain embodiments, VOC can be defined as an increase in LDH as compared to control. In certain embodiments, VOC can be defined as a decrease in the levels of at least one of Hct, Hb, MCV, and MCH as compared to control and/or an increase the levels of at least one of CHCM, HDW, neutrophil numbers, and LDH as compared to control. In certain embodiments, VOC can be defined as a decrease in the levels of Hct, Hb, MCV, and MCH as compared to control and/or an increase the levels of CHCM, HDW, neutrophil numbers, and LDH as compared to control.

In certain embodiments, the composition comprising ADAMTS13 variants, including compositions with ADAMTS13, is administered to the subject within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 60, 72, 84, 96, 108, or 120 hours after the onset of the VOC. In some embodiments, the composition comprising ADAMTS13 variants, including compositions with ADAMTS13, is administered to the subject within about 1-2 hours, about 1-5 hours, about 1-10 hours, about 1-12 hours, about 1-24 hours, about 1-36 hours, about 1-48 hour, about 1-60 hours, about 1-72 hours, about 1-84 hours, about 1-96 hours, about 1-108 hours, or about 1-120 hours after the onset of the VOC. In some embodiments, the composition comprising ADAMTS13 variant, with or without ADAMTS13 protein, is administered to the subject within about 2-5 hours, about 5-10 hours, about 10-20 hours, about 20-40 hours, about 30-60 hours, about 40-80 hours, about 50-100 hours, or about 60-120 hours after the onset of the VOC. In some embodiments, the composition is administered within 1 week of the VOC. In some embodiments, the composition is administered daily after the VOC. In some embodiments, the composition is administered weekly after the VOC. In some embodiments, the composition is administered every day. In some embodiments, the composition is administered every other day. In some embodiments, the composition is administered every third day. In some embodiments, the composition is administered twice a week. In some embodiments, the composition is administered until the clinical manifestations resolve. In some embodiments, the composition is administered until a day after clinical manifestations resolve. In some embodiments, the composition is administered for at least two days after clinical manifestations resolve. In some embodiments, the composition is administered for at least three days after clinical manifestations resolve. In some embodiments, the composition is administered for at least a week after clinical manifestations resolve.

In certain embodiments, the composition comprising ADAMTS13 variants, including compositions with ADAMTS13, is administered to the subject to prevent the onset of VOC. In such preventative treatment, the ADAMTS13 variant, with or without ADAMTS13 protein, is administered in a singular bolus injection or in multiple doses to maintain a circulating level of total ADAMTS13 effective to prevent the onset of the VOC. In such aspects, the composition comprising ADAMTS13 variants, including compositions with ADAMTS13, is administered monthly, every two weeks, weekly, twice a week, every other day, daily, every 12 hours, every 8 hours, every six hours, every four hours, every two hours, or every hour. In particular aspects, the injection is administered subcutaneously. In other aspects, the injection is administered intravenously.

In one embodiment, the disclosure provides a method for treating VOC in SCD in a mammal in need thereof, the method including administering a therapeutically effective amount of a composition comprising ADAMTS13 variant, with or without ADAMTS13 protein to the mammal, where the therapeutically effective amount of total ADAMTS13 is from about 1 to about 4000 units of FRETS-VWF73 activity per kilogram body weight of the mammal (IU/kg). In a specific embodiment, the mammal is a human. In one embodiment, the about 1 IU/kg to about 4000 IU/kg total ADAMTS13 is administered about once a month. In one embodiment, the about 1 IU/kg to about 4000 IU/kg total ADAMTS13 is administered about twice a month. In one embodiment, the about 1 IU/kg to about 4000 IU/kg total ADAMTS13 is administered about once a week. In one embodiment, the about 1 IU/kg to about 4000 IU/kg total ADAMTS13 is administered about twice a week. In one embodiment, the about 1 IU/kg to about 4000 IU/kg total ADAMTS13 is administered about three times a week. In one embodiment, the about 1 IU/kg to about 4000 IU/kg total ADAMTS13 is administered about once every 48 hours. In one embodiment, the about 1 IU/kg to about 4000 IU/kg total ADAMTS13 is administered about once every 24 hours. In one embodiment, the about 1 IU/kg to about 4000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating VOC in SCD in a mammal in need thereof, the method including administering a therapeutically effective amount of a composition comprising ADAMTS13 variant, with or without ADAMTS13 protein to the mammal, where the therapeutically effective amount of total ADAMTS13 is from about 5 to about 4000 units of FRETS-VWF73 activity per kilogram body weight of the mammal (IU/kg). In a specific embodiment, the mammal is a human. In one embodiment, the about 5 IU/kg to about 4000 IU/kg total ADAMTS13 is administered about once a month. In one embodiment, the about 5 IU/kg to about 4000 IU/kg total ADAMTS13 is administered about twice a month. In one embodiment, the about 5 IU/kg to about 4000 IU/kg total ADAMTS13 is administered about once a week. In one embodiment, the about 5 IU/kg to about 4000 IU/kg total ADAMTS13 is administered about twice a week. In one embodiment, the about 5 IU/kg to about 4000 IU/kg total ADAMTS13 is administered about three times a week. In one embodiment, the about 5 IU/kg to about 4000 IU/kg total ADAMTS13 is administered about once every 48 hours. In one embodiment, the about 5 IU/kg to about 4000 IU/kg total ADAMTS13 is administered about once every 24 hours. In one embodiment, the about 5 IU/kg to about 4000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating VOC in SCD in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 2000 units of FRETS-VWF73 activity per kilogram body weight of the mammal (IU/kg). In a specific embodiment, the mammal is a human. In one embodiment, the about 20 IU/kg to about 2000 IU/kg total ADAMTS13 is administered about once a month. In one embodiment, the about 20 IU/kg to about 2000 IU/kg total ADAMTS13 is administered about twice a month. In one embodiment, the about 20 IU/kg to about 2000 IU/kg total ADAMTS13 is administered about once a week. In one embodiment, the about 20 IU/kg to about 2000 IU/kg total ADAMTS13 is administered about twice a week. In one embodiment, the about 20 IU/kg to about 2000 IU/kg total ADAMTS13 is administered about three times a week. In one embodiment, the about 20 IU/kg to about 2000 IU/kg total ADAMTS13 is administered about once every 48 hours. In one embodiment, the about 20 IU/kg to about 2000 IU/kg total ADAMTS13 is administered about once every 24 hours. In one embodiment, the about 20 IU/kg to about 2000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating VOC in SCD in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 500 units of FRETS-VWF73 activity per kilogram body weight of the mammal (IU/kg). In a specific embodiment, the mammal is a human. In one embodiment, the about 20 IU/kg to about 500 IU/kg total ADAMTS13 is administered about once a month. In one embodiment, the about 20 IU/kg to about 500 IU/kg total ADAMTS13 is administered about twice a month. In one embodiment, the about 20 IU/kg to about 500 IU/kg total ADAMTS13 is administered about once a week. In one embodiment, the about 20 IU/kg to about 500 IU/kg total ADAMTS13 is administered about twice a week. In one embodiment, the about 20 IU/kg to about 500 IU/kg total ADAMTS13 is administered about three times a week. In one embodiment, the about 20 IU/kg to about 500 IU/kg total ADAMTS13 is administered about once every 48 hours. In one embodiment, the about 20 IU/kg to about 500 IU/kg total ADAMTS13 is administered about once every 24 hours. In one embodiment, the about 20 IU/kg to about 500 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating VOC in SCD in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 160 units of FRETS-VWF73 activity per kilogram body weight of the mammal (IU/kg). In a specific embodiment, the mammal is a human. In one embodiment, the about 20 IU/kg to about 160 IU/kg total ADAMTS13 is administered about once a month. In one embodiment, the about 20 IU/kg to about 160 IU/kg total ADAMTS13 is administered about twice a month. In one embodiment, the about 20 IU/kg to about 160 IU/kg total ADAMTS13 is administered about once a week. In one embodiment, the about 20 IU/kg to about 160 IU/kg total ADAMTS13 is administered about twice a week. In one embodiment, the about 20 IU/kg to about 160 IU/kg total ADAMTS13 is administered about three times a week. In one embodiment, the about 20 IU/kg to about 160 IU/kg total ADAMTS13 is administered about once every 48 hours. In one embodiment, the about 20 IU/kg to about 160 IU/kg total ADAMTS13 is administered about once every 24 hours. In one embodiment, the about 20 IU/kg to about 160 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating VOC in SCD in a mammal in need thereof, the method including administering a therapeutically effective amount of a composition comprising ADAMTS13 variant, with or without ADAMTS13 protein to the mammal, where the therapeutically effective amount of total ADAMTS13 is from about 40 to about 160 units of FRETS-VWF73 activity per kilogram body weight of the mammal (IU/kg). In a specific embodiment, the mammal is a human. In one embodiment, the about 40 IU/kg to about 160 IU/kg total ADAMTS13 is administered about once a month. In one embodiment, the about 40 IU/kg to about 160 IU/kg total ADAMTS13 is administered about twice a month. In one embodiment, the about 40 IU/kg to about 160 IU/kg total ADAMTS13 is administered about once a week. In one embodiment, the about 40 IU/kg to about 160 IU/kg total ADAMTS13 is administered about twice a week. In one embodiment, the about 40 IU/kg to about 160 IU/kg total ADAMTS13 is administered about three times a week. In one embodiment, the about 40 IU/kg to about 160 IU/kg total ADAMTS13 is administered about once every 48 hours. In one embodiment, the about 40 IU/kg to about 160 IU/kg total ADAMTS13 is administered about once every 24 hours. In one embodiment, the about 40 IU/kg to about 160 IU/kg total ADAMTS13 is administered about once every 12 hours. In certain embodiments, the dose of total ADAMTS13 is about 20 IU/kg, about 40 IU/kg, about 50 IU/kg, about 60 IU/kg, about 70 IU/kg, about 75 IU/kg, about 80 IU/kg, about 90 IU/kg, about 100 IU/kg, about 100 IU/kg, about 120 IU/kg, about 125 IU/kg, about 130 IU/kg, about 140 IU/kg, about 150 IU/kg, or about 160 IU/kg. In certain embodiments, the dose of total ADAMTS13 is about 40 IU/kg, about 80 IU/kg, or about 160 IU/kg. In certain embodiments, the dose is administered for treatment and/or prophylaxis. In certain aspects, the injection is administered intravenously. In other aspects, the injection is administered subcutaneously.

In certain embodiments, the disclosure provides a method for treating and/or preventing acute lung injury (ALI) and acute respiratory distress syndrome (ARDS), including the resultant ventilator-associated lung injury. Pathogenesis of ALI/ARDS is explained by injury to both the vascular endothelium and alveolar epithelium. Phase III clinical trials by the NHLBI ARDS Network have resulted in improvement in survival and a reduction in the duration of mechanical ventilation with a lung-protective ventilation strategy and fluid conservative protocol. However, there is a strong unmet medical need for additional treatments because there are no existing specific pharmacologic therapies for ALI/ARDS. Therefore, the use of ADAMTS13 in the treatment of ALI/ARDS represents a breakthrough in the treatment of ALI/ARDS.

ALI, in certain embodiments, is a disorder of acute inflammation that causes disruption of the lung endothelial and epithelial barriers. Cellular characteristics of ALI include loss of alveolar-capillary membrane integrity, excessive transepithelial neutrophil migration, and release of pro-inflammatory, cytotoxic mediators. Several studies have documented increased release of VWF and upregulation of intracellular adhesion molecule-1 (ICAM-1) following endothelial injury (Johnson, supra). Transepithelial neutrophil migration is an important feature of ALI because neutrophils are the primary perpetrators of inflammation. Prolonged activation of neutrophils contributes to basement membrane destruction and increased permeability of the alveolar-capillary barrier. (Johnson, supra).

ARDS, in certain embodiments, includes acute onset tachypnea, hypoxemia, diffuse pulmonary infiltrates, and loss of lung compliance characterized by high short-term mortality in adults (Walkey, supra). Therapeutic strategies for ARDS focus upon treating the underlying etiology and providing supportive care that reduces the progression of lung injury. Most patients with ARDS develop respiratory failure severe enough to require mechanical ventilatory support. Mechanical ventilation can cause further injury to the lungs called ventilator-associated lung injury (VALI) from the combined mechanistic forces of overdistension and cyclic recruitment. VALI produces “biotrauma” from systemic release of inflammatory cytokines. Currently, the primary goal for management of ARDS is the reduction of VALI. (Walkey, supra).

ADAMTS13 variants or compositions thereof, including compositions with ADAMTS13, can be used in treating or ameliorating lung damage resulting from acute lung injury characterized by the sudden onset of pulmonary edema (including inflammatory pulmonary edema) secondary to myriad local or systemic insults, including bilateral, inflammatory pulmonary infiltrates and impaired oxygenation or hypoxemia.

In certain embodiments, ALI and/or ARDS can be defined by one of more, but not limited to, ischemia, abnormal breathing (e.g., tachypnea and shortness of breath), non-cardiogenic pulmonary edema, pulmonary infiltrates, decreased oxygenation, and decreased ventilation associated with ALI/ARDS. The disclosure includes methods for reducing symptoms of ALI/ARDS including, but not limited to, at least one of ischemia, abnormal breathing (e.g., tachypnea and shortness of breath), non-cardiogenic pulmonary edema, pulmonary infiltrates, decreased oxygenation, decreased ventilation, and combinations thereof associated with ALI/ARDS.

In certain embodiments, ALI and/or ARDS can be defined as having an increase in peripheral neutrophils as compared to a control. In certain embodiments, ALI and/or ARDS can be defined as an increase in pulmonary vascular leakage (e.g., increased number of leukocytes in a bronchoalveolar lavage (BAL) and/or protein content (BAL protein (mg/mL)) as compared to a control.

In certain embodiments, increased levels of vascular activation in an organ, as compared to control, is a marker for ALI and/or ARDS. In certain embodiments, increased levels of inflammatory vasculopathy in an organ, as compared to control, is a marker for ALI and/or ARDS. In certain embodiments, increased levels of vascular activation and inflammatory vasculopathy in a tissue, as compared to control, is a marker for ALI and/or ARDS. In certain embodiments, the organ is lung and/or kidney.

In certain embodiments, ALI and/or ARDS can be defined as the increased expression, levels, and/or activation of NF-kB (wherein activation of NF-kB is measured by P-NF-kB or the ratio of P-NF-kB/NF-kB), VCAM-1, and/or ICAM-1 as compared to control. In certain embodiments, ALI and/or ARDS can be defined as increased expression or level of at least one of endothelin-1 (ET-1), thromboxane synthase (TXAS), and heme-oxygenase-1 (HO-1) as compared to control. In certain embodiments, these increases are seen in lung tissue. In certain embodiments, these increases are seen in kidney tissue. In certain embodiments, increased expression and/or levels of TXAS and ET-1 and activation of NF-kB in the kidney tissue are markers for ALI and/or ARDS.

In certain embodiments, ALI and/or ARDS can be defined by hematology parameters. In certain embodiments, ALI and/or ARDS can be defined as an increase in neutrophil numbers as compared to control. In certain embodiments, ALI and/or ARDS can be defined as an increase in neutrophils as compared to control.

In some embodiments, compositions comprising ADAMTS13 variants, including compositions with ADAMTS13, is administered to the subject within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 60, 72, 84, 96, 108, or 120 hours after the onset of the ALI or ARDS. In some embodiments, compositions comprising ADAMTS13 variants, including compositions with ADAMTS13, is administered to the subject within about 1-2 hours, about 1-5 hours, about 1-10 hours, about 1-12 hours, about 1-24 hours, about 1-36 hours, about 1-48 hour, about 1-60 hours, about 1-72 hours, about 1-84 hours, about 1-96 hours, about 1-108 hours, or about 1-120 hours after the onset of the ALI or ARDS. In some embodiments, compositions comprising ADAMTS13 variants, including compositions with ADAMTS13, is administered to the subject within about 2-5 hours, about 5-10 hours, about 10-20 hours, about 20-40 hours, about 30-60 hours, about 40-80 hours, about 50-100 hours, or about 60-120 hours after the onset of the ALI or ARDS. In some embodiments, the composition is administered within 4 hours, within 8 hours, within 12 hours, within 1 day, within 2 days, within 3 days, within 4 days, within 5 days, within 6 days after the onset or diagnosis of the ALI or ARDS. In some embodiments, the composition is administered within 1 week after the onset or diagnosis of the ALI or ARDS. In some embodiments, the composition is administered daily after the onset or diagnosis of ALI or ARDS. In some embodiments, the composition is administered weekly after the onset or diagnosis of ALI or ARDS. In some embodiments, the composition is administered every day. In some embodiments, the composition is administered every other day. In some embodiments, the composition is administered every third day. In some embodiments, the composition is administered twice a week. In some embodiments, the composition is administered until the clinical manifestations resolve. In some embodiments, the composition is administered until a day after clinical manifestations resolve. In some embodiments, the composition is administered for at least two days after clinical manifestations resolve. In some embodiments, the composition is administered for at least three days after clinical manifestations resolve. In some embodiments, the composition is administered for at least a week after clinical manifestations resolve.

In certain embodiments, compositions comprising ADAMTS13 variants, including compositions with ADAMTS13, is administered to the subject to prevent the onset of ALI or ARDS. In such preventative treatment, ADAMTS13 variants, including compositions with ADAMTS13, is administered in a singular bolus injection or in multiple doses to maintain a circulating level of ADAMTS13 variants effective to prevent the onset of the ALI or ARDS. In such aspects, the composition comprising ADAMTS13 variant, with or without ADAMTS13 protein, is administered monthly, every two weeks, weekly, twice a week, every other day, daily, every 12 hours, every 8 hours, every six hours, every four hours, every two hours, or every hour. In certain embodiments, the injection is administered subcutaneously. In other aspects, the injection is administered intravenously.

In some embodiments, compositions comprising ADAMTS13 variants, including compositions with ADAMTS13, is administered to the subject before the onset of the ALI or ARDS to prevent the ALI or ARDS. In such aspects of the disclosure, the composition is administered in a therapeutically effective amount or dose sufficient to maintain an effective level of ADAMTS13 activity in the subject or in the blood of the subject.

In one embodiment, the disclosure provides a method for treating ALI and/or ARDS in a mammal in need thereof, the method including administering a therapeutically effective amount of a composition comprising ADAMTS13 variant, with or without ADAMTS13 protein to the mammal, where the therapeutically effective amount of total ADAMTS13 is from about 1 to about 4000 units of FRETS-VWF73 activity per kilogram body weight of the mammal (IU/kg). In a specific embodiment, the mammal is a human. In one embodiment, the about 1 IU/kg to about 4000 IU/kg total ADAMTS13 is administered about once a month. In one embodiment, the about 1 IU/kg to about 4000 IU/kg total ADAMTS13 is administered about twice a month. In one embodiment, the about 1 IU/kg to about 4000 IU/kg total ADAMTS13 is administered about once a week. In one embodiment, the about 1 IU/kg to about 4000 IU/kg total ADAMTS13 is administered about twice a week. In one embodiment, the about 1 IU/kg to about 4000 IU/kg total ADAMTS13 is administered about three times a week. In one embodiment, the about 1 IU/kg to about 4000 IU/kg total ADAMTS13 is administered about once every 48 hours. In one embodiment, the about 1 IU/kg to about 4000 IU/kg total ADAMTS13 is administered about once every 24 hours. In one embodiment, the about 1 IU/kg to about 4000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating ALI and/or ARDS in a mammal in need thereof, the method including administering a therapeutically effective amount of a composition comprising ADAMTS13 variant, with or without ADAMTS13 protein to the mammal, where the therapeutically effective amount of total ADAMTS13 is from about 5 to about 4000 units of FRETS-VWF73 activity per kilogram body weight of the mammal (IU/kg). In a specific embodiment, the mammal is a human. In one embodiment, the about 5 IU/kg to about 4000 IU/kg total ADAMTS13 is administered about once a month. In one embodiment, the about 5 IU/kg to about 4000 IU/kg total ADAMTS13 is administered about twice a month. In one embodiment, the about 5 IU/kg to about 4000 IU/kg total ADAMTS13 is administered about once a week. In one embodiment, the about 5 IU/kg to about 4000 IU/kg total ADAMTS13 is administered about twice a week. In one embodiment, the about 5 IU/kg to about 4000 IU/kg total ADAMTS13 is administered about three times a week. In one embodiment, the about 5 IU/kg to about 4000 IU/kg total ADAMTS13 is administered about once every 48 hours. In one embodiment, the about 5 IU/kg to about 4000 IU/kg total ADAMTS13 is administered about once every 24 hours. In one embodiment, the about 5 IU/kg to about 4000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating ALI and/or ARDS in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 2000 units of FRETS-VWF73 activity per kilogram body weight of the mammal (IU/kg). In a specific embodiment, the mammal is a human. In one embodiment, the about 20 IU/kg to about 2000 IU/kg total ADAMTS13 is administered about once a month. In one embodiment, the about 20 IU/kg to about 2000 IU/kg total ADAMTS13 is administered about twice a month. In one embodiment, the about 20 IU/kg to about 2000 IU/kg total ADAMTS13 is administered about once a week. In one embodiment, the about 20 IU/kg to about 2000 IU/kg total ADAMTS13 is administered about twice a week. In one embodiment, the about 20 IU/kg to about 2000 IU/kg total ADAMTS13 is administered about three times a week. In one embodiment, the about 20 IU/kg to about 2000 IU/kg total ADAMTS13 is administered about once every 48 hours. In one embodiment, the about 20 IU/kg to about 2000 IU/kg total ADAMTS13 is administered about once every 24 hours. In one embodiment, the about 20 IU/kg to about 2000 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating ALI and/or ARDS in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 500 units of FRETS-VWF73 activity per kilogram body weight of the mammal (IU/kg). In a specific embodiment, the mammal is a human. In one embodiment, the about 20 IU/kg to about 500 IU/kg total ADAMTS13 is administered about once a month. In one embodiment, the about 20 IU/kg to about 500 IU/kg total ADAMTS13 is administered about twice a month. In one embodiment, the about 20 IU/kg to about 500 IU/kg total ADAMTS13 is administered about once a week. In one embodiment, the about 20 IU/kg to about 500 IU/kg total ADAMTS13 is administered about twice a week. In one embodiment, the about 20 IU/kg to about 500 IU/kg total ADAMTS13 is administered about three times a week. In one embodiment, the about 20 IU/kg to about 500 IU/kg total ADAMTS13 is administered about once every 48 hours. In one embodiment, the about 20 IU/kg to about 500 IU/kg total ADAMTS13 is administered about once every 24 hours. In one embodiment, the about 20 IU/kg to about 500 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating ALI and/or ARDS in a mammal in need thereof, the method including administering a therapeutically effective amount of a composition comprising ADAMTS13 variant, with or without ADAMTS13 protein to the mammal, where the therapeutically effective amount of total ADAMTS13 is from about 20 to about 160 units of FRETS-VWF73 activity per kilogram body weight of the mammal (IU/kg). In a specific embodiment, the mammal is a human. In one embodiment, the about 20 IU/kg to about 160 IU/kg total ADAMTS13 is administered about once a month. In one embodiment, the about 20 IU/kg to about 160 IU/kg total ADAMTS13 is administered about twice a month. In one embodiment, the about 20 IU/kg to about 160 IU/kg total ADAMTS13 is administered about once a week. In one embodiment, the about 20 IU/kg to about 160 IU/kg total ADAMTS13 is administered about twice a week. In one embodiment, the about 20 IU/kg to about 160 IU/kg total ADAMTS13 is administered about three times a week. In one embodiment, the about 20 IU/kg to about 160 IU/kg total ADAMTS13 is administered about once every 48 hours. In one embodiment, the about 20 IU/kg to about 160 IU/kg total ADAMTS13 is administered about once every 24 hours. In one embodiment, the about 20 IU/kg to about 160 IU/kg total ADAMTS13 is administered about once every 12 hours. In certain embodiments, the dose is administered for treatment and/or prophylaxis. In certain aspects, the injection is administered intravenously. In other aspects, the injection is administered subcutaneously.

In one embodiment, the disclosure provides a method for treating ALI and/or ARDS in a mammal in need thereof, the method including administering a therapeutically effective amount of a composition comprising ADAMTS13 variant, with or without ADAMTS13 protein to the mammal, where the therapeutically effective amount of total ADAMTS13 is from about 40 to about 160 units of FRETS-VWF73 activity per kilogram body weight of the mammal (IU/kg). In a specific embodiment, the mammal is a human. In one embodiment, the about 40 IU/kg to about 160 IU/kg total ADAMTS13 is administered about once a month. In one embodiment, the about 40 IU/kg to about 160 IU/kg total ADAMTS13 is administered about twice a month. In one embodiment, the about 40 IU/kg to about 160 IU/kg total ADAMTS13 is administered about once a week. In one embodiment, the about 40 IU/kg to about 160 IU/kg total ADAMTS13 is administered about twice a week. In one embodiment, the about 40 IU/kg to about 160 IU/kg total ADAMTS13 is administered about three times a week. In one embodiment, the about 40 IU/kg to about 160 IU/kg total ADAMTS13 is administered about once every 48 hours. In one embodiment, the about 40 IU/kg to about 160 IU/kg total ADAMTS13 is administered about once every 24 hours. In one embodiment, the about 40 IU/kg to about 160 IU/kg total ADAMTS13 is administered about once every 12 hours. In certain embodiments, the dose of total ADAMTS13 is about 20 IU/kg, about 40 IU/kg, about 50 IU/kg, about 60 IU/kg, about 70 IU/kg, about 75 IU/kg, about 80 IU/kg, about 90 IU/kg, about 100 IU/kg, about 100 IU/kg, about 120 IU/kg, about 125 IU/kg, about 130 IU/kg, about 140 IU/kg, about 150 IU/kg, or about 160 IU/kg. In certain embodiments, the dose of total ADAMTS13 is about 40 IU/kg, about 80 IU/kg, or about 160 IU/kg. In certain embodiments, the dose is administered for treatment and/or prophylaxis. In certain aspects, the injection is administered intravenously. In other aspects, the injection is administered subcutaneously.

In certain embodiments, the present disclosure provides a method for treating or preventing a blood clotting disorders associated with cardiovascular disease in a subject, the method comprising administering to the subject in need thereof a therapeutically effective amount of an ADAMTS13 variant or composition thereof, including compositions with ADAMTS13. In certain embodiments of the methods provided herein, the blood clotting disorder includes, but is not limited to, myocardial infarction, myocardial ischemia, deep vein thrombosis, peripheral vascular disease, stroke, transient ischemic attack, and medical device associated thrombosis.

In certain embodiments, the present disclosure provides a method for treating or preventing hematologic disease in a subject, the method comprising administering to the subject in need thereof a therapeutically effective amount of an ADAMTS13 variant or composition thereof, including compositions with ADAMTS13. In certain embodiments of the methods provided herein, hematologic disease includes, but is not limited to, TTP (inherited and acquired), thrombotic microangiopathy, and sickle cell disease.

In certain embodiments, the disclosure provides methods for recanalization of occluded blood vessels in a subject having an infarction (e.g. a cerebral infarction), as described in WO 2016/191565, which is incorporated herein in its entirety for all purposes. ADAMTS13 variants and compositions thereof, including compositions with ADAMTS13, advantageously exerts their effect in a dose dependent manner and these effects are observed even at prolonged periods after blood vessel occlusion.

The subject method includes a step of administering to the subject a therapeutically effective amount of an ADAMTS13 variant, with or without ADAMTS13 protein, at particular dosages and ranges of times after detection of the infarction.

The subject methods are suitable for the treatment of any infarction caused by a blood vessel occlusion. Such infarctions include, but are not limited to, a myocardial infarction, a cerebral infarction, a pulmonary infarction, a splenic infarction, a limb infarction, a bone infarction, a testicle infarction, and an eye infarction.

In exemplary embodiments, the subject methods are for the recanalization of an occluded blood vessel in a subject having a cerebral infarction. “Cerebral infarction” refers to a type of ischemic stroke resulting from a blockage in the blood vessels supplying blood to the brain, which results in the death of brain tissue. Symptoms of cerebral infarction are determined by the parts of the brain affected. For example, infarcts in the primary motor cortex can cause contralateral hemiparesis. Brainstem infarcts cause brainstem syndromes including Wallenberg's syndrome, Weber's syndrome, Millard-Bubler syndrome, and Benedikt syndrome.

Recanalization of occluded blood vessels can be measured using any suitable technique. For example, recanalization can be measure by as a percentage of blood flow compared to a control baseline value (e.g., the blood flow of a control individual not having the occluded blood vessel or infarction). Blood flow can be measure, for example, using videocapillary microscoping with frame-to-frame analysis or laser Doppler anemometry techniques. See, e.g., Stucker et al. Microvascular Research 52(2): 188-192 (1996), which is incorporated herein by reference. In some embodiments, the subject methods increase the blood flow by at least 10%, 15%, 20%, 25%, 30%, 35%, 40%, 45%, 50%, 55%, 60%, 65%, 70%, 75%, 80%, 85%, 90%, 95%, or 99% as compared to a control baseline value (e.g., the blood flow of a control subject not having the occluded blood vessel or infarction).

Without being bound by any particular theory of operation, it is believed recanalization of occluded blood vessels via the disclosed composition reduces infarct volume. In some embodiments, administration of a composition disclosed herein reduces the infarct volume in the subject by at least 5% 10%, 15%, 20%, 25%, 30%, 35%, 40%, 45%, 50%, 55%, 60%, 65%, 70%, 75%, 80%, 85%, 90%, 95%, or 99% of the infarct volume of a control subject that was not administered the ADAMTS13 variant, alone or with ADAMTS13 protein.

In one embodiment, ADAMTS13 variant compositions, including compositions with ADAMTS13, is administered to reduce inflammation caused by the clotting disorder (e.g., an infarction), thereby preventing or reducing tissue damage (e.g., damage to the cerebral damage) and/or to reduce reperfusion injury by preventing leukocyte infiltration and damage. In one embodiment, ADAMTS13 compositions, including those with ADAMTS13, is administered to protect against secondary injury to infarct tissue (e.g., cerebral tissue and myocardial tissue) caused by reperfusion. In particular aspects, the injection is administered subcutaneously. In other aspects, the injection is administered intravenously.

### V. ADAMTS13 Variant Expression

In certain embodiments, an ADAMTS13 variant(s) and/or ADAMTS13 protein used in the compositions provided herein may be expressed, produced, or purified according to a method disclosed previously, for example, in U.S. Pat. Nos. 6,926,894, 8,313,926, US 2005/0266528, US 2007/0015703, US 2009/0317375, and WO 2002/42441, all of which are hereby incorporated by reference in their entirety for all purposes.

A. Host Cells and Vectors

Recombinant ADAMTS13 variant(s) and/or ADAMTS13 protein can be produced by expression in any suitable prokaryotic or eukaryotic host system. Examples of eukaryotic cells include, without limitation, mammalian cells, such as CHO (e.g., CHO DBX-11, CHOZN (Sigma)), COS, HEK 293, BHK, SK-Hep, and HepG2; insect cells, for example SF9 cells, SF21 cells, S2 cells, and High Five cells; and yeast cells, for example Saccharomyces or Schizosaccharomyces cells. In one embodiment, the ADAMTS13 proteins can be expressed in bacterial cells, yeast cells, insect cells, avian cells, mammalian cells, and the like. For example, in a human cell line, a hamster cell line, or a murine cell line. In one particular embodiment, the cell line is a CHO, BHK, or HEK cell line. In certain embodiments, the cell line is a CHO cell line.

In certain embodiments, serine protease inhibitors (e.g., aprotinin, antipain, chymostatin, elastatinal, phenylmethylsulfonyl fluoride (PMSF), APMSF, TLCK, TPCK, leupeptin and soybean trypsin inhibitor) can be added in upstream (e.g., during cultivation and harvesting) and downstream (e.g., during purification) manufacturing to prevent truncation of ADAMTS13 variants and/or ADAMTS13 protein. In certain embodiments, the serine protease inhibitor is aprotinin.

In one embodiment, the cells may be any mammalian cell that can be cultured, in a manufacturing process (i.e., at least 1 liter), to produce a desired ADAMTS13 protein such as an ADAMTS13 variant(s) and/or ADAMTS13 protein. Examples include the monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells/-DHFR, such as the DUKX-B11 subclone (CHO, Uriaub and Chasin, Proc. Natl. Acad. Sci. USA, 77:4216 (1980)); mouse Sertoli cells (TM4, Mather, Biol. Reprod, 23:243-251 (1980)); monkey kidney cells (CV1 ATCC CCL 70); African green monkey kidney cells (VERO-76, ATCC CRL-1587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N. Y. Acad. Sci., 383:44-68 (1982)); MRC 5 cells; FS4 cells; and the human hepatoma line (Hep G2). In certain embodiments, the cell line is a rodent cell line, especially a hamster cell line such as CHO or BHK.

A wide variety of vectors can be used for the expression of an ADAMTS13 variant (e.g., SEQ ID NO: 2) and/or a ADAMTS13 protein (e.g., SEQ ID NO: 1) and can be selected from eukaryotic and prokaryotic expression vectors. In certain embodiments, a plasmid vector is contemplated for use in expressing an ADAMTS13 variants and/or ADAMTS13 protein. In general, plasmid vectors containing replicon and control sequences which are derived from species compatible with the host cell are used in connection with these hosts. The vector can carry a replication site, as well as marking sequences which are capable of providing phenotypic selection in transformed cells. The plasmid will comprise a nucleotide sequence encoding an ADAMTS13 variant(s) and/or ADAMTS13 protein operable linked to one or more control sequences, for example, a promoter.

One embodiment entails a method of preparing stable CHO cell clones expressing a recombinant ADAMTS13 variant(s) and/or ADAMTS13 protein is as follows. A DHFR deficient CHO cell line DUKX-B11 is transfected with a DHFR expression vector to allow for expression of the relevant recombinant protein, essentially as described in U.S. Pat. No. 5,250,421 (Kaufman et al., Genetics Institute, Inc.). Selection is carried out by growth in Hypoxanthine/Thymidine (HT) free media and amplification of the relevant region coding for expression of the recombinant ADAMTS13 variant(s) and/or ADAMTS13 protein and DHFR gene is achieved by propagation of the cells in increasing concentrations of methotrexate. Where appropriate, CHO cell lines may be adapted for growth in serum and/or protein free medium, essentially as described in U.S. Pat. No. 6,100,061 (Reiter et al. lmmuno Aktiengesellschaft).

In certain embodiments, the recombinant ADAMTS13 variant(s) and/or ADAMTS13 protein proteins can be produced by expression in a CHO cell line that has been engineered to not express glutamine synthase (GS) and cultured to grow in a chemically defined and/or animal component free media, optionally without glutamine and/or hypoxanthine/thymidine. In certain embodiments, the CHO cell line is an engineered CHO K1 cell line engineered to not express glutamine synthase (GS) and cultured to grow in a chemically defined and/or animal component free media, optionally without glutamine and/or hypoxanthine/thymidine. In certain embodiments, the source of glutamine to maintain the cell line comes from the expression of exogenous glutamine linked to the expression of the recombinant ADAMTS13 variant(s) and/or ADAMTS13 protein proteins. In certain embodiments, the cell line can be a cell line as described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, 6,479,626, US20030232410, and/or US20090203140, which are each incorporated herein by reference in their entirety for all purposes. In certain embodiments, the chemically defined media can be, but not limited to EX-Cell media (e.g., EX-Cell CD CHO fusion media, EX-Cell Advanced CHO Fed-batch media) or Cellvento 4Feed). In certain embodiments the cell line can be, but is not limited to, CHOZN GS^{-/-} cell line (Sigma). In certain embodiments the cell line is CHOZN GS^{-/-} cell line. In certain embodiments, the cell line is cultured in EX-Cell Advanced CHO Fed-batch media.

In certain embodiments, the recombinant ADAMTS13 variant(s) and/or ADAMTS13 protein proteins can be produced by expression in a CHOZN GS^{-/-} cell line produced in EX-Cell Advanced CHO Fed-batch media. In certain embodiments, serine protease inhibitors (e.g., aprotinin, antipain, chymostatin, elastatinal, phenylmethylsulfonyl fluoride (PMSF), APMSF, TLCK, TPCK, leupeptin and soybean trypsin inhibitor) can be added in upstream (e.g., during cultivation and harvesting) and downstream (e.g., during purification) manufacturing to prevent truncation of ADAMTS13 variants and/or ADAMTS13 protein. In certain embodiments, the serine protease inhibitor is aprotinin.

In certain embodiments, stable HEK293 cells are prepared by transfecting with a construct containing a hygromycin selectable marker and selecting transformants by antibiotic resistance.

In some embodiments, the ADAMTS13 protein is glycosylated at one or more glycosylation sites. The glycosylation can occur, for example, at an O-glycosylation site of the ADAMTS13 protein, including at serine residues 5399, 5698, 5757, 5907, 5965, S1027 or S1087 of the amino acid sequence set forth in SEQ ID NO: 1. In certain embodiments, the ADAMTS13 protein can be 0-glycosylated with a disaccharide (e.g., a Fuc-Glc disaccharide) or a mucin-type 0-glycan (e.g., having the structure HexNAc-Hex-NeuAc₀₋₂).

In certain embodiments, the ADAMTS13 protein is glycosylated at an N-glycosylation site. The glycosylation can occur, for example, at one or more N-glycosylation sites of the ADAMTS13 protein, including asparagine residues N142, N146, N552, N579, N614, N667, N707, N828, N1235 or N1354 of the amino acid sequence set forth in SEQ ID NO: 1. In certain embodiments, the ADAMTS13 protein is glycosylated with high mannose-type N-glycan. In other embodiments, the ADAMTS13 is glycosylated with a hybrid-type N-glycan. In certain embodiments, the ADAMTS13 protein is glycosylated with a complex-type N-glycan, which may comprise, e.g., a core-fucose residue and/or one or more sialic acid residues. In certain embodiments, the ADAMTS13 protein is modified with N-glycans that are monosialayted, disialylated, trisialylated, or tetrasialylated. In certain embodiments, the sialylation is via an α2,6-linkage or an α2,3-linkage.

In certain embodiments, the ADAMTS13 protein is glycosylated at tryptophan residues at one or more C-mannosylation sites, such as, for example, W387 or W390 of the amino acid sequence of ADAMTS13 set forth in SEQ ID NO: 1.

In some embodiments, the ADAMTS13 or variant thereof comprises an N-glycan signature that has at least about 75%, about 76%, about 77%, about 78%, about 79%, about 80%, about 81%, about 82%, about 83%, about 84%, about 85%, or about 86% neutral, mono- and di-sialylated N-glycans combined. In some embodiments, the ADAMTS13 or variant thereof comprises an N-glycan signature that has at least about 5%, about 6%, about 7%, about 8%, about 9%, about 10%, about 11%, about 12%, about 13%, about 14% or about 15% tri- and tetra-sialylated glycans combined. In some embodiments, the ADAMTS13 or variant thereof comprises an N-glycan signature that has about 10% to about 35%, about 11% to about 34%, about 12% to about 33%, about 13% to about 32%, about 14% to about 31%, about 15% to about 30%, about 16% to about 29%, about 17% to about 28%, about 18% to about 27%, about 19% to about 26%, about 20% to about 25%, about 21% to about 24%, about 22% to about 24%, or 23% to about 24% neutral N-glycans. In some embodiments, the ADAMTS13 or variant thereof comprises an N-glycan signature that has about 20% to about 50%, about 21% to about 49%, about 22% to about 48%, about 23% to about 47%, about 24% to about 46%, about 25% to about 45%, about 26% to about 44%, about 27% to about 43%, about 28% to about 42%, about 29% to about 41%, about 30% to about 40%, about 31% to about 39%, about 32% to about 38%, about 33% to about 37%, about 34% to about 36% or about 35% monosialayted N-glycans. In some embodiments, the ADAMTS13 or variant thereof comprises an N-glycan signature that has about 10% to about 40%, about 11% to about 39%, about 12% to about 38%, about 13% to about 37%, about 14% to about 36%, about 15% to about 35%, about 16% to about 34%, about 17% to about 33%, about 18% to about 32%, about 19% to about 31%, about 20% to about 30%, about 22% to about 30%, about 24% to about 30%, about 25% to about 29%, about 26% to about 29%, about 27%, or about 28% disialylated N-glycans. In some embodiments, the ADAMTS13 or variant thereof comprises an N-glycan signature that has about 1% to about 25%, about 2% to about 24%, about 3% to about 23%, about 4% to about 22%, about 5% to about 20%, about 6% to about 19%, about 7% to about 18%, about 8% to about 17%, about 9% to about 16%, about 10% to about 15%, about 11% to about 14%, or about 11% to about 12% trisialylated N-glycans. In some embodiments, the ADAMTS13 or variant thereof comprises an N-glycan signature that has about 0.1% to about 10%, about 0.5% to about 8%, about 1% to about 7%, about 1% to about 5%, about 1% to about 4%, about 2% to about 6%, about 2% to about 4%, or about 3% tetrasialylated N-glycans. In some embodiments, the ADAMTS13 or variant thereof comprises an N-glycan signature that has about a N-glycan index of about 110 to about 160, about 111 to about 159, about 112 to about 158, about 113 to about 157, about 114 to about 156, about 115 to about 155, about 116 to about 154, about 117 to about 153, about 118 to about 152, about 119 to about 151, about 120 to about 150, about 121 to about 149, about 122 to about 148, about 123 to about 147, about 124 to about 146, about 125 to about 145, about 126 to about 144, about 127 to about 143, about 128 to about 142, about 129 to about 141, about 130 to about 140, about 133 to about 139, about 134, about 135, about 136, about 137 or about 138. In some embodiments, the ADAMTS13 or variant thereof is produced in a CHO, COS, HEK 293, BHK, SK-Hep, or HepG2 cell line. In some embodiments, the ADAMTS13 or variant thereof is produced in a CHO DBX-11 or CHOZN cell line. In some embodiments, the ADAMTS13 or variant thereof is produced in a CHOZN glutamine synthetase (GS)^{-/-} cell line.

In some embodiments, the ADAMTS13 or variant thereof of comprises a sialic acid signature that comprises a ratio of about 1% to about 15%, about 2% to about 12%, about 2% to about 10%, about 3% to about 9%, about 3% to about 8%, about 3% to about 6%, or about 4% to about 5%, or about 1%, about 2%, about 3%, about 4%, about 5%, about 6%, about 7%, about 8%, about 9%, about 10%, about 11%, about 12%, about 13%, about 14%, or about 15% of % NGNA relative to NANA. In some embodiments, the ADAMTS13 or variant thereof is produced in a CHO, COS, HEK 293, BHK, SK-Hep, or HepG2 cell line. In some embodiments, the ADAMTS13 or variant thereof is produced in a CHO DBX-11 or CHOZN cell line. In some embodiments, the ADAMTS13 or variant thereof is produced in a CHOZN glutamine synthetase (GS)−/− cell line.

In some embodiments, the ADAMTS13 or variant thereof of comprises an N-glycan signature that has at least about 80%, about 81%, about 82%, about 83%, about 84%, about 85%, or about 86% neutral, mono- and di-sialylated N-glycans combined. In some embodiments, the ADAMTS13 or variant thereof of comprises an N-glycan signature that has at least about 16%, about 17%, about 18%, or about 19% tri- and tetra-sialylated glycans combined. In some embodiments, the ADAMTS13 or variant thereof of comprises an N-glycan signature that has at least about 16%, about 17%, about 18%, or about 19% tri- and tetra-sialylated glycans combined. In some embodiments, the ADAMTS13 or variant thereof of comprises an N-glycan signature that has about 5% to about 30%, about 6% to about 28%, about 7% to about 26%, about 8% to about 25%, about 9% to about 22%, about 10% to about 20%, about 11% to about 18%, about 12% to about 17%, or about 13% to about 16% neutral N-glycans. In some embodiments, the ADAMTS13 or variant thereof of comprises an N-glycan signature that has about 10% to about 45%, about 11% to about 44%, about 12% to about 43%, about 13% to about 42%, about 14% to about 41%, about 15% to about 40%, about 16% to about 39%, about 17% to about 38%, about 18% to about 37%, about 19% to about 36%, about 20% to about 35%, about 22% to about 34%, about 24% to about 34%, about 26% to about 33%, about 27%, about 28%, about 29%, about 30%, about 31%, or about 32% monosialayted N-glycans. In some embodiments, the ADAMTS13 or variant thereof of comprises an N-glycan signature that has about 20% to about 55%, about 21% to about 54%, about 22% to about 53%, about 23% to about 52%, about 24% to about 51%, about 25% to about 50%, about 26% to about 49%, about 27% to about 48%, about 28% to about 47%, about 29% to about 46%, about 30% to about 45%, about 35% to about 44%, about 36% to about 44%, about 37% to about 43%, about 38%, about 39%, about 40%, about 41%, or about 42% disialylated N-glycans. In some embodiments, the ADAMTS13 or variant thereof of comprises an N-glycan signature that has about 1% to about 30%, about 2% to about 29%, about 3% to about 28%, about 4% to about 27%, about 5% to about 25%, about 6% to about 24%, about 7% to about 23%, about 8% to about 22%, about 9% to about 21%, about 10% to about 20%, about 11% to about 18%, about 12% to about 15%, about 13%, or about 14% trisialylated N-glycans. In some embodiments, the ADAMTS13 or variant thereof of comprises an N-glycan signature that has about 0.1% to about 15%, 0.5% to about 12%, about 1% to about 10%, 1% to about 9%, about 2% to about 8%, 2% to about 7%, about 3% to about 6%, or about 4%, or about 5% tetrasialylated N-glycans. In some embodiments, the ADAMTS13 or variant thereof of comprises an N-glycan signature that has a N-glycan index of about 130 to about 190, about 132 to about 189, about 134 to about 188, about 136 to about 186, about 140 to about 185, about 141 to about 183, about 142 to about 181, about 143 to about 179, about 144 to about 177, about 145 to about 175, about 147 to about 174, about 149 to about 173, about 151 to about 172, about 152 to about 171, about 153 to about 170, about 154, about 155, about 156, about 157, about 158, about 159, about 160, about 161, about 163, about 163, about 164, about 165, about 166, about 167, about 168, or about 169. In some embodiments, the ADAMTS13 or variant thereof is produced in a CHO, COS, HEK 293, BHK, SK-Hep, or HepG2 cell line. In some embodiments, the ADAMTS13 or variant thereof is produced in a CHO DBX-11 or CHOZN cell line. In some embodiments, the ADAMTS13 or variant thereof is produced in a CHO DBX-11 cell line.

In some embodiments, the ADAMTS13 or variant thereof comprises a monosaccharides signature that has about 10% to about 40%, about 11% to about 39%, about 12% to about 38%, about 13% to about 37%, about 14% to about 36%, about 15% to about 35%, about 16% to about 34%, about 17% to about 33%, about 18% to about 32%, about 19% to about 31%, about 20% to about 30%, about 21% to about 30%, about 23% to about 30%, about 25% to about 29%, about 26% to about 29%, about 27% to about 29% or about 28% GlcNAc. In some embodiments, the ADAMTS13 or variant thereof comprises a monosaccharides signature that has about 0.1% to about 10%, about 1% to about 8%, about 5% to about 6% or about 6% GalNAc. In some embodiments, the ADAMTS13 or variant thereof comprises a monosaccharides signature that has about 10% to about 35%, about 11% to about 34%, about 12% to about 33%, about 13% to about 32%, about 14% to about 31%, about 15% to about 30%, about 16% to about 29%, about 17% to about 28%, about 18% to about 27%, about 19% to about 26%, about 20% to about 25%, about 21% to about 25%, about 22% to about 24%, or about 23% to about 24% Gal. In some embodiments, the ADAMTS13 or variant thereof comprises about 10% to about 35%, about 11% to about 34%, about 12% to about 33%, about 13% to about 32%, about 14% to about 31%, about 15% to about 30%, about 16% to about 29%, about 18% to about 28%, about 20% to about 28%, about 21% to about 27%, about 22% to about 26%, about 23% to about 25%, about 23%, about 24%, or about 25% Man. In some embodiments, the ADAMTS13 or variant thereof comprises about 0.1% to about 20%, about 0.5% to about 18%, about 1% to about 15%, about 2% to about 14%, about 3% to about 13%, about 4% to about 12%, about 5% to about 10%, about 5% to about 8%, about 6% to about 7%, about 6%, or about 7% Glc. In some embodiments, the ADAMTS13 or variant thereof comprises about 5% to about 20%, about 6% to about 19%, about 7% to about 18%, about 8% to about 17%, about 9% to about 16%, about 10% to about 15%, about 10% to about 14%, about 11% to about 13%, about 11% to about 12%, about 10%, about 11%, or about 12% Fuc. In some embodiments, the ADAMTS13 or variant thereof is produced in a CHO, COS, HEK 293, BHK, SK-Hep, or HepG2 cell line. In some embodiments, the ADAMTS13 or variant thereof is produced in a CHO DBX-11 or CHOZN cell line. In some embodiments, the ADAMTS13 or variant thereof is produced in a CHO DBX-11 cell line.

In some embodiments, the ADAMTS13 or variant thereof comprises a monosaccharides signature that has about 100 nmol to about 200 nmol, about 105 nmol to about 190 nmol, about 110 nmol to about 180 nmol, about 118 nmol to about 175 nmol, about 120 nmol to about 172, about 125 nmol to about 170 nmol, about 130 nmol to about 169 nmol, about 135 nmol, about 140 nmol, about 145 nmol, about 150 nmol, about 155 nmol, about 160 nmol, or about 165 nmol NANA/mg. In some embodiments, the ADAMTS13 or variant thereof comprises a monosaccharides signature that has about 0.01 nmol to about 1 nmol, about 0.02 nmol to about 0.75 nmol, about 0.04 nmol to about 0.60 nmol, about 0.05 nmol to about 0.50 nmol, about 0.06 nmol to about 0.40 nmol, about 0.07 nmol to about 0.35 nmol, about 0.08 nmol to about 0.30 nmol, about 0.1 nmol to about 0.3 nmol, about 0.1 nmol, about 0.2 nmol, or about 0.3 nmol NGNA/mg. In some embodiments, the ADAMTS13 or variant thereof comprises a monosaccharides signature that has a ratio of about 0.01% to about 1%, about 0.02% to about 0.75%, about 0.04% to about 0.6%, about 0.05% to about 0.5%, about 0.06% to about 0.4%, about 0.06% to about 0.2%, about 0.08% to about 0.2%, about 0.1% to about 0.2%, about 0.1%, about 0.2%, or about 0.15% of % NGNA relative to NANA. In some embodiments, the ADAMTS13 or variant thereof is produced in a CHO, COS, HEK 293, BHK, SK-Hep, or HepG2 cell line. In some embodiments, the ADAMTS13 or variant thereof is produced in a CHO DBX-11 or CHOZN cell line. In some embodiments, the ADAMTS13 or variant thereof is produced in a CHO DBX-11 cell line.

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Accordingly, in certain embodiments, a viral vector is used to introduce a nucleotide sequence encoding an ADAMTS13 variant(s) and/or ADAMTS13 protein into a host cell for expression. The viral vector will comprise a nucleotide sequence encoding an ADAMTS variant and/or ADAMTS13 operable linked to one or more control sequences, for example, a promoter. Alternatively, the viral vector may not contain a control sequence and will instead rely on a control sequence within the host cell to drive expression of the ADAMTS13 variants and/or ADAMTS13 protein. Non-limiting examples of virus vectors that may be used to deliver a nucleic acid include adenoviral vectors, AAV vectors, and retroviral vectors.

In one embodiment, an adenovirus expression vector include those constructs containing adenovirus sequences sufficient to support packaging of the construct and to ultimately express an ADAMTS construct that has been cloned therein. Adenoviral vectors allow for the introduction of foreign sequences up to 7 kb (Grunhaus et al., Seminar in Virology, 200(2):535-546, 1992)).

In another embodiment, an adeno-associated virus (AAV) can be used to introduce a nucleotide sequence encoding an ADAMTS13 protein (e.g., ADAMTS13) into a host cell for expression. AAV systems have been described previously and are generally well known in the art (Kelleher and Vos, Biotechniques, 17(6):1110-7, 1994; Cotten et al., Proc Natl Acad Sci USA, 89(13):6094-6098, 1992; Curiel, Nat Immun, 13(2-3):141-64, 1994; Muzyczka, Curr Top Microbiol Immunol, 158:97-129, 1992). Details concerning the generation and use of rAAV vectors are described, for example, in U.S. Pat. Nos. 5,139,941 and 4,797,368, each incorporated herein by reference in their entireties for all purposes.

In one embodiment, a retroviral expression vector can be used to introduce a nucleotide sequence encoding an ADAMTS13 variant(s) and/or ADAMTS13 protein into a host cell for expression. These systems have been described previously and are generally well known in the art (Mann et al., Cell, 33:153-159, 1983; Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986). In a specific embodiment, the retroviral vector is a lentiviral vector (see, for example, Naldini et al., Science, 272(5259):263-267, 1996; Zufferey et al., Nat Biotechnol, 15(9):871-875, 1997; Blomer et al., J Virol., 71(9):6641-6649, 1997; U.S. Pat. Nos. 6,013,516 and 5,994,136).

Non-limiting examples of vectors for prokaryotic expression include plasmids such as pRSET, pET, pBAD, etc., wherein the promoters used in prokaryotic expression vectors include lac, trc, trp, recA, araBAD, etc. Examples of vectors for eukaryotic expression include: (i) for expression in yeast, vectors such as pAO, pPIC, pYES, pMET, using promoters such as AOX1, GAP, GAL1, AUG1, etc.; (ii) for expression in insect cells, vectors such as pMT, pAc5, pIB, pMIB, pBAC, etc., using promoters such as PH, p10, MT, Ac5, OpIE2, gp64, polh, etc., and (iii) for expression in mammalian cells, vectors such as pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

In certain embodiments, the cell-culture expression of ADAMTS13 variant(s) and/or ADAMTS13 protein may comprise the use of a microcarrier. The present invention provides, among other aspect, methods of large-scale ADAMTS13 variant(s) and/or ADAMTS13 protein expression. In some embodiments, the cell-cultures of the embodiments can be performed in large bioreactors under conditions suitable for providing high volume-specific culture surface areas to achieve high cell densities and protein expression. One means for providing such growth conditions is to use microcarriers for cell-culture in stirred tank bioreactors. In another embodiment, these growth requirements are met via the use of a suspension cell culture.

B. Cultivation Methods

In certain embodiments, ADAMTS13 variant(s) and/or ADAMTS13 protein expression can comprise the use of a cell culture system operated under a batch or continuous mode of operation. For example, when batch cell cultures are utilized, they may be operated under single batch, fed-batch, or repeated-batch mode. Likewise, continuous cell cultures may be operated under, for example, perfusion, turbidostat or chemostat mode. Batch and continuous cell cultivation may be performed under either suspension or adherence conditions. When operated under suspension conditions, the cells will be freely suspended and mixed within the culture medium. Alternatively, under adherence conditions, the cells will be bound to a solid phase, for example, a microcarrier, a porous microcarrier, disk carrier, ceramic cartridge, hollow fiber, flat sheet, gel matrix, and the like.

A batch culture is typically a large scale cell culture in which a cell inoculum is cultured to a maximum density in a tank or fermenter, and harvested and processed as a single batch. A fed-batch culture it typically a batch culture which is supplied with either fresh nutrients (e.g., growth-limiting substrates) or additives (e.g., precursors to products). The feed solution is usually highly concentrated to avoid dilution of the bioreactor. In a repeated-batch culture, the cells are placed in a culture medium and grown to a desired cell density. To avoid the onset of a decline phase and cell death, the culture is then diluted with complete growth medium before the cells reach their maximum concentration. The amount and frequency of dilution varies widely and depends on the growth characteristics of the cell line and convenience of the culture process. The process can be repeated as many times as required and, unless cells and medium are discarded at subculture, the volume of culture will increase stepwise as each dilution is made. The increasing volume may be handled by having a reactor of sufficient size to allow dilutions within the vessel or by dividing the diluted culture into several vessels. The rationale of this type of culture is to maintain the cells in an exponentially growing state. Serial subculture is characterized in that the volume of culture is always increasing stepwise, there can be multiple harvests, the cells continue to grow and the process can continue for as long as desired. In certain embodiments, an ADAMTS13 variant(s) and/or ADAMTS13 protein may be recovered after harvesting the supernatant of a batch culture.

A continuous culture can be a suspension culture that is continuously supplied with nutrients by the inflow of fresh medium, wherein the culture volume is usually kept constant by the concomitant removal of spent medium. In chemostat and turbidostat methods, the extracted medium contains cells. Thus, the cells remaining in the cell culture vessel must grow to maintain a steady state. In the chemostat method, the growth rate is typically controlled by controlling the dilution rate, i.e., the rate at which fresh medium is added. The growth rate of the cells in the culture may be controlled, for example, at a sub-maximal growth rate, by alteration of the dilution rate. In contrast, in the turbidostat method, the dilution rate is set to permit the maximum growth rate that the cells can achieve at the given operating conditions, such as pH and temperature.

In a perfusion culture, the extracted medium is depleted of cells, which are retained in the culture vessel, for example, by filtration or by centrifugal methods that lead to the reintroduction of the cells into the culture. However, typically membranes used for filtration do not retain 100% of cells, and so a proportion are removed when the medium is extracted. It may not be crucial to operate perfusion cultures at very high growth rates, as the majority of the cells are retained in the culture vessel.

Stirred-tank reactor system can be used for batch and continuous cell cultures operated under suspension or adherent modes. Generally, the stirred-tank reactor system can be operated as any conventional stirred-tank reactor with any type of agitator such as a Rushton, hydrofoil, pitched blade, or marine.C. Culture Mediums

ADAMTS13 variant(s) and/or ADAMTS13 protein may be expressed in culture mediums which are free of exogenously added protein. “Protein free culture medium” and related terms refers to culture medium lacking protein that is from a source exogenous to or other than the cells in the culture, which naturally shed proteins during growth. In one embodiment, an ADAMTS13 variant(s) and/or ADAMTS13 protein can be expressed in a medium which is free of exogenously added protein (i.e., protein-free) and is supplemented with zinc, calcium, and/or nicotinamide (vitamin B3). In certain embodiments, the protein free culture medium contains a polyamine. For example, at a concentration of at least 2 mg/L, or at or about between 2 mg/L and 30 mg/L, or at or about between 2 mg/L and 8 mg/L. In a specific embodiment, the polyamine is putrescine. Exemplary protein free culture mediums are taught in U.S. Pat. Nos. 6,171,825, 6,936,441, 8,313,926; WO 2007/077217; and U.S. Patent Application Publication Numbers 2008/0009040 and 2007/0212770, the disclosures of which are incorporated herein by reference in their entireties for all purposes.

Methods of preparing animal protein-free and chemically defined culture mediums are known in the art, for example in U.S. Pat. Nos. 6,171,825 and 6,936,441, WO 2007/077217, and U.S. Patent Application Publication Numbers 2008/0009040 and 2007/0212770, the disclosures of which are incorporated herein by reference in their entireties for all purposes. In one embodiment, the culture medium used to express an ADAMTS13 protein is animal protein-free or oligopeptide-free medium. In certain embodiments, the culture medium may be chemically defined. In certain embodiments, the culture media may contain at least one polyamine at a concentration of about 0.5 mg/L to about 10 mg/L.

ADAMTS13 variant(s) and/or ADAMTS13 protein can also be expressed in culture mediums which are free of exogenously added oligopeptides. In one embodiment, ADAMTS13 variant(s) and/or ADAMTS13 protein is expressed in a culture medium which is free of exogenously added oligopeptides (i.e., polypeptide-free) and is supplemented with zinc, calcium, and/or nicotinamide (vitamin B3). In certain embodiments, the oligopeptide free culture medium contains a polyamine. For example, at a concentration of at least 2 mg/L, or at or about between 2 mg/L and 30 mg/L, or at or about between 2 mg/L and 8 mg/L. In a specific embodiment, the polyamine is putrescine. Exemplary oligopeptide free culture mediums are taught in U.S. Pat. Nos. 6,171,825, 6,936,441, 8,313,926; WO 2007/077217; and U.S. Patent Application Publication Numbers 2008/0009040 and 2007/0212770, the disclosures of which are incorporated herein by reference in their entireties for all purposes.

ADAMTS13 variant(s) and/or ADAMTS13 protein can also be expressed in culture mediums which are free of serum. In one embodiment, ADAMTS13 variant(s) and/or ADAMTS13 protein is expressed in a culture medium which is free of exogenously added serum (i.e., serum-free) and is supplemented with zinc, calcium, and/or nicotinamide (vitamin B3). In certain embodiments, the serum-free culture medium contains a polyamine. For example, at a concentration of at least 2 mg/L, or at or about between 2 mg/L and 30 mg/L, or at or about between 2 mg/L and 8 mg/L. In a specific embodiment, the polyamine is putrescine. Exemplary serum-free culture mediums are taught in U.S. Pat. Nos. 6,171,825, 6,936,441, 8,313,926; WO 2007/077217; and U.S. Patent Application Publication Numbers 2008/0009040 and 2007/0212770, the disclosures of which are incorporated herein by reference in their entireties for all purposes.

ADAMTS13 variant(s) and/or ADAMTS13 protein can also be expressed in culture mediums which are free of animal proteins. In one embodiment, ADAMTS13 variant(s) and/or ADAMTS13 protein is expressed in a culture medium which is free of exogenously added animal proteins or polypeptides (i.e., animal protein free) and is supplemented with zinc, calcium, and/or nicotinamide (vitamin B3). In certain embodiments, the animal protein free culture medium contains a polyamine. For example, at a concentration of at least 2 mg/L, or at or about between 2 mg/L and 30 mg/L, or at or about between 2 mg/L and 8 mg/L. In a specific embodiment, the polyamine is putrescine. Exemplary animal protein free culture mediums are taught in U.S. Pat. Nos. 6,171,825, 6,936,441, 8,313,926; WO 2007/077217; and U.S. Patent Application Publication Numbers 2008/0009040 and 2007/0212770, the disclosures of which are incorporated herein by reference in their entireties for all purposes.

ADAMTS13 variant(s) and/or ADAMTS13 protein can also be expressed in culture mediums supplemented with additional calcium, zinc, and/or vitamin B3, as described in U.S. Pat. No. 8,313,926, the disclosure of which is incorporated herein by reference in its entirety for all purposes. In certain embodiments, the medium may be an animal protein-free, oligopeptide-free, or chemically defined medium. In certain embodiments, the animal protein-free or oligopeptide free medium is prepared as taught in U.S. Pat. Nos. 6,171,825 and 6,936,441, WO 2007/077217, and U.S. Patent Application Publication Numbers 2008/0009040 and 2007/0212770, the disclosures of which are incorporated herein by reference in their entireties for all purposes, both of which are incorporated herein by reference in their entireties for all purposes, and supplemented with additional calcium, zinc, and/or vitamin B3. In a specific embodiment, the chemically defined culture medium may be similar to a Dulbecco's Modified Eagle's Media (DMEM), which has been supplemented with additional calcium, zinc, and/or vitamin B3, in order to increase the specific activity of an ADAMTS variant and/or ADAMTS13 expressed in a cell cultured in the medium. In yet other embodiments, the culture medium is animal component free. In another embodiment, the culture medium contains protein, e.g., animal protein from serum such as fetal calf serum. In another embodiment, the culture has recombinant proteins exogenously added. In another embodiment, the proteins are from a certified pathogen free animal.

### VI. ADAMTS13 Variant Kits

In another aspect, kits are provided for the treatment of a disease or condition associated with ADAMTS13 or VWF dysfunction. In one embodiment, the kit comprises a composition of ADAMTS13 variants and/or ADAMTS13 protein. In some embodiments, the kits provided herein may contain one or more dose of a liquid or lyophilized composition as provided herein. When the kits comprise a lyophilized ADAMTS13 variant(s) and/or ADAMTS13 protein composition, generally the kits will also contain a suitable liquid for reconstitution of the liquid composition, for example, sterile water or a pharmaceutically acceptable buffer. In some embodiments, a kit includes an ADAMTS13 variant composition, including a composition with ADAMTS13 composition prepackaged in a syringe for subcutaneous administration by a health care professional or for home use.

In some embodiments, the ADAMTS13 variant comprises the amino acid sequence set forth in SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the nucleotide sequence that encodes the ADAMTS13 variant comprises the nucleotide sequence that encodes the amino acid sequence of SEQ ID NO: 2, or a variant thereof having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2 while still maintaining R⁹⁷. In certain embodiments, the ADAMTS13 variant comprises the amino acid sequence set forth in SEQ ID NO: 2. In certain embodiments, the ADAMTS13 variant consists of the amino acid sequence set forth in SEQ ID NO: 2. In certain embodiments, the ADAMTS13 variant consists essentially of the amino acid sequence set forth in SEQ ID NO: 2.

In some embodiments, the ADAMTS13 protein comprises the amino acid sequence set forth in SEQ ID NO: 1, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 1. In certain embodiments, the nucleotide sequence that encodes the ADAMTS13 protein comprises the nucleotide sequence that encodes the amino acid sequence of SEQ ID NO: 1, or a sequence having at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, sequence identity with SEQ ID NO: 2. In certain embodiments, the ADAMTS13 protein comprises the amino acid sequence set forth in SEQ ID NO: 1. In certain embodiments, the ADAMTS13 protein consists of the amino acid sequence set forth in SEQ ID NO: 1. In certain embodiments, the ADAMTS13 protein consists essentially of the amino acid sequence set forth in SEQ ID NO: 1.

In one embodiment, a kit is provided comprising between about 1 unit of FRETS-VWF73 activity and about 10,000 units of FRETS-VWF73 activity. In other embodiments, the kit may provide, for example, between about 20 units of FRETS-VWF73 (U_{FV73}) activity and about 8,000 units of FRETS-VWF73 activity, or between about 30 U_{FV73} and about 6,000 U_{FV73}, or between about 40 U_{FV73} and about 4,000 U_{FV73}, or between about 50 U_{FV73} and about 3,000 U_{FV73}, or between about 75 U_{FV73} and about 2,500 U_{FV73}, or between about 100 U_{FV73} and about 2,000 U_{FV73}, or between about 200 U_{FV73} and about 1,500 U_{FV73}, or between about other ranges therein. In certain embodiments, a kit may provide about 10 units of FRETS-VWF73 activity, or about 1, 5, 10, 15, 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000, 11,000, 12,000, 13,000, 14,000, 15,000, 16,000, 17,000, 18,000, 19,000, 20,000 or more units of FRETS-VWF73 activity.

In certain embodiments, the kit is for a single administration or dose of ADAMTS13 variants and/or ADAMTS13 protein. In other embodiments, the kit may contain multiple doses of ADAMTS13 variant(s) and/or ADAMTS13 protein for administration. In one embodiment, the kit may comprise an ADAMTS13 variant(s) and/or ADAMTS13 protein composition prepackaged in a syringe for administration by a health care professional or for home use.

### VII. Embodiments

This application provides the following non-limiting embodiments.
1. A recombinant ADAMTS13 variant, wherein the ADAMTS13 variant comprises an amino acid sequence with at least one amino acid substitution as compared to an ADAMTS13 protein.
2. The recombinant ADAMTS13 variant of embodiment 1, wherein the ADAMTS13 protein is a human ADAMTS13.
3. The recombinant ADAMTS13 variant of embodiment 1, wherein the ADAMTS13 protein comprises the amino acid sequence of SEQ ID NO: 1.
4. The recombinant ADAMTS13 variant of any of the preceding embodiments, wherein at least one of the single amino acid substitutions is within the ADAMTS13 catalytic domain as compared to the ADAMTS13 protein.
5. The recombinant ADAMTS13 variant of any one of embodiments 1-3, wherein the single amino acid substitution is not I⁷⁹M, V⁸⁸M, H⁹⁶D, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid position in an ADAMTS13.
6. The recombinant ADAMTS13 variant of any one of embodiments 1-3, wherein the single amino acid substitution is at amino acid Q⁹⁷ as denoted in SEQ ID NO: 1, or the equivalent amino acid position in an ADAMTS13.
7. The recombinant ADAMTS13 variant of embodiment 6, wherein the single amino acid change is from a Q to a D, E, K, H, L, N, P, or R.
8. The recombinant ADAMTS13 variant of embodiment 6, wherein the single amino acid change is from a Q to an R.
9. The recombinant ADAMTS13 variant of embodiment 8, wherein the ADAMTS13 variant comprises the amino acid sequence of SEQ ID NO: 2, or an amino acid sequence having at least 80% sequence identity thereof.
10. The recombinant ADAMTS13 variant of embodiment 8, wherein the ADAMTS13 consists essentially of the amino acid sequence of SEQ ID NO: 2.
11. The recombinant ADAMTS13 variant of embodiment 8, wherein the ADAMTS13 consists of the amino acid sequence of SEQ ID NO: 2.
12. A pharmaceutical composition comprising at least one ADAMTS13 variant of any one of embodiments 1-11 and a pharmaceutically acceptable carrier or excipient.
13. The pharmaceutical composition of embodiment 12, further comprising an ADAMTS13 protein.
14. The pharmaceutical composition of embodiment 13, wherein the ADAMTS13 protein comprises the amino acid sequence of SEQ ID NO: 1, or an amino acid sequence having at least 80% sequence identity thereof
15. The pharmaceutical composition of embodiment 13, wherein the ADAMTS13 protein consists of the amino acid sequence of SEQ ID NO: 1.
16. The pharmaceutical composition of any one of embodiments 13-15, wherein the ADAMTS13 protein is recombinantly produced.
17. The pharmaceutical composition of any one of embodiments 13-15, wherein the ADAMTS13 protein is plasma derived.
18. The pharmaceutical composition of any one of embodiments 13-15, wherein the ratio of ADAMTS13 variant to ADAMTS13 protein is about 1:1 to about 3:1, about 1:1 or about 3:2.
19. The pharmaceutical composition of any one of embodiments 13-18, wherein the ADAMTS13 variant constitutes between about 52% to about 72% or between about 47% to about 84% of total amount of all ADAMTS13 proteins and variants in the composition.
20. The pharmaceutical composition of embodiment 18 or embodiment 19, wherein the ratio or percentage is determined by peptide mapping method.
21. The pharmaceutical composition of embodiment 18 or embodiment 19, wherein the ratio or percentage is determined by HPLC analysis of tryptic peptides separated by liquid chromatography followed by mass spectrometry analysis.
22. The pharmaceutical composition of any one of embodiments 18-21, wherein the ratio or percentage is determined based on intensities in extracted ion chromatograms.
23. The pharmaceutical composition of any one of embodiments 18-22, wherein the ratio or percentage is determined based on the peak area of tryptic peptides of the ADAMTS13 variant in relation to the sum of the peak areas of all ADAMTS13 proteins and variants in the composition.
24. The pharmaceutical composition of embodiment 23, wherein the tryptic peptides of all ADAMTS13 proteins and variants in the composition being measured are specific to the at least one amino acid difference between the ADAMTS13 variant as compared to all other ADAMTS13 proteins and variants in the composition.
25. The pharmaceutical composition of embodiment 24, wherein the tryptic peptide(s) measured for the ADAMTS13 variant is AAGGILHLELLVAVGPDVFQAHR (SEQ ID NO: 115) or a combination of AAGGILHLELLVAVGPDVFQAHR (SEQ ID NO: 115) and EDTER (SEQ ID NO: 5).
26. The pharmaceutical composition of embodiment 24 or embodiment 25, wherein the tryptic peptide measured for the ADAMTS13 protein is

(SEQ ID NO: 6)
AAGGILHLELLVAVGPDVFQAHQEDTER.

27. The pharmaceutical composition of embodiment 18 or embodiment 19, wherein the ratio or percentage is determined based on total weight of ADAMTS13 variant in relation to the sum total weight of all ADAMTS13 proteins and variants in the composition.
28. A method for treating or preventing a blood clotting disorder in a subject suffering from or at risk of suffering from a blood clotting disorder, comprising administering to the subject in need thereof a therapeutically effective amount of an ADAMTS13 variant of any one of embodiments 1-11 or pharmaceutical composition of any one of embodiments 12-27.
29. The method of embodiment 28, wherein the therapeutically effective amount of total ADAMTS13 is from about 1 to about 4,000 IU/kg body weight.
30. The method of embodiment 28, wherein the therapeutically effective amount of total ADAMTS13 is from about 5 to about 4,000 IU/kg body weight.
31. The method of embodiment 28, wherein the therapeutically effective amount of total ADAMTS13 is from about 10 to about 2,000 IU/kg body weight.
32. The method of embodiment 28, wherein the therapeutically effective amount of total ADAMTS13 is from about 20 to about 200 IU/kg body weight.
33. The method of embodiment 28, wherein the therapeutically effective amount of total ADAMTS13 is from about 10 to about 200 IU/kg body weight.
34. The method of embodiment 28, wherein the therapeutically effective amount of total ADAMTS13 is from about 0.1 to about 4,000 IU/kg/day via continuous infusion.
35. The method of embodiment 28, wherein the therapeutically effective amount of total ADAMTS13 is from about 1 to about 200 IU/kg/day via continuous infusion.
36. The method of any one of embodiments 28-35, wherein the blood clotting disorder is inherited TTP, acquired TTP, infarction, cerebral infarction, myocardial infarction, ischemic/reperfusion injury, deep vein thrombosis, or sepsis-related disseminated intravascular coagulation.
37. The method according to any one of embodiments 28-36, wherein the blood clotting disorder is inherited TTP.
38. The method of embodiment 37, wherein the therapeutically effective amount of total ADAMTS13 comprises at least about 1 to about 4,000 IU/kg body weight.
39. The method of embodiment 37, wherein the therapeutically effective amount of total ADAMTS13 comprises at least about 5 to about 4,000 IU/kg body weight.
40. The method of embodiment 37, wherein the therapeutically effective amount of total ADAMTS13 comprises at least about 5 to about 500 IU/kg body weight.
41. The method of embodiment 37, wherein the therapeutically effective amount of total ADAMTS13 comprises at least about 10 to about 1,500 IU/kg body weight.
42. The method of embodiment 37, wherein the therapeutically effective amount of total ADAMTS13 comprises at least about 10 to about 160 IU/kg body weight.
43. The method of embodiment 37, wherein the therapeutically effective amount of total ADAMTS13 comprises at least about 10 to about 40 IU/kg body weight.
44. The method of embodiment 37, wherein the therapeutically effective amount of total ADAMTS13 comprises at least about 20 to about 40 IU/kg body weight.
45. The method of embodiment 37, wherein the therapeutically effective amount of total ADAMTS13 comprises at least about 20 to about 160 IU/kg body weight.
46. The method of embodiment 37, wherein the therapeutically effective amount of total ADAMTS13 is from about 20 to about 160 IU/kg body weight.
47. The method of embodiment 37, wherein the therapeutically effective amount of total ADAMTS13 is about 20 IU/kg body weight.
48. The method of embodiment 37, wherein the therapeutically effective amount of total ADAMTS13 is about 40 IU/kg body weight.
49. The method according to any one of embodiments 28-36, wherein the blood clotting disorder is acquired TTP.
50. The method of embodiment 49, wherein the therapeutically effective amount of total ADAMTS13 is from about 1 to about 4,000 IU/kg body weight.
51. The method of embodiment 49, wherein the therapeutically effective amount of total ADAMTS13 is from about 5 to about 4,000 IU/kg body weight.
52. The method of embodiment 49, wherein the therapeutically effective amount of total ADAMTS13 is from about 10 to about 1,500 IU/kg body weight.
53. The method of embodiment 49, wherein the therapeutically effective amount of total ADAMTS13 is from about 10 to about 80 IU/kg body weight.
54. The method of embodiment 49, wherein the therapeutically effective amount of total ADAMTS13 is from about 20 to about 80 IU/kg body weight.
55. The method of embodiment 49, wherein the therapeutically effective amount of total ADAMTS13 is from about 20 to about 40 IU/kg body weight.
56. The method of embodiment 49, wherein the therapeutically effective amount of total ADAMTS13 is from about 40 to about 2,000 IU/kg body weight.
57. The method according to any one of embodiments 28-36, wherein the blood clotting disorder is cerebral infarction and/or ischemia reperfusion injury.
58. The method of embodiment 57, wherein the therapeutically effective amount of total ADAMTS13 is from about 1 to about 4,000 IU/kg body weight.
59. The method of embodiment 57, wherein the therapeutically effective amount of total ADAMTS13 is from about 5 to about 4,000 IU/kg body weight.
60. The method of embodiment 57, wherein the therapeutically effective amount of total ADAMTS13 is from about 10 to about 2,000 IU/kg body weight.
61. The method of embodiment 57, wherein the therapeutically effective amount of total ADAMTS13 is from about 10 to about 1,500 IU/kg body weight.
62. The method of embodiment 57, wherein the therapeutically effective amount of total ADAMTS13 is from about 40 to about 4,000 IU/kg body weight.
63. The according to any one of embodiments 28-36, wherein the blood clotting disorder is myocardial infarction and/or ischemia reperfusion injury.
64. The method of embodiment 63, wherein the therapeutically effective amount of total ADAMTS13 is from about 1 to about 4,000 IU/kg body weight.
65. The method of embodiment 63, wherein the therapeutically effective amount of total ADAMTS13 is from about 5 to about 4,000 IU/kg body weight.
66. The method of embodiment 63, wherein the therapeutically effective amount of total ADAMTS13 is from about 10 to about 2,000 IU/kg body weight.
67. The method of embodiment 63, wherein the therapeutically effective amount of total ADAMTS13 is from about 10 to about 1,500 IU/kg body weight.
68. The method of embodiment 63, wherein the therapeutically effective amount of total ADAMTS13 is from about 40 to about 2,000 IU/kg body weight.
69. A method for treating or preventing a bleeding episode in a subject in a subject suffering from or at risk of suffering from a bleeding disorder, comprising administering to the subject in need thereof a therapeutically effective amount of an ADAMTS13 variant of any one of embodiments 1-11 or pharmaceutical composition of any one of embodiments 12-27.
70. The method of embodiment 69, wherein the bleeding episode is associated with inherited TTP, acquired TTP, infarction, cerebral infarction, myocardial infarction, ischemic/reperfusion injury, deep vein thrombosis, or sepsis-related disseminated intravascular coagulation.
71. A method for treating or preventing a vaso-occlusive crisis in a subject suffering from sickle cell disease, comprising administering to the subject in need thereof a therapeutically effective amount of the ADAMTS13 variant of any one of embodiments 1-11 or pharmaceutical composition of any one of embodiments 12-27.
72. The method of embodiment 71, wherein the subject is administered the ADAMTS13 variant or composition after symptoms of a vaso-occlusive crisis are present.
73. The method of embodiment 71, wherein the subject is administered the ADAMTS13 variant or composition before symptoms of a vaso-occlusive crisis are present.
74. The method of any one of embodiments 71-73, wherein administering the ADAMTS13 variant or composition reduces at least one of inflammation, vasoconstriction, platelet aggregation, or a combination of any thereof as compared to control or without treatment.
75. The method of any one of embodiments 71-74 wherein administering the ADAMTS13 variant or composition results in at least one of improved survival, improved lung function, reduced organ damage, reduced pulmonary vascular leakage, or a combination of any thereof as compared to control or without treatment.
76. The method of any one of embodiments 71-75, wherein administering the ADAMTS13 variant or composition reduces and/or prevents at least one of impaired blood flow, blood coagulation, vascular inflammation, thrombosis, ischemic cell damage, or organ damage, or a combination of any thereof as compared to control or without treatment.
77. The method of any one of embodiments 71-76, wherein administering the ADAMTS13 variant or composition reduces and/or prevents pain or severity of the pain as compared to control or without treatment.
78. The method of any one of embodiments 71-77, wherein administering the ADAMTS13 variant or composition reduces the frequency of occurrence of VOC and/or duration of VOC episodes as compared to without treatment.
79. The method of any one of embodiments 71-78, wherein the therapeutically effective amount of total ADAMTS13 is from about 1 to about 4,000 IU/kg body weight.
80. The method of any one of embodiments 71-78, wherein the therapeutically effective amount of total ADAMTS13 is from about 5 to about 4,000 IU/kg body weight.
81. The method of any one of embodiments 71-78, wherein the therapeutically effective amount of total ADAMTS13 is from about 20 to about 2,000 IU/kg body weight.
82. The method of any one of embodiments 71-78, wherein the therapeutically effective amount of total ADAMTS13 is from about 20 to about 500 IU/kg body weight.
83. The method of any one of embodiments 71-78, wherein the therapeutically effective amount of total ADAMTS13 is from about 20 to about 80 IU/kg body weight.
84. The method of any one of embodiments 71-78, wherein the therapeutically effective amount of total ADAMTS13 is from about 40 to about 160 IU/kg body weight.
85. The method of any one of embodiments 71-78, wherein the therapeutically effective amount of total ADAMTS13 is from about 40 to about 80 IU/kg body weight.
86. The method of any one of embodiments 71-78, wherein the therapeutically effective amount of total ADAMTS13 is from about 40 IU/kg body weight, about 80 IU/kg body weight or about 160 IU/kg body weight.
87. The method of any one of embodiments 71-78, wherein the therapeutically effective amount of total ADAMTS13 is from about 10 to about 6,000 IU/kg body weight.
88. The method of any one of embodiments 71-78, wherein the therapeutically effective amount of total ADAMTS13 is from about 20 to about 4,000 IU/kg body weight.
89. The method of any one of embodiments 71-78, wherein the therapeutically effective amount of total ADAMTS13 is from about 50 to about 500 IU/kg body weight.
90. The method of any one of embodiments 71-78, wherein the therapeutically effective amount of total ADAMTS13 is from about 100 to about 3,000 IU/kg body weight.
91. The method of any one of embodiments 71, 72, or 74-90, wherein the ADAMTS13 variant or composition is administered to the subject within 48 hours after the onset of the vaso-occlusive crisis.
92. The method of any one of embodiments 71-91, wherein the ADAMTS13 variant or composition for preventing the vaso-occlusive crisis is sufficient to maintain an effective level of ADAMTS13 activity in the subject.
93. A method for treating or preventing lung injury in a subject suffering from or at risk of suffering from acute lung injury (ALI) and/or acute respiratory distress syndrome (ARDS), the method comprising administering to the subject in need thereof a therapeutically effective amount of the ADAMTS13 variant of any one of embodiments 1-11 or pharmaceutical composition of any one of embodiments 12-27.
94. The method of embodiment 93, wherein the subject suffers from a condition or a combination of the conditions selected from the group consisting of inflammatory pulmonary edema, inflammatory pulmonary infiltrates, impaired oxygenation, and hypoxemia.
95. The method of embodiment 93 or embodiment 94, wherein administering the ADAMTS13 variant or composition results in at least one of improved survival, improved lung function, reduced organ damage, reduced pulmonary vascular leakage, or a combination of any thereof as compared to control or without treatment.
96. The method of any one of embodiments 93-95, wherein administering the ADAMTS13 variant or composition reduces at least one of inflammation, vasoconstriction, platelet aggregation, or a combination of any thereof as compared to control or without treatment.
97. The method of any one of embodiments 93-96, wherein administering the ADAMTS13 variant or composition reduces and/or prevents at least one of impaired blood flow, blood coagulation, vascular inflammation, thrombosis, ischemic cell damage, organ damage, or a combination of any thereof as compared to control or without treatment.
98. The method of any one of embodiments 93-97, wherein administering the ADAMTS13 variant or composition reduces and/or prevents pain or severity of the pain as compared to control or without treatment.
99. The method of any one of embodiments 93-98, wherein administering the ADAMTS13 variant or composition reduces the frequency of occurrence of ALI and/or ARDS and/or duration of ALI and/or ARDS episodes as compared to without treatment.
100. The method of any one of embodiments 93-99, wherein the therapeutically effective amount of total ADAMTS13 is from about 1 to about 4,000 IU/kg body weight.
101. The method of any one of embodiments 93-99, wherein the therapeutically effective amount of total ADAMTS13 is from about 5 to about 4,000 IU/kg body weight.
102. The method of any one of embodiments 93-99, wherein the therapeutically effective amount of total ADAMTS13 is from about 20 to about 2,000 IU/kg body weight.
103. The method of any one of embodiments 93-99, wherein the therapeutically effective amount of total ADAMTS13 is from about 40 to about 160 IU/kg body weight.
104. The method of any one of embodiments 93-99, wherein the therapeutically effective amount of total ADAMTS13 is from about 40 IU/kg body weight, about 80 IU/kg body weight or about 160 IU/kg body weight.
105. The method of any one of embodiments 93-99, wherein the therapeutically effective amount of total ADAMTS13 is from about 10 to about 6,000 IU/kg body weight.
106. The method of any one of embodiments 93-99, wherein the therapeutically effective amount of total ADAMTS13 is from about 20 to about 4,000 IU/kg body weight.
107. The method of any one of embodiments 93-99, wherein the therapeutically effective amount of total ADAMTS13 is from about 50 to about 500 IU/kg body weight.
108. The method of any one of embodiments 93-99, wherein the therapeutically effective amount of total ADAMTS13 is from about 100 to about 3,000 IU/kg body weight.
109. The method of any one of embodiments 93-108, wherein the ADAMTS13 variant or composition is administered to the subject within 48 hours after the detection of inflammatory pulmonary edema, inflammatory pulmonary infiltrates, impaired oxygenation, or hypoxemia.
110. The method of any one of embodiments 93-109, wherein the ADAMTS13 variant or composition is sufficient to maintain an effective level of ADAMTS13 activity in the subject.
111. A method for recanalization of an occluded blood vessel in a subject having a cerebral infarction, comprising administering to the subject in need thereof a therapeutically effective amount of the ADAMTS13 variant of any one of embodiments 1-11 or pharmaceutical composition of any one of embodiments 12-27, thereby recanalizing the occluded blood vessel.
112. The method of embodiment 111, wherein the ADAMTS13 variant or composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 IU/kg and/or within 15, 30, 60, 90, 120, 180, 210, 240, 270 or 300 minutes of detection of the infarction.
113. A method for treating a cerebral infarction in a subject by recanalization of an occluded blood vessel in the subject, comprising administering to the subject in need thereof a therapeutically effective amount of the ADAMTS13 variant of any one of embodiments 1-11 or pharmaceutical composition of any one of embodiments 12-27, thereby recanalizing the occluded blood vessel.
114. The method of embodiment 113, wherein the ADAMTS13 variant or composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 IU/kg and/or within 15, 30, 60, 90, 120, 180, 210, 240, 270 or 300 minutes detection of the infarction.
115. The method of any one of embodiments 111-114, wherein the ADAMTS13 variant or composition is administered to the subject at a dose of about 40, 50, 60, 70, 80, 90, 100, 150, 200, 250, 300, 350, 400, 450, 500, 550, 600, 650, 700, 750, 800, 850, 900, 950, 1,000, 1,250, 1,500, 1,750, or 2,000 IU/kg; and within 15, 30, 60, 90, 120, 180, 210, 240, 270 or 300 minutes of detection of the infarction.
116. A method for recanalization of an occluded blood vessel in a subject having a cerebral infarction, comprising the step of administering to the subject a pharmaceutical composition comprising administering to the subject in need thereof a therapeutically effective amount of the ADAMTS13 variant of any one of embodiments 1-11 or pharmaceutical composition of any one of embodiments 12-27, thereby recanalizing the occluded blood vessel, wherein the pharmaceutical composition is administered to the subject at an amount that increases the level of total ADAMTS13 protein in the subject 1.1, 1.2, 1.3, 1.4, 1.5, 1.6, 1.7, 1.8, 1.9, 2, 3, 4, 5, 6, 7, 8, 9, 10, 15, or 20-fold greater than the level of total ADAMTS13 protein in the subject prior to the administering.
117. The method of embodiment 116, wherein the ADAMTS13 variant or composition is administered to the subject within 15, 30, 60, 90, 120, 180, 210, 240, 270 or 300 minutes of detection of the infarction.
118. The method of any one of embodiments 111-117, wherein the regional cerebral blood flow in the subject is improved by at least 25% as compared to a control subject not having a cerebral infarction.
119. The method of any one of embodiments 111-118, wherein the regional cerebral blood flow is improved by at least 50% as compared to the regional cerebral blood flow in the control subject.
120. The method of any one of embodiments 111-118, wherein the regional cerebral blood flow is improved by at least 75% as compared to the regional cerebral blood flow in the control subject.
121. The method of any one of embodiments 111-120, wherein the ADAMTS13 variant or composition is administered multiple times or by continuous infusion.
122. The method of any one of embodiments 111-121, wherein said administration does not increase the level of hemorrhage, as compared to the level of hemorrhage in a subject not receiving the ADAMTS13 variant or composition.
123. The method of any one of embodiments 111-122, wherein said administration reduces infarct volume.
124. The method of embodiment 123, wherein the infract volume is reduced by at least 50% compared to the infract volume in a control subject not having a cerebral infarction.
125. A method of improving the recovery of sensorimotor function in a subject that has experienced a cerebral infarction comprising administering to the subject in need thereof a therapeutically effective amount of the ADAMTS13 variant of any one of embodiments 1-11 or pharmaceutical composition of any one of embodiments 12-27, thereby improving the recovery of sensorimotor function, wherein the regional cerebral blood flow in the subject is improved by at least 25% as compared to the regional cerebral blood flow in a control subject not having a cerebral infarction.
126. The method according to any one of embodiments 25-125, wherein the ADAMTS13 variant or composition is administered in a single bolus injection, monthly, every two weeks, weekly, twice a week, daily, every 12 hours, every 8 hours, every six hours, every four hours, every two hours, or every hour.
127. A method for treating or preventing a blood clotting disorder associated with cardiovascular disease in a subject, comprising administering to the subject in need thereof a therapeutically effective amount of an ADAMTS13 variant of any one of embodiments 1-11 or pharmaceutical composition of any one of embodiments 12-27.
128. The method of embodiment 127, wherein the blood clotting disorder associated with cardiovascular disease is associated with myocardial infarction, myocardial ischemia, deep vein thrombosis, peripheral vascular disease, stroke, transient ischemic attack, or medical device associated thrombosis.
129. A method for treating or preventing hematologic disease in a subject, comprising administering to the subject in need thereof a therapeutically effective amount of an ADAMTS13 variant of any one of embodiments 1-11 or pharmaceutical composition of any one of embodiments 12-27.
130. The method of embodiment 129, wherein the hematologic disease is inherited TTP acquired TTP, thrombotic microangiopathy, or sickle cell disease.
131. The method of any one of embodiments 28-130, wherein the ADAMTS13 variant or composition is administered intravenously or subcutaneously.
132. The method of embodiment 131, wherein the ADAMTS13 variant or composition thereof is administered intravenously.
133. The method of embodiment 132, wherein the ADAMTS13 variant or composition is administered subcutaneously.
134. The method of embodiment 133, wherein the ADAMTS13 variant or composition thereof is administered subcutaneously and the therapeutically effective amount is the therapeutic amount for intravenous usage, which must be adjusted if administered subcutaneously.
135. The method of embodiment 134, wherein the bioavailability of the ADAMTS13 variant or composition after subcutaneous administration is 50-80% as compared to intravenous administration normalized for the same dose.
136. The method of embodiment 134, wherein the therapeutically effective amount of ADAMTS13 variant or composition comprises at least 120-300% of the amount of an intravenous dose for a specific indication as measured in activity units per kilogram.
137. The method of any one of embodiments 28-136, wherein the ADAMTS13 variant and/or ADAMTS13 protein is recombinantly produced.
138. The method of any one of embodiments 28-137, wherein the ADAMTS13 variant and/or ADAMTS13 protein is recombinantly produced by HEK293 cells.
139. The method of any one of embodiments 28-137, wherein the ADAMTS13 variant and/or ADAMTS13 protein is recombinantly produced by CHO cells.
140. The method of any one of embodiments 28-139, wherein the ADAMTS13 variant and/or ADAMTS13 protein is glycosylated.
141. The method of any one of embodiments 28-140, wherein the ADAMTS13 variant has a plasma half-life of more than 1 hour.
142. The method of any one of embodiments 28-141, wherein the subject is a mammal. 143. The method of any one of embodiments 28-142, wherein the subject is a human. 144. The method according to any one of embodiments 28-143, wherein the composition is lyophilized.
145. The method of embodiment 144, wherein the composition is reconstituted with a pharmaceutically acceptable vehicle suitable for injection prior to administration.
146. The method of any one of embodiments 28-143, wherein the composition is in a stable aqueous solution ready for administration.
147. Use of a composition comprising an ADAMTS13 variant of any one of embodiments 1-11 or pharmaceutical composition of any one of embodiments 12-27 for treating or preventing a blood clotting disorder in a subject.
148. A composition comprising an ADAMTS13 variant of embodiments any one of 1-11 or pharmaceutical composition of any one of embodiments 12-27 for use as a medicament for the treatment or prevention of a blood clotting disorder in a subject.
149. The use of embodiment 147 or the compositing of embodiment 148, wherein the blood clotting disorder is inherited TTP, acquired TTP, cerebral infarction, myocardial infarction, ischemic/reperfusion injury, deep vein thrombosis, or sepsis-related disseminated intravascular coagulation.
150. Use of a composition comprising an ADAMTS13 variant of embodiments any one of 1-11 or pharmaceutical composition of any one of embodiments 12-27 for treating or preventing a bleeding episode in a subject.
151. A composition comprising an ADAMTS13 variant of any one of embodiments 1-11 or pharmaceutical composition of any one of embodiments 12-27 for use as a medicament for the treatment or prevention of a bleeding episode in a subject.
152. The use of embodiment 150 or the compositing of embodiment 151, wherein the bleeding episode is associated with inherited TTP, acquired TTP, cerebral infarction, myocardial infarction, ischemic/reperfusion injury, deep vein thrombosis, or sepsis-related disseminated intravascular coagulation.
153. Use of a composition comprising an ADAMTS13 variant of any one of embodiments 1-11 or pharmaceutical composition of any one of embodiments 12-27 for treating or preventing a vaso-occlusive crisis in a subject suffering from sickle cell disease.
154. A composition comprising an ADAMTS13 variant of any one of embodiments 1-11 or pharmaceutical composition of any one of embodiments 12-27 for use as a medicament for the treatment or prevention of a vaso-occlusive crisis in a subject suffering from sickle cell disease.
155. Use of a composition comprising an ADAMTS13 variant of any one of embodiments 1-11 or pharmaceutical composition of any one of embodiments 12-27 for treating, ameliorating or preventing lung injury in a subject suffering from or at risk of suffering from acute lung injury (ALI) and/or acute respiratory distress syndrome (ARDS).
156. A composition comprising an ADAMTS13 variant of any one of embodiments 1-11 or pharmaceutical composition of any one of embodiments 12-27 for use as a medicament for the treatment, amelioration, or prevention of a lung injury in a subject suffering from or at risk of suffering from acute lung injury (ALI) and/or acute respiratory distress syndrome (ARDS).
157. Use of a composition comprising an ADAMTS13 variant of any one of embodiments 1-11 or pharmaceutical composition of any one of embodiments 12-27 for improving the recovery of sensorimotor function in a subject that has experienced a cerebral infarction, wherein the regional cerebral blood flow in the subject is improved by at least 25% as compared to the regional cerebral blood flow in a control subject not having a cerebral infarction.
158. A composition comprising an ADAMTS13 variant of any one of embodiments 1-11 or pharmaceutical composition of any one of embodiments 12-27 for use as a medicament for improving the recovery of sensorimotor function in a subject that has experienced a cerebral infarction, wherein the regional cerebral blood flow in the subject is improved by at least 25% as compared to the regional cerebral blood flow in a control subject not having a cerebral infarction.
159. A nucleic acid molecule encoding the ADAMTS13 variant of any one of any one of embodiments 1-11.
160. A vector comprising the nucleic acid molecule of embodiment 159.
161. The vector of embodiment 160, wherein the vector is an expression vector wherein the polynucleotide sequence encoding the ADAMTS13 variant is operably linked to a promoter that is capable of mediating expression of the ADAMTS13 variant in a host cell.
162. A host cell comprising the nucleic acid molecule of embodiment 159.
163. A host cell comprising the vector of embodiment 160 or embodiment 161.
164. A host cell line comprising cells modified to express the ADAMTS13 variant of any one of embodiments 1-11 and at least one ADAMTS13 protein.
165. The host cell line of embodiment 164, wherein the ADAMTS13 variant comprises the amino acid sequence of SEQ ID NO: 2, or an amino acid sequence having at least 80% sequence identity thereof
166. The host cell line of embodiment 164 or embodiment 165, wherein the amino acid sequence of the ADAMTS13 protein comprises the amino acid sequence of SEQ ID NO: 1, or an amino acid sequence having at least 80% sequence identity thereof.
167. The host cell line of any one of embodiments 164-166, wherein the amino acid sequence of the ADAMTS13 protein consists of the amino acid sequence of SEQ ID NO: 1.
168. The host cell line of any one of embodiments 164-168, wherein the ADAMTS13 variant and the ADAMTS13 protein are expressed in different cells in the host cell line.
169. The host cell line of any one of embodiments 164-169, wherein the ADAMTS13 variant and the ADAMTS13 protein are expressed in the same cell.
170. The host cell or host cell line of any one of embodiments 162-169, wherein the cell is a CHO, COS, HEK 293, BHK, SK-Hep, or HepG2 cell.
171. The host cell or host cell line of embodiment 170, wherein the CHO cell is a CHO DBX-11 or CHOZN cell line.
172. The host cell or host cell line of embodiment 171, wherein the CHOZN cell is a CHO DBX-11 cell line.
173. The host cell or host cell line of embodiment 171, wherein the CHOZN cell is a CHOZN glutamine synthetase (GS)^{-/-} cell line.
174. An ADAMTS13 protein or variant thereof, comprising at one or more glycosylation sites.
175. The ADAMTS13 or variant thereof of embodiment 174, wherein the ADAMTS13 variant comprises the ADAMTS13 variant of any one of embodiments 1-11.
176. The ADAMTS13 or variant thereof of embodiment 174, wherein the ADAMTS13 protein comprises the amino acid sequence of SEQ ID NO: 1, or an amino acid sequence having at least 80% sequence identity thereof 177. The ADAMTS13 or variant thereof of any one of embodiments 174-176, which is glycosylated at an O-glycosylation site.
178. The ADAMTS13 or variant thereof of embodiment 177, which is glycosylated at serine residues at one or more O-glycosylation sites 5399, 5698, 5757, 5907, 5965, 51027 or S1087.
179. The ADAMTS13 or variant thereof of embodiment 177, which is glycosylated with the disaccharide Fuc-Glc.
180. The ADAMTS13 or variant thereof of embodiment 177, which is glycosylated with a mucin-type 0-glycan.
181. The ADAMTS13 or variant thereof of embodiment 180, wherein the mucin-type 0-glycan has the structure HexNAc-Hex-NeuAc₀₋₂.
182. The ADAMTS13 or variant thereof of any one of embodiments 174-181, which is glycosylated at an N-glycosylation site.
183. The ADAMTS13 or variant thereof of embodiment 182, that is glycosylated at asparagine residues at one or more N-glycosylation sites N142, N146, N552, N579, N614, N667, N707, N828, N1235 or N1354.
184. The ADAMTS13 or variant thereof of embodiment 182, which is glycosylated with high mannose-type N-glycan.
185. The ADAMTS13 or variant thereof of embodiment 182, which is glycosylated with a hybrid-type N-glycan.
186. The ADAMTS13 or variant thereof of embodiment 182, which is glycosylated with a complex-type N-glycan.
187. The ADAMTS13 or variant thereof of embodiment 186, wherein the complex-type N-linked glycan comprises a core-fucose residue.
188. The ADAMTS13 or variant thereof of embodiment 186, wherein the complex-type N-linked glycan comprises one or more sialic acid residues.
189. The ADAMTS13 or variant thereof of embodiment 186, wherein the complex-type N-linked glycan comprises one sialic acid residue.
190. The ADAMTS13 or variant thereof of embodiment 186, wherein the complex-type N-linked glycan comprises two sialic acid residues.
191. The ADAMTS13 or variant thereof of embodiment 186, wherein the complex-type N-linked glycan comprises three sialic acid residues.
192. The ADAMTS13 or variant thereof of embodiment 186, wherein the complex-type N-linked glycan comprises four sialic acid residues.
193. The ADAMTS13 or variant thereof of embodiment 188, wherein the sialic acid residue is linked via an α2,6-linkage.
194. The ADAMTS13 or variant thereof of embodiment 188, wherein the sialic acid residue is linked via an α2,3-linkage.
195. The ADAMTS13 or variant thereof of any one of embodiments 174-181, which is glycosylated at tryptophan residues at one or more C-mannosylation sites.
196. The ADAMTS13 or variant thereof of embodiment 187, which is glycosylated at tryptophan residues at one or more C-mannosylation sites W387 or W390.
197. The ADAMTS13 or variant thereof of embodiment 187, which is glycosylated with a mannosyl residue.
198. The ADAMTS13 or variant thereof of any one of embodiments 174-181, wherein the N-glycan index of the ADAMTS13 variant is substantially similar to the N-glycan index of the ADAMTS13.
199. The ADAMTS13 or variant thereof of any one of embodiments 174-181, wherein the N-glycan index is between about 120 to about 190.
200. The ADAMTS13 or variant thereof of any one of embodiments 174-181, wherein the N-glycan index is below 140.
201. The ADAMTS13 or variant thereof of any one of embodiments 174-181, wherein the amount of sialic acid residue is between about 130 to about 169 nmol sialic acid per mg ADAMTS13 protein.
202. The ADAMTS13 or variant thereof of any one of embodiments 174-176, comprising an N-glycan signature (i.e., total sum of 100%) that matches one or more of the following parameters:
   a. has at least about 75% neutral, mono- and di-sialylated N-glycans combined;
   b. has at least about 5% tri- and tetra-sialylated glycans combined;
   c. has about 10% to about 35% neutral N-glycans;
   d. has about 20% to about 50% monosialayted N-glycans;
   e. has about 10% to about 40% disialylated N-glycans;
   f. has about 1% to about 25% trisialylated N-glycans;
   g. has about 0.1% to about 10% tetrasialylated N-glycans; and/or
   h. has a N-glycan index of about 110 to about 160.
203. The ADAMTS13 or variant thereof of embodiment 202, comprising an N-glycan signature that matches one or more of the following parameters:
   a. has at least about 80% neutral, mono- and di-sialylated N-glycans combined;
   b. has at least about 10% tri- and tetra-sialylated glycans combined;
   c. has about 15% to about 30% neutral N-glycans;
   d. has about 25% to about 45% monosialayted N-glycans;
   e. has about 15% to about 35% disialylated N-glycans;
   f. has about 5% to about 20% trisialylated N-glycans;
   g. has about 1% to about 5% tetrasialylated N-glycans; and/or
   h. has a N-glycan index of about 120 to about 150.
204. The ADAMTS13 or variant thereof of embodiment 202, comprising an N-glycan signature that matches one or more of the following parameters:
   a. has at least about 85% neutral, mono- and di-sialylated N-glycans combined;
   b. has at least about 14% tri- and tetra-sialylated glycans combined;
   c. has about 20% to about 25% neutral N-glycans;
   d. has about 30% to about 40% monosialayted N-glycans;
   e. has about 20% to about 30% disialylated N-glycans;
   f. has about 10% to about 15% trisialylated N-glycans;
   g. has about 2% to about 4% tetrasialylated N-glycans; and/or
   h. has a N-glycan index of about 130 to about 140.
205. The ADAMTS13 or variant thereof of embodiment 202, comprising an N-glycan signature that matches one or more of the following parameters:
   a. has at least about 86% neutral, mono- and di-sialylated N-glycans combined;
   b. has at least about 15% tri- and tetra-sialylated glycans combined;
   c. has about 23% to about 24% neutral N-glycans;
   d. has about 34% to about 36% monosialayted N-glycans;
   e. has about 26% to about 29% disialylated N-glycans;
   f. has about 11% to about 12% trisialylated N-glycans;
   g. has about 3% tetrasialylated N-glycans; and/or
   h. has a N-glycan index of about 133 to about 139.
206. The ADAMTS13 or variant thereof of any one of embodiments 174-176, comprising a sialic acid signature that comprises a ratio of about 1% to about 15% of % NGNA relative to NANA.
207. The ADAMTS13 or variant thereof of embodiment 206, comprising a sialic acid signature that comprises a ratio of about 2% to about 10% of % NGNA relative to NANA.
208. The ADAMTS13 or variant thereof of embodiment 206, comprising a sialic acid signature that comprises a ratio of about 3% to about 8% of % NGNA relative to NANA.
209. The ADAMTS13 or variant thereof of embodiment 206, comprising a sialic acid signature that comprises a ratio of about 4% to about 5% of % NGNA relative to NANA.
210. The ADAMTS13 or variant thereof of any one of embodiments 174-176, comprising an N-glycan signature (i.e., total sum of 100%) that matches one or more of the following parameters:
   a. has at least about 80% neutral, mono- and di-sialylated N-glycans combined;
   b. has at least about 16% tri- and tetra-sialylated glycans combined;
   c. has about 5% to about 30% neutral N-glycans;
   d. has about 10% to about 45% monosialayted N-glycans;
   e. has about 20% to about 55% disialylated N-glycans;
   f. has about 1% to about 30% trisialylated N-glycans;
   g. has about 0.1% to about 15% tetrasialylated N-glycans; and/or
   h. has a N-glycan index of about 130 to about 190.
211. The ADAMTS13 or variant thereof of embodiment 210, comprising an N-glycan signature that matches one or more of the following parameters:
   a. has at least about 83% neutral, mono- and di-sialylated N-glycans combined;
   b. has at least about 17% tri- and tetra-sialylated glycans combined;
   c. has about 8% to about 25% neutral N-glycans;
   d. has about 15% to about 40% monosialayted N-glycans;
   e. has about 25% to about 50% disialylated N-glycans;
   f. has about 5% to about 25% trisialylated N-glycans;
   g. has about 1% to about 10% tetrasialylated N-glycans; and/or
   h. has a N-glycan index of about 140 to about 185.
212. The ADAMTS13 or variant thereof of embodiment 210, comprising an N-glycan signature that matches one or more of the following parameters:
   a. has at least about 85% neutral, mono- and di-sialylated N-glycans combined;
   b. has at least about 18% tri- and tetra-sialylated glycans combined;
   c. has about 10% to about 20% neutral N-glycans;
   d. has about 20% to about 35% monosialayted N-glycans;
   e. has about 30% to about 45% disialylated N-glycans;
   f. has about 10% to about 20% trisialylated N-glycans;
   g. has about 2% to about 8% tetrasialylated N-glycans; and/or
   h. has a N-glycan index of about 145 to about 175.
213. The ADAMTS13 or variant thereof of embodiment 210, comprising an N-glycan signature that matches one or more of the following parameters:
   a. has at least about 86% neutral, mono- and di-sialylated N-glycans combined;
   b. has at least about 19% tri- and tetra-sialylated glycans combined;
   c. has about 13% to about 16% neutral N-glycans;
   d. has about 26% to about 33% monosialayted N-glycans;
   e. has about 37% to about 43% disialylated N-glycans;
   f. has about 12% to about 15% trisialylated N-glycans;
   g. has about 3% to about 6% tetrasialylated N-glycans; and/or
   h. has a N-glycan index of about 153 to about 170.
214. The ADAMTS13 or variant thereof of any one of embodiments 174-176, comprising a monosaccharides signature (i.e., total sum of 100%) that matches one or more of the following parameters:
   a. has about 10% to about 40% GlcNAc;
   b. has about 0.1% to about 10% GalNAc;
   c. has about 10% to about 35% Gal;
   d. has about 10% to about 35% Man;
   e. has about 0.1% to about 20% Glc; and/or
   f. has about 5% to about 20% Fuc.
215. The ADAMTS13 or variant thereof of embodiment 214, comprising a monosaccharides signature that matches one or more of the following parameters:
   a. has about 15% to about 35% GlcNAc;
   b. has about 1% to about 8% GalNAc;
   c. has about 15% to about 30% Gal;
   d. has about 15% to about 30% Man;
   e. has about 1% to about 15% Glc; and/or
   f. has about 10% to about 15% Fuc.
216. The ADAMTS13 or variant thereof of embodiment 214, comprising a monosaccharides signature that matches one or more of the following parameters:
   a. has about 20% to about 30% GlcNAc;
   b. has about 5% to about 6% GalNAc;
   c. has about 20% to about 25% Gal;
   d. has about 20% to about 28% Man;
   e. has about 5% to about 10% Glc; and/or
   f. has about 11% to about 12% Fuc.
217. The ADAMTS13 or variant thereof of embodiment 214, comprising a monosaccharides signature that matches one or more of the following parameters:
   a. has about 27% to about 29% GlcNAc;
   b. has about 6% GalNAc;
   c. has about 23% to about 24% Gal;
   d. has about 23% to about 25% Man;
   e. has about 6% to about 7% Glc; and/or
   f. has about 12% Fuc.
218. The ADAMTS13 or variant thereof of any one of embodiments 174-176, comprising a sialic acid signature that matches one or more of the following parameters:
   a. has about 100 nmol NANA/mg to about 200 nmol NANA/mg;
   b. has about 0.01 nmol NGNA/mg to about 1 nmol NGNA/mg; and/or
   c. has a ratio of about 0.01% to about 1% of % NGNA relative to NANA.
219. The ADAMTS13 or variant thereof of embodiment 218, comprising a sialic acid signature that matches one or more of the following parameters:
   a. has about 110 nmol NANA/mg to about 180 nmol NANA/mg;
   b. has about 0.05 nmol NGNA/mg to about 0.50 nmol NGNA/mg; and/or
   c. has a ratio of about 0.05% to about 0.5% of % NGNA relative to NANA.
220. The ADAMTS13 or variant thereof of embodiment 218, comprising a sialic acid signature that matches one or more of the following parameters:
   a. has about 125 nmol NANA/mg to about 170 nmol NANA/mg;
   b. has about 0.08 nmol NGNA/mg to about 0.30 nmol NGNA/mg; and/or
   c. has a ratio of about 0.06% to about 0.2% of % NGNA relative to NANA.
221. The ADAMTS13 or variant thereof of embodiment 218, comprising a sialic acid signature that matches one or more of the following parameters:
   a. has about 130 nmol NANA/mg to about 169 nmol NANA/mg;
   b. has about 0.1 nmol NGNA/mg to about 0.3 nmol NGNA/mg; and/or
   c. has a ratio of about 0.1% to about 0.2% of % NGNA relative to NANA.
222. The ADAMTS13 or variant thereof of any one of embodiments 174-221, wherein the ADAMTS13 or variant thereof is produced in a CHO, COS, HEK 293, BHK, SK-Hep, or HepG2 cell line.
223. The ADAMTS13 or variant thereof of embodiment 222, wherein the CHO cell is a CHO DBX-11 or CHOZN cell line.
224. The ADAMTS13 or variant thereof of embodiment 223, wherein the CHOZN cell is a CHOZN glutamine synthetase (GS)^{-/-} cell line.
225. The ADAMTS13 or variant thereof of any one of embodiments 174-201 or 210-221 wherein the ADAMTS13 or variant thereof is produced in a CHO cell line.
226. The ADAMTS13 or variant thereof of embodiment 225, wherein the CHOZN cell is a CHOZN glutamine synthetase (GS)^{-/-} cell line.
227. The ADAMTS13 or variant thereof of any one of embodiments 174-209 wherein the ADAMTS13 or variant thereof is produced in a CHO cell line.
228. The ADAMTS13 or variant thereof of embodiment 227, wherein the CHOZN cell is a CHO DBX-11.

### VIII. Examples

### Example 1: Evaluation of VWF Cleavage for Plasma Derived ADAMTS13 (pdADAMTS13) Versus a rADAMTS13 Composition Comprising a Combination of Wildtype rADAMTS13 and Q⁹⁷R rADAMTS13 Variant

Specific activity provides information about the quality and potency of a protein, and a lower activity would be indicative of a reduced protein quality and potency. Therefore, the specific activity data (FRETS U/antigen U) of 50 rADAMTS13 batches composed of a mixture of wildtype rADAMTS13 and Q⁹⁷R variants were compared with the specific activity data (FRETS U/antigen U) of plasma derived (pdADAMTS13) in from 80 healthy donors. A single coding nucleotide exchange at position 290 of the coding sequence of rADAMTS13 cDNA (mRNA) from adenine to guanine results in the Q⁹⁷R ADAMTS13 variant.

The specific activity was determined by calculating the ADAMTS13 Activity (FRETS-VWF73) per mg of total protein determined by UV absorption spectrophotometry. VWF contained in the sample (=ristocetin cofactor) causes agglutination of stabilized platelets in the presence of ristocetin, both contained in the “von Willebrand reagent” (Behring Coagulation System, BCS, Siemens, Germany). The agglutination reduces the turbidity of the reagent preparation, and the change in optical density is measured by the coagulation system analyzer (Behring Coagulation System, BCS, Siemens, Germany). The VWF:RCo activity was calculated from a reference curve constructed by different dilutions of a reference plasma, calibrated against the WHO standard.

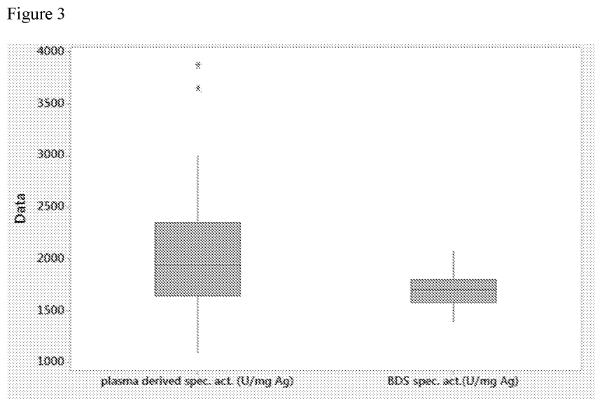

Plasma samples from individual healthy donors show a higher variation in specific activity of ADAMTS13 compared to the analyzed recombinant mixed wildtype and Q⁹⁷R variant batches (FIG. 3). However, the average specific activity of the mixed rADAMTS13 batches is comparable with the specific activity of pdADAMTS13, indicating that the potency of the mixed rADAMTS13 batches is also comparable to the potency of pdADAMTS13 as well.

Full length VWF is cleaved by human ADAMTS13 at a single peptide bond within the A2 domain, between Tyr1605 and Met1606, thereby generating an N-terminal 140 kDa fragment and a C-terminal 176 kDa fragment. Therefore, cleavage of VWF is another indicator for potency of ADAMTS13. Data from degradation kinetic experiments have shown that mixed rADAMTS13 batches and pdADAMTS13 have similar cleavage rates, indicating that the activity of the mixed rADAMTS13 batches and pdADAMTS13 is comparable (see Table 1).

TABLE 1
In vitro cleavage of VWF by rADAMTS13 and pdADAMTS13,
cleavage with 0.1, 0.5, and 1.0 U/ml ADAMTS13.

| | | ADAMTS13 (0.1 U/ml) | | ADAMTS13 (0.5 U/ml) | | ADAMTS13 (1.0 U/ml) | |
|---|---|---|---|---|---|---|---|
| | | recombinant | plasmatic | recombinant | plasmatic | recombinant | plasmatic |
| Time of incubation | | IU VWF RCo/ml | | IU VWF RCo/ml | | IU VWF RCo/ml | |
| 0 | h | 0.72 | 0.75 | 0.78 | 0.78 | 0.56 | 0.26 |
| 15 | seconds | 0.33 | 0.38 | <0.17 | <0.17 | <0.17 | <0.17 |
| 30 | seconds | 0.34 | 0.37 | <0.17 | <0.17 | <0.17 | <0.17 |
| 1 | minute | 0.30 | 0.21 | <0.17 | <0.17 | <0.17 | <0.17 |
| 2 | minutes | 0.24 | <0.17 | <0.17 | <0.17 | <0.17 | <0.17 |
| 5 | minutes | <0.17 | <0.17 | <0.17 | <0.17 | <0.17 | <0.17 |

As such, there is no difference in the specific activity and VWF cleavage for pdADAMTS13 and the mixed rADAMTS13 batches.

### Example 2: Structure Function Relationship of the Q⁹⁷R rADAMTS13 Variant

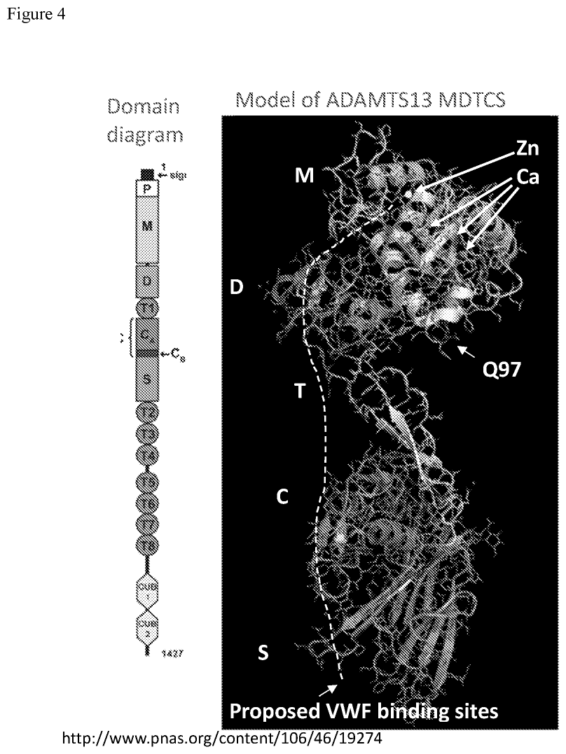

The Q⁹⁷R rADAMTS13 variant was modeled in order to predict whether it has an effect on the structure/function of the rADAMTS13 (FIG. 4). This study modeled the 3-dimensional structure of ADAMTS13 N-terminal MDTCS domains and reviewed published data on the C-terminal-domain regulated ADAMTS13 activity in the M domain. It was found that Q⁹⁷ in M domain was solvent exposed and located away from VWF cleavage site and metal ion binding sites, ruling out a role in regulation of these binding domains. However, this study did not rule out the possibility that Q⁹⁷ might be involved in C-terminal CUB1-2 interaction.

The study was designed to generate a computational model of ADAMTS13 using in silico tools and to map Q⁹⁷ on the model. The intention was to show, based on the computation model and knowledge from publications, whether Q⁹⁷R might cause changes to protein folding, stability, or activation of the protease. A model of human ADAMTS13 MDTCS domains was built by piecing together crystal structure of ADAMTS13 DTCS domains with a model of MD domains.

Residues 80-383, covering MD domains, of human ADAMTS13 (Uniprot Q76LX8) were used to generate a computational model. The modeling was performed on Dell Linux workstation (system desktop management information: 44454C4C-4300-1038-804B-B6C04F584732) with homolog modeling tool in program MOE version 2016.0802. Crystal structure of MD domains of ADAMTS4, PDB code 2JRP [2], was input as modeling template.

The output model of ADAMTS13 MD domains was superimposed to human ADAMTS13 DCTS domains, PDB code 3JHN [1], by aligning the D domains with SSM alignment in program Coot version 0.8.6. The modeled M domain was then pieced together to DTCS domains, by manually connecting Asp298 on M domain to Ala299 on D domain, to make a complete model of MDTCS domains.

The MDTCS model was then loaded to program Pymol version 1.8.2.2 for visualization, mapping of Q⁹⁷, and figure preparation.

Q⁹⁷ is exposed on M protease domain and located in a short two residue turn in a stable helix-turn-helix structure. Q⁹⁷ flanking helices are stabilized by folding within M domain or mediating MD domain interface interactions. Q⁹⁷ and E98 do not mediate folding or metal ion binding in M domain, and Q⁹⁷ is away from proposed VWF binding sites. Current knowledge cannot rule out the possibility that Q⁹⁷ is involved in CUB domains regulation. Two recent publications show, by small angle X-ray scattering and electron microscopy, that CUB1-2 domains fold back to N-terminal MDTCS domains and regulate ADAMTS13 activity (Muia et al., supra; South et al., supra, each of which is herein incorporated herein by reference in their entirety for all purposes). In situ-modelling provided evidence that the Q⁹⁷R mutation does not impact ADAMTS13 functionality.

It was determined that Q⁹⁷ is located on the surface of M domain and does not mediate protein folding. This residue is also distant from proposed functional sites including VWF binding and cleavage sites, zinc and calcium binding sites, and domain interface between M and DTCS. Computational model of MDTCS domain suggests Q⁹⁷ does not mediate structure or function of N-terminal domains of ADAMTS13. However, published data shows allosteric activation of ADAMTS13, in which C-terminal CUB1-2 domains interact with MDTCS domains to keep protease at a low activity state before VWF binding. It remains unknown whether Q⁹⁷ would potentially be involved in domain interactions between CUB1-2 and MDTCS.

### Example 3: Evaluation of rADAMTS13 Compositions Comprising Different Ratios of Q⁹⁷R rADAMTS13 Variant to Wildtype rADAMTS13

In total 35 batches were investigated for this example. For assessment, samples were subjected to Peptide Mapping based on Trypsin digestion. In particular, the samples of purified rADAMTS13 BDS were reduced with di-thio-threitol (DTT) and the free sulfhydryl groups were blocked with iodoacetamide. The samples were desalted and then incubated for 18 hours with sequencing grade trypsin. The resulting peptide mixture was separated by reversed phase chromatography, and the eluted peptides were detected by on-line UV detection at 214 nm and on-line electrospray ionization mass spectrometry. To determine the abundance of Q⁹⁷R variant, the peptide carrying the mutated amino acid was analyzed.

The N-terminal tryptic peptide of the native protein contains 28 amino acids:

Position 75-102:
(SEQ ID NO: 6)
AAGGILHLELLVAVGPDVFQAHQEDTER

The change of amino acid from “Q” to “R” introduces an additional tryptic cleavage site in the sequence Q⁹⁷R variant. Therefore, the corresponding peptide results in two tryptic peptides:

Position 75-97:
(SEQ ID NO: 115)
AAGGILHLELLVAVGPDVFQAHR
Position 98-102:
(SEQ ID NO: 5)
EDTER

The miss-cleaved form of the Q⁹⁷R variant has to be considered as well:

Position 75-102:
(SEQ ID NO: 7)
AAGGILHLELLVAVGPDVFQAHREDTER

For the Q⁹⁷R variant, the peptide of interest was also found in the miss-cleaved form, from position 75-102. The miss-cleaved form was present in lower abundance than the correctly cleaved peptide, but the level varied due to method variation. The variation also includes the variability of the enzymatic activity of trypsin, which cannot be controlled by the analyst. Therefore, to determine the amount of Q⁹⁷R correctly, the peak areas of the correctly cleaved tryptic peptide (75-97) and of the miss-cleaved form (98-102) were summed up.

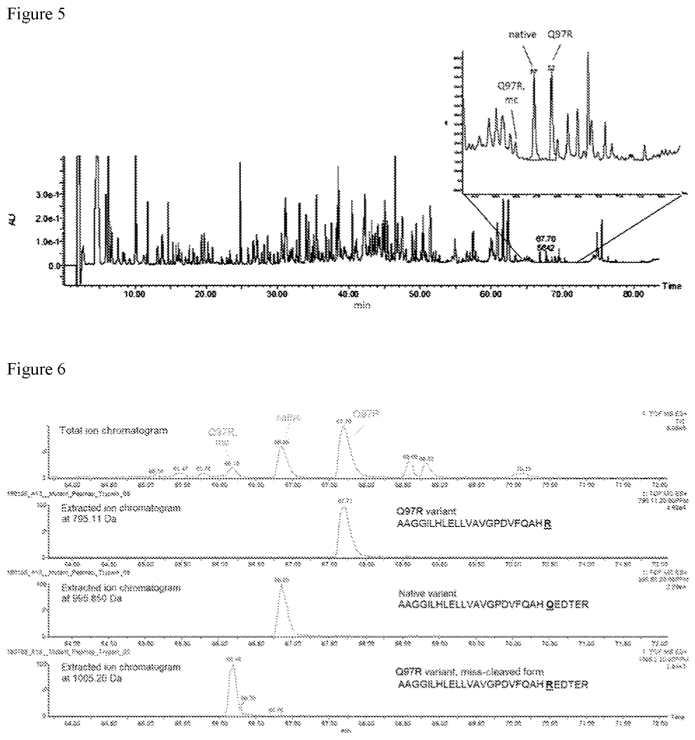

HPLC data show that the tryptic peptides of the variants can be separated by liquid chromatography. The following mass spectrometry analysis has confirmed that the tryptic peptide of the native variant elutes before the one from Q⁹⁷R variant (FIG. 5). The miss-cleaved tryptic peptide of the Q⁹⁷R variant elutes even before the peptide of the native variant. The abundance of these peptides in a single composition is comparable, and determined based on intensities in extracted ion chromatograms (FIG. 6), indicating a comparable abundance of both variants across the manufacturing campaigns. Extracted ion chromatograms of the native and Q⁹⁷R variant were generated, integrated, and relative peak areas were calculated (Table 2).

The relative abundance of the Q⁹⁷R variant was determined based on the peak areas of the tryptic peptides calculated in relation to the sum of the peak areas of both the Q⁹⁷ peptide and the Q⁹⁷R variant. Extracted ion chromatograms of the Q⁹⁷ peptide and Q⁹⁷R variant (correctly cleaved one and miss-cleaved one) were generated, integrated and relative peak areas were calculated.

TABLE 2
Results of the abundance of Q⁹⁷R variant composition batches.

| | Area [%] | Area [%] | specific | cleavage of |
|---|---|---|---|---|
| | native | Q⁹⁷R | activity U/mg | multimeric rVWF |
| Sample | variant | variant ¹ | (UV) | [%] |
| 1 | 41 | 59 | 1642 | 58 |
| 2 | 36 | 64 | 1585 | 52 |
| 3 | 41 | 59 | 1397 | 51 |
| 4 | 44 | 56 | 1459 | 60 |
| 5 | 45 | 55 | 1361 | 52 |
| 6 | 36 | 64 | 1469 | 57 |
| 7 | 34 | 66 | 2044 | 62 |
| 8 | 30 | 70 | 1611 | n.a. ² |
| 9 | 33 | 67 | 1479 | 62 |
| 10 | 33 | 67 | 1447 | 60 |
| 11 | 40 | 60 | 1584 | 61 |
| 12 | 40 | 60 | 1617 | 59 |
| 13 | 29 | 71 | 1970 | 60 |
| 14 | 41 | 59 | 1392 | 55 |
| 15 | 48 | 52 | 1333 | 59 |
| 16 | 43 | 57 | 1409 | 66 |
| 17 | 39 | 61 | 1385 | 58 |
| 18 | 37 | 63 | 1443 | 55 |
| 19 | 46 | 54 | 1663 | 78 |
| 20 | 36 | 64 | 1700 | 78 |
| 21 | 36 | 64 | 1692 | 75 |
| 22 | 34 | 66 | 1620 | 65 |
| 23 | 39 | 61 | 1545 | 66 |
| 24 | 38 | 62 | 1512 | 63 |
| 25 | 39 | 61 | 1531 | 63 |
| 26 | 39 | 61 | 1757 | 67 |
| 27 | 41 | 59 | 1856 | 67 |
| 28 | 40 | 60 | 1753 | 67 |
| 29 | 45 | 55 | 1729 | 68 |
| 30 | 40 | 60 | 1850 | 67 |
| 31 | 36 | 64 | 1801 | 71 |
| 32 | 40 | 60 | 1747 | 62 |
| 33 | 35 | 65 | 1619 | 78 |
| 34 | 39 | 61 | 1716 | 65 |
| 35 | 28 | 72 | 1649 | 66 |

¹ The relative peak area of the Q⁹⁷R variant was calculated in relation to the sum of the peak areas of both variants.
² Sample 8 was not measured for multimeric rVWF.

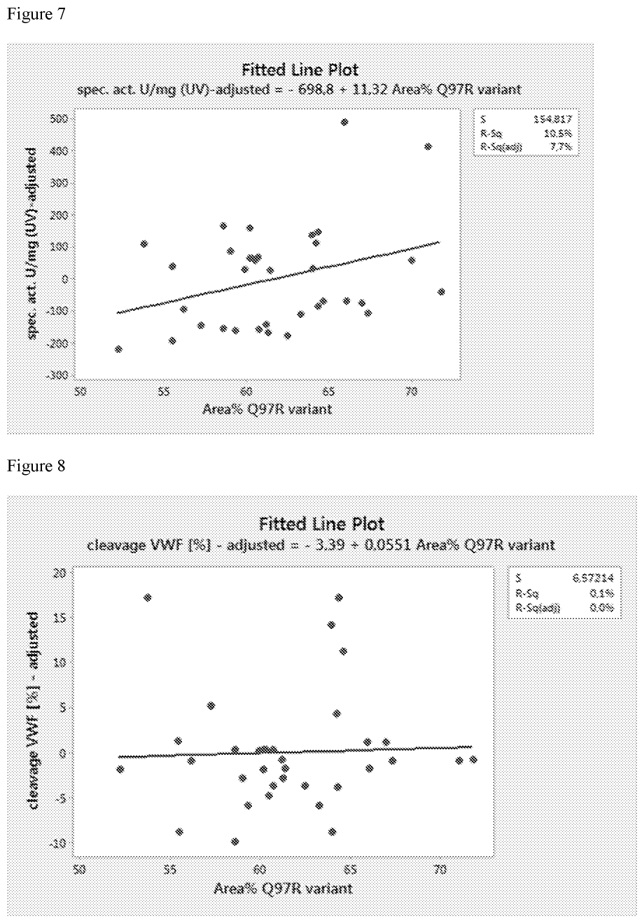

To evaluate Q⁹⁷R variant functionality, the specific activity (FRETS U/mg UV protein) was set in relation to the relative abundance of Q⁹⁷R using a regression analysis (FIG. 7). As demonstrated in FIG. 7, there is no correlation between the variant ratio (i.e., ratio of Q⁹⁷R rADAMTS13 variant to wildtype ADAMTS13) and the specific activity, suggesting that the Q⁹⁷R rADAMTS13 variant has similar activity to that of wildtype ADAMTS13.

Full length VWF is cleaved by human ADAMTS13 at a single peptide bond within the A2 domain, between Tyr1605 and Met1606, thereby generating an N-terminal 140 kDa fragment and a C-terminal 176 kDa fragment. The reduction of abundancy of multimeric VWF is detected using agarose gel electrophoresis. Therefore, cleavage of VWF is another indicator for potency of ADAMTS13.

The impact on functionality was also evaluated using a full length VWF cleavage assay. Therefore, data of VWF cleavage was plotted against the abundance of Q⁹⁷R variant (FIG. 8). The analysis resulted in a p value of 0.832, which indicates no correlation between the abundance of Q⁹⁷R variant and activity of VWF cleavage.

### Example 4: Determination of the Relative Amount of Q⁹⁷R Variant

In total 12 batches were analyzed with regards to their relative proportion of Q⁹⁷R protein variant. These 12 batches include comparatively low and high Q⁹⁷R portions as revealed by reevaluations of data obtained during biochemical characterization with a tryptic digestion method and to consider potential variations due to the nature of the production process.

For the determination of the relative amount of Q⁹⁷R variant, the samples were tested using a method based on thrombin protein digestion and subsequent analysis with reversed phase high performance liquid chromatography in combination with fluorescence detection (RP-HPLC-FLD). Sample preparation included acetone precipitation, reduction with Dithiothreitol (DTT), alkylation with Iodoacetamide (IAA) and incubation with PNGase F over night to cleave N-linked oligosaccharides. Finally, proteins were digested using Thrombin for 60 min, whereupon equally cleaved fragments were generated from both protein variants containing the site of amino acid exchange. 20 μL of the samples was analyzed with RP-HPLC on an Agilent Zorbax 300SB-C8 column (1.8 μm; 2.1×100 mm) run at 0.4 mL/min at 70° C. column temperature. Gradient elution is performed from 12% to 44% B in 5.8 min followed by fluorescence detection with excitation wavelength of 280 nm and emission wavelength of 340 nm. For data analysis corresponding peaks for Q⁹⁷ and Q⁹⁷R variant were integrated and relative peak areas in % are reported in Table 3.

TABLE 3
Results of the abundance of Q⁹⁷R variant composition batches.

| | Q97R | Q97 |
|---|---|---|
| | Area | Area |
| Sample | % | % |
| 1 | 66.3 | 33.7 |
| 2 | 65.7 | 34.3 |
| 3 | 66.4 | 33.6 |
| 4 | 66.7 | 33.3 |
| 5 | 60.4 | 39.6 |
| 6 | 60.8 | 39.2 |
| 7 | 64.8 | 35.2 |
| 8 | 60.4 | 39.6 |
| 9 | 70.9 | 29.1 |
| 10 | 66.6 | 33.4 |
| 11 | 63.0 | 37.0 |
| 12 | 72.9 | 27.1 |

With a confidence interval of 95% and a population probability of 99.73%, the relative portion of Q⁹⁷R variant ranges from 46.5% to 84.3%, which equals mean±4.8×standard deviation. The average amount of Q⁹⁷R was 65.4%.

### Example 5: Expression and Characterization of Q⁹⁷ ADAMTS13 and Q⁹⁷R ADAMTS13 Variant

The overall strategy was to express Q⁹⁷ ADAMTS13 and the Q⁹⁷R ADAMTS13 variant individually for structural and functional characterization. An analytical characterization of the two individual proteins (Q⁹⁷ and R⁹⁷) was performed. The focus of the characterization was on functionality, primary structure, and higher order structure. Results of the characterization are summarized in Table 5 and Table 8.

The two cell lines were produced in clone pools based on the SAFC CHOZN GS^{-/-} host cell system and maintained in EX-Cell Advanced CHO Fed-batch media. Aprotinin was used in upstream and downstream processes to prevent truncation of the two ADAMTS13 proteins.

The two individual ADAMTS13 proteins were produced in two different cell systems which led to different protein concentrations. The difference in protein concentration expectedly results in differences in activity and antigen levels. The protein concentrations of the samples appear in Table 4, which were used to correlate activity. The relevant parameter is the specific activity value, which is independent of the protein concentration, and these results were highly comparable for the two individual variants.

TABLE 4
Samples of the two individual ADAMTS13 proteins.

| | Protein |
|---|---|
| | Concentration |
| Sample | [mg/ml] |
| Q⁹⁷ ADAMTS13 | 1.262 |
| Q⁹⁷R ADAMTS13 | 0.786 |

TABLE 5
Q⁹⁷ ADAMTS13 and Q⁹⁷R ADAMTS13 Characterization Results

| Testing | Test | | | | | |
|---|---|---|---|---|---|---|
| parameter | method | Characterization | Q97 | | Q⁹⁷R | |
| rADAMTS13 | FRETS | Comparable | 2208 | IU/mL² | 1370 | IU/mL² |
| Activity | VWF73 | activity | | | | |
| Specific | Calculation | Comparable | 1749 | IU/mg | 1742 | IU/mg |
| rADAMTS13 | | specific activity | | | | |
| activity¹ | | | | | | |
| rADAMTS13 | ELISA | Comparable | 1323 | μg/mL² | 959 | μg/mL² |
| Antigen | | antigen levels | | | | |

| | | | | |
|---|---|---|---|---|
| Dimers and | SE-HPLC | Comparable | 97.7% Monomers | 97.3% Monomers |
| Aggregates | | monomer levels | | |
| SDS-PAGE/ | SDS-PAGE | Yes | One major band + | One major band + |
| Western Blot | | | truncation band | truncation band |
| N-glycan | HPLC | Comparable N- | 133 | 139 |
| index³ | | glycan index | | |
| Relative | RP-HPLC- | Comparable peak | (pure native) | (pure variant) |
| percentage of | FLD | pattern of either | 96% Q⁹⁷/4% Q⁹⁷- | 100% Q⁹⁷R |
| rADAMTS13 | | R97 or Q97 variant | related species | |
| variant | | | (co-eluting with Q⁹⁷R | |
| | | | peak) | |

¹Calculation of Specific Activity is performed using Activity & Total Protein value determined by UV (Q⁹⁷: 1.262 mg/ml, Q⁹⁷R: 0.786 mg/ml)
²Differences between the two variants are tracking with differences in Total Protein (protein concentration) delivered by the manufacturing processes of the two variants
³~130 is a typical N-Glycan index for CHOZN cell line

The size exclusion high performance liquid chromatography (SE-HPLC) method was used to determine the oligomeric structure of the ADAMTS13 forms. The majority of both samples are present in monomeric form. The monomeric peaks show a small shoulder, indicating the truncated form of the protein. However, the shoulder was present in both protein variant samples. The proteins were also present in low amounts in dimeric form and in aggregated form (Table 6). The oligomeric structures of the two ADAMTS13 proteins were comparable.

TABLE 6
Detailed results of the size exclusion chromatography
of the two ADAMTS13 protein forms

| | Aggregates | Dimer | Monomer |
|---|---|---|---|
| Sample | [Area %] | [Area %] | [Area %] |
| Q⁹⁷ ADAMTS13 | 0.4 | 1.9 | 97.7 |
| Q⁹⁷R ADAMTS13 | 0.4 | 2.2 | 97.3 |

An extended characterization program was conducted for both ADAMTS13 proteins. The characterization test panel was performed, as discussed below, and comparability was assessed based on the listed criteria. A side-by-side testing of both proteins was used for the extended characterization program to reduce influence of assay variability.

For primary structure analysis, the primary structure of rADAMTS13 was investigated using a peptide mapping approach. Samples of purified rADAMTS13 were reduced with dithiothreitol and the free sulfhydryl groups were blocked with iodoacetamide. Reagents were removed using Zeba spin columns and mass spectrometry grade trypsin was added and allowed to react at +37° C. for 18 h. The resulting peptide mixture was separated by RP-HPLC, and eluting peptides were detected by online UV detection at 214 nm and identified using mass spectrometric detection. The peptide mapping data have shown that the two ADAMTS13 proteins were present in their pure form, no contamination with the other form was detected.

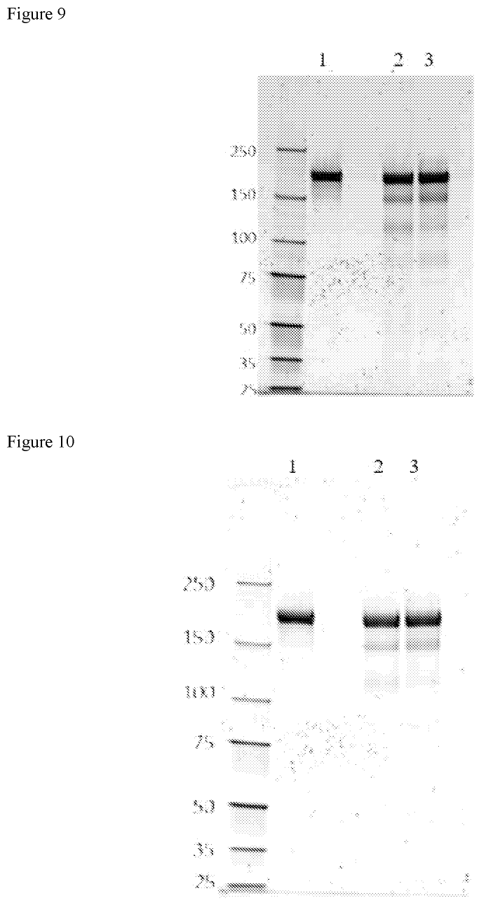

For protein composition analysis, rADAMTS13 BDS batches were analyzed by SDS PAGE using 3-8% Tris-Acetate gels under reducing conditions. Gels were stained with flamingo fluorescence gel stain to evaluate the protein composition of the analyzed batches (FIG. 9). For Western blotting, proteins were transferred to a nitrocellulose membrane following staining with an anti-ADAMTS13 antibody. Comparability was assessed by visual comparison of the stained gels/membranes (FIG. 10). For the determination of the mean molecular mass of rADAMTS13, samples were mixed with sinapinic acid as the matrix molecule. Sample/matrix mixtures were spotted onto the target, and molecular mass was determined in linear positive ion mode using Matrix Assisted Laser Desorption Ionization Mass Spectrometry (MALDI MS) with a model 4800 MALDI TOF/TOF instrument (Applied Biosystems) equipped with a HM-1 high mass detector (CovalX).

For post-translational modification analysis, protein-bound oligosaccharides of rADAMTS13 were determined by releasing the N-linked sugars with PNGase F and labeling the reducing end of the liberated oligosaccharides with 2-AB (2-aminobenzamide). Separation and relative quantification of the labeled oligosaccharides were performed by normal phase HPLC and fluorescence detection. Therefore, samples were denaturated, and enzymatic deglycosylation was performed using PNGase F. The released N-glycans were washed and lyophilized before labeling with 2-aminobenzamide by reductive amination. Oligosaccharides were separated on a Luna Amino 3μ column using a water/acetonitrile/250 mM ammonium acetate pH 4.5 gradient and detected using fluorescence detection. The N-glycans are grouped based on their charge related to the number of sialic acids into five charge clusters (neutral, monosialylated, disialylated, trisialylated and tetrasialylated N-glycans). In addition to the comparison of the chromatographic profiles, the relative abundance of each charge state cluster and the N-glycan index was calculated. N-glycan index was calculated from the relative area (%) of the different charge state clusters by the following formula: N-glycan index=Neutral*0+Monosialo*1+Disialo*2+Trisialo*3+Tetrasialo*4. Protein-bound sialic acids were liberated from rADAMTS13 by mild acid hydrolysis, labeled using DMB (1,2-diamino-4,5-methylenoxybenzene) and then quantified by RP-HPLC with FLD. Briefly, rADAMTS13 BDS samples were desalted by acetone precipitation. The desalted samples were reconstituted in MilliQ water and adjusted to a final concentration of 2 M acetic acid and incubated at +80° C. for 2.5 h. Liberated sialic acids were labeled with DMB and separated by RP-HPLC on a Jupiter 5μ C18 column using an acetonitrile/methanol/water gradient. Calibration was performed with standard preparations of N-acetyl neuraminic acid (NANA) and N-glycolyl neuraminic acid (NGNA).

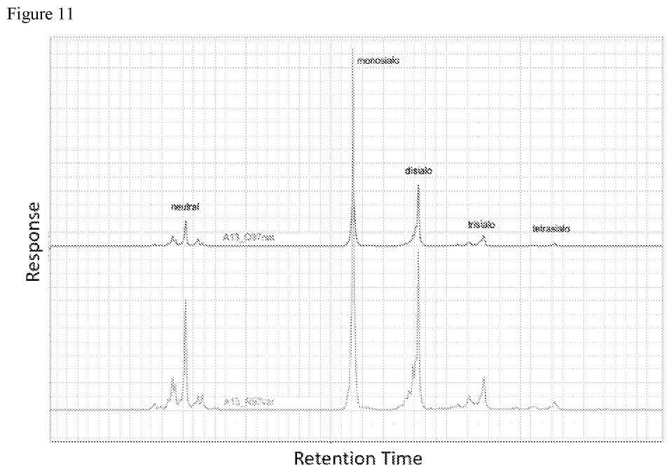

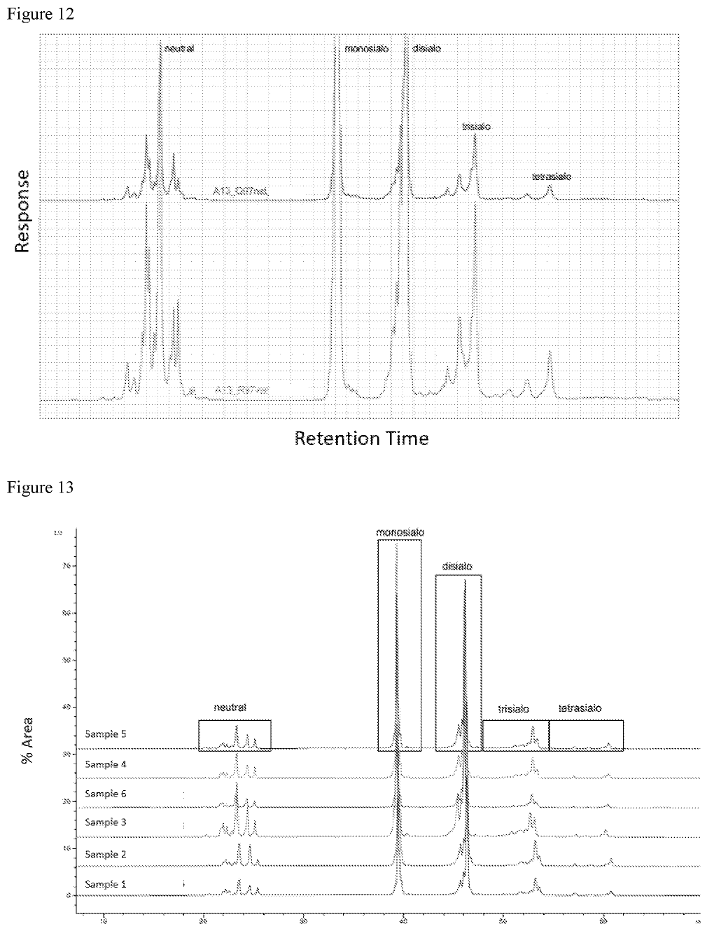

Protein-bound oligosaccharides of rADAMTS13 were determined by releasing the N-linked sugars with PNGase F, and subsequent labeling of the liberated oligosaccharides. An overlay of the chromatograms are shown in FIG. 11 and FIG. 12. In addition, the N-glycan mapping data have been analyzed according to their subgroups, which are summarized in Table 7. The data show a good comparability between the two samples, for the individual subgroups, as well as for the N-glycan index. Specifically, the N-glycan index of the two samples were substantially similar and below the N-glycan acceptance range of 140-185.

TABLE 7
Relative peak areas of N-glycan subgroup of the two ADAMTS13 protein forms

| | Natural | Monosialo | Disialo | Trisialo | Tetrasialo | N-glycan |
|---|---|---|---|---|---|---|
| Sample | Area % | Area % | Area % | Area % | Area % | Index |
| Q⁹⁷ | 24.2 | 35.6 | 26.1 | 11.2 | 2.9 | 132.9 |
| ADAMTS13 | | | | | | |
| Q⁹⁷R | 22.5 | 33.9 | 28.7 | 11.9 | 3.1 | 139.2 |
| ADAMTS13 | | | | | | |

For higher order structure analysis, the tertiary structure of protein molecules can be assessed by CD analysis in the near UV wavelength region (250 to 350 nm). At these wavelengths, aromatic amino acids and disulfide bonds are optically active, and their composite signals produce spectral features specific to the three-dimensional conformation of a given protein. The secondary structure of proteins can be determined by CD spectroscopy in the far UV wavelength region (190 to 250 nm). In this region, the peptide bond (amide bond) produced CD signals characteristic of well-ordered secondary structures (e.g., helices and sheets). SV AUC determines protein size and conformation directly from a sample in solution. Protein size variants, e.g., monomer and dimer, were separated based on differences in their sedimentation coefficients. The sedimentation coefficient of a protein is a function of both the molecular weight and shape of the species. Protein sedimentation was accomplished through centrifugation at high angular velocity (typically 40,000-60,000 rpm). The concentration of each protein size variant was measured as a function of time and radial position using absorbance optics. The concentration profiles were subsequently analyzed providing information about the protein size distribution, plotted as a c(s) distribution. Each peak in the c(s) distribution can be integrated and its area (as a percentage of the total area) represents the relative concentration of that species. DSC provides information about the thermal and conformational stability of proteins by quantitatively monitoring the unfolding of proteins as a function of temperature. A sample cell and a reference cell (identical matrix, but lacking protein) were simultaneously heated from 20 to 100° C., at a scan rate of 60° C./h. As the temperature increases, the power required to heat both cells were continuously measured, and the difference in power between the cells was used to determine the sample heat capacity. The heat capacity of the protein was plotted as a function of temperature. Analysis of this profile, called a thermogram, provides thermodynamic information about the sample, including the enthalpy of unfolding and the transition (i.e., melting) temperature (Tm) for each unfolding event.

For functionality analysis, the functional characterization assay was measured under static conditions using a full-length VWF substrate. Such a setup required the addition of a denaturing agent (urea) to unfold the VWF substrate and make it susceptible for ADAMTS13 cleavage. All rADAMTS13 samples were diluted to 30 mIU/mL according to their assigned FRETS-VWF73 activity. The diluted samples were activated with BaCl₂ in the presence of 5 mM Tris and 1.5 M urea, pH 8.0, at 37° C. for 30 minutes. Activated ADAMTS13 was mixed with 1 VWF:Ag IU/mL of recombinant VWF and further incubated at 37° C. for 2 hours. The reaction was stopped by the addition of Na₂SO₄ (8.25 mM final concentration). As a control, rVWF treated with buffer instead of rADAMTS13 was included in the experimental setup, and the same procedure was followed as that with rADAMTS13. The samples were centrifuged at 2500×g for 5 minutes and the supernatant was used for further analysis. Changes in multimeric structure of rVWF after incubation with rADAMTS13 was analyzed by horizontal SDS agarose gel electrophoresis under low-resolution conditions to analyze the size distribution of VWF. The VWF multimers were visualized in the separation gel by immunostaining using a polyclonal rabbit antihuman VWF antibody. The amino acid sequence of rADAMTS13 contains 23 methionine residues, which represent potential sites of oxidation, resulting in modification of methionine to methionine-sulfoxide. Oxidized variants of rADAMTS13 were determined by tryptic peptide. For the respective non-oxidized and oxidized peptides reconstructed ion chromatograms were generated, integrated and the relative areas calculated. Six methionines were selected for quantitative data evaluation, based on historic forced degradation data.

TABLE 8
Detailed results of the size exclusion chromatography of the two ADAMTS13 protein forms

| | Test | Evaluation | | |
|---|---|---|---|---|
| Purpose | method | Criteria | Q97 | Q⁹⁷R |
| Primary | Tryptic | Confirmation of | Native only | Variant only |
| Structure | peptide | expected amino | | |
| | mapping | acid sequence | | |
| | with LC- | | | |
| | MS | | | |
| Protein | SDS-PAGE | Comparable band | Comparable | Comparable |
| composition | | pattern | | |
| | Molecular | Comparable | 172785 Da | 174005 Da |
| | Weight by | molecular weight | | |
| | MALDI MS | | | |
| Post- | N-glycan | Comparable peak | Comparable peak | Comparable peak |
| translational | analysis by | pattern | pattern | pattern |
| modifications | HPLC | | | |
| | Sialic acids | Comparable sialic | 4.81% NANA/NGNA | 4.35% NANA/NGNA |
| | by RP- | acid levels <1.0% | | |
| | HPLC | N-glycolyl | | |
| | | neuraminic acid | | |
| Higher order | NUV CD/ | Criteria based on | Comparable | Comparable |
| structure | FUV CD | similarity scoring | | |
| | | will be derived | | |
| | | from method | | |
| | | qualification | | |
| | AUC | Comparable levels | Comparable | Comparable |
| | | of high molecular | | |
| | | weight species | | |
| | | Main peak | | |
| | | sedimentation | | |
| | | coefficient of the | | |
| | | two processes are | | |
| | | within ± EAC | | |
| | | established during | | |
| | | method | | |
| | | qualification | | |
| | DSC | Criteria based on | 54.5 | Comparable |
| | | thermograms and | | |
| | | melting | | |
| | | temperature will be | | |
| | | derived from | | |
| | | method | | |
| | | qualification | | |
| Functionality | Degradation | Comparable band | Comparable | Comparable |
| | of full- | pattern | | |
| | length | | | |
| | rVWF | | | |
| | under | | | |
| | denaturing | | | |
| | conditions | | | |
| | (VWF | | | |
| | multimer | | | |
| | analysis) | | | |
| Impurities | Oxidation | Comparable | Comparable | Comparable |
| | | oxidation levels | | |

Results for the individual variants were highly comparable indicating similarity in the primary, secondary, and higher order structures, as well as the functionality of the both protein variants. Based on the results summarized in this example, it can be concluded that Q⁹⁷ ADAMTS13 and the Q⁹⁷R ADAMTS13 have the same physicochemical, biophysical, and biological properties, and will continue to do so whether produced in the same cell or different cells of the same background.

### Example 6: Glycosylation Analysis of Q⁹⁷ ADAMTS13 and Q⁹⁷R ADAMTS13

This example uses several analytical methods to determine the degree of glycosylation and the principle nature of the ADAMTS13 glycosylation pattern.

Both Q⁹⁷ ADAMTS13 and Q⁹⁷R ADAMTS13 were expressed together in a CHO DBX-11 host cell line (i.e., one cell line produces both ADAMTS13 proteins together).

For the tryptic peptide mapping, samples of purified ADAMTS13 were reduced with di-thio-threitol (DTT) and the free sulfhydryl groups were blocked with iodoacetamide. Reagents were removed by dialysis and rADAMTS13 was collected and lyophilized. Sequencing grade trypsin or LysC was added and allowed to react for 2 hours followed by a second addition of enzyme and incubation for 18 hours. In addition, N-glycans were removed with PNGase F to obtain more complete sequence coverage. The resulting peptide mixture was separated by reversed phase chromatography. Eluting peptides were detected by on-line UV detection at 214 nm and in addition detection by on-line mass spectrometry only for tryptic peptide mixture. The peptides covering the amino acid sequence of ADAMTS13 are listed in Table 9.

TABLE 9
Identified Signals of the Peptide Mapping of ADAMTS13 Sorted by Sequence

| | | | | | Monoisotopic | | Mass |
|---|---|---|---|---|---|---|---|
| | Range | | | tR | Exp. | Theor. | accuracy |
| Peptide¹ | AA¹ | Peptide Sequence | Modification² | [min] | [Da] | [Da] | [ppm] |
| T001 | 1-28 | AAGGILHLELLVAVGP | — | 67.0 | 2984.5437 | 2984.5354 | 2.8 |
| | | DVFQAHQEDTER | | | | | |
| | | (SEQ ID NO: 6) | | | | | |
| T002 | 29-42 | YVLTNLNIGAELLR | — | 60.1 | 1587.9023 | 1587.8984 | 2.5 |
| | | (SEQ ID NO: 8) | | | | | |
| T002 | 29-42 | YVLTNLNIGAELLR | — | 60.9 | 1587.8956 | 1587.8984 | −1.8 |
| | | (SEQ ID NO: 8) | | | | | |
| T003 | 43-51 | DPSLGAQFR | — | 34.4 | 989.4910 | 989.4930 | −2.0 |
| | | (SEQ ID NO: 9) | | | | | |
| T004 | 52-56 | VHLVK | — | 24.9 | 594.3858 | 594.3853 | 0.8 |
| | | (SEQ ID NO: 10) | | | | | |
| T005* | 57-106 | MVILTEPEGAPNITAN | Carbamidomethyl | 75.5 | 5425.9915 | 5425.6222 | 4.3 |
| | | LTSSLLSVCGWSQTIN | C(1), Deamidation | | | | |
| | | PEDDTDPGHADLVLYI | N(2) | | | | |
| | | TR | | | | | |
| | | (SEQ ID NO: 11) | | | | | |
| T006 | 107-116 | FDLELPDGNR | — | 44.9 | 1174.5627 | 1174.5618 | 0.8 |
| | | (SEQ ID NO: 12) | | | | | |
| 1006 | 107-116 | FDLELPDGNR | — | 45.3 | 1174.5631 | 1174.5618 | 1.1 |
| | | (SEQ ID NO: 12) | | | | | |
| T007 | 117-119 | QVR | — | 10.2 | 401.23 | 401.23 | 1.7 |
| T008* | 120-183 | GVTQLGGACSPTWSCL | Carbamidomethyl | 75.0 | 6460.8896 | 6460.9272 | −5.8 |
| | | ITEDTGFDLGVTIAHE | C(3) | | | | |
| | | IGHSFGLEHDGAPGSG | | | | | |
| | | CGPSGHVMASDGAAPR | | | | | |
| | | (SEQ ID NO: 13) | | | | | |
| T009* | 184-193 | AGLAWSPCSR | Carbamidomethyl | 37.1 | 1103.5157 | 1103.5182 | −2.3 |
| | | (SEQ ID NO: 14) | C(1) | | | | |
| T010- | 194-204 | RQLLSLLSAGR | — | 49.8 | 1212.7236 | 1212.7302 | −5.4 |
| 011 | | (SEQ ID NO: 15) | | | | | |
| T011 | 195-204 | QLLSLLSAGR | | 51.5 | 1056.6265 | 1056.6292 | −2.6 |
| | | (SEQ ID NO: 16) | | | | | |
| T012 | 205-206 | AR | | n.d. | n.d. | 245.14 | n.d. |
| T013- | 207-238 | CVWDPPRPQPGSAGHP | Carbamidomethyl | 42.2 | 3606.6201 | 3606.6157 | 1.2 |
| 014* | | PDAQPGLYYSANEQCR | C(2) | | | | |
| | | (SEQ ID NO: 17) | | | | | |
| T015 | 239-244 | VAFGPK | | 27.2 | 617.3556 | 617.3537 | 3.1 |
| | | (SEQ ID NO: 18) | | | | | |
| T016* | 245-252 | AVACTFAR | Carbamidomethyl | 27.2 | 894.4427 | 894.4382 | 5.0 |
| | | (SEQ ID NO: 19) | C(1) | | | | |
| T016* | 245-252 | AVACTFAR | Carbamidomethyl | 62.6 | 894.4373 | 894.4382 | −1.0 |
| | | (SEQ ID NO: 19) | C(1) | | | | |
| T017* | 253-275 | EHLDMCQALSCHTDPL | Carbamidomethyl | 42.4 | 2746.1299 | 2746.1204 | 3.5 |
| | | DQSSCSR | C(3) | | | | |
| | | (SEQ ID NO: 20) | | | | | |
| T018* | 276-290 | LLVPLLDGTECGVEK | Carbamidomethyl | 55.0 | 1641.8633 | 1641.8647 | −0.9 |
| | | (SEQ ID NO: 21) | C(1) | | | | |
| T018* | 276-290 | LLVPLLDGTECGVEK | Carbamidomethyl | 53.3 | 1641.8685 | 1641.8647 | 2.3 |
| | | (SEQ ID NO: 21) | C(1) | | | | |
| T019* | 291-294 | WCSK | Carbamidomethyl | 14.7 | 579.2490 | 579.2476 | 2.4 |
| | | (SEQ ID NO: 114) | C(1) | | | | |
| T020 | 295-296 | GR | | n.d. | n.d. | 231.1331 | n.d. |
| T021 | 297-298 | CR | | n.d. | n.d. | 334.1423 | n.d. |
| T022 | 299-312 | SLVELTPIAAVHGR | | 49.0 | 1461.8273 | 1461.8303 | −2.1 |
| | | (SEQ ID NO: 22) | | | | | |
| T022 | 299-312 | SLVELTPIAAVHGR | | 48.6 | 1461.8248 | 1461.8303 | −3.8 |
| | | (SEQ ID NO: 22) | | | | | |
| T023 | 313-319 | WSSWGPR | | 35.6 | 874.4049 | 874.4086 | −4.2 |
| | | (SEQ ID NO: 23) | | | | | |
| T023* | 313-319 | WSSWGPR | C-Mannosylation | 30.7 | 1036.4602 | 1036.4614 | −1.2 |
| | | (SEQ ID NO: 23) | W(1) | | | | |
| T024* | 320-324 | SPCSR | Carbamidomethyl | 7.8 | 605.2609 | 605.2592 | 2.8 |
| | | (SEQ ID NO: 24) | C(1) | | | | |
| T025* | 325-333 | SCGGGVVTR | Carbamidomethyl | 17.7 | 1199.5342 | 1199.5339 | 0.3 |
| | | (SEQ ID NO: 25) | C(1), | | | | |
| | | | O-Fucosylation Glc- | | | | |
| | | | Fuc | | | | |
| T025* | 325-333 | SCGGGVVTR | Carbamidomethyl | 17.7 | 891.4241 | 891.4233 | 0.9 |
| | | (SEQ ID NO: 25) | C(1) | | | | |
| T025* | 325-333 | SCGGGVVTR | Carbamidomethyl | 18.3 | 1199.5288 | 1199.5339 | −4.3 |
| | | (SEQ ID NO: 25) | C(1), | | | | |
| | | | O-Fucosylation Glc- | | | | |
| | | | Fuc | | | | |
| | | | ST(1) | | | | |
| T026 | 334-334 | R | | n.d. | n.d. | 174.1117 | n.d. |
| T027- | 335-341 | RQCNNPR | Carbamidomethyl | 9.9 | 943.4395 | 943.4407 | −1.3 |
| 028* | | (SEQ ID NO: 20) | C(1) | | | | |
| T028* | 330-341 | QCNNPR | Carbamidomethyl | 8.0 | 787.3404 | 787.3395 | 1.1 |
| | | (SEQ ID NO: 27) | C(1) | | | | |
| T029 | 342-347 | PAFGGR | | 21.0 | 003.3132 | 003.3129 | 0.5 |
| | | (SEQ ID NO: 28) | | | | | |
| T030* | 348-300 | ACVGADLQAEMCNTQA | Carbamidomethyl | 30.9 | 2154.8833 | 2154.8804 | 1.3 |
| | | CEK | C(3) | | | | |
| | | (SEQ ID NO: 29) | | | | | |
| T031* | 307-378 | TQLEFMSQQCAR | Carbamidomethyl | 37.5 | 1497.0735 | 1497.0705 | 2.0 |
| | | (SEQ ID NO: 30) | C(1) | | | | |
| T032 | 379-385 | TDGQPLR | | 18.2 | 785.4031 | 785.4031 | 0.0 |
| | | (SEQ ID NO: 31) | | | | | |
| T033* | 380-410 | SSPGGASFYHWGAAVP | Carbamidomethyl | 45.2 | 2014.1919 | 2014.1772 | 5.0 |
| | | HSQGDALCR | C(1) | | | | |
| | | (SEQ ID NO: 32) | | | | | |
| T034* | 411-414 | HMCR | Carbamidomethyl | 11.3 | 002.2433 | 002.2418 | 2.5 |
| | | (SEQ ID NO: 33) | C(1) | | | | |
| T035 | 415-423 | AIGESFIMK | | 42.4 | 994.5152 | 994.5157 | −0.5 |
| | | (SEQ ID NO: 34) | | | | | |
| T037- | 424-433 | RGDSFLDGTR | | 32.8 | 1122.5411 | 1122.5417 | −0.5 |
| 037 | | (SEQ ID NO: 35) | | | | | |
| T036- | 424-433 | RGDSFLDGTR | | 30.7 | 1122.5444 | 1122.5417 | 2.4 |
| 037 | | (SEQ ID NO: 35) | | | | | |
| T037 | 425-433 | GDSFLDGTR | | 32.8 | 900.4382 | 900.4407 | −2.0 |
| | | (SEQ ID NO: 30) | | | | | |
| T037 | 425-433 | GDSFLDGTR | | 30.0 | 900.4375 | 900.4407 | −3.3 |
| | | (SEQ ID NO: 30) | | | | | |
| T038* | 434-440 | CMPSGPR | Carbamidomethyl | 20.3 | 803.3391 | 803.3419 | −3.5 |
| | | (SEQ ID NO: 37) | C(1) | | | | |
| T039* | 441-454 | EDGTLSLCVSGSCR | Carbamidomethyl | 35.9 | 1539.6646 | 1539.6658 | −0.8 |
| | | (SEQ ID NO: 38) | C(2) | | | | |
| T039* | 441-454 | EDGTLSLCVSGSCR | Carbamidomethyl | 35.5 | 1539.6672 | 1539.6658 | 0.9 |
| | | (SEQ ID NO: 38) | C(2) | | | | |
| T040* | 455-461 | TFGCDGR | Carbamidomethyl | 19.4 | 811.3272 | 811.3283 | −1.4 |
| | | (SEQ ID NO: 39) | C(1) | | | | |
| T041 | 462-470 | MDSQQVWDR | | 31.2 | 1163.5045 | 1163.5029 | 1.4 |
| | | (SEQ ID NO: 40) | | | | | |
| T042* | 471-484 | CQVCGGDNSTCSPR | Carbamidomethyl | 16.2 | 3656.3515 | 3656.3428 | −2.4 |
| | | (SEQ ID NO: 41) | C(3), | | | | |
| | | | N-Glycosylation | | | | |
| | | | biFucSA | | | | |
| | | | N(1) | | | | |
| T043- | 485-492 | KGSFTAGR | | 19.8 | 822.4337 | 822.4348 | −1.3 |
| 044 | | (SEQ ID NO: 42) | | | | | |
| T044 | 486-492 | GSFTAGR | | 19.8 | 694.3402 | 694.3398 | 0.6 |
| | | (SEQ ID NO: 43) | | | | | |
| T045 | 493-494 | AR | | n.d. | n.d. | 245.1488 | n.d. |
| T046* | 495-515 | EYVTFLTVTPNLTSVY | N-Glycosylation | 56.7 | 4788.1118 | 4788.0942 | 3.7 |
| | | IANHR | biFucSA2 N(1) | | | | |
| | | (SEQ ID NO: 44) | | | | | |
| T047 | 516-524 | PLFTHLAVR | | 45.6 | 1052.6162 | 1052.6130 | 3.0 |
| | | (SEQ ID NO: 45) | | | | | |
| T048 | 525-528 | IGGR | | 7.0 | 401.2378 | 401.2386 | −2.0 |
| | | (SEQ ID NO: 46) | | | | | |
| T048 | 525-528 | IGGR | | 19.3 | 401.2385 | 401.2386 | −0.2 |
| | | (SEQ ID NO: 46) | | | | | |
| T048 | 525-528 | IGGR | | 7.2 | 401.2373 | 401.2386 | −3.2 |
| | | (SEQ ID NO: 46) | | | | | |
| T049 | 529-534 | YWAGK | | 19.9 | 635.3650 | 635.3643 | 1.1 |
| | | (SEQ ID NO: 47) | | | | | |
| T050* | 535-551 | MSISPNTTYPSLLEDG | N-Glycosylation | 44.1 | 3744.5572 | 3744.5327 | −6.5 |
| | | R | Man4 | | | | |
| | | (SEQ ID NO: 48) | N(1) | | | | |
| T051 | 552-555 | VERY | | 15.4 | 565.2887 | 565.2860 | 4.8 |
| | | (SEQ ID NO: 49) | | | | | |
| T052 | 556-562 | VALTEDR | | 23.4 | 802.4168 | 802.4185 | −2.1 |
| | | (SEQ ID NO: 50) | | | | | |
| T053 | 563-565 | LPR | | 16.2 | 384.2485 | 384.2485 | 0.0 |
| T053 | 563-565 | LPR | | 18.2 | 384.2488 | 384.2485 | 0.8 |
| T054 | 566-570 | LEEIR | | 24.4 | 658.3666 | 658.3650 | 2.4 |
| | | (SEQ ID NO: 51) | | | | | |
| T055 | 571-585 | IWGPLQEDADIQVYR | | 51.6 | 1801.8981 | 1801.8999 | −1.0 |
| | | (SEQ ID NO: 52) | | | | | |
| T056 | 586-586 | R | | n.d. | n.d. | 174.1117 | n.d. |
| T057- | 587-607 | YGEEYGNLTRPDITFT | N-Glycosylation | 50.5 | 4889.0439 | 4889.0371 | 1.4 |
| 058* | | YFQPK | biFucSA2 | | | | |
| | | (SEQ ID NO: 53) | N(1) | | | | |
| T058 | 597-607 | PDITFTYFQPK | | 49.8 | 1355.6742 | 1355.6761 | −1.4 |
| | | (SEQ ID NO: 54) | | | | | |
| T059 | 608-609 | PR | | n.d. | n.d. | 271.1644 | n.d. |
| T060 | 610-618 | QAWVWAAVR | | 49.6 | 1085.5771 | 1085.5770 | 0.1 |
| | | (SEQ ID NO: 55) | | | | | |
| T061* | 619-630 | GPCSVSCGAGLR | Carbamidomethyl | 26.9 | 1527.6554 | 1527.6545 | 0.6 |
| | | (SEQ ID NO: 56) | C(2), | | | | |
| | | | O-Fucosylation Glc- | | | | |
| | | | Fuc | | | | |
| | | | ST(1) | | | | |
| T061* | 619-630 | GPCSVSCGAGLR | Carbamidomethyl | 26.9 | 1219.5457 | 1219.5438 | 1.6 |
| | | (SEQ ID NO: 56) | C(2) | | | | |
| T062* | 631-641 | WVNYSCLDQAR | Carbamidomethyl | 38.6 | 1410.6318 | 1410.6350 | −2.3 |
| | | (SEQ ID NO: 57) | C(1) | | | | |
| T062* | 631-641 | WVNYSCLDQAR | N-Glycosylation | 35.2 | 3761.4629 | 3761.4653 | −0.6 |
| | | (SEQ ID NO: 57) | biFucSA2 | | | | |
| | | | N(1), | | | | |
| | | | Carbamidomethyl | | | | |
| | | | C(1) | | | | |
| T062* | 631-641 | WVNYSCLDQAR | N-Glycosylation | 34.9 | 4417.7368 | 4417.6930 | −9.9 |
| | | (SEQ ID NO: 57) | triFucSA3 N(1), | | | | |
| | | | Carbamidomethyl | | | | |
| | | | C(1) | | | | |
| T063- | 642-689 | KELVETVQCQGSQQPP | Carbamidomethyl | 60.2 | 5639.4849 | 5639.5088 | −4.2 |
| 064* | | AWPEACVLEPCPPYWA | C(5), O- | | | | |
| | | VGDFGPCSASCGGGLR | Fucosylation | | | | |
| | | (SEQ ID NO: 58) | Glc-Fuc | | | | |
| | | | ST(1) | | | | |
| T064* | 643-689 | ELVETVQCQGSQQPPA | Carbamidomethyl | 61.0 | 5511.4258 | 5511.4141 | 2.1 |
| | | WPEACVLEPCPPYWAV | C(5), O- | | | | |
| | | GDFGPCSASCGGGLR | Fucosylation | | | | |
| | | (SEQ ID NO: 59) | Glc-Fuc | | | | |
| | | | ST(1) | | | | |
| T065- | 690-694 | ERPVR | | 13.3 | 655.3758 | 655.3765 | −1.1 |
| 066 | | (SEQ ID NO: 60) | | | | | |
| T067* | 695-704 | CVEAQGSLLK | Carbamidomethyl | 32.2 | 1103.5646 | 1103.5646 | 0.0 |
| | | (SEQ ID NO: 61) | C(1) | | | | |
| T067- | 695-710 | CVEAQGSLLKTLPPAR | Carbamidomethyl | 46.2 | 1738.9391 | 1738.9399 | −0.5 |
| 068* | | (SEQ ID NO: 62) | C(1) | | | | |
| T068 | 705-710 | TLPPAR | | 20.8 | 653.3878 | 653.3860 | 2.8 |
| | | (SEQ ID NO: 63) | | | | | |
| T068 | 705-710 | TLPPAR | | 20.0 | 653.3823 | 653.3860 | −5.7 |
| | | (SEQ ID NO: 63) | | | | | |
| T068 | 705-710 | TLPPAR | | 20.3 | 653.3851 | 653.3860 | −1.4 |
| | | (SEQ ID NO: 63) | | | | | |
| T068 | 705-710 | TLPPAR | | 21.1 | 653.3869 | 653.3860 | 1.4 |
| | | (SEQ ID NO: 63) | | | | | |
| T068 | 705-710 | TLPPAR | | 20.4 | 653.3839 | 653.3860 | −3.2 |
| | | (SEQ ID NO: 63) | | | | | |
| T068- | 705-712 | TLPPARCR | Carbamidomethyl | 47.4 | 969.5197 | 969.5178 | 2.0 |
| 069* | | (SEQ ID NO: 64) | C(1) | | | | |
| T070* | 713-733 | AGAQQPAVALETCNPQ | Carbamidomethyl | 33.2 | 2235.0525 | 2235.0525 | 0.0 |
| | | PCPAR | C(2) | | | | |
| | | (SEQ ID NO: 65) | | | | | |
| T071* | 734-778 | WEVSEPSSCTSAGGAG | Carbamidomethyl | 43.4 | 7814.2805 | 7814.1704 | −14.1 |
| | | LALENETCVPGADGLE | C(2), | | | | |
| | | APVTEGPGSVDEK | O-Glycosylation | | | | |
| | | (SEQ ID NO: 66) | NeuAc2- | | | | |
| | | | Hex-HexNAc | | | | |
| | | | ST(1), | | | | |
| | | | N-Glycosylation | | | | |
| | | | biFucSA2 | | | | |
| | | | N(1) | | | | |
| T071* | 734-778 | WEVSEPSSCTSAGGAG | Carbamidomethyl | 43.0 | 8470.2893 | 8470.3980 | 12.8 |
| | | LALENETCVPGADGLE | C(2), | | | | |
| | | APVTEGPGSVDEK | O-Glycosylation | | | | |
| | | (SEQ ID NO: 66) | NeuAc2- | | | | |
| | | | Hex-HexNAc | | | | |
| | | | ST(1), | | | | |
| | | | N-Glycosylation | | | | |
| | | | triFucSA3 | | | | |
| | | | N(1) | | | | |
| T071* | 734-778 | WEVSEPSSCTSAGGAG | Carbamidomethyl | 46.9 | 5464.3141 | 5464.3240 | 1.8 |
| | | LALENETCVPGADGLE | C(2), | | | | |
| | | APVTEGPGSVDEK | O-Glycosylation | | | | |
| | | (SEQ ID NO: 66) | NeuAc2- | | | | |
| | | | Hex-HexNAc | | | | |
| | | | ST(1), | | | | |
| | | | Deamidation | | | | |
| | | | N(1) | | | | |
| T072* | 779-805 | LPAPEPCVGMSCPPGW | Carbamidomethyl | 45.6 | 2836.2776 | 2836.2620 | 5.5 |
| | | GHLDATSAGEK | C(2), | | | | |
| | | (SEQ ID NO: 67) | Oxidation M(1) | | | | |
| T072* | 779-805 | LPAPEPCVGMSCPPGW | Carbamidomethyl | 47.0 | 2820.2888 | 2820.2671 | 7.7 |
| | | GHLDATSAGEC | C(2) | | | | |
| | | (SEQ ID NO: 68) | | | | | |
| T072* | 779-805 | LPAPEPCVGMSCPPGW | Carbamidomethyl | 44.4 | 3767.6038 | 3767.5901 | 3.6 |
| | | GHLDATSAGEK | C(2), O- | | | | |
| | | (SEQ ID NO: 67) | Glycosylation | | | | |
| | | | NeuAc2- Hex- | | | | |
| | | | HexNAc ST(1) | | | | |
| T072* | 779-805 | LPAPEPCVGMSCPPGW | Carbamidomethyl | 43.0 | 3783.5906 | 3783.5850 | 1.5 |
| | | GHLDATSAGEK | C(2), | | | | |
| | | (SEQ ID NO: 67) | Oxidation M(1), | | | | |
| | | | O-Glycosylation | | | | |
| | | | NeuAc2- | | | | |
| | | | Hex-HexNAc | | | | |
| | | | ST(1) | | | | |
| T073 | 806-814 | APSPWGSIR | | 38.7 | 969.5008 | 969.5032 | −2.5 |
| | | (SEQ ID NO: 69) | | | | | |
| T074* | 815-836 | TGAQAAHVWTPAAGSC | Carbamidomethyl | 34.5 | 2230.0056 | 2230.0007 | 2.2 |
| | | SVSCGR | C(2) | | | | |
| | | (SEQ ID NO: 70) | | | | | |
| T074* | 815-836 | TGAQAAHVWTPAAGSC | Carbamidomethyl | 34.5 | 2538.1042 | 2538.1116 | −2.9 |
| | | SVSCGR | C(2), | | | | |
| | | (SEQ ID NO: 70) | O-Fucosylation Glc- | | | | |
| T075 | 837-842 | GLMELR | | 37.7 | 717.3845 | 717.3843 | 0.3 |
| | | (SEQ ID NO: 71) | | | | | |
| T076* | 843-851 | FLCMDSALR | Carbamidomethyl | 44.1 | 1111.5166 | 1111.5155 | 1.0 |
| | | (SEQ ID NO: 72) | C(1) | | | | |
| T076* | 843-851 | FLCMDSALR | Carbamidomethyl | 39.5 | 1111.5233 | 1111.5155 | 7.0 |
| | | (SEQ ID NO: 72) | C(1) | | | | |
| T077* | 852-864 | VPVQEELCGLASK | Carbamidomethyl | 41.1 | 1428.7297 | 1428.7283 | 1.0 |
| | | (SEQ ID NO: 73) | C(1) | | | | |
| T078 | 865-868 | PGSR | | 5.1 | 415.2180 | 415.2179 | −0.2 |
| | | (SEQ ID NO: 74) | | | | | |
| T079- | 869-880 | REVCQAVPCPAR | Carbamidomethyl | 27.4 | 1441.6904 | 1441.6919 | −1.0 |
| 080* | | (SEQ ID NO: 75) | C(2) | | | | |
| T080* | 870-880 | EVCQAVPCPAR | Carbamidomethyl | 26.7 | 1285.5908 | 1285.5908 | 0.0 |
| | | (SEQ ID NO: 76) | C(2) | | | | |
| T081 | 881-884 | WQYK | | 24.8 | 623.3077 | 623.3068 | 1.4 |
| | | (SEQ ID NO: 77) | | | | | |
| T082* | 885-894 | LAACSVSCGR | Carbamidomethyl | 22.3 | 1387.5975 | 1387.5959 | 1.2 |
| | | (SEQ ID NO: 78) | C(2), | | | | |
| | | | O-Fucosylation Glc- | | | | |
| | | | Fuc | | | | |
| | | | ST(1) | | | | |
| T082* | 885-894 | LAACSVSCGR | Carbamidomethyl | 22.3 | 1079.4866 | 1079.4852 | 1.3 |
| | | (SEQ ID NO: 78) | C(2) | | | | |
| T083 | 895-898 | GVVR | | 14.1 | 429.2697 | 429.2700 | −0.7 |
| | | (SEQ ID NO: 79) | | | | | |
| T084 | 899-899 | R | | n.d. | n.d. | 174.1117 | n.d. |
| T085* | 900-905 | ILYCAR | Carbamidomethyl | 27.3 | 794.4083 | 794.4109 | −3.3 |
| | | (SEQ ID NO: 80) | C(1) | | | | |
| T086* | 906-926 | AHGEDDGEEILLDTQC | Carbamidomethyl | 44.2 | 2352.0686 | 2352.0652 | 1.4 |
| | | QGLPR | | | | | |
| | | (SEQ ID NO: 81) | C(1) | | | | |
| T086- | 906-941 | AHGEDDGEEILLDTQC | Carbamidomethyl | 47.6 | 4099.8262 | 4099.8311 | −1.2 |
| 087* | | QGLPRPEPQEACSLEP | C(3) | | | | |
| | | CPPR | | | | | |
| | | (SEQ ID NO: 82) | | | | | |
| T087* | 927-941 | PEPQEACSLEPCPPR | Carbamidomethyl | 32.2 | 1765.7749 | 1765.7764 | −0.8 |
| | | (SEQ ID NO: 83) | C(2) | | | | |
| T087* | 927-941 | PEPQEACSLEPCPPR | Carbamidomethyl | 46.3 | 1765.7804 | 1765.7764 | 2.3 |
| | | (SEQ ID NO: 83) | C(2) | | | | |
| T088 | 942-943 | WK | | 13.9 | 332.1848 | 332.1848 | 0.0 |
| T089* | 944-960 | VMSLGPCSASCGLGTA | Carbamidomethyl | 37.7 | 2030.8949 | 2030.8959 | −0.5 |
| | | R | C(2), | | | | |
| | | (SEQ ID NO: 84) | O-Fucosylation Glc- | | | | |
| | | | Fuc | | | | |
| | | | ST(1) | | | | |
| T089* | 944-960 | VMSLGPCSASCGLGTA | Carbamidomethyl | 37.7 | 1722.7849 | 1722.7852 | −0.2 |
| | | R | C(2) | | | | |
| | | (SEQ ID NO: 84) | | | | | |
| T089* | 944-960 | VMSLGPCSASCGLGTA | Carbamidomethyl | 32.7 | 2046.8887 | 2046.8907 | −1.0 |
| | | R | C(2), | | | | |
| | | (SEQ ID NO: 84) | Oxidation | | | | |
| | | | M(1),O- | | | | |
| | | | Fucosylation | | | | |
| | | | Glc-Fuc ST(1) | | | | |
| T090 | 961-961 | R | | n.d. | n.d. | 174.1117 | n.d. |
| T091- | 962- | SVACVQLDQGQDVEVD | Carbamidomethyl | 56.7 | 4435.0591 | 4435.0552 | 0.9 |
| 092* | 1001 | EAACAALVRPEASVPC | C(4) | | | | |
| | | LIADCTYR | | | | | |
| | | (SEQ ID NO: 85) | | | | | |
| T092* | 987- | PEASVPCLIADCTYR | Carbamidomethyl | 45.2 | 1750.8051 | 1750.8019 | 1.8 |
| | 1001 | (SEQ ID NO: 86) | C(2) | | | | |
| T093* | 1002- | WHVGTWMECSVSCGDG | Carbamidomethyl | 46.7 | 2572.0864 | 2572.0669 | 7.6 |
| | 1020 | IQR | C(2), | | | | |
| | | (SEQ ID NO: 87) | O-Fucosylation Glc- | | | | |
| | | | Fuc | | | | |
| | | | ST(1) | | | | |
| T093* | 1002- | WHVGTWMECSVSCGDG | Carbamidomethyl | 46.7 | 2263.9734 | 2263.9563 | 7.6 |
| | 1020 | IQR | C(2) | | | | |
| | | (SEQ ID NO: 87) | | | | | |
| T093* | 1002- | WHVGTWMECSVSCGDG | Carbamidomethyl | 46.4 | 2572.0776 | 2572.0669 | 4.2 |
| | 1020 | IQR | C(2), | | | | |
| | | (SEQ ID NO: 87) | O-Fucosylation Glc- | | | | |
| | | | Fuc | | | | |
| | | | ST(1) | | | | |
| T094 | 1021- | R | | n.d. | n.d. | 174.11 | n.d. |
| T095- | 1022- | RDTCLGPQAQAPVPAD | Carbamidomethyl | 44.3 | 2605.2664 | 2605.2529 | 5.2 |
| 096* | 1044 | FCQHLPK | C(2) | | | | |
| | | (SEQ ID NO: 88) | | | | | |
| T096* | 1023- | DTCLGPQAQAPVPADF | Carbamidomethyl | 45.0 | 2449.1646 | 2449.1519 | 5.2 |
| | 1044 | CQHLPK | C(2) | | | | |
| | | (SEQ ID NO: 89) | | | | | |
| T096- | 1023- | DTCLGPQAQAPVPADF | Carbamidomethyl | 48.0 | 3001.4922 | 3001.4902 | 0.7 |
| 097* | 1049 | CQHLPKPVTVR | C(2) | | | | |
| | | (SEQ ID NO: 90) | | | | | |
| T097 | 1045- | PVTVR | | 18.4 | 570.3502 | 570.3489 | 2.3 |
| | 1049 | (SEQ ID NO: 91) | | | | | |
| T098- | 1050- | GCWAGPCVGQGTPSLV | Carbamidomethyl | 42.6 | 7129.9219 | 7129.9868 | −9.1 |
| 099* | 1091 | PHEEAAAPGRTTATPA | C(2), | | | | |
| | | GASLEWSQAR | O-Glycosylation | | | | |
| | | (SEQ ID NO: 92) | NeuAc2- Hex- | | | | |
| | | | HexNAc ST(3) | | | | |
| T098- | 1050- | GCWAGPCVGQGTPSLV | Carbamidomethyl | 43.4 | 6838.9045 | 6838.8915 | −1.9 |
| 099* | 1091 | PHEEAAAPGRTTATPA | C(2), | | | | |
| | | GASLEWSQAR | O-Glycosylation | | | | |
| | | (SEQ ID NO: 92) | NeuAc- | | | | |
| | | | Hex-HexNAc | | | | |
| | | | ST(1), | | | | |
| | | | O-Glycosylation | | | | |
| | | | NeuAc2-Hex- | | | | |
| | | | HexNAc ST(2) | | | | |
| T098- | 1050- | GCWAGPCVGQGTPSLV | Carbamidomethyl | 38.7 | 8077.2681 | 8077.3099 | 5.2 |
| 099* | 1091 | PHEEAAAPGRTTATPA | C(2), | | | | |
| | | GASLEWSQAR | O-Glycosylation | | | | |
| | | (SEQ ID NO: 92) | NeuAc2-Hex- | | | | |
| | | | HexNAc ST(4) | | | | |
| T098- | 1050- | GCWAGPCVGQGTPSLV | Carbamidomethyl | 39.4 | 7786.0997 | 7786.2145 | 14.7 |
| 099* | 1091 | PHEEAAAPGRTTATPA | C(2), O- | | | | |
| | | GASLEWSQAR | Glycosylation | | | | |
| | | (SEQ ID NO: 92) | NeuAc- | | | | |
| | | | Hex-HexNAc | | | | |
| | | | ST(1), O- | | | | |
| | | | Glycosylation | | | | |
| | | | NeuAc2-Hex- | | | | |
| | | | HexNAc ST(3) | | | | |
| T099 | 1076- | TTATPAGASLEWSQAR | | 54.4 | 1645.7960 | 1645.8060 | −6.1 |
| | 1091 | (SEQ ID NO: 93) | | | | | |
| T100* | 1092- | GLLFSPAPQPR | | 38.4 | 1181.6520 | 1181.6556 | −3.0 |
| | 1102 | (SEQ ID NO: 94) | | | | | |
| T100* | 1092- | GLLFSPAPQPR | | 39.6 | 1181.6561 | 1181.6556 | 0.4 |
| | 1102 | (SEQ ID NO: 94) | | | | | |
| T100* | 1092- | GLLFSPAPQPR | O-Glycosylation | 38.4 | 2128.9768 | 2128.9788 | −0.9 |
| | 1102 | (SEQ ID NO: 94) | NeuAc2- | | | | |
| | | | Hex-HexNAc | | | | |
| | | | ST(1) | | | | |
| T100* | 1092- | GLLFSPAPQPR | O-Glycosylation | 39.8 | 1837.9018 | 1837.8833 | −10.1 |
| | 1102 | (SEQ ID NO: 94) | NeuAc- | | | | |
| T100* | 1092- | GLLFSPAPQPR | O-Glycosylation | 39.6 | 1837.8752 | 1837.8833 | 4.4 |
| | 1102 | (SEQ ID NO: 94) | NeuAc- | | | | |
| | | | Hex-HexNAc | | | | |
| | | | ST(1) | | | | |
| T100* | 1092- | GLLFSPAPQPR | O-Glycosylation | 40.9 | 1546.8016 | 1546.7879 | −8.9 |
| | 1102 | (SEQ ID NO: 94) | Hex- | | | | |
| | | | HexNAc ST(1) | | | | |
| T101 | 1103- | R | | n.d. | n.d. | 174.1117 | n.d. |
| | 1103 | | | | | | |
| T102* | 1104- | LLPGPQENSVQSSACG | Carbamidomethyl | 31.0 | 1798.8619 | 1798.8632 | −0.7 |
| | 1120 | R | C(1) | | | | |
| | | (SEQ ID NO: 95) | | | | | |
| T103 | 1121- | QHLEPTGTIDMR | | 34.5 | 1396.6764 | 1396.6769 | −0.4 |
| | 1132 | (SEQ ID NO: 96) | | | | | |
| T104* | 1133- | GPGQADCAVAIGR | Carbamidomethyl | 28.7 | 1270.6053 | 1270.6088 | −2.8 |
| | 1145 | (SEQ ID NO: 97) | C(1) | | | | |
| T105 | 1146- | PLGEVVTLR | | 41.2 | 982.5804 | 982.5811 | −0.7 |
| | 1154 | (SEQ ID NO: 98) | | | | | |
| T105 | 1146- | PLGEVVTLR | | 45.4 | 982.5829 | 982.5811 | 1.8 |
| | 1154 | (SEQ ID NO: 98) | | | | | |
| T106* | 1155- | VLESSLNCSAGDMLLL | N-Glycosylation | 57.6 | 4470.8794 | 4470.8696 | 2.2 |
| | 1173 | WGR | biFucSA2 | | | | |
| | | (SEQ ID NO: 99) | N(1), | | | | |
| | | | Carbamidomethyl | | | | |
| | | | C(1) | | | | |
| T106* | 1155- | VLESSLNCSAGDMLLL | N-Glycosylation | 57.9 | 4179.7741 | 4179.7744 | 0.1 |
| | 1173 | WGR | biFucSA | | | | |
| | | (SEQ ID NO: 99) | N(1), | | | | |
| | | | Carbamidomethyl | | | | |
| | | | C(1) | | | | |
| T107 | 1174- | LTWR | | 31.3 | 574.3236 | 574.3228 | 1.4 |
| | 1177 | (SEQ ID NO: 100) | | | | | |
| T108 | 1178- | K | | n.d. | n.d. | 146.1055 | n.d. |
| | 1178 | | | | | | |
| T109* | 1179- | MCR | Carbamidomethyl | 9.3 | 465.1831 | 465.1829 | 0.4 |
| | 1181 | | C(1) | | | | |
| T110- | 1182- | KLLDMTFSSK | | 43.7 | 1168.6167 | 1168.6162 | 0.4 |
| 111 | 1191 | (SEQ ID NO: 101) | | | | | |
| T111 | 1183- | LLDMTFSSK | | 43.7 | 1040.5210 | 1040.5212 | −0.2 |
| | 1191 | (SEQ ID NO: 102) | | | | | |
| T111* | 1183- | LLDMTFSSK | Oxidation M(1) | 34.3 | 1056.5184 | 1056.5161 | 2.2 |
| | 1191 | (SEQ ID NO: 102) | | | | | |
| T112 | 1192- | TNTLVVR | | 27.9 | 801.4710 | 801.4708 | 0.2 |
| | 1198 | (SEQ ID NO: 103) | | | | | |
| T113 | 1199- | QR | | n.d. | n.d. | 302.1703 | n.d. |
| | 1200 | | | | | | |
| T114- | 1201- | CGRPGGGVLLR | Carbamidomethyl | 34.0 | 1140.6196 | 1140.6187 | 0.8 |
| 115* | 1211 | (SEQ ID NO: 104) | C(1) | | | | |
| T115 | 1204- | PGGGVLLR | | 33.2 | 767.4647 | 767.4653 | −0.8 |
| | 1211 | (SEQ ID NO: 105) | | | | | |
| T116 | 1212- | YGSQLAPETFYR | | 40.6 | 1430.6863 | 1430.6830 | 2.3 |
| | 1223 | (SEQ ID NO: 106) | | | | | |
| T117* | 1224- | ECDMQLFGPWGEIVSP | Carbamidomethyl | 62.4 | 3122.3948 | 3122.3896 | 1.7 |
| | 1252 | SLSPATSNAGGCR | C(2) | | | | |
| | | (SEQ ID NO: 107) | | | | | |
| T118 | 1253- | LFINVAPHAR | | 42.9 | 1136.6484 | 1136.6454 | 2.6 |
| | 1262 | (SEQ ID NO: 108) | | | | | |
| T119* | 1263- | IAIHALATNMGAGTEG | N-Glycosylation | 50.6 | 4878.1577 | 4878.1519 | 1.2 |
| | 1287 | ANASYILIR | biFucSA2 N(1) | | | | |
| | | (SEQ ID NO: 109) | | | | | |
| T120 | 1288- | DTHSLR | | 15.9 | 727.3631 | 727.3613 | 2.5 |
| | 1293 | (SEQ ID NO: 110) | | | | | |
| T121 | 1294- | TTAFHGQQVLYWESES | | 62.5 | 3365.5249 | 3365.5186 | 1.9 |
| | 1322 | SQAEMEFSEGFLK | | | | | |
| | | (SEQ ID NO: 111) | | | | | |
| T122 | 1323- | AQASLR | | 16.6 | 644.3618 | 644.3605 | 2.0 |
| | 1328 | (SEQ ID NO: 112) | | | | | |
| T123 | 1329- | GQYWTLQSWVPEMQDP | | 61.8 | 2493.1362 | 2493.1423 | −2.4 |
| | 1348 | QSWK | | | | | |
| | | (SEQ ID NO: 113) | | | | | |
| T124 | 1349- | GK | | n.d. | n.d. | 203.1270 | n.d. |
| | 1350 | | | | | | |
| T125 | 1351- | EGT | | n.d. | n.d. | 305.1223 | n.d. |
| | 1353 | | | | | | |

¹numbering without signal-and propeptide;
²no modification on this peptide; n.d. not detected.

The overlay of tryptic peptide maps of several samples revealed high comparability of the samples as no major difference could be observed in the peak pattern. Thus, similar posttranslational modifications occurred across the samples.

The recombinant ADAMTS13 shows a C-mannosylation that is typical for the Thrombospondin type-1 (TSP-1) motif containing the sequence WXXW. This modification could be detected and was found approximately 30% modified and 70% unmodified.

The main N-glycan variants that were found by the peptide mapping were a biantennary, monosialyated fucosylated glycan and a biantennary, disialylated fucosylated glycan.

The seven 0-fucosylations on the TSP-1 motifs were all found to be completely occupied by the disaccharide Fuc-Glc. Furthermore, several mucine type 0-glycans with the structure HexNAc-Hex-NeuAc₀₋₂ could be identified. In Table 10 these 0-glycosylations with their approximate occupancy are summarized. Peptide T071 was found to be completely modified with HexNAc-Hex-NeuA_{c2}. For peptide T100 the major part (88%) is also modified with this structure and only minor amounts are not modified. For Peptide T072 60% were detected in the unmodified form and approximately 40% were found to be modified with the 0-glycan HexNac-Hex-NeuA_{c2}. The tryptic peptide T098-099 was detected with up to four 0-glycans (30%) of the same type with different levels of sialic acid, around 70% were found bearing three 0-glycans and no unmodified peptide could be detected.

TABLE 10
Identified O-Linked Glycosylation of ADAMTS13

| | | | | Monoisotopic | |
|---|---|---|---|---|---|
| | | | | mass | Mass |
| | | | | Experimental | Accuracy |
| Peptide | AA range | Peptide | Area% | [Da] | [ppm] |
| T071 | 734-778 | WEVSEPSSCTSAGGAGLALENETCVPGADGLEAPV | n.d. | n.d. | n.d. |
| | | TEGPGSVDEK (SEQ ID NO: 66) | | | |
| | | + HexNac-Hex-NeuAc2 | 100 | 5464.3141 | 1.8246 |
| T072 | 779-805 | LPAPEPCVGMSCPPGWGHLDATSAGEK | 29.6 | 2820.2764 | −3.3388 |
| | | (SEQ ID NO: 67) | | | |
| | | ox | 28.9 | 2836.2649 | −1.0582 |
| | | ox + HexNac-Hex-NeuAc2 | 10.6 | 3783.5791 | 1.5404 |
| | | + HexNac-Hex-NeuAc2 | 30.8 | 3767.6209 | −8.1980 |
| T098-099 | 1050-1091 | GCWAGPCVGQGTPSLVPHEEAAAPGRTTATPAGAS | n.d. | n.d. | n.d. |
| | | LEWSQAR (SEQ ID NO: 92) | | | |
| | | + 4x HexNac-Hex-NeuAc2 | 14.4 | 8077.2681 | 5.1805 |
| | | + HexNac-Hex-NeuAc + 3x HexNac- | 18.6 | 7786.0997 | 14.7477 |
| | | Hex-NeuAc2 | | | |
| | | + 3x HexNac-Hex-NeuAc2 | 47.3 | 7130.0397 | −7.4032 |
| | | + HexNac-Hex-NeuAc + 2x HexNac- | 19.7 | 6838.9045 | −1.9011 |
| | | Hex-NeuAc2 | | | |
| T100 | 1092-1102 | GLLFSPAPQPR (SEQ ID NO: 94) | 0.2 | 1181.6600 | −3.6853 |
| | | + HexNac-Hex-NeuAc2 | 88.8 | 2128.9902 | −5.4135 |
| | | + HexNac-Hex-NeuAc | 10.3 | 1837.8752 | 4.3847 |
| | | + HexNac-Hex | 0.7 | 1546.8016 | −8.8944 |

*n.d. = not detected

ADAMTS13 glycosylation was also evaluated by monosaccharide analysis of the liberated, hydrolyzed rADAMTS13 glycans. The procedure involved acid hydrolysis, labeling using 2-AA and separation/detection/quantification by reversed phase HPLC, linked with fluorescence detection. rADAMTS13 samples were desalted by acetone precipitation. The desalted samples were resuspended in 6.75 M trifluoro-acetic acid and incubated at 100° C. for 1.5 hours. Liberated monosaccharides were labeled with 2-AA (anthranilic acid) by reductive amination and separated by reversed phase HPLC on a Jupiter 5μ C18 column using an acetonitrile gradient with phosphoric acid and butylamine as modifier. Calibration was done with the appropriate mixture of standard monosaccharides. Table 11 summarizes the data for all samples analyzed.

TABLE 11
Monosaccharide Analysis for ADAMTS13

| | GlcNAc | GalNAc | Gal | Man | Glc | Fuc |
|---|---|---|---|---|---|---|
| Sample | [%] | [%] | [%] | [%] | [%] | [%] |
| 1 | 27.0 | 5.8 | 23.9 | 24.5 | 6.6 | 12.3 |
| 2 | 28.7 | 5.9 | 24.3 | 23.3 | 5.8 | 11.9 |
| 3 | 28.3 | 5.8 | 24.1 | 23.8 | 5.9 | 12.1 |
| mean | 28.0 | 5.8 | 24.1 | 23.8 | 6.1 | 12.1 |
| SD | 0.9 | 0.0 | 0.2 | 0.6 | 0.4 | 0.2 |
| 4 | 28.3 | 5.8 | 23.4 | 24.2 | 5.9 | 12.3 |
| 5 | 27.6 | 5.8 | 24.0 | 24.4 | 5.9 | 12.3 |
| 6 | 28.4 | 5.7 | 23.5 | 24.4 | 5.9 | 12.2 |
| mean | 28.1 | 5.8 | 23.6 | 24.3 | 5.9 | 12.3 |
| SD | 0.4 | 0.1 | 0.3 | 0.1 | 0.0 | 0.1 |

ADAMTS13 glycosylation was also assessed through 2-AB glycan profiling involving glycan release of the N-linked oligosaccharides with PNGase F and labeling of the reducing end with 2-AB (2-aminobenzamide). Separation and relative quantification of the labeled oligosaccharides was performed by normal phase HPLC and fluorescence detection. Briefly, samples were denaturated with urea and bound to AssayMap cartridges. Enzymatic deglycosylation was done using PNGase F and released N-glycans are washed from the cartridges and lyophilized before labeling with 2-aminobenzamide by reductive amination. Oligosaccharides were separated on a Luna Amino 3μ column using a water/acetonitrile/250 mM ammonium acetate pH 4.5 gradient and detected using fluorescence detection. The N-linked glycans are grouped based on their charge related to the number of sialic acids into five charge clusters (neutral, monosialylated, disialylated, trisialylated and tetrasialylated N-glycans). In addition to the comparison of the chromatographic profiles the relative abundance of each charge state cluster and the N-glycan index is calculated. N-glycan index is calculated from the relative area (%) of the different charge state clusters by the following formula: N-glycan index=Neutral*0+Monosialo*1+Disialo*2+Trisialo*3+Tetrasialo*4.

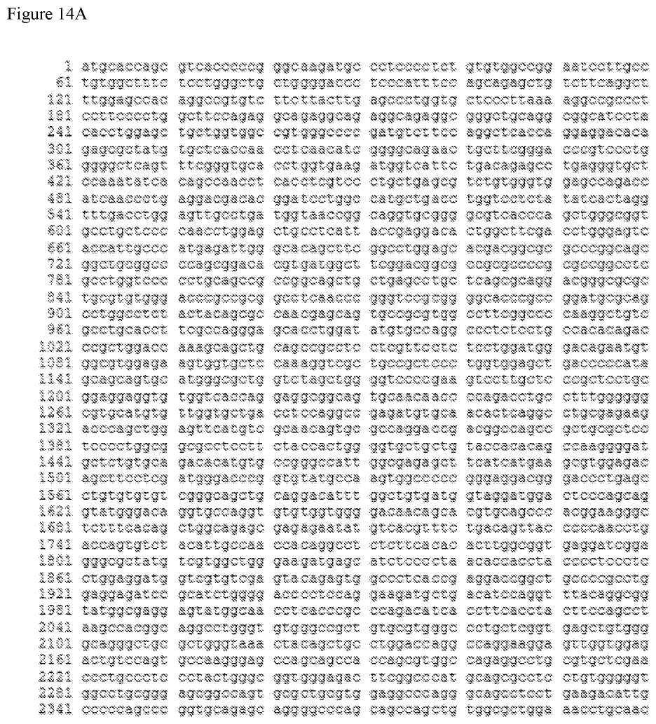

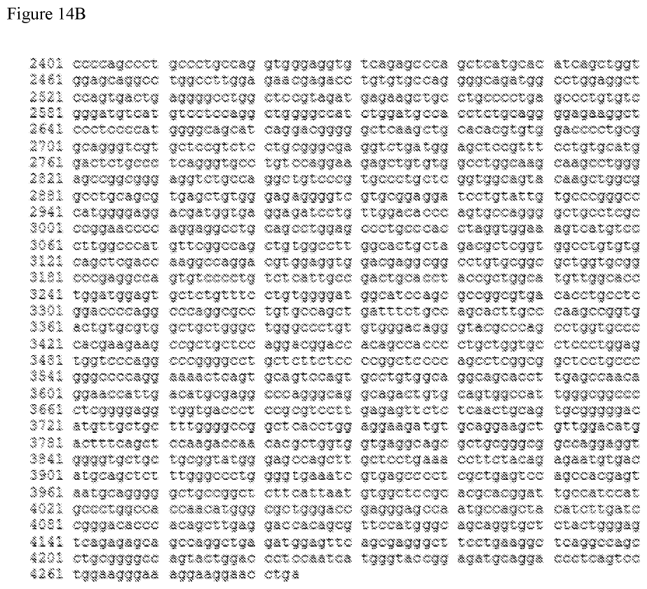

The main N-glycan variants that were found via peptide mapping were a biantennary, monosialylated fucosylated and a biantennary, disialylated fucosylated glycan which is in accordance with the data from N-glycan mapping (FIG. 13). Table 12 summarizes the N-glycan mapping data for all samples analyzed. The main N-glycan structure that can be found on ADAMTS13 is a biantennary, core fucosylated complex type oligosaccharide with one or two sialic acids.

TABLE 12
N-Glycan Mapping Data

| | | | | | | N- |
|---|---|---|---|---|---|---|
| | Neutral | Monosialo | Disisalo | Trisialo | Tetrasialo | glycan |
| Sample | [Area %] | [Area %] | [Area %] | [Area %] | [Area %] | index |
| 1 | 14.0 | 28.3 | 40.0 | 14.0 | 3.7 | 165.1 |
| 2 | 13.5 | 26.2 | 41.5 | 14.9 | 3.9 | 169.5 |
| 3 | 16.0 | 32.7 | 36.8 | 11.7 | 2.8 | 152.6 |
| Mean | 14.5 | 29.1 | 39.4 | 13.5 | 3.5 | 162.4 |
| SD | 1.3 | 3.3 | 2.4 | 1.7 | 0.6 | 8.8 |
| 4 | 13.7 | 30.0 | 40.3 | 13.0 | 3.0 | 161.7 |
| 5 | 12.8 | 28.8 | 41.7 | 13.5 | 3.2 | 165.6 |
| 6 | 13.7 | 28.4 | 41.1 | 13.4 | 3.4 | 164.5 |
| Mean | 13.4 | 29.1 | 41.0 | 13.3 | 3.2 | 163.9 |
| SD | 0.5 | 0.8 | 0.7 | 0.3 | 0.2 | 2.0 |
| 7 | 13.69 | 29.96 | 40.34 | 12.97 | 3.04 | 162 |
| 8 | 12.77 | 28.83 | 41.70 | 13.48 | 3.22 | 166 |
| 9 | 13.69 | 28.40 | 41.06 | 13.41 | 3.44 | 165 |
| Mean | 13.38 | 29.06 | 41.03 | 13.29 | 3.23 | 164 |
| SD | 0.53 | 0.80 | 0.68 | 0.28 | 0.20 | 2 |
| 10 | 13.69 | 25.52 | 43.19 | 12.22 | 5.39 | 170 |
| 11 | 13.63 | 25.75 | 43.27 | 11.91 | 5.44 | 170 |
| 12 | 13.65 | 26.95 | 40.40 | 13.31 | 5.71 | 170 |
| Mean | 13.66 | 26.07 | 42.29 | 12.48 | 5.51 | 170 |
| SD | 0.03 | 0.77 | 1.63 | 0.74 | 0.17 | 0.3 |

Protein-bound sialic acids were liberated from the rADAMTS13 by mild acid hydrolysis, labeled using DMB and then quantified by reversed phase HPLC with fluorescence detection. ADAMTS13 samples were desalted by acetone precipitation, reconstitution in MilliQ-water to a final concentration of 2 M acetic acid and incubated at 80° C. for 2.5 hours. Liberated sialic acids were labeled with 1,2-diamino-4,5-methylenedioxybenzene (DMB) and separated by reversed phase HPLC on a Jupiter 5μ C18 column using an acetonitrile/methanol/water gradient. Calibration was performed with standard preparations of N-acetyl neuraminic acid (NANA) and N-glycolyl neuraminic acid (NGNA). Table 13 summarizes the data for all samples analyzed.

TABLE 13
Data for NANA and NGNA from Quantitative Sialic Acid Analysis

| | nmol NANA/mg | nmol NGNA/mg | % NGNA |
|---|---|---|---|
| Sample | rADAMTS13 | rADAMTS13 | (relative to NANA) |
| 1 | 132.4 | 0.10 | 0.07 |
| 2 | 168.6 | 0.31 | 0.18 |
| 3 | 160.5 | 0.12 | 0.07 |
| Mean | 153.8 | 0.17 | 0.11 |
| SD | 19.0 | 0.12 | 0.06 |
| 4 | 147.6 | 0.26 | 0.17 |
| 5 | 156.0 | 0.25 | 0.16 |
| 6 | 151.8 | 0.22 | 0.15 |
| Mean | 151.8 | 0.24 | 0.16 |
| SD | 4.2 | 0.02 | 0.01 |
| 7 | 135.8 | 0.13 | 0.10 |
| 8 | 137.8 | 0.11 | 0.08 |
| 9 | 131.3 | 0.16 | 0.12 |
| Mean | 135.0 | 0.14 | 0.10 |
| SD | 3.3 | 0.02 | 0.02 |
| 10 | 148.9 | 0.16 | 0.11 |
| 11 | 130.1 | 0.11 | 0.09 |
| 12 | 140.1 | 0.08 | 0.06 |
| Mean | 139.7 | 0.12 | 0.08 |
| SD | 9.4 | 0.04 | 0.03 |

The detected amounts of NANA in the samples were highly comparable and in the range of about 130 to 169 nmol NANA per mg rADAMTS13 protein.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety for all purposes as if physically present in this specification.

1. A recombinant ADAMTS13 variant, wherein the ADAMTS13 variant consists of a single amino acid substitution Q97R as denoted in SEQ ID NO: 1, or an equivalent amino acid position in an ADAMTS13 protein.

2.-4. (canceled)

5. The recombinant ADAMTS13 variant of claim 1, wherein the ADAMTS13 variant comprises the amino acid sequence of SEQ ID NO: 2.

6. The recombinant ADAMTS13 variant of claim 5, wherein the ADAMTS13 variant consists essentially of the amino acid sequence of SEQ ID NO: 2.

7. The recombinant ADAMTS13 variant of claim 5, wherein the ADAMTS13 variant consists of the amino acid sequence of SEQ ID NO: 2.

8. The recombinant ADAMTS13 variant of claim 1, wherein the ADAMTS13 protein is a human ADAMTS13.

9.-11. (canceled)

12. A pharmaceutical composition comprising at least one ADAMTS13 variant of claim 1 and a pharmaceutically acceptable carrier or excipient.

13. The pharmaceutical composition of claim 12, further comprising an ADAMTS13 protein.

14. The pharmaceutical composition of claim 13, wherein the ADAMTS13 protein comprises the amino acid sequence of SEQ ID NO: 1, or an amino acid sequence having at least 80% sequence identity thereof.

15. The pharmaceutical composition of claim 13, wherein the ADAMTS13 protein consists of the amino acid sequence of SEQ ID NO: 1.

16. The pharmaceutical composition of claim 13, wherein the ADAMTS13 protein is recombinantly produced.

17. The pharmaceutical composition of claim 13, wherein the ADAMTS13 protein is plasma derived.

18. The pharmaceutical composition of claim 13, wherein the ratio of ADAMTS13 variant to ADAMTS13 protein is about 4:1 to about 1:4, about 1:1 to about 3:1, about 1:1, or about 3:2.

19. The pharmaceutical composition of claim 13, wherein the ADAMTS13 variant constitutes between about 40% to about 90%, between about 52% to about 72%, or between about 47% to about 84% of total amount of all ADAMTS13 proteins and variants in the composition.

20. The pharmaceutical composition of claim 18, wherein the ratio is determined by peptide mapping method.

21. The pharmaceutical composition of claim 18, wherein the ratio is determined by HPLC analysis of tryptic peptides separated by liquid chromatography followed by mass spectrometry analysis.

22. The pharmaceutical composition of claim 18, wherein the ratio is determined based on intensities in extracted ion chromatograms.

23. The pharmaceutical composition of claim 18, wherein the ratio is determined based on a peak area of tryptic peptides of the ADAMTS13 variant in relation to a sum of a peak areas of all ADAMTS13 proteins and variants in the composition.

24. (canceled)

25. The pharmaceutical composition of claim 23, wherein the tryptic peptide(s) measured for the ADAMTS13 variant is
AAGGILHLELLVAVGPDVFQAHR or a combination of
AAGGILHLELLVAVGPDVFQAHR and EDTER.

26. The pharmaceutical composition of claim 23, wherein the tryptic peptide measured for the ADAMTS13 protein is
AAGGILHLELLVAVGPDVFQAHQEDTER.

27. The pharmaceutical composition of claim 18, wherein the ratio is determined based on total weight of ADAMTS13 variant in relation to a sum total weight of all ADAMTS13 proteins and variants in the composition.

28. A method for treating or preventing a blood clotting disorder in a subject suffering from or at risk of suffering from a blood clotting disorder, comprising administering to the subject in need thereof a therapeutically effective amount of an ADAMTS13 variant of claim 1.

29. The method of claim 28, wherein the blood clotting disorder is inherited TTP, acquired TTP, infarction, cerebral infarction, myocardial infarction, ischemic/reperfusion injury, deep vein thrombosis, or sepsis-related disseminated intravascular coagulation.

30. A method for treating or preventing a bleeding episode in a subject in a subject suffering from or at risk of suffering from a bleeding disorder, comprising administering to the subject in need thereof a therapeutically effective amount of an ADAMTS13 variant of claim 1.

31. The method of claim 30, wherein the bleeding episode is associated with inherited TTP, acquired TTP, infarction, cerebral infarction, myocardial infarction, ischemic/reperfusion injury, deep vein thrombosis, or sepsis-related disseminated intravascular coagulation.

32. A method for treating or preventing a vaso-occlusive crisis in a subject suffering from sickle cell disease, comprising administering to the subject in need thereof a therapeutically effective amount of the ADAMTS13 variant of claim 1.

33. A method for treating or preventing lung injury in a subject suffering from or at risk of suffering from acute lung injury (ALI) and/or acute respiratory distress syndrome (ARDS), the method comprising administering to the subject in need thereof a therapeutically effective amount of the ADAMTS13 variant of claim 1.

34. A method for treating a cerebral infarction in a subject by recanalization of an occluded blood vessel in the subject, comprising administering to the subject in need thereof a therapeutically effective amount of the ADAMTS13 variant of claim 1, thereby recanalizing the occluded blood vessel.

35. A method for treating or preventing a blood clotting disorder associated with cardiovascular disease in a subject, comprising administering to the subject in need thereof a therapeutically effective amount of an ADAMTS13 variant of claim 1.

36. The method of claim 35, wherein the blood clotting disorder associated with cardiovascular disease is associated with myocardial infarction, myocardial ischemia, deep vein thrombosis, peripheral vascular disease, stroke, transient ischemic attack, or medical device associated thrombosis.

37. A method for treating or preventing hematologic disease in a subject, comprising administering to the subject in need thereof a therapeutically effective amount of an ADAMTS13 variant of claim 1.

38. (canceled)

39. The method of claim 28, wherein the subject is a mammal.

40. The method of claim 28, wherein the subject is a human.

41.-43. (canceled)

44. A nucleic acid molecule encoding the ADAMTS13 variant of claim 1.

45. A vector comprising the nucleic acid molecule of claim 44.

46. The vector of claim 45, wherein the vector is an expression vector wherein the polynucleotide sequence encoding the ADAMTS13 variant is operably linked to a promoter that is capable of mediating expression of the ADAMTS13 variant in a host cell.

47. A host cell comprising the nucleic acid molecule of claim 44.

48. A host cell comprising the vector of claim 45.

49. A host cell line comprising cells modified to express the ADAMTS13 variant of claim 1 and at least one ADAMTS13 protein.

50.-52. (canceled)

53. The host cell line of claim 49, wherein the ADAMTS13 variant and the ADAMTS13 protein are expressed in different cells in the host cell line.

54. The host cell line of claim 49, wherein the ADAMTS13 variant and the ADAMTS13 protein are expressed in the same cell.

55. The host cell of claim 47, wherein the cell is a CHO, COS, HEK 293, BHK, SK-Hep, or HepG2 cell.

56. The host cell or host cell line of claim 55, wherein the CHO cell is a CHO DBX-11 or CHOZN cell line.

57. (canceled)

58. The host cell or host cell line of claim 56, wherein the CHOZN cell is a CHOZN glutamine synthetase (GS)^{-/-} cell line.

59. A method for detecting an ADAMTS13 variant of claim 1, comprising:
a. subjecting a plasma sample to trypsin digestion to form a peptide mixture;
b. separating the peptide mixture by reversed phase chromatography; and
c. detecting the ADAMTS13 variant via detecting
AAGGILHLELLVAVGPDVFQAHR (SEQ ID NO: 115) or a combination of
AAGGILHLELLVAVGPDVFQAHR (SEQ ID NO: 115) and EDTER (SEQ ID NO: 5).

60. A method of determining the ratio or percentage of an ADAMTS13 variant of claim 1, comprising:
a. subjecting a plasma sample to trypsin digestion to form a peptide mixture;
b. separating the peptide mixture by reversed phase chromatography;
c. detecting AAGGILHLELLVAVGPDVFQAHR (SEQ ID NO: 115) or a combination of AAGGILHLELLVAVGPDVFQAHR (SEQ ID NO: 115) and EDTER (SEQ ID NO: 5) to determine ADAMTS13 variant intensities;
d. detecting presence of non-variant ADAMTS13 intensities; and
e. determining the ratio or percentage of the ADAMTS13 variant intensities as compared to non-variant ADAMTS13 intensities.


SEQUENCE LISTING (115 sequences)

SEQ ID NO: 1 - protein, 1427 aa, Homo sapiens
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 2 - protein, 1427 aa, Artificial Sequence
  source: /note=“Description of Artificial Sequence Synthetic polypeptide”
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHREDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 3 - protein, 1431 aa, Gorilla sp.
MHQRHPRARCPPLCVAGILACGFLLGCWGPSHFQQSCLQALEPQAVSSYLSPGAPLKGRPPSPGFQRQRQRQRQRQRRAAGGILHLELLVAVGPDVFQAHQEDTERYVLTNLNIGAELLRDPSLGAQFRVHLVKMVILTEPEGAPNITANLTSSLLSVCGWSRTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRRLLPCRSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPHSPCSRSCGGGVVTRRRQCNNPRPAFGGRACVGADLQAEMCNTQACEKTQLEFMSEQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVVLETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRQEVCQAVPCPARWRYKLAACSVSCGGGVVRRILYCARAHGEDDGEEILLDTQCQGLPRPEPQEACSLEPCPPRWKVMSLGPCSASCGLGTARRSVACVQLDQGQDVEVDEAACVALVRPQASVPCLIADCTYRWHVGTWMECSVSCGDGIQRRRDTCLGPQAQAPVPADFCQHLPKPVTVRGCWAGPCVGQGMPSLVPHEEAAAPGRTTATPAGASLEWSQARALLFSPAPQPQRLLPGPQENSAQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYISIRDTHSLRTTAFHGQQVLYWESESSQAEMEFSEGFLKAQASLRGQYWTLQSWVPEVQDPQSWKGKEGT

SEQ ID NO: 4 - DNA, 4284 bp, Homo sapiens
atgcaccagcgtcacccccgggcaagatgccctcccctctgtgtggccggaatccttgcctgtggctttctcctgggctgctggggaccctcccatttccagcagagctgtcttcaggctttggagccacaggccgtgtcttcttacttgagccctggtgctcccttaaaaggccgccctccttcccctggcttccagaggcagaggcagaggcagaggcgggctgcaggcggcatcctacacctggagctgctggtggccgtgggccccgatgtcttccaggctcaccaggaggacacagagcgctatgtgctcaccaacctcaacatcggggcagaactgcttcgggacccgtccctgggggctcagtttcgggtgcacctggtgaagatggtcattctgacagagcctgagggtgctccaaatatcacagccaacctcacctcgtccctgctgagcgtctgtgggtggagccagaccatcaaccctgaggacgacacggatcctggccatgctgacctggtcctctatatcactaggtttgacctggagttgcctgatggtaaccggcaggtgcggggcgtcacccagctgggcggtgcctgctccccaacctggagctgcctcattaccgaggacactggcttcgacctgggagtcaccattgcccatgagattgggcacagcttcggcctggagcacgacggcgcgcccggcagcggctgcggccccagcggacacgtgatggcttcggacggcgccgcgccccgcgccggcctcgcctggtccccctgcagccgccggcagctgctgagcctgctcagcgcaggacgggcgcgctgcgtgtgggacccgccgcggcctcaacccgggtccgcggggcacccgccggatgcgcagcctggcctctactacagcgccaacgagcagtgccgcgtggccttcggccccaaggctgtcgcctgcaccttcgccagggagcacctggatatgtgccaggccctctcctgccacacagacccgctggaccaaagcagctgcagccgcctcctcgttcctctcctggatgggacagaatgtggcgtggagaagtggtgctccaaaggtcgctgccgctccctggtggagctgacccccatagcagcagtgcatgggcgctggtctagctggggtccccgaagtccttgctcccgctcctgcggaggaggtgtggtcaccaggaggcggcagtgcaacaaccccagacctgcctttggggggcgtgcatgtgttggtgctgacctccaggccgagatgtgcaacactcaggcctgcgagaagacccagctggagttcatgtcgcaacagtgcgccaggaccgacggccagccgctgcgctcctcccctggcggcgcctccttctaccactggggtgctgctgtaccacacagccaaggggatgctctgtgcagacacatgtgccgggccattggcgagagcttcatcatgaagcgtggagacagcttcctcgatgggacccggtgtatgccaagtggcccccgggaggacgggaccctgagcctgtgtgtgtcgggcagctgcaggacatttggctgtgatggtaggatggactcccagcaggtatgggacaggtgccaggtgtgtggtggggacaacagcacgtgcagcccacggaagggctctttcacagctggcagagcgagagaatatgtcacgtttctgacagttacccccaacctgaccagtgtctacattgccaaccacaggcctctcttcacacacttggcggtgaggatcggagggcgctatgtcgtggctgggaagatgagcatctcccctaacaccacctacccctccctcctggaggatggtcgtgtcgagtacagagtggccctcaccgaggaccggctgccccgcctggaggagatccgcatctggggacccctccaggaagatgctgacatccaggtttacaggcggtatggcgaggagtatggcaacctcacccgcccagacatcaccttcacctacttccagcctaagccacggcaggcctgggtgtgggccgctgtgcgtgggccctgctcggtgagctgtggggcagggctgcgctgggtaaactacagctgcctggaccaggccaggaaggagttggtggagactgtccagtgccaagggagccagcagccaccagcgtggccagaggcctgcgtgctcgaaccctgccctccctactgggcggtgggagacttcggcccatgcagcgcctcctgtgggggtggcctgcgggagcggccagtgcgctgcgtggaggcccagggcagcctcctgaagacattgcccccagcccggtgcagagcaggggcccagcagccagctgtggcgctggaaacctgcaacccccagccctgccctgccaggtgggaggtgtcagagcccagctcatgcacatcagctggtggagcaggcctggccttggagaacgagacctgtgtgccaggggcagatggcctggaggctccagtgactgaggggcctggctccgtagatgagaagctgcctgcccctgagccctgtgtcgggatgtcatgtcctccaggctggggccatctggatgccacctctgcaggggagaaggctccctccccatggggcagcatcaggacgggggctcaagctgcacacgtgtggacccctgcggcagggtcgtgctccgtctcctgcgggcgaggtctgatggagctccgtttcctgtgcatggactctgccctcagggtgcctgtccaggaagagctgtgtggcctggcaagcaagcctgggagccggcgggaggtctgccaggctgtcccgtgccctgctcggtggcagtacaagctggcggcctgcagcgtgagctgtgggagaggggtcgtgcggaggatcctgtattgtgcccgggcccatggggaggacgatggtgaggagatcctgttggacacccagtgccaggggctgcctcgcccggaaccccaggaggcctgcagcctggagccctgcccacctaggtggaaagtcatgtcccttggcccatgttcggccagctgtggccttggcactgctagacgctcggtggcctgtgtgcagctcgaccaaggccaggacgtggaggtggacgaggcggcctgtgcggcgctggtgcggcccgaggccagtgtcccctgtctcattgccgactgcacctaccgctggcatgttggcacctggatggagtgctctgtttcctgtggggatggcatccagcgccggcgtgacacctgcctcggaccccaggcccaggcgcctgtgccagctgatttctgccagcacttgcccaagccggtgactgtgcgtggctgctgggctgggccctgtgtgggacagggtacgcccagcctggtgccccacgaagaagccgctgctccaggacggaccacagccacccctgctggtgcctccctggagtggtcccaggcccggggcctgctcttctccccggctccccagcctcggcggctcctgcccgggccccaggaaaactcagtgcagtccagtgcctgtggcaggcagcaccttgagccaacaggaaccattgacatgcgaggcccagggcaggcagactgtgcagtggccattgggcggcccctcggggaggtggtgaccctccgcgtccttgagagttctctcaactgcagtgcgggggacatgttgctgctttggggccggctcacctggaggaagatgtgcaggaagctgttggacatgactttcagctccaagaccaacacgctggtggtgaggcagcgctgcgggcggccaggaggtggggtgctgctgcggtatgggagccagcttgctcctgaaaccttctacagagaatgtgacatgcagctctttgggccctggggtgaaatcgtgagcccctcgctgagtccagccacgagtaatgcagggggctgccggctcttcattaatgtggctccgcacgcacggattgccatccatgccctggccaccaacatgggcgctgggaccgagggagccaatgccagctacatcttgatccgggacacccacagcttgaggaccacagcgttccatgggcagcaggtgctctactgggagtcagagagcagccaggctgagatggagttcagcgagggcttcctgaaggctcaggccagcctgcggggccagtactggaccctccaatcatgggtaccggagatgcaggaccctcagtcctggaagggaaaggaaggaacctga

SEQ ID NO: 5 - protein, 5 aa, Homo sapiens
EDTER

SEQ ID NO: 6 - protein, 28 aa, Homo sapiens
AAGGILHLELLVAVGPDVFQAHQEDTER

SEQ ID NO: 7 - protein, 28 aa, Artificial Sequence
  source: /note=“Description of Artificial Sequence Synthetic peptide”
AAGGILHLELLVAVGPDVFQAHREDTER

SEQ ID NO: 8 - protein, 14 aa, Homo sapiens
YVLTNLNIGAELLR

SEQ ID NO: 9 - protein, 9 aa, Homo sapiens
DPSLGAQFR

SEQ ID NO: 10 - protein, 5 aa, Homo sapiens
VHLVK

SEQ ID NO: 11 - protein, 50 aa, Homo sapiens
MVILTEPEGAPNITANLTSSLLSVCGWSQTINPEDDTDPGHADLVLYITR

SEQ ID NO: 12 - protein, 10 aa, Homo sapiens
FDLELPDGNR

SEQ ID NO: 13 - protein, 64 aa, Homo sapiens
GVTQLGGACSPTWSCLITEDTGFDLGVTIAHEIGHSFGLEHDGAPGSGCGPSGHVMASDGAAPR

SEQ ID NO: 14 - protein, 10 aa, Homo sapiens
AGLAWSPCSR

SEQ ID NO: 15 - protein, 11 aa, Homo sapiens
RQLLSLLSAGR

SEQ ID NO: 16 - protein, 10 aa, Homo sapiens
QLLSLLSAGR

SEQ ID NO: 17 - protein, 32 aa, Homo sapiens
CVWDPPRPQPGSAGHPPDAQPGLYYSANEQCR

SEQ ID NO: 18 - protein, 6 aa, Homo sapiens
VAFGPK

SEQ ID NO: 19 - protein, 8 aa, Homo sapiens
AVACTFAR

SEQ ID NO: 20 - protein, 23 aa, Homo sapiens
EHLDMCQALSCHTDPLDQSSCSR

SEQ ID NO: 21 - protein, 15 aa, Homo sapiens
LLVPLLDGTECGVEK

SEQ ID NO: 22 - protein, 14 aa, Homo sapiens
SLVELTPIAAVHGR

SEQ ID NO: 23 - protein, 7 aa, Homo sapiens
WSSWGPR

SEQ ID NO: 24 - protein, 5 aa, Homo sapiens
SPCSR

SEQ ID NO: 25 - protein, 9 aa, Homo sapiens
SCGGGVVTR

SEQ ID NO: 26 - protein, 7 aa, Homo sapiens
RQCNNPR

SEQ ID NO: 27 - protein, 6 aa, Homo sapiens
QCNNPR

SEQ ID NO: 28 - protein, 6 aa, Homo sapiens
PAFGGR

SEQ ID NO: 29 - protein, 19 aa, Homo sapiens
ACVGADLQAEMCNTQACEK

SEQ ID NO: 30 - protein, 12 aa, Homo sapiens
TQLEFMSQQCAR

SEQ ID NO: 31 - protein, 7 aa, Homo sapiens
TDGQPLR

SEQ ID NO: 32 - protein, 25 aa, Homo sapiens
SSPGGASFYHWGAAVPHSQGDALCR

SEQ ID NO: 33 - protein, 4 aa, Homo sapiens
HMCR

SEQ ID NO: 34 - protein, 9 aa, Homo sapiens
AIGESFIMK

SEQ ID NO: 35 - protein, 10 aa, Homo sapiens
RGDSFLDGTR

SEQ ID NO: 36 - protein, 9 aa, Homo sapiens
GDSFLDGTR

SEQ ID NO: 37 - protein, 7 aa, Homo sapiens
CMPSGPR

SEQ ID NO: 38 - protein, 14 aa, Homo sapiens
EDGTLSLCVSGSCR

SEQ ID NO: 39 - protein, 7 aa, Homo sapiens
TFGCDGR

SEQ ID NO: 40 - protein, 9 aa, Homo sapiens
MDSQQVWDR

SEQ ID NO: 41 - protein, 14 aa, Homo sapiens
CQVCGGDNSTCSPR

SEQ ID NO: 42 - protein, 8 aa, Homo sapiens
KGSFTAGR

SEQ ID NO: 43 - protein, 7 aa, Homo sapiens
GSFTAGR

SEQ ID NO: 44 - protein, 21 aa, Homo sapiens
EYVTFLTVTPNLTSVYIANHR

SEQ ID NO: 45 - protein, 9 aa, Homo sapiens
PLFTHLAVR

SEQ ID NO: 46 - protein, 4 aa, Homo sapiens
IGGR

SEQ ID NO: 47 - protein, 6 aa, Homo sapiens
YVVAGK

SEQ ID NO: 48 - protein, 17 aa, Homo sapiens
MSISPNTTYPSLLEDGR

SEQ ID NO: 49 - protein, 4 aa, Homo sapiens
VERY

SEQ ID NO: 50 - protein, 7 aa, Homo sapiens
VALTEDR

SEQ ID NO: 51 - protein, 5 aa, Homo sapiens
LEEIR

SEQ ID NO: 52 - protein, 15 aa, Homo sapiens
IWGPLQEDADIQVYR

SEQ ID NO: 53 - protein, 21 aa, Homo sapiens
YGEEYGNLTRPDITFTYFQPK

SEQ ID NO: 54 - protein, 11 aa, Homo sapiens
PDITFTYFQPK

SEQ ID NO: 55 - protein, 9 aa, Homo sapiens
QAWVWAAVR

SEQ ID NO: 56 - protein, 12 aa, Homo sapiens
GPCSVSCGAGLR

SEQ ID NO: 57 - protein, 11 aa, Homo sapiens
WVNYSCLDQAR

SEQ ID NO: 58 - protein, 48 aa, Homo sapiens
KELVETVQCQGSQQPPAWPEACVLEPCPPYWAVGDFGPCSASCGGGLR

SEQ ID NO: 59 - protein, 47 aa, Homo sapiens
ELVETVQCQGSQQPPAWPEACVLEPCPPYWAVGDFGPCSASCGGGLR

SEQ ID NO: 60 - protein, 5 aa, Homo sapiens
ERPVR

SEQ ID NO: 61 - protein, 10 aa, Homo sapiens
CVEAQGSLLK

SEQ ID NO: 62 - protein, 16 aa, Homo sapiens
CVEAQGSLLKTLPPAR

SEQ ID NO: 63 - protein, 6 aa, Homo sapiens
TLPPAR

SEQ ID NO: 64 - protein, 8 aa, Homo sapiens
TLPPARCR

SEQ ID NO: 65 - protein, 21 aa, Homo sapiens
AGAQQPAVALETCNPQPCPAR

SEQ ID NO: 66 - protein, 45 aa, Homo sapiens
WEVSEPSSCTSAGGAGLALENETCVPGADGLEAPVTEGPGSVDEK

SEQ ID NO: 67 - protein, 27 aa, Homo sapiens
LPAPEPCVGMSCPPGWGHLDATSAGEK

SEQ ID NO: 68 - protein, 27 aa, Homo sapiens
LPAPEPCVGMSCPPGWGHLDATSAGEC

SEQ ID NO: 69 - protein, 9 aa, Homo sapiens
APSPWGSIR

SEQ ID NO: 70 - protein, 22 aa, Homo sapiens
TGAQAAHVWTPAAGSCSVSCGR

SEQ ID NO: 71 - protein, 6 aa, Homo sapiens
GLMELR

SEQ ID NO: 72 - protein, 9 aa, Homo sapiens
FLCMDSALR

SEQ ID NO: 73 - protein, 13 aa, Homo sapiens
VPVQEELCGLASK

SEQ ID NO: 74 - protein, 4 aa, Homo sapiens
PGSR

SEQ ID NO: 75 - protein, 12 aa, Homo sapiens
REVCQAVPCPAR

SEQ ID NO: 76 - protein, 11 aa, Homo sapiens
EVCQAVPCPAR

SEQ ID NO: 77 - protein, 4 aa, Homo sapiens
WQYK

SEQ ID NO: 78 - protein, 10 aa, Homo sapiens
LAACSVSCGR

SEQ ID NO: 79 - protein, 4 aa, Homo sapiens
GVVR

SEQ ID NO: 80 - protein, 6 aa, Homo sapiens
ILYCAR

SEQ ID NO: 81 - protein, 21 aa, Homo sapiens
AHGEDDGEEILLDTQCQGLPR

SEQ ID NO: 82 - protein, 36 aa, Homo sapiens
AHGEDDGEEILLDTQCQGLPRPEPQEACSLEPCPPR

SEQ ID NO: 83 - protein, 15 aa, Homo sapiens
PEPQEACSLEPCPPR

SEQ ID NO: 84 - protein, 17 aa, Homo sapiens
VMSLGPCSASCGLGTAR

SEQ ID NO: 85 - protein, 40 aa, Homo sapiens
SVACVQLDQGQDVEVDEAACAALVRPEASVPCLIADCTYR

SEQ ID NO: 86 - protein, 15 aa, Homo sapiens
PEASVPCLIADCTYR

SEQ ID NO: 87 - protein, 19 aa, Homo sapiens
WHVGTWMECSVSCGDGIQR

SEQ ID NO: 88 - protein, 23 aa, Homo sapiens
RDTCLGPQAQAPVPADFCQHLPK

SEQ ID NO: 89 - protein, 22 aa, Homo sapiens
DTCLGPQAQAPVPADFCQHLPK

SEQ ID NO: 90 - protein, 27 aa, Homo sapiens
DTCLGPQAQAPVPADFCQHLPKPVTVR

SEQ ID NO: 91 - protein, 5 aa, Homo sapiens
PVTVR

SEQ ID NO: 92 - protein, 42 aa, Homo sapiens
GCWAGPCVGQGTPSLVPHEEAAAPGRTTATPAGASLEWSQAR

SEQ ID NO: 93 - protein, 16 aa, Homo sapiens
TTATPAGASLEWSQAR

SEQ ID NO: 94 - protein, 11 aa, Homo sapiens
GLLFSPAPQPR

SEQ ID NO: 95 - protein, 17 aa, Homo sapiens
LLPGPQENSVQSSACGR

SEQ ID NO: 96 - protein, 12 aa, Homo sapiens
QHLEPTGTIDMR

SEQ ID NO: 97 - protein, 13 aa, Homo sapiens
GPGQADCAVAIGR

SEQ ID NO: 98 - protein, 9 aa, Homo sapiens
PLGEVVTLR

SEQ ID NO: 99 - protein, 19 aa, Homo sapiens
VLESSLNCSAGDMLLLWGR

SEQ ID NO: 100 - protein, 4 aa, Homo sapiens
LTWR

SEQ ID NO: 101 - protein, 10 aa, Homo sapiens
KLLDMTFSSK

SEQ ID NO: 102 - protein, 9 aa, Homo sapiens
LLDMTFSSK

SEQ ID NO: 103 - protein, 7 aa, Homo sapiens
TNTLVVR

SEQ ID NO: 104 - protein, 11 aa, Homo sapiens
CGRPGGGVLLR

SEQ ID NO: 105 - protein, 8 aa, Homo sapiens
PGGGVLLR

SEQ ID NO: 106 - protein, 12 aa, Homo sapiens
YGSQLAPETFYR

SEQ ID NO: 107 - protein, 29 aa, Homo sapiens
ECDMQLFGPWGEIVSPSLSPATSNAGGCR

SEQ ID NO: 108 - protein, 10 aa, Homo sapiens
LFINVAPHAR

SEQ ID NO: 109 - protein, 25 aa, Homo sapiens
IAIHALATNMGAGTEGANASYILIR

SEQ ID NO: 110 - protein, 6 aa, Homo sapiens
DTHSLR

SEQ ID NO: 111 - protein, 29 aa, Homo sapiens
TTAFHGQQVLYWESESSQAEMEFSEGFLK

SEQ ID NO: 112 - protein, 6 aa, Homo sapiens
AQASLR

SEQ ID NO: 113 - protein, 20 aa, Homo sapiens
GQYWTLQSWVPEMQDPQSWK

SEQ ID NO: 114 - protein, 4 aa, Homo sapiens
WCSK

SEQ ID NO: 115 - protein, 23 aa, Artificial Sequence
  source: /note=“Description of Artificial Sequence Synthetic peptide”
AAGGILHLELLVAVGPDVFQAHR